SEQUENCE LISTING (94 sequences)

SEQ ID NO: 1 - protein, 128 aa, Pseudomonas aeruginosa
AECSVDIQGNDQMQFNTNAITVDKSCKQFTVNLSHPGNLPKNVMGHNWVLSTAADMQGVVTDGMASGLDKDYLKPDDSRVIAHTKLIGSGEKDSVTFDVSKLKEGEQYMFFCTFPGHSALMKGTLTLK

SEQ ID NO: 2 - protein, 105 aa, Phormidium laminosum
ETFTVKMGADSGLLQFEPANVTVHPGDTVKWVNNKLPPHNILFDDKQVPGASKELADKLSHSQLMFSPGESYEITFSSDFPAGTYTYYCAPHRGAGMVGKITVEG

SEQ ID NO: 3 - protein, 155 aa, Thiobacillus ferrooxidans
GTLDTTWKEATLPQVKAMLEKDTGKVSGDTVTYSGKTVHVVAAAVLPGFPFPSFEVHDKKNPTLEIPAGATVDVTFINTNKGFGHSFDITKKGPPYAVMPVIDPIVAGTGFSPVPKDGKFGYTDFTWHPTAGTYYYVCQIPGHAATGMFGKIVVK

SEQ ID NO: 4 - protein, 124 aa, Achromobacter cycloclastes
ADFEVHMLNKGKDGAMVFEPASLKVAPGDTVTFIPTDKGHNVETIKGMIPDGAEAFKSKINENYKVTFTAPGVYGVKCTPHYGMGMVGVVQVGDAPANLEAVKGAKNPKKAQERLDAALAALGN

SEQ ID NO: 5 - protein, 128 aa, Alcaligenes faecalis
ACDVSIEGNDSMQFNTKSIVVDKTCKEFTINLKHTGKLPKAAMGHNVVVSKKSDESAVATDGMKAGLNNDYVKAGDERVIAHTSVIGGGETDSVTFDVSKLKEGEDYAFFCSFPGHWSIMKGTIELGS

SEQ ID NO: 6 - protein, 129 aa, Achromobacter xylosoxidans
AQCEATIESNDAMQYNLKEMVVDKSCKQFTVHLKHVGKMAKVAMGHNWVLTKEADKQGVATDGMNAGLAQDYVKAGDTRVIAHTKVIGGGESDSVTFDVSKLTPGEAYAYFCSFPGHWAMMKGTLKLSN

SEQ ID NO: 7 - protein, 129 aa, Bordetella bronchiseptica
AECSVDIAGTDQMQFDKKAIEVSKSCKQFTVNLKHTGKLPRNVMGHNWVLTKTADMQAVEKDGIAAGLDNQYLKAGDTRVLAHTKVLGGGESDSVTFDVAKLAAGDDYTFFCSFPGHGALMKGTLKLVD

SEQ ID NO: 8 - protein, 129 aa, Methylomonas sp.
ASCETTVTSGDTMTYSTRSISVPASCAEFTVNFEHKGHMPKTGMGHNWVLAKSADVGDVAKEGAHAGADNNFVTPGDKRVIAFTPIIGGGEKTSVKFKVSALSKDEAYTYFCSYPGHFSMMRGTLKLEE

SEQ ID NO: 9 - protein, 166 aa, Neisseria meningitides
CSQEPAAPAAEATPAAEAPASEAPAAEAAPADAAEAPAAGNCAATVESNDNMQFNTKDIQVSKACKEFTITLKHTGTQPKTSMGHNIVIGKTEDMDGIFKDGVGAADTDYVKPDDARVVAHTKLIGGGEESSLTLDPAKLADGEYKFACTFPGHGALMNGKVTLVD

SEQ ID NO: 10 - protein, 128 aa, Pseudomonas fluorescen
AECKTTIDSTDQMSFNTKAIEIDKACKTFTVELTHSGSLPKNVMGHNLVISKQADMQPIATDGLSAGIDKNYLKEGDTRVIAHTKVIGAGEKDSLTIDVSKLNAAEKYGFFCSFPGHISMMKGTVTLK

SEQ ID NO: 11 - protein, 136 aa, Pseudomonas syringae
MASGQLLAAECSATVDSTDQMMYDTKAIQIDKSCKEFTLNLTHSGSLPKNVMGHNWVLSKKADASAITTDGMSVGIDKDYVKPDDTRVIAHTKIIGAGENDSVTFDVSKLDPAEDYQFFCTFPGHISMMKGAVTLK

SEQ ID NO: 12 - protein, 129 aa, Xylella fastidiosa
KTCAVTISANDQMKFDQNTIKIAAECTHVNLTLTHTGKKSARVMGHNWVLTKTTDMQAVALAGLHATLADNYVPKADPRVIAHTAIIGGGERTSITFPTNTLSKNVSYTFFCSFPGHWALMKGTLNFGG

SEQ ID NO: 13 - protein, 138 aa, Cucumis sativus
MQSTVHIVGDNTGWSVPSSPNFYSQWAAGKTFRVGDSLQFNFPANAHNVHEMETKQSFDACNFVNSDNDVERTSPVIERLDELGMHYFVCTVGTHCSNGQKLSINVVAANATVSMPPPSSSPPSSVMPPPVMPPPSPS

SEQ ID NO: 14 - protein, 162 aa, Chloroflexus aurantiacus
MKITLRMMVLAVLTAMAMVLAACGGGGSSGGSTGGGSGSGPVTIEIGSKGEELAFDKTELTVSAGQTVTIRFKNNSAVQQHNWILVKGGEAEAANIANAGLSAGPAANYLPADKSNIIAESPLANGNETVEVTFTAPAAGTYLYICTVPGHYPLMQGKLVVN

SEQ ID NO: 15 - protein, 140 aa, Chloroflexus aurantiacus
AANAPGGSNVVNETPAQTVEVRAAPDALAFAQTSLSLPANTVVRLDFVNQNNLGVQHNWVLVNGGDDVAAAVNTAAQNNADALFVPPPDTPNALAWTAMLNAGESGSVTFRTPAPGTYLYICTFPGHYLAGMKGTLTVTP

SEQ ID NO: 16 - protein, 96 aa, Cucumis sativus
AVYVVGGSGGWTFNTESWPKGKRFRAGDILLFNYNPSMHNVVVVNQGGFSTCNTPAGAKVYTSGRDQIKLPKGQSYFICNFPGHCQSGMKIAVNAL

SEQ ID NO: 17 - protein, 166 aa, Neisseria gonorrhoeae
CSQEPAAPAAEATPAGEAPASEAPAAEAAPADAAEAPAAGNCAATVESNDNMQFNTKDIQVSKACKEFTITLKHTGTQPKASMGHNLVIAKAEDMDGVFKDGVGAADTDYVKPDDARVVAHTKLIGGGEESSLTLDPAKLADGDYKFACTFPGHGALMNGKVTLVD

SEQ ID NO: 18 - protein, 150 aa, Vibrio parahaemolyticus
MSLRILAATLALAGLSFGAQASAECEVSIDANDMMQFSTKTLSVPATCKEVTLTLNHTGKMPAQSMGHNVVIADTANIQAVGTDGMSAGADNSYVKPDDERVYAHTKVVGGGESTSITFSTEKMTAGGDYSFFCSFPGHWAIMQGKFEFK

SEQ ID NO: 19 - protein, 104 aa, Homo sapiens
GDVEKGKKIFIMKCSQCHTVEKGGKHKTGPNLHGLFGRKTGQAPGYSYTAANKNKGIIWGEDTLMEYLENPKKYIPGTKMIFVGIKKKEERADLIAYLKKATNE

SEQ ID NO: 20 - protein, 338 aa, Phormidium laminosum
MNFKVCSFPSRRQSIAAFVRVLMVILLTLGALVSSDVLLPQPAAAYPFWAQQNYANPREATGRIVCANCHLAAKPAEIEVPQAVLPDSVFKAVVKIPYDHSVQQVQADGSKGPLNVGAVLMLPEGFTIAPEDRIPEEMKEEVGPSYLFQPYADDKQNIVLVGPLPGDQYEEIVFPVLSPNPATNKSVAFGKYSIHLGANRGRGQIYPTGEKSNNAVYNASAAGVITAIAKADDGSAEVKIRTEDGTTIVDKIPAGPELIVSEGEEVAAGAALTNNPNVGGFGQKDTEIVLQSPNRVKGRIAFLAAITLTQILLVLKKKQVERVQAGRDDLLKAAFIAG

SEQ ID NO: 21 - protein, 82 aa, Pseudomonas aeruginosa
EDPEVLFKNKGCVACHAIDTKMVGPAYKDVAAKFAGQAGAEAELAQRIKNGSQGVWGPIPMPPNAVSDDEAQTLAKWVLSQK

SEQ ID NO: 22 - protein, 39 aa, Neisseria gonorrhoeae
CSQEPAAPAAEATPAGEAPASEAPAAEAAPADAAEAPAA

SEQ ID NO: 23 - protein, 129 aa, Bordetella pertussis; identical to SEQ ID NO: 7

SEQ ID NO: 24 - protein, 33 aa, Chloroflexus aurantiacus
HNWVLVNGGDDVAAAVNTAAQNNADALFVPPPD

SEQ ID NO: 25 - protein, 27 aa, Pseudomonas syringae
SKKADASAITTDGMSVGIDKDYVKPDD

SEQ ID NO: 26 - protein, 27 aa, Neisseria meningitides
IGKTEDMDGIFKDGVGAADTDYVKPDD

SEQ ID NO: 27 - protein, 27 aa, Vibrio parahaemolyticus
ADTANIQAVGTDGMSAGADNSYVKPDD

SEQ ID NO: 28 - protein, 27 aa, Bordetella bronchiseptica
TKTADMQAVEKDGIAAGLDNQYLKAGD

SEQ ID NO: 29 - protein, 28 aa, Pseudomonas aeruginosa
LSTAADMQGVVTDGMASGLDKDYLKPDD

SEQ ID NO: 30 - protein, 18 aa, Pseudomonas aeruginosa
LSTAADMQGVVTDGMASG

SEQ ID NO: 31 - protein, 93 aa, Pseudomonas aeruginosa
PGNLPKNVMGHNWVLSTAADMQGVVTDGMASGLDKDYLKPDDSRVIAHTKLIGSGEKDSVTFDVSKLKEGEQYMFFCTFPGHSALMKGTLTLK

SEQ ID NO: 32 - protein, 54 aa, Pseudomonas aeruginosa
PGNLPKNVMGHNWVLSTAADMQGVVTDGMASGLDKDYLKPDDSRVIAHTKLIGS

SEQ ID NO: 33 - protein, 42 aa, Pseudomonas aeruginosa
PGNLPKNVMGHNWVLSTAADMQGVVTDGMASGLDKDYLKPDD

SEQ ID NO: 34 - DNA, 33 bp, Artificial Sequence
  Description of Artificial Sequence Synthetic primer
ccggaattccggcagggatgttgtaaatatccg

SEQ ID NO: 35 - DNA, 38 bp, Artificial Sequence
  Description of Artificial Sequence Synthetic primer
ggggtaccgccgtggcaggcatacagcatttcaatcgg

SEQ ID NO: 36 - DNA, 27 bp, Artificial Sequence
  Description of Artificial Sequence Synthetic primer
ggcagcaggggcttcggcagcatctgc

SEQ ID NO: 37 - DNA, 26 bp, Artificial Sequence
  Description of Artificial Sequence Synthetic primer
ctgcaggtcgactctagaggatcccg

SEQ ID NO: 38 - DNA, 25 bp, Artificial Sequence
  Description of Artificial Sequence Synthetic primer
gccgagtgctcggtggacatccagg

SEQ ID NO: 39 - DNA, 27 bp, Artificial Sequence
  Description of Artificial Sequence Synthetic primer
tactcgagtcacttcagggtcagggtg

SEQ ID NO: 40 - DNA, 37 bp, Artificial Sequence
  Description of Artificial Sequence Synthetic primer
cgggatccccggcaacctgccgaagaacgtcatgggc

SEQ ID NO: 41 - DNA, 28 bp, Artificial Sequence
  Description of Artificial Sequence Synthetic primer
cggaattcgcatcacttcagggtcaggg

SEQ ID NO: 42 - DNA, 38 bp, Artificial Sequence
  Description of Artificial Sequence Synthetic primer
ccaagctgatcggctcgtgagagaaggactcggtgacc

SEQ ID NO: 43 - DNA, 38 bp, Artificial Sequence
  Description of Artificial Sequence Synthetic primer
ggtcaccgagtccttctctcacgagccgatcagcttgg

SEQ ID NO: 44 - DNA, 29 bp, Artificial Sequence
  Description of Artificial Sequence Synthetic primer
cggggatccccggctcgggcgagaaggac

SEQ ID NO: 45 - DNA, 29 bp, Artificial Sequence
  Description of Artificial Sequence Synthetic primer
cgggaattctccacttcagggtcagggtg

SEQ ID NO: 46 - DNA, 29 bp, Artificial Sequence
  Description of Artificial Sequence Synthetic oligonucleotide
gttcttctgcacctagccgggccactccg

SEQ ID NO: 47 - DNA, 29 bp, Artificial Sequence
  Description of Artificial Sequence Synthetic oligonucleotide
cggagtggcccggctaggtgcagaagaac

SEQ ID NO: 48 - protein, 18 aa, Pseudomonas aeruginosa
TFDVSKLKEGEQYMFFCT

SEQ ID NO: 49 - protein, 26 aa, Pseudomonas aeruginosa
GSGEKDSVTFDVSKLKEGEQYMFFCT

SEQ ID NO: 50 - protein, 53 aa, Pseudomonas aeruginosa
PGNLPKNVMGHNWVLSTAADMQGVVTDGMASGLDKDYLKPDDSRVIAHTKLIG

SEQ ID NO: 51 - protein, 28 aa, Artificial Sequence
  Description of Artificial Sequence Synthetic peptide
LSTAADMQAVVTDTMASGLDKDYLKPDD

SEQ ID NO: 52 - protein, 28 aa, Artificial Sequence
  Description of Artificial Sequence Synthetic peptide
LSTAADLQGVVTDGLASGLDKDYLKPDD

SEQ ID NO: 53 - protein, 28 aa, Artificial Sequence
  Description of Artificial Sequence Synthetic peptide
LSTAADVQGVVTDGVASGLDKDYLKPDD

SEQ ID NO: 54 - protein, 28 aa, Artificial Sequence
  Description of Artificial Sequence Synthetic peptide
DDPKLYDKDLGSAMGDTVVGQMDAATSL

SEQ ID NO: 55 - protein, 28 aa, Artificial Sequence
  Description of Artificial Sequence Synthetic peptide
LSTAADMQGVVTDGMASGLDKDYLKPDD

SEQ ID NO: 56 - protein, 6 aa, Artificial Sequence
  Description of Artificial Sequence Synthetic peptide
VSPPAR

SEQ ID NO: 57 - protein, 6 aa, Artificial Sequence
  Description of Artificial Sequence Synthetic peptide
YTPPAL

SEQ ID NO: 58 - protein, 6 aa, Artificial Sequence
  Description of Artificial Sequence Synthetic peptide
FSFFAF

SEQ ID NO: 59 - protein, 28 aa, Artificial Sequence
  Description of Artificial Sequence Synthetic peptide
LSTAADMQGVVTDGMASGLDKDYLTPGC

SEQ ID NO: 60 - protein, 28 aa, Artificial Sequence
  Description of Artificial Sequence Synthetic peptide
LSTAADCQGVVTDGMASGLDKDYLKPDD

SEQ ID NO: 61 - protein, 28 aa, Artificial Sequence
  Description of Artificial Sequence Synthetic peptide
LSTAACMQGVVTDGMASGLDKDYLKPDD

SEQ ID NO: 62 - protein, 28 aa, Artificial Sequence
  Description of Artificial Sequence Synthetic peptide
LSTACDMQGVVTDGMASGLDKDYLKPDD

SEQ ID NO: 63 - protein, 28 aa, Artificial Sequence
  Description of Artificial Sequence Synthetic peptide
LSTAATMQCVVTDGMASGLDKDYLKPDD

SEQ ID NO: 64 - protein, 28 aa, Artificial Sequence
  Description of Artificial Sequence Synthetic peptide
LSTAATMQGCVTDGMASGLDKDYLKPDD

SEQ ID NO: 65 - protein, 28 aa, Artificial Sequence
  Description of Artificial Sequence Synthetic peptide
LSTAANTQGCVTDGMASGLDKDYLKPDD

SEQ ID NO: 66 - protein, 28 aa, Artificial Sequence
  Description of Artificial Sequence Synthetic peptide
LSTAANTQGVCTDGMASGLDKDYLKPDD

SEQ ID NO: 67 - protein, 28 aa, Artificial Sequence
  Description of Artificial Sequence Synthetic peptide
LSTAADMTAVCTDGMASGLDKDYLKPDD

SEQ ID NO: 68 - protein, 28 aa, Artificial Sequence
  Description of Artificial Sequence Synthetic peptide
LSTAADMTAVVCDGMASGLDKDYLKPDD

SEQ ID NO: 69 - protein, 28 aa, Artificial Sequence
  Description of Artificial Sequence Synthetic peptide
LSTAADMQTVVCDGMASGLDKDYLKPDD

SEQ ID NO: 70 - protein, 28 aa, Artificial Sequence
  Description of Artificial Sequence Synthetic peptide
LSTAADMQTVVTCGMASGLDKDYLKPDD

SEQ ID NO: 71 - protein, 28 aa, Artificial Sequence
  Description of Artificial Sequence Synthetic peptide
LSTAADMQATVTCGMASGLDKDYLKPDD

SEQ ID NO: 72 - protein, 28 aa, Artificial Sequence
  Description of Artificial Sequence Synthetic peptide
LSTAADMQATVTDCMASGLDKDYLKPDD

SEQ ID NO: 73 - protein, 28 aa, Artificial Sequence
  Description of Artificial Sequence Synthetic peptide
LSTAADMQGVTADCMASGLDKDYLKPDD

SEQ ID NO: 74 - protein, 28 aa, Artificial Sequence
  Description of Artificial Sequence Synthetic peptide
LSTAADMQGVTADGCASGLDKDYLKPDD

SEQ ID NO: 75 - protein, 28 aa, Artificial Sequence
  Description of Artificial Sequence Synthetic peptide
LSTAADMQGVVTNGCASGLDKDYLKPDD

SEQ ID NO: 76 - protein, 28 aa, Artificial Sequence
  Description of Artificial Sequence Synthetic peptide
LSTAADMQGVVTATMGSGLCKDYLKPDD

SEQ ID NO: 77 - protein, 28 aa, Artificial Sequence
  Description of Artificial Sequence Synthetic peptide
LSTAADMQGVVTDLTASGLCKDYLKPDD

SEQ ID NO: 78 - protein, 28 aa, Artificial Sequence
  Description of Artificial Sequence Synthetic peptide
LSWAADMQGVVTDGMASGLDKDYLKPDD

SEQ ID NO: 79 - protein, 28 aa, Artificial Sequence
  Description of Artificial Sequence Synthetic peptide
LSTAADMWGVVTDGMASGLDKDYLKPDD

SEQ ID NO: 80 - protein, 28 aa, Artificial Sequence
  Description of Artificial Sequence Synthetic peptide
LSTAADMQGVVWDGMASGLDKDYLKPDD

SEQ ID NO: 81 - protein, 28 aa, Artificial Sequence
  Description of Artificial Sequence Synthetic peptide
LSTAADMQGVVTDWMASGLDKDYLKPDD

SEQ ID NO: 82 - protein, 28 aa, Artificial Sequence
  Description of Artificial Sequence Synthetic peptide
LSWAADMWGVVTDGMASGLDKDYLKPDD

SEQ ID NO: 83 - protein, 28 aa, Artificial Sequence
  Description of Artificial Sequence Synthetic peptide
LSWAADMQGVVWDGMASGLDKDYLKPDD

SEQ ID NO: 84 - protein, 28 aa, Artificial Sequence
  Description of Artificial Sequence Synthetic peptide
LSWAADMQGVVTDWMASGLDKDYLKPDD

SEQ ID NO: 85 - protein, 28 aa, Artificial Sequence
  Description of Artificial Sequence Synthetic peptide
LSTAADMWGVVWDGMASGLDKDYLKPDD

SEQ ID NO: 86 - protein, 28 aa, Artificial Sequence
  Description of Artificial Sequence Synthetic peptide
LSTAADMWGVVTDWMASGLDKDYLKPDD

SEQ ID NO: 87 - protein, 28 aa, Artificial Sequence
  Description of Artificial Sequence Synthetic peptide
LSTAADMQGVVWDWMASGLDKDYLKPDD

SEQ ID NO: 88 - protein, 28 aa, Artificial Sequence
  Description of Artificial Sequence Synthetic peptide
LSWAADMWGVVWDWMASGLDKDYLKPDD

SEQ ID NO: 89 - protein, 28 aa, Artificial Sequence
  Description of Artificial Sequence Synthetic peptide
LSXAADXXXVVXDXXASGLDKDYLKPDD

SEQ ID NO: 90 - protein, 28 aa, Artificial Sequence
  Description of Artificial Sequence Synthetic peptide
DDPKLYDKDLGSAXXDXVVXXXDAAXSL

SEQ ID NO: 91 - protein, 18 aa, Pseudomonas aeruginosa
VTDGMASGLDKDYLKPDD

SEQ ID NO: 92 - protein, 10 aa, Pseudomonas aeruginosa
LDKDYLKPDD

SEQ ID NO: 93 - protein, 12 aa, Pseudomonas aeruginosa
SGLDKDYLKPDD

SEQ ID NO: 94 - protein, 8 aa, Artificial Sequence
  Description of Artificial Sequence Synthetic peptide
RRRRRRRR

(19) United States
(12) Patent Application Publication
(10) Pub. No.: US 2009/0208476 A1
(43) Pub. Date: Aug. 20, 2009

(54) Compositions and methods to concurrently treat or prevent multiple diseases with cupredoxins

(76) Inventors: Tapas Das Gupta, River Forest, IL (US); Ananda Chakrabarty, Villa Park, IL (US)

Correspondence Address: DON J. PELTO;Sheppard, Mullin, Richter & Hampton LLP, 1300 I STREET, NW, 11TH FLOOR EAST, WASHINGTON, DC, 20005, US

(21) Appl. No.: 12/314,703

(22) Filed: Dec. 15, 2008

Related U.S. Application Data
(63) Continuation-in-part of application No. 11/943,034, filed on Nov. 20, 2007, now Pat. No. 7,511,117.
Application No. 11/943,034 is a division of application No. 11/436,591, filed on May 19, 2006, now Pat. No. 7,301,010.
Application No. 11/436,591 is a continuation-in-part of application No. 11/861,536, filed on Sep. 26, 2007.
Application No. 11/861,536 is a division of application No. 11/436,590, filed on May 19, 2006, now Pat. No. 7,338,766.
Application No. 11/436,590 is a continuation-in-part of application No. 11/488,693, filed on Jul. 19, 2006, now Pat. No. 7,556,810.
(60) Provisional application No. 61/013,709, filed on Dec. 14, 2007.

(51) Int. Cl. A61K 38/44; C12N 9/02; C07K 14/00; A61K 38/16; A61P 35/00; A61P 31/18; A61P 33/06

(52) U.S. Cl. 424/94.4; 435/191; 530/350; 514/12

(57) ABSTRACT

The present invention relates to compositions and methods to administer compositions comprising cupredoxin and/or cytochrome and/or variants, derivatives, truncations and structural equivalents of cupredoxin and cytochrome to treat and/or prevent two or more conditions in a mammalian cell. The invention also relates to compositions and methods to administer compositions comprising cupredoxin and/or cytochrome and/or variants, derivatives, truncations and structural equivalents of cupredoxin and cytochrome to concurrently treat and/or prevent two or more conditions in a patient such as HIV, cancer, malaria and inappropriate angiogenesis.

### CROSS REFERENCE TO RELATED APPLICATIONS

This application claims priority under 35 U.S.C. §§ 119 and 120 to U.S. Provisional Patent Application Ser. No. 61/013,709, filed on Dec. 14, 2007, and is a continuation in part of U.S. patent application Ser. No. 11/943,034, filed on Nov. 20, 2007, which is a divisional of U.S. application Ser. No. 11/436,591, filed on May 19, 2006, which issued as U.S. Pat. No. 7,301,010, and is also a continuation in part of U.S. application Ser. No. 11/861,536, filed on Sep. 26, 2007, which is a divisional of U.S. patent application Ser. No. 11/436,590, filed on May 19, 2006, which issued as U.S. Pat. No. 7,338,766, and is also a continuation in part of U.S. application Ser. No. 11/488,693, filed on Jul. 19, 2006, and is also a continuation in part of U.S. application Ser. No. 12/013,122, filed on Jan. 11, 2008, which is a continuation in part of U.S. application Ser. No. 11/436,592, filed on May 19, 2006, which issued as U.S. Pat. No. 7,381,701. Each of these documents is hereby incorporated by reference in its entirety herein.

### STATEMENT OF GOVERNMENTAL INTEREST

The subject matter of this application has been supported by the RAID program of the National Cancer Institute, Rockville, Md., U.S.A., (Grant Number NSC-745104). The government may have certain rights in this invention.

This application contains one (1) disc containing a sequence listing. The materials recorded in the compact disc are incorporated herein by reference in their entirety. The compact disc contains a single file named “PR144618.txt” (53KB, created on Feb. 9, 2009). The compact disc was created on Feb. 9, 2009.

### FIELD OF THE INVENTION

The present invention relates to compositions comprising variants, derivatives and structural equivalents of cupredoxins that concurrently treat and/or prevent two or more conditions in a patient.

### BACKGROUND OF THE INVENTION

Human immunodeficiency virus (HIV) infection, which results in AIDS, is a relatively new infection in the human population, and has quickly risen to the foremost health problem in the world. HIV/AIDS is now the leading cause of death in sub-Saharan Africa, and is the fourth biggest killer worldwide. At the end of 2001, it was estimated that 40 million people were living with HIV infection world wide. The Centers for Disease Control (CDC) estimates that nearly 800,000 people are living with AIDS in the US, and 40,000 new cases diagnosed each year. While better treatment methods are now known to prolong the life of patients with HIV infection, there is still no cure.

Modern anti-HIV drugs target several different stages of the HIV life cycle, and several of the enzymes that HIV requires to replicate and survive. Some of the commonly used anti-HIV drugs include nucleoside reverse transcriptase inhibitors such as AZT, ddI, ddC, d4T, 3TC, and abacavir; nucleotide reverse transcriptase inhibitors such as tenofovir; non-nucleoside reverse transcriptase inhibitors such as nevirapine, efavirenz and delavirdine; protease inhibitors such as saquinavir, ritonavir, indinavir, nelfinavir, amprinavir, lopinavir and atazanavir; and fusion inhibitors such as enfuvirtide. However, in some HIV infected patients, none of these antiviral drugs, alone or in combination, is effective to prevent the progression of chronic infection or treat acute AIDS. The high mutation rate of the HIV virus and associated emergence of HIV strains resistant to drugs may be one large factor that results in the inability to effectively treat HIV infection.

About one quarter of the world's population is exposed to the risk of malaria and more than a million people die of malaria each year. Of the four species of malarial parasites that infect humans, the two major species are Plasmodium falciparum and P. vivax.

The P. falciparum blood stage merozoites bind to and parasitize the erythrocytes using a variety of surface proteins (Cowman et al., FEBS Lett. 476:84-88 (2000); Baum et al., J. Biol. Chem. 281:5197-5208 (2006)), a major antigenic member of which is called Merozoite Surface Protein 1 (MSP1), a 195 kDa protein. MSP1 is present in all the erythrocyte-invasive species of Plasmodium, anchored to the merozoite surface by a glycosyl-phosphatidylinositol linkage. During early stages of the erythrocyte invasion process, soon after release from infected erythrocytes, the merozoite MSP1 protein undergoes proteolytic cleavage, producing a C-terminal cleavage product MSP1-42, which subsequently undergoes a second cleavage, producing an 11 kDa peptide MSP1-19, which remains attached to the parasite surface as it enters the erythrocyte. The formation of the cleavage product MSP1-19 is very important for successful invasion by the parasite since inhibition of its proteolytic formation or its neutralization by monoclonal antibodies prevents entry of the parasite to the erythrocytes (Blackman et al., J. Exptl., Med. 180:389-393 (1994)).

The MSP1-19 peptide is one of the most important malaria vaccine candidates available. MSP1-19-specific antibodies from malaria-resistant human sera react with the antigen and include a major erythrocyte-invasion inhibitory component (Holder & Riley, Parasitol. Today, 12: 173-174 (1996); O'Donnell et al., J. Expt. Med. 193: 1403-1412 (2001)). Serum from donors in malaria-endemic regions usually demonstrates strong antibody reactivity towards Pf MSP1-19. (Nwuba et al., Infect. Immun. 70: 5328-5331 (2002))

The monoclonal antibody (mAb) G17.12 was raised against recombinant Pf MSP1-19 and recognizes its epitope on the parasite surface, demonstrating that this region of the antigen is accessible on the native MSP1 polypeptide complex (Pizarro et al., J. Mol. Biol. 328:1091-1103 (2003)). Interestingly, erythrocyte invasion experiments in vitro showed that infection is not inhibited in the presence of G17.12, even at 200 μg/ml concentration and G17.12 does not inhibit in vitro secondary processing of MSP1. Id. The presence of antibodies that block the binding of invasion-inhibitory antibodies, thereby facilitating parasite survival, has also been demonstrated (Guevara Patino et al., J. Expt. Med. 186: 1689-1699 (1997)), and may be responsible for the failure of G17.12 mAb to inhibit erythrocyte invasion by M. falciparum.

Cerebral malaria, a rare but fatal infection restricted to P. falciparum invasion of brain capillaries because of the sequestration of parasitized erythrocytes, is often untreatable because most drugs cannot cross the blood-brain barrier to reach the brain capillaries. Adhesion of P. falciparum-infected erythrocytes to brain capillaries is mediated by the interaction of parasite ligands Pf Emp-1 family of proteins expressed on the surface of infected erythrocytes with ICAM-1 and CD36 expressed on the surface of capillary endothelium cells in cerebral vessels. (Smith et al., Proc. Natl. Acad. Sci. USA 97:1766-1771 (2000); Franke-Fayard et al., Proc. Natl. Acad. Sci. USA 102, 11468-11473 (2005).

Although a few drugs, such as chloroquine that targets the heme detoxification pathway, are used to treat malaria, there are increasing incidence of parasite resistance to drugs and mosquito vector resistance to insecticides. Chloroquine antagonizes heme polymerization mediated by parasite-induced HRPs (histidine-rich proteins), as heme monomers are highly toxic for malaria parasites. The polymerization of heme allows detoxification, which is reversed by chloroquine. Another drug, artemisinin, is effective against chloroquine-resistant P. falciparum in cerebral malaria. Artemisinin forms adducts with globin-bound heme in hemoglobin, which binds HRPs to prevent heme polymerization.

A cancer is a malignant tumor of potentially unlimited growth. It is primarily the pathogenic replication (a loss of normal regulatory control) of various types of cells found in the human body. Initial treatment of the disease is often surgery, radiation treatment or the combination of these treatments, but locally recurrent and metastatic disease is frequent. Chemotherapeutic treatments for some cancers are available but these seldom induce long term regression. Hence, they are often not curative. Commonly, tumors and their metastases become refractory to chemotherapy, in an event known as the development of multidrug resistance. In many cases, tumors are inherently resistant to some classes of chemotherapeutic agents. In addition, such treatments threaten noncancerous cells, are stressful to the human body, and produce many side effects.

Angiogenesis is the formation of new blood vessels from preexisting endothelial vasculature. Folkman, et al., J. Exp. Med. 133:275-288, (1971). Most tumors require angiogenesis to sustain growth beyond a critical volume of 1-2 mm, when the supply of nutrients and metabolites becomes insufficient due to the limits of diffusional exchange. Folkman, J. Nat. Cancer Inst. 82:4-6 (1990). Tumors deprived of angiogenesis remain dormant indefinitely, only to rapidly grow when a blood supply is acquired. Brem et al., Cancer Res. 36:2807-2812 (1976). The degree of angiogenesis often increases with tumor progression. Dome et al., J. Pathol. 197:355-362 (2002). Further, invasion and metastatic spread of tumors are also thought to be angiogenesis-dependant events. Folkman, Ann Surg. 175:409-416 (1972). The newly formed blood vessels provide a route for cancer cells to enter the circulatory system and spread to distant parts of the body. Fidler and Ellis, Cell 79:185-188 (1994).

Because angiogenesis is an integral process in the growth and spread of tumors, it is an important focus of cancer therapy. Anti-angiogenesis therapy is effective not only for solid tumors, but also hematopoietic tumors, leukemia and myeloma, Bellamy et al., Cancer Res. 59:728-733 (1999); Rajkumar et al., Leukemia. 13:469-472 (1999). Endothelial cells are thought to be better targets for therapy than tumor cells because they have a longer generation time and more genetic stability that tumor cells. Endothelial cells are therefore less likely to “escape” therapy by developing drug resistance to the therapy administered. Boehn-Vaiswanathan, Curr. Opin. Oncol. 12:89-94 (2000).

Other conditions suffered by mammals are also related to inappropriate angiogenesis. Wet macular degeneration occurs when blood capillaries inappropriately grow into the retina. Inappropriate angiogenesis has also been implicated as a fundamental characteristic of diabetic retinopathy, psoriasis and rheumatoid arthritis, among other diseases. Bussolino et al., Trends Biochem. Sci. 22:251-256 (1997); Folkman, Nat. Med. 1: 27-31 (1995).

Numerous diseases, such as those discussed above, may occur concurrently in a patient, or one disease may cause or increase the probability of causing another disease in a patient. For example, an HIV infected patient is associated with an increased risk of acquiring large cell lymphoma or Kaposi's sarcoma. The Merck Manual of Diagnosis and Therapy, (Beers et al., 18^{th} edition, Merck Research Laboratories, 2006). For another example, a female patient that acquires human papilloma-virus has an increased risk of acquiring cervical carcinoma. Id.

Numerous diseases also have a high probability to infect a patient concurrently due to environmental factors. Environmental factors may include a patient's lifestyle, eating habits and/or geographic location. For example, co-infections with HIV and malaria are very common in many areas of the world, and in particular sub-Saharan Africa.

Genetic predisposition may also play a factor in a patient acquiring two diseases concurrently. For example, it is known that when a person carries a particular cystic fibrosis transmembrane regulator (CFTR) mutation, that person has a higher risk for cystic fibrosis and pancreatic cancer. Weiss et al., Gut; 54: 1456-1460 (2005).

Because so many factors can cause or increase the probability of a patient acquiring two or more diseases or conditions, it would be practical to have one-compound or a group of related compounds that could inhibit, or treat and/or prevent two or more diseases or conditions concurrently.

### SUMMARY OF THE INVENTION

The present invention relates to compositions comprising peptides that may be cupredoxin or cytochrome or variants, derivatives, truncations and structural equivalents of cupredoxin or cytochrome that treat and/or prevent two or more conditions in a mammalian cell.

The present invention further relates to compositions that may comprise cupredoxin or cytochrome, and/or variants, derivatives, truncations, or structural equivalents of cupredoxin or cytochrome, that retain the ability to concurrently treat and/or prevent two or more conditions such as cancer, inappropriate angiogenesis, HIV and malaria in a patient. These compositions may be isolated peptides or pharmaceutical compositions, among others.

In one embodiment of the present invention, the cupredoxin may be azurin, pseudoazurin, plastocyanin, rusticyanin, Laz, auracyanin, stellacyanin and cucumber basic protein, and specifically may be azurin. The cupredoxin may be from an organism such as Pseudomonas aeruginosa, Alcaligenes faecalis, Ulva pertussis, Achromobacter xylosoxidan, Bordetella bronchiseptica, Methylomonas sp., Neisseria meningitidis, Neisseria gonorrhea, Pseudomonas fluorescens, Bordetella pertussis, Pseudomonas syringae, Pseudomonas chlororaphis, Xylella fastidiosa and Vibrio parahaemolyticus, and specifically may be Pseudomonas aeruginosa.

In another embodiment of the present invention, the isolated peptide may inhibit parasitemia by malaria in P. falciparum-infected human red blood cells.

In another embodiment, the isolated peptide may be fused to a H.8 region of Laz.

In another embodiment of the present invention, the isolated peptide may be a structural equivalent of monoclonal antibody G17.12.

In another embodiment of the present invention, the isolated peptide may be a cytochrome selected from one or more of the group consisting of cytochrome c, cytochrome f and cytochrome c₅₅₁.

In another embodiment of the present invention, the isolated peptide of cytochrome c may be from an organism selected from the group consisting of human and Pseudomonas aeruginosa.

In another embodiment of the present invention, the isolated peptide of cytochrome f may be from cyanobacteria.

In another embodiment, the isolated peptide may be part of SEQ ID NOS: 1, 5-12, 18 and 23, a mutant of SEQ ID NOS: 1, 5-12, 18 and 23, or have at least 90% amino acid sequence identity to SEQ ID NOS: 1, 5-12, 18 and 23. In another embodiment, the isolated peptide may be a truncation of a peptide selected from one or more of the group consisting of SEQ ID NOS: 1, 5-12, 18 and 23. In another embodiment, the isolated peptide may be a truncation of a cupredoxin. The isolated peptide may be any suitable length, including from 10 to 100 residues, 18 to 100 residues, or 18 to 28 residues. The isolated peptide may comprise or consist of a sequence and/or the equivalent residues of a cupredoxin as a region selected from the group consisting of Pseudomonas aeruginosa azurin residues 50-77 (SEQ ID NO: 29), Pseudomonas aeruginosa azurin residues 50-67 (SEQ ID NO: 30), Pseudomonas aeruginosa azurin residues 36-88 (SEQ ID NO: 50), Pseudomonas aeruginosa azurin residues 36-128 (SEQ ID NO: 31), Pseudomonas aeruginosa azurin residues 88-113 (SEQ ID NO: 49), Pseudomonas aeruginosa azurin residues 36-89 (SEQ ID NO: 32), and Pseudomonas aeruginosa azurin residues 96-113 (SEQ ID NO: 48), Vibrio parahaemolyticus azurin residues 52-78 (SEQ ID NO: 27), Pseudomonas syringae azurin residues 51-77 (SEQ ID NO: 25), Bordetella bronchiseptica azurin residues 51-77 (SEQ ID NO: 28), and Pseudomonas aeruginosa azurin residues 36-77 (SEQ ID NO: 33). The isolated peptide may also be a truncation of any of those sequences or a truncation of a larger sequence that comprises those sequences.

The isolated peptide may comprise equivalent residues of a region of the isolated peptide, wherein the peptide comprises the sequence and/or the equivalent residues of a cupredoxin as a region selected from the group consisting of Pseudomonas aeruginosa azurin residues 50-77 (SEQ ID NO: 29), Pseudomonas aeruginosa azurin residues 50-67 (SEQ ID NO: 30), Pseudomonas aeruginosa azurin residues 36-88 (SEQ ID NO: 50), Pseudomonas aeruginosa azurin residues 36-128 (SEQ ID NO: 31), Pseudomonas aeruginosa azurin residues 88-113 (SEQ ID NO: 49), Pseudomonas aeruginosa azurin residues 36-89 (SEQ ID NO: 32), and Pseudomonas aeruginosa azurin residues 96-113 (SEQ ID NO: 48), Vibrio parahaemolyticus azurin residues 52-78 (SEQ ID NO: 27), Pseudomonas syringae azurin residues 51-77 (SEQ ID NO: 25), Bordetella bronchiseptica azurin residues 51-77 (SEQ ID NO: 28), and Pseudomonas aeruginosa azurin residues 36-77 (SEQ ID NO: 33). The isolated peptide may also be a truncation of any of those sequences or a truncation of a larger sequence that comprises those sequences.

In another embodiment of the present invention, the compositions may comprise one or at least two cupredoxins, cytochromes or peptides in a pharmaceutical composition. In some the embodiments, the pharmaceutical compositions may comprise the isolated peptides of the present invention.

In another embodiment, the cupredoxin in a pharmaceutical composition may be from an organism such as Pseudomonas aeruginosa, Alcaligenes faecalis, Ulva pertussis, Achromobacter xylosoxidan, Bordetella bronchiseptica, Methylomonas sp., Neisseria meningitidis, Neisseria gonorrhea, Pseudomonas fluorescens, Bordetella pertussis, Pseudomonas syringae, Pseudomonas chlororaphis, Xylella fastidiosa and Vibrio parahaemolyticus, and specifically may be Pseudomonas aeruginosa.

In another embodiment of the present invention, the cupredoxin in a pharmaceutical composition may be selected from one or more of the group consisting of SEQ ID NOS: 1, 5-12, 18, 23, 25, 27-33 and 48-50. In another embodiment of the present invention, the cupredoxin in a pharmaceutical composition may comprise SEQ ID NO: 30.

In another embodiment of the present invention, the composition may be administered to a patient for the concurrent prevention and/or treatment of two or more conditions selected from the group consisting of interstitial cystitis (IC), lesions associated with inflammatory bowel disease (IBD), HIV infection, AIDS, central nervous system disorders, peripheral vascular diseases, viral diseases, degeneration of the central nervous system (Christopher Reeve's disease), Alzheimer's disease, malaria, inappropriate angiogenesis, cardiovascular disease, hypertension, Cytomegalovirus infection, human papilloma virus infection; Muscular Dystrophy, encephalopathy, dementia, Parkinson's disease, neuropathy, macular degeneration, diabetic retinopathy, rheumatoid arthritis, psoriasis, herpes simplex virus (HSV), Ebola virus, cytomegalovirus (CMV), Para influenza viruses types A, B and C, hepatitis virus A, B, C, and G, the delta hepatitis virus (HDV), mumps virus, measles virus, respiratory syncytial virus, bunyvirus, arena virus, Dhori virus, poliovirus, rubella virus, dengue virus; SIV, Mycobacterium tuberculosis and cancer.

In another embodiment of the present invention, the composition may comprise a therapeutic agent for the concurrent prevention and/or treatment of cancer selected from the group consisting of melanoma, leukemia, breast cancer, ovarian cancer, lung cancer, mesenchymal cancer, colon cancer, aerodigestive tract cancer, cervical cancer, brain tumors, and prostate cancer.

In another embodiment of the present invention, the compositions may be administered to a patient for the concurrent prevention and/or treatment of two or more conditions selected from the group consisting of HIV, malaria, cancer and inappropriate angiogenesis.

In another embodiment of the present invention, the compositions may comprise a therapeutic agent for the treatment of malaria, wherein the patient is additionally suffering from one or more of the group consisting of HIV, cancer or inappropriate angiogenesis.

In another embodiment of the present invention, the compositions may comprise a therapeutic agent for the treatment of malaria, wherein the patient has a higher risk than the general population of acquiring a condition selected from one or more of the group consisting of HIV, cancer or inappropriate angiogenesis.

In another embodiment of the present invention, the compositions may comprise a therapeutic agent for the treatment of HIV, wherein the patient is additionally suffering from one or more of the group consisting of malaria, cancer or inappropriate angiogenesis.

In another embodiment of the present invention, the compositions may comprise a therapeutic agent for the treatment of HIV, wherein the patient has a higher risk than the general population of acquiring a condition selected from one or more of the group consisting of malaria, cancer or inappropriate angiogenesis.

In another embodiment of the present invention, the compositions may comprise a therapeutic agent for the treatment of cancer, wherein the patient is additionally suffering from one or more of the group consisting of HIV, malaria or inappropriate angiogenesis.

In another embodiment of the present invention, the compositions may comprise a therapeutic agent for the treatment of cancer, wherein the patient has a higher risk than the general population of acquiring a condition selected from one or more of the group consisting of HIV, malaria or inappropriate angiogenesis.

In another embodiment of the present invention, the compositions may comprise a therapeutic agent for the treatment of inappropriate angiogenesis, wherein the patient is additionally suffering from one or more of the group consisting of HIV, cancer or malaria.

In another embodiment of the present invention, the compositions may comprise a therapeutic agent for the treatment of inappropriate angiogenesis, wherein the patient has a higher risk than the general population of acquiring a condition selected from one or more of the group consisting of HIV, cancer or malaria.

In another embodiment of the present invention, the compositions may comprise another drug selected from the group consisting of an anti-malarial drug, an anti-HIV drug, an anti-cancer drug and an anti-angiogenesis drug.

In another embodiment of the present invention, the pharmaceutical composition may be administered by intravenous injection, intramuscular injection, subcutaneous injection, inhalation, topical administration, transdermal patch, suppository, vitreous injection or oral.

In another embodiment of the present invention, the pharmaceutical composition may be co-administered with at least one other drug. In another embodiment, the pharmaceutical composition may be co-administered with one other drug selected from the group consisting of an anti-malarial drug, an anti-HIV drug, an anti-cancer drug and an anti-angiogenesis drug.

In another embodiment of the present invention, the pharmaceutical composition may be administered at about the same time with another drug. The other drug may be an anti-malarial drug, an anti-HIV drug, an anti-cancer drug and an anti-angiogenesis drug.

In another embodiment of the present invention, the methods may include administering to a patient the composition comprising one or at least two cupredoxins, cytochromes or peptides in a pharmaceutical composition. In another embodiment, the patient is human.

In another embodiment of the present invention, the methods may include administering the compositions to a patient for the concurrent prevention and/or treatment of two or more conditions selected from the group consisting of interstitial cystitis (IC), lesions associated with inflammatory bowel disease (IBD), HIV infection, AIDS, central nervous system disorders, peripheral vascular diseases, viral diseases, degeneration of the central nervous system (Christopher Reeve's disease), Alzheimer's disease, malaria, inappropriate angiogenesis, cardiovascular disease, hypertension, Cytomegalovirus infection, human papilloma virus infection; Muscular Dystrophy, encephalopathy, dementia, Parkinson's disease, neuropathy, macular degeneration, diabetic retinopathy, rheumatoid arthritis, psoriasis, herpes simplex virus (HSV), Ebola virus, cytomeglovirus (CMV), Para influenza viruses types A, B and C, hepatitis virus A, B, C, and G, the delta hepatitis virus (HDV), mumps virus, measles virus, respiratory syncytial virus, bunyvirus, arena virus, Dhori virus, poliovirus, rubella virus, dengue virus; SIV, Mycobacterium tuberculosis and cancer. The cancer may be selected from the group consisting of melanoma, leukemia, breast cancer, ovarian cancer, lung cancer, mesenchymal cancer, colon cancer, aerodigestive tract cancer, cervical cancer, brain tumors, and prostate cancer.

In another embodiment of the present invention, the methods may include administering the compositions to a patient for the concurrent prevention and/or treatment of two or more conditions selected from the group consisting of HIV, malaria, cancer and inappropriate angiogenesis.

In another embodiment of the present invention, the methods may utilize a therapeutic agent for the treatment of malaria, wherein the patient is additionally suffering from one or more of the group consisting of HIV, cancer or inappropriate angiogenesis.

In another embodiment of the present invention, the methods may utilize a therapeutic agent for the treatment of malaria, wherein the patient has a higher risk than the general population of acquiring a condition selected from one or more of the group consisting of HIV, cancer or inappropriate angiogenesis.

In another embodiment of the present invention, the methods may utilize a therapeutic agent for the treatment of HIV, wherein the patient is additionally suffering from one or more of the group consisting of malaria, cancer or inappropriate angiogenesis.

In another embodiment of the present invention, the methods may utilize a therapeutic agent for the treatment of HIV, wherein the patient has a higher risk than the general population of acquiring a condition selected from one or more of the group consisting of malaria, cancer or inappropriate angiogenesis.

In another embodiment of the present invention, the methods may utilize a therapeutic agent for the treatment of cancer, wherein the patient is additionally suffering from one or more of the group consisting of HIV, malaria or inappropriate angiogenesis.

In another embodiment of the present invention, the methods may utilize a therapeutic agent for the treatment of cancer, wherein the patient has a higher risk than the general population of acquiring a condition selected from one or more of the group consisting of HIV, malaria or inappropriate angiogenesis.

In another embodiment of the present invention, the methods may utilize a therapeutic agent for the treatment of inappropriate angiogenesis, wherein the patient is additionally suffering from one or more of the group consisting of HIV, cancer or malaria.

In another embodiment of the present invention, the methods may utilize a therapeutic agent for the treatment of inappropriate angiogenesis, wherein the patient has a higher risk than the general population of acquiring a condition selected from one or more of the group consisting of HIV, cancer or malaria.

In another embodiment of the present invention, the methods may utilize compositions wherein the composition is administered to a patient at a higher risk to develop cancer than the general population.

In another embodiment of the present invention, the methods may utilize compositions wherein the composition is administered to a patient at a higher risk to develop HIV than the general population.

In another embodiment of the present invention, the methods may utilize compositions wherein the composition is administered to a patient at a higher risk to develop malaria than the general population.

In another embodiment of the present invention, the methods may utilize compositions wherein the composition is administered to a patient at a higher risk to develop inappropriate angiogenesis than the general population.

In another embodiment of the present invention, the methods may utilize compositions wherein the composition is administered to a patient that has a higher risk than the general population of acquiring one or more of the group consisting of HIV, cancer, angiogenesis and malaria.

In another embodiment of the present invention, the methods may utilize compositions wherein the composition is administered to a patient that has at least one high risk feature.

In another embodiment of the present invention, the methods may utilize a pharmaceutical composition administered by intravenous injection, intramuscular injection, subcutaneous injection, inhalation, topical administration, transdermal patch, suppository, vitreous injection or oral, and specifically may be administered by intravenous injection.

In another embodiment of the present invention, the methods may utilize a pharmaceutical composition co-administered with at least one other drug. In another embodiment, the other drug may be an anti-malarial drug, an anti-HIV drug, an anti-cancer drug and an anti-angiogenesis drug.

In another embodiment of the present invention, the methods may utilize a pharmaceutical composition administered at about the same time with at least one other drug. In another embodiment, the methods may utilize a pharmaceutical composition administered at about the same time with at least one other drug selected from the group consisting of an anti-malarial drug, an anti-HIV drug, an anti-cancer drug and an anti-angiogenesis drug.

In another embodiment of the present invention, the composition may be a kit comprising the pharmaceutical composition of the invention. In another embodiment of the present invention, the kit may be designed for intravenous administration.

In another embodiment of the present invention, the composition be an isolated peptide that can bind a protein selected from the group consisting of CD4, gp120, ICAM3, DC-SIGN, PFMSP1-19 and PFMSP1-42.

These and other aspects, advantages, and features of the invention will become apparent from the following figures and detailed description of the specific embodiments.

### DESCRIPTION OF THE SEQUENCES

SEQ ID NO: 1.
Amino acid sequence of azurin from Pseudomonas
aeruginosa
(Ala Glu Cys Ser Val Asp Ile Gln Gly Asn Asp Gln
Met Gln Phe Asn Thr Asn Ala Ile Thr Val Asp Lys
Ser Cys Lys Gln Phe Thr Val Asn Leu Ser His Pro
Gly Asn Leu Pro Lys Asn Val Met Gly His Asn Trp
Val Leu Ser Thr Ala Ala Asp Met Gln Gly Val Val
Thr Asp Gly Met Ala Ser Gly Leu Asp Lys Asp Tyr
Leu Lys Pro Asp Asp Ser Arg Val Ile Ala His Thr
Lys Leu Ile Gly Ser Gly Glu Lys Asp Ser Val Thr
Phe Asp Val Ser Lys Leu Lys Glu Gly Glu Gln Tyr
Met Phe Phe Cys Thr Phe Pro Gly His Ser Ala Leu
Met Lys Gly Thr Leu Thr Leu Lys).
SEQ ID NO: 2.
Amino acid sequence of plastocyanin from
Phormidium laminosum
(Glu Thr Phe Thr Val Lys Met Gly Ala Asp Ser Gly
Leu Leu Gln Phe Glu Pro Ala Asn Val Thr Val His
Pro Gly Asp Thr Val Lys Trp Val Asn Asn Lys Leu
Pro Pro His Asn Ile Leu Phe Asp Asp Lys Gln Val
Pro Gly Ala Ser Lys Glu Leu Ala Asp Lys Leu Ser
His Ser Gln Leu Met Phe Ser Pro Gly Glu Ser Tyr
Glu Ile Thr Phe Ser Ser Asp Phe Pro Ala Gly Thr
Tyr Thr Tyr Tyr Cys Ala Pro His Arg Gly Ala Gly
Met Val Gly Lys Ile Thr Val Glu Gly).
SEQ ID NO: 3.
Amino acid sequence of rusticyanin from
Thiobacillus ferrooxidans
(Gly Thr Leu Asp Thr Thr Trp Lys Glu Ala Thr Leu
Pro Gln Val Lys Ala Met Leu Glu Lys Asp Thr Gly
Lys Val Ser Gly Asp Thr Val Thr Tyr Ser Gly Lys
Thr Val His Val Val Ala Ala Ala Val Leu Pro Gly
Phe Pro Phe Pro Ser Phe Glu Val His Asp Lys Lys
Asn Pro Thr Leu Glu Ile Pro Ala Gly Ala Thr Val
Asp Val Thr Phe Ile Asn Thr Asn Lys Gly Phe Gly
His Ser Phe Asp Ile Thr Lys Lys Gly Pro Pro Tyr
Ala Val Met Pro Val Ile Asp Pro Ile Val Ala Gly
Thr Gly Phe Ser Pro Val Pro Lys Asp Gly Lys Phe
Gly Tyr Thr Asp Phe Thr Trp His Pro Thr Ala Gly
Thr Tyr Tyr Tyr Val Cys Gln Ile Pro Gly His Ala
Ala Thr Gly Met Phe Gly Lys Ile Val Val Lys).
SEQ ID NO: 4.
Amino acid sequence of pseudoazurin from
Achromobacter cycloclastes
(Ala Asp Phe Glu Val His Met Leu Asn Lys Gly Lys
Asp Gly Ala Met Val Phe Glu Pro Ala Ser Leu Lys
Val Ala Pro Gly Asp Thr Val Thr Phe Ile Pro Thr
Asp Lys Gly His Asn Val Glu Thr Ile Lys Gly Met
Ile Pro Asp Gly Ala Glu Ala Phe Lys Ser Lys Ile
Asn Glu Asn Tyr Lys Val Thr Phe Thr Ala Pro Gly
Val Tyr Gly Val Lys Cys Thr Pro His Tyr Gly Met
Gly Met Val Gly Val Val Gln Val Gly Asp Ala Pro
Ala Asn Leu Glu Ala Val Lys Gly Ala Lys Asn Pro
Lys Lys Ala Gln Glu Arg Leu Asp Ala Ala Leu Ala
Ala Leu Gly Asn).
SEQ ID NO: 5.
Amino acid sequence of azurin from Alcaligenes
faecalis
(Ala Cys Asp Val Ser Ile Glu Gly Asn Asp Ser Met
Gln Phe Asn Thr Lys Ser Ile Val Val Asp Lys Thr
Cys Lys Glu Phe Thr Ile Asn Leu Lys His Thr Gly
Lys Leu Pro Lys Ala Ala Met Gly His Asn Val Val
Val Ser Lys Lys Ser Asp Glu Ser Ala Val Ala Thr
Asp Gly Met Lys Ala Gly Leu Asn Asn Asp Tyr Val
Lys Ala Gly Asp Glu Arg Val Ile Ala His Thr Ser
Val Ile Gly Gly Gly Glu Thr Asp Ser Val Thr Phe
Asp Val Ser Lys Leu Lys Glu Gly Glu Asp Tyr Ala
Phe Phe Cys Ser Phe Pro Gly His Trp Ser Ile Met
Lys Gly Thr Ile Glu Leu Gly Ser).
SEQ ID NO: 6.
Amino acid sequence of azurin from Achromobacter
xylosoxidans ssp. denitrgficans I
(Ala Gln Cys Glu Ala Thr Ile Glu Ser Asn Asp Ala
Met Gln Tyr Asn Leu Lys Glu Met Val Val Asp Lys
Ser Cys Lys Gln Phe Thr Val His Leu Lys His Val
Gly Lys Met Ala Lys Val Ala Met Gly His Asn Trp
Val Leu Thr Lys Glu Ala Asp Lys Gln Gly Val Ala
Thr Asp Gly Met Asn Ala Gly Leu Ala Gln Asp Tyr
Val Lys Ala Gly Asp Thr Arg Val Ile Ala His Thr
Lys Val Ile Gly Gly Gly Glu Ser Asp Ser Val Thr
Phe Asp Val Ser Lys Leu Thr Pro Gly Glu Ala Tyr
Ala Tyr Phe Cys Ser Phe Pro Gly His Trp Ala Met
Met Lys Gly Thr Leu Lys Leu Ser Asn).
SEQ ID NO: 7.
Amino acid sequence of azurin from Bordetella
bronchiseptica
(Ala Glu Cys Ser Val Asp Ile Ala Gly Thr Asp Gln
Met Gln Phe Asp Lys Lys Ala Ile Glu Val Ser Lys
Ser Cys Lys Gln Phe Thr Val Asn Leu Lys His Thr
Gly Lys Leu Pro Arg Asn Val Met Gly His Asn Trp
Val Leu Thr Lys Thr Ala Asp Met Gln Ala Val Glu
Lys Asp Gly Ile Ala Ala Gly Leu Asp Asn Gln Tyr
Leu Lys Ala Gly Asp Thr Arg Val Leu Ala His Thr
Lys Val Leu Gly Gly Gly Glu Ser Asp Ser Val Thr
Phe Asp Val Ala Lys Leu Ala Ala Gly Asp Asp Tyr
Thr Phe Phe Cys Ser Phe Pro Gly His Gly Ala Leu
Met Lys Gly Thr Leu Lys Leu Val Asp).
SEQ ID NO: 8.
Amino acid sequence of azurin from Methylomonas
sp. J
(Ala Ser Cys Glu Thr Thr Val Thr Ser Gly Asp Thr
Met Thr Tyr Ser Thr Arg Ser Ile Ser Val Pro Ala
Ser Cys Ala Glu Phe Thr Val Asn Phe Glu His Lys
Gly His Met Pro Lys Thr Gly Met Gly His Asn Thr
Val Leu Ala Lys Ser Ala Asp Val Gly Asp Val Ala
Lys Glu Gly Ala His Ala Gly Ala Asp Asn Asn Phe
Val Thr Pro Gly Asp Lys Arg Val Ile Ala Phe Thr
Pro Ile Ile Gly Gly Gly Glu Lys Thr Ser Val Lys
Phe Lys Val Ser Ala Leu Ser Lys Asp Glu Ala Tyr
Thr Tyr Phe Cys Ser Tyr Pro Gly His Phe Ser Met
Met Arg Gly Thr Leu Lys Leu Glu Glu).
SEQ ID NO: 9.
Amino acid sequence of azurin from Neisseria
meningitides Z2491
(Cys Ser Gln Glu Pro Ala Ala Pro Ala Ala Glu Ala
Thr Pro Ala Ala Glu Ala Pro Ala Ser Glu Ala Pro
Ala Ala Glu Ala Ala Pro Ala Asp Ala Ala Glu Ala
Pro Ala Ala Gly Asn Cys Ala Ala Thr Val Glu Ser
Asn Asp Asn Met Gln Phe Asn Thr Lys Asp Ile Gln
Val Ser Lys Ala Cys Lys Glu Phe Thr Ile Thr Leu
Lys His Thr Gly Thr Gln Pro Lys Thr Ser Met Gly
His Asn Ile Val Ile Gly Lys Thr Glu Asp Met Asp
Gly Ile Phe Lys Asp Gly Val Gly Ala Ala Asp Thr
Asp Tyr Val Lys Pro Asp Asp Ala Arg Val Val Ala
His Thr Lys Leu Ile Gly Gly Gly Glu Glu Ser Ser
Leu Thr Leu Asp Pro Ala Lys Leu Ala Asp Gly Glu
Tyr Lys Phe Ala Cys Thr Phe Pro Gly His Gly Ala
Leu Met Asn Gly Lys Val Thr Leu Val Asp).
SEQ ID NO: 10.
Amino acid sequence of azurin from Pseudomonas
fluorescen
(Ala Glu Cys Lys Thr Thr Ile Asp Ser Thr Asp Gln
Met Ser Phe Asn Thr Lys Ala Ile Glu Ile Asp Lys
Ala Cys Lys Thr Phe Thr Val Glu Leu Thr His Ser
Gly Ser Leu Pro Lys Asn Val Met Gly His Asn Leu
Val Ile Ser Lys Gln Ala Asp Met Gln Pro Ile Ala
Thr Asp Gly Leu Ser Ala Gly Ile Asp Lys Asn Tyr
Leu Lys Glu Gly Asp Thr Arg Val Ile Ala His Thr
Lys Val Ile Gly Ala Gly Glu Lys Asp Ser Leu Thr
Ile Asp Val Ser Lys Leu Asn Ala Ala Glu Lys Tyr
Gly Phe Phe Cys Ser Phe Pro Gly His Ile Ser Met
Met Lys Gly Thr Val Thr Leu Lys).
SEQ ID NO: 11.
Amino acid sequence of azurin from Pseudomonas
syringae
(Met Ala Ser Gly Gln Leu Leu Ala Ala Glu Cys Ser
Ala Thr Val Asp Ser Thr Asp Gln Met Met Tyr Asp
Thr Lys Ala Ile Gln Ile Asp Lys Ser Cys Lys Glu
Phe Thr Leu Asn Leu Thr His Ser Gly Ser Leu Pro
Lys Asn Val Met Gly His Asn Trp Val Leu Ser Lys
Lys Ala Asp Ala Ser Ala Ile Thr Thr Asp Gly Met
Ser Val Gly Ile Asp Lys Asp Tyr Val Lys Pro Asp
Asp Thr Arg Val Ile Ala His Thr Lys Ile Ile Gly
Ala Gly Glu Asn Asp Ser Val Thr Phe Asp Val Ser
Lys Leu Asp Pro Ala Glu Asp Tyr Gln Phe Phe Cys
Thr Phe Pro Gly His Ile Ser Met Met Lys Gly Ala
Val Thr Leu Lys).
SEQ ID NO: 12.
Amino acid sequence of azurin from Xylella
fastidiosa 9a5c
(Lys Thr Cys Ala Val Thr Ile Ser Ala Asn Asp Gln
Met Lys Phe Asp Gln Asn Thr Ile Lys Ile Ala Ala
Glu Cys Thr His Val Asn Leu Thr Leu Thr His Thr
Gly Lys Lys Ser Ala Arg Val Met Gly His Asn Trp
Val Leu Thr Lys Thr Thr Asp Met Gln Ala Val Ala
Leu Ala Gly Leu His Ala Thr Leu Ala Asp Asn Tyr
Val Pro Lys Ala Asp Pro Arg Val Ile Ala His Thr
Ala Ile Ile Gly Gly Gly Glu Arg Thr Ser Ile Thr
Phe Pro Thr Asn Thr Leu Ser Lys Asn Val Ser Tyr
Thr Phe Phe Cys Ser Phe Pro Gly His Trp Ala Leu
Met Lys Gly Thr Leu Asn Phe Gly Gly).
SEQ ID NO: 13
Amino acid sequence of stellacyanin from Cucumis
sativus
(Met Gln Ser Thr Val His Ile Val Gly Asp Asn Thr
Gly Trp Ser Val Pro Ser Ser Pro Asn Phe Tyr Ser
Gln Trp Ala Ala Gly Lys Thr Phe Arg Val Gly Asp
Ser Leu Gln Phe Asn Phe Pro Ala Asn Ala His Asn
Val His Glu Met Glu Thr Lys Gln Ser Phe Asp Ala
Cys Asn Phe Val Asn Ser Asp Asn Asp Val Glu Arg
Thr Ser Pro Val Ile Glu Arg Leu Asp Glu Leu Gly
Met His Tyr Phe Val Cys Thr Val Gly Thr His Cys
Ser Asn Gly Gln Lys Leu Ser Ile Asn Val Val Ala
Ala Asn Ala Thr Val Ser Met Pro Pro Pro Ser Ser
Ser Pro Pro Ser Ser Val Met Pro Pro Pro Val Met
Pro Pro Pro Ser Pro Ser).
SEQ ID NO: 14.
Amino acid sequence of auracyanin A from
Chloroflexus aurantiacus
(Met Lys Ile Thr Leu Arg Met Met Val Leu Ala Val
Leu Thr Ala Met Ala Met Val Leu Ala Ala Cys Gly
Gly Gly Gly Ser Ser Gly Gly Ser Thr Gly Gly Gly
Ser Gly Ser Gly Pro Val Thr Ile Glu Ile Gly Ser
Lys Gly Glu Glu Leu Ala Phe Asp Lys Thr Glu Leu
Thr Val Ser Ala Gly Gln Thr Val Thr Ile Arg Phe
Lys Asn Asn Ser Ala Val Gln Gln His Asn Trp Ile
Leu Val Lys Gly Gly Glu Ala Glu Ala Ala Asn Ile
Ala Asn Ala Gly Leu Ser Ala Gly Pro Ala Ala Asn
Tyr Leu Pro Ala Asp Lys Ser Asn Ile Ile Ala Glu
Ser Pro Leu Ala Asn Gly Asn Glu Thr Val Glu Val
Thr Phe Thr Ala Pro Ala Ala Gly Thr Tyr Leu Tyr
Ile Cys Thr Val Pro Gly His Tyr Pro Leu Met Gln
Gly Lys Leu Val Val Asn).
SEQ ID NO: 15.
Amino acid sequence of auracyanin B from
Chloroflexus aurantiacus
(Ala Ala Asn Ala Pro Gly Gly Ser Asn Val Val Asn
Glu Thr Pro Ala Gln Thr Val Glu Val Arg Ala Ala
Pro Asp Ala Leu Ala Phe Ala Gln Thr Ser Leu Ser
Leu Pro Ala Asn Thr Val Val Arg Leu Asp Phe Val
Asn Gln Asn Asn Leu Gly Val Gln His Asn Trp Val
Leu Val Asn Gly Gly Asp Asp Val Ala Ala Ala Val
Asn Thr Ala Ala Gln Asn Asn Ala Asp Ala Leu Phe
Val Pro Pro Pro Asp Thr Pro Asn Ala Leu Ala Trp
Thr Ala Met Leu Asn Ala Gly Glu Ser Gly Ser Val
Thr Phe Arg Thr Pro Ala Pro Gly Thr Tyr Leu Tyr
Ile Cys Thr Phe Pro Gly His Tyr Leu Ala Gly Met
Lys Gly Thr Leu Thr Val Thr Pro).
SEQ ID NO: 16.
Amino acid sequence of cucumber basic protein from
Cucumis sativus
(Ala Val Tyr Val Val Gly Gly Ser Gly Gly Trp Thr
Phe Asn Thr Glu Ser Trp Pro Lys Gly Lys Arg Phe
Arg Ala Gly Asp Ile Leu Leu Phe Asn Tyr Asn Pro
Ser Met His Asn Val Val Val Val Asn Gln Gly Gly
Phe Ser Thr Cys Asn Thr Pro Ala Gly Ala Lys Val
Tyr Thr Ser Gly Arg Asp Gln Ile Lys Leu Pro Lys
Gly Gln Ser Tyr Phe Ile Cys Asn Phe Pro Gly His
Cys Gln Ser Gly Met Lys Ile Ala Val Asn Ala Leu).
SEQ ID NO: 17.
Amino acid sequence of Laz from Neisseria
gonorrhoeae F62
(Cys Ser Gln Glu Pro Ala Ala Pro Ala Ala Glu Ala
Thr Pro Ala Gly Glu Ala Pro Ala Ser Glu Ala Pro
Ala Ala Glu Ala Ala Pro Ala Asp Ala Ala Glu Ala
Pro Ala Ala Gly Asn Cys Ala Ala Thr Val Glu Ser
Asn Asp Asn Met Gln Phe Asn Thr Lys Asp Ile Gln
Val Ser Lys Ala Cys Lys Glu Phe Thr Ile Thr Leu
Lys His Thr Gly Thr Gln Pro Lys Ala Ser Met Gly
His Asn Leu Val Ile Ala Lys Ala Glu Asp Met Asp
Gly Val Phe Lys Asp Gly Val Gly Ala Ala Asp Thr
Asp Tyr Val Lys Pro Asp Asp Ala Arg Val Val Ala
His Thr Lys Leu Ile Gly Gly Gly Glu Glu Ser Ser
Leu Thr Leu Asp Pro Ala Lys Leu Ala Asp Gly Asp
Tyr Lys Phe Ala Cys Thr Phe Pro Gly His Gly Ala
Leu Met Asn Gly Lys Val Thr Leu Val Asp).
SEQ ID NO: 18.
Amino acid sequence of the azurin from Vibrio
parahaemolyticus
(Met Ser Leu Arg Ile Leu Ala Ala Thr Leu Ala Leu
Ala Gly Leu Ser Phe Gly Ala Gln Ala Ser Ala Glu
Cys Glu Val Ser Ile Asp Ala Asn Asp Met Met Gln
Phe Ser Thr Lys Thr Leu Ser Val Pro Ala Thr Cys
Lys Glu Val Thr Leu Thr Leu Asn His Thr Gly Lys
Met Pro Ala Gln Ser Met Gly His Asn Val Val Ile
Ala Asp Thr Ala Asn Ile Gln Ala Val Gly Thr Asp
Gly Met Ser Ala Gly Ala Asp Asn Ser Tyr Val Lys
Pro Asp Asp Glu Arg Val Tyr Ala His Thr Lys Val
Val Gly Gly Gly Glu Ser Thr Ser Ile Thr Phe Ser
Thr Glu Lys Met Thr Ala Gly Gly Asp Tyr Ser Phe
Phe Cys Ser Phe Pro Gly His Trp Ala Ile Met Gln
Gly Lys Phe Glu Phe Lys).
SEQ ID NO: 19.
Amino acid sequence of cytochrome c from Homo
sapiens
(Gly Asp Val Glu Lys Gly Lys Lys Ile Phe Ile Met
Lys Cys Ser Gln Cys His Thr Val Glu Lys Gly Gly
Lys His Lys Thr Gly Pro Asn Leu His Gly Leu Phe
Gly Arg Lys Thr Gly Gln Ala Pro Gly Tyr Ser Tyr
Thr Ala Ala Asn Lys Asn Lys Gly Ile Ile Trp Gly
Glu Asp Thr Leu Met Glu Tyr Leu Glu Asn Pro Lys
Lys Tyr Ile Pro Gly Thr Lys Met Ile Phe Val Gly
Ile Lys Lys Lys Glu Glu Arg Ala Asp Leu Ile Ala
Tyr Leu Lys Lys Ala Thr Asn Glu).
SEQ ID NO: 20.
Amino acid sequence of cytochrome f from
cyanobacteria Phormidium laminosum
(Met Asn Phe Lys Val Cys Ser Phe Pro Ser Arg Arg
Gln Ser Ile Ala Ala Phe Val Arg Val Leu Met Val
Ile Leu Leu Thr Leu Gly Ala Leu Val Ser Ser Asp
Val Leu Leu Pro Gln Pro Ala Ala Ala Tyr Pro Phe
Trp Ala Gln Gln Asn Tyr Ala Asn Pro Arg Glu Ala
Thr Gly Arg Ile Val Cys Ala Asn Cys His Leu Ala
Ala Lys Pro Ala Glu Ile Glu Val Pro Gln Ala Val
Leu Pro Asp Ser Val Phe Lys Ala Val Val Lys Ile
Pro Tyr Asp His Ser Val Gln Gln Val Gln Ala Asp
Gly Ser Lys Gly Pro Leu Asn Val Gly Ala Val Leu
Met Leu Pro Glu Gly Phe Thr Ile Ala Pro Glu Asp
Arg Ile Pro Glu Glu Met Lys Glu Glu Val Gly Pro
Ser Tyr Leu Phe Gln Pro Tyr Ala Asp Asp Lys Gln
Asn Ile Val Leu Val Gly Pro Leu Pro Gly Asp Gln
Tyr Glu Glu Ile Val Phe Pro Val Leu Ser Pro Asn
Pro Ala Thr Asn Lys Ser Val Ala Phe Gly Lys Tyr
Ser Ile His Leu Gly Ala Asn Arg Gly Arg Gly Gln
Ile Tyr Pro Thr Gly Glu Lys Ser Asn Asn Ala Val
Tyr Asn Ala Ser Ala Ala Gly Val Ile Thr Ala Ile
Ala Lys Ala Asp Asp Gly Ser Ala Glu Val Lys Ile
Arg Thr Glu Asp Gly Thr Thr Ile Val Asp Lys Ile
Pro Ala Gly Pro Glu Leu Ile Val Ser Glu Gly Glu
Glu Val Ala Ala Gly Ala Ala Leu Thr Asn Asn Pro
Asn Val Gly Gly Phe Gly Gln Lys Asp Thr Glu Ile
Val Leu Gln Ser Pro Asn Arg Val Lys Gly Arg Ile
Ala Phe Leu Ala Ala Ile Thr Leu Thr Gln Ile Leu
Leu Val Leu Lys Lys Lys Gln Val GIu Arg Val Gln
Ala Gly Arg Asp Asp Leu Leu Lys Ala Ala Phe Ile
Ala Gly).
SEQ ID NO: 21.
Amino acid sequence of cytochrome c₅₅₁ from
Pseudomonas aeruginosa
(Glu Asp Pro Glu Val Leu Phe Lys Asn Lys Gly Cys
Val Ala Cys His Ala Ile Asp Thr Lys Met Val Gly
Pro Ala Tyr Lys Asp Val Ala Ala Lys Phe Ala Gly
Gln Ala Gly Ala Glu Ala Glu Leu Ala Gln Arg Ile
Lys Asn Gly Ser Gln Gly Val Trp Gly Pro Ile Pro
Met Pro Pro Asn Ala Val Ser Asp Asp Glu Ala Gln
Thr Leu Ala Lys Trp Val Leu Ser Gln Lys).
SEQ ID NO: 22.
Amino acid sequence of the H.8 region of Laz from
Neisseria gonorrhoeae F62
(Cys Ser Gln Glu Pro Ala Ala Pro Ala Ala Glu Ala
Thr Pro Ala Gly Glu Ala Pro Ala Ser Glu Ala Pro
Ala Ala Glu Ala Ala Pro Ala Asp Ala Ala Glu Ala
Pro Ala Ala).
SEQ ID NO: 23
is the amino acid sequence of the azurin from
Bordetella pertussis
(Ala Glu Cys Ser Val Asp Ile Ala Gly Thr Asp Gln
Met Gln Phe Asp Lys Lys Ala Ile Glu Val Ser Lys
Ser Cys Lys Gln Phe Thr Vai Asn Leu Lys His Thr
Gly Lys Leu Pro Arg Asn Val Met Gly His Asn Trp
Val Leu Thr Lys Thr Ala Asp Met Gln Ala Val Glu
Lys Asp Gly Ile Ala Ala Gly Leu Asp Asn Gln Tyr
Leu Lys Ala Gly Asp Thr Arg Val Leu Ala His Thr
Lys Val Leu Gly Gly Gly Glu Ser Asp Ser Val Thr
Phe Asp Val Ala Lys Leu Ala Ala Gly Asp Asp Tyr
Thr Phe Phe Cys Ser Phe Pro Gly His Gly Ala Leu
Met Lys Gly Thr Leu Lys Leu Val Asp).
SEQ ID NO: 24.
Amino acid sequence of amino acids 57-89 of
auracyanin B of Chloroflexus aurantiacus
(His Asn Trp Val Leu Val Asn Gly Gly Asp Asp Val
Ala Ala Ala Val Asn Thr Ala Ala Gln Asn Asn Ala
Asp Ala Leu Phe Val Pro Pro Pro Asp).
SEQ ID NO: 25.
Amino acid sequence of amino acids 51-77 of
Pseudomonas syringae azurin
(Ser Lys Lys Ala Asp Ala Ser Ala Ile Thr Thr Asp
Gly Met Ser Val Gly Ile Asp Lys Asp Tyr Val Lys
Pro Asp Asp).
SEQ ID NO: 26.
Amino acid sequence of amino acids 89-115 of
Neisseria meningitides Laz
(Ile Gly Lys Thr Glu Asp Met Asp Gly Ile Phe Lys
Asp Gly Val Gly Ala Ala Asp Thr Asp Tyr Val Lys
Pro Asp Asp).
SEQ ID NO: 27.
Amino acid sequence of amino acids 52-78 of Vibrio
parahaemolyticus azurin
(Ala Asp Thr Ala Asn Ile Gln Ala Val Gly Thr Asp
Gly Met Ser Ala Gly Ala Asp Asn Ser Tyr Val Lys
Pro Asp Asp).
SEQ ID NO: 28.
Amino acid sequence of amino acids 51-77 of
Bordetella bronchiseptica azurin
(Thr Lys Thr Ala Asp Met Gln Ala Val GIu Lys Asp
Gly Ile Ala Ala Gly Leu Asp Asn Gln Tyr Leu Lys
Ala Gly Asp).
SEQ ID NO: 29
is the amino acid sequence of the 50-77 amino acid
fragment of wt-azurin from Pseudomonas aeruginosa
(Leu Ser Thr Ala Ala Asp Met Gln Gly Val Val Thr
Asp Gly Met Ala Ser Gly Leu Asp Lys Asp Tyr Leu
Lys Pro Asp Asp).
SEQ ID NO: 30
is the amino acid sequence of the 50-67 amino acid
fragment of wt-azurin from Pseudomonas aeruginosa
(Leu Ser Thr Ala Ala Asp Met Gln Gly Val Val Thr
Asp Gly Met Ala Ser Gly).
SEQ ID NO: 31
is the amino acid sequence of the 36-128 amino acid
fragment of wt-azurin from Pseudomonas aeruginosa
(Pro Gly Asn Leu Pro Lys Asn Val Met Gly His Asn
Trp Val Leu Ser Thr Ala Ala Asp Met Gln Gly Val
Val Thr Asp Gly Met Ala Ser Gly Leu Asp Lys Asp
Tyr Leu Lys Pro Asp Asp Ser Arg Val Ile Ala His
Thr Lys Leu Ile Gly Ser Gly Glu Lys Asp Ser Val
Thr Phe Asp Val Ser Lys Leu Lys Glu Gly GIu Gln
Tyr Met Phe Phe Cys Thr Phe Pro Gly His Ser Ala
Leu Met Lys Gly Thr Leu Thr Leu Lys).
SEQ ID NO: 32
is the amino acid sequence of the 36-89 amino acid
fragment of wt-azurin from Pseudomonas aeruginosa
(Pro Gly Asn Leu Pro Lys Asn Val Met Gly His Asn
Trp Val Leu Ser Thr Ala Ala Asp Met Gln Gly Val
Val Thr Asp Gly Met Ala Ser Gly Leu Asp Lys Asp
Tyr Leu Lys Pro Asp Asp Ser Arg Val Ile Ala His
Thr Lys Leu Ile Gly Ser).
SEQ ID NO: 33
is the amino acid sequence of the 36-77 amino acid
fragment of wt-azurin from Pseudomonas aeruginosa
(Pro Gly Asn Leu Pro Lys Asn Val Met Gly His Asn
Trp Val Leu Ser Thr Ala Ala Asp Met Gln Gly Val
Val Thr Asp Gly Met Ala Ser Gly Leu Asp Lys Asp
Tyr Leu Lys Pro Asp Asp).
SEQ ID NO: 34
is the forward primer to PCR amplify the Laz-
encoding gene (laz) of Neisseria gonorrhoeae
(ccggaattcc ggcagggatg ttgtaaatat ccg).
SEQ ID NO: 35
is the reverse primer to PCR amplify the Laz-
encoding gene (laz) of Neisseria gonorrhoeae
(ggggtaccgc cgtggcaggc atacagcatt tcaatcgg).
SEQ ID NO: 36
is the forward primer to PCR amplify a 3.1 kb
fragment of pUC 18-laz
(ggcagcaggg gcttcggcag catctgc).
SEQ ID NO: 37
is the reverse primer to PCR amplify a 3.1 kb
fragment of puc 18-laz
(ctgcaggtcg actctagagg atcccg).
SEQ ID NO: 38
is the forward primer to PCR amplify a 0.4 kb
fragment of pUC19-paz
(gccgagtgct cggtggacat ccagg).
SEQ ID NO: 39
is the reverse primer to PCR amplify a 0.4 kb
fragment of pUC 19-paz
(tactcgagtc acttcagggt cagggtg).
SEQ ID NO: 40
is the forward primer for pGST-azu 36-128
(cgggatcccc ggcaacctgc cgaagaacgt catgggc).
SEQ ID NO: 41
is the reverse primer for pGST-azu 36-128
(cggaattcgc atcacttcag ggtcaggg).
SEQ ID NO: 42
is the forward primer for pGST-azu 36-89
(ccaagctgat cggctcgtga gagaaggact cggtgacc).
SEQ ID NO: 43
is the reverse primer for pGST-azu 36-89
(ggtcaccgag tccttctctc acgagccgat cagcttgg).
SEQ ID NO: 44
is the forward primer for pGST-azu 88-113
(cggggatccc cggctcgggc gagaaggac).
SEQ ID NO: 45
is the reverse primer for pGST-azu 88-113
(cgggaattct ccacttcagg gtcagggtg).
SEQ ID NO: 46
is an oligonucleotide for site directed
mutagenesis for the preparation of pGST-azu 88-113
(gttcttctgc acctagccgg gccactccg).
SEQ ID NO: 47
is an oligonucleotide for site directed
mutagenesis for the preparation of pGST-azu 88-113
(cggagtggcc cggctaggtg cagaagaac).
SEQ ID NO: 48
is the amino acid sequence of the 96-113 amino
acid fragment of wt-azurin from Pseudomonas
aeruginosa
(Thr Phe Asp Val Ser Lys Leu Lys Glu Gly Glu Gln
Tyr Met Phe Phe Cys Thr).
SEQ ID NO: 49
is the amino acid sequence of the 88-113 amino
acid fragment of wt-azurin from Pseudomonas
aeruginosa
(Gly Ser Gly Glu Lys Asp Ser Val Thr Phe Asp Val
Ser Lys Leu Lys Glu Gly Glu Gln Tyr Met Phe Phe
Cys Thr).
SEQ ID NO: 50
is the amino acid sequence of the 36-88 amino acid
fragment of wt-azurin from Pseudomonas aeruginosa
(Pro Gly Asn Leu Pro Lys Asn Val Met Gly His Asn
Trp Val Leu Ser Thr Ala Ala Asp Met Gln Gly Val
Val Thr Asp Gly Met Ala Ser Gly Leu Asp Lys Asp
Tyr Leu Lys Pro Asp Asp Ser Arg Val Ile Ala His
Thr Lys Leu Ile Gly).
SEQ ID NO: 51
is the amino acid sequence of a variant of the
azurin truncation p28
(Leu Ser Thr Ala Ala Asp Met Gln Ala Val Val Thr
Asp Thr Met Ala Ser Gly Leu Asp Lys Asp Tyr Leu
Lys Pro Asp Asp).
SEQ ID NO: 52
is the amino acid sequence of a variant of the
azurin truncation p28
(Leu Ser Thr Ala Ala Asp Leu Gln Gly Val Val Thr
Asp Gly Leu Ala Ser Gly Leu Asp Lys Asp Tyr Leu
Lys Pro Asp Asp).
SEQ ID NO: 53
is the amino acid sequence of a variant of the
azurin truncation p28
(Leu Ser Thr Ala Ala Asp Val Gln Gly Val Val Thr
Asp Gly Val Ala Ser Gly Leu Asp Lys Asp Tyr Leu
Lys Pro Asp Asp).
SEQ ID NO: 54
is the amino acid sequence of a modified
cupredoxin derived peptide
(Asp Asp Pro Lys Leu Tyr Asp Lys Asp Leu Gly Ser
Ala Met Gly Asp Thr Val Val Gly Gln Met Asp Ala
Ala Thr Ser Leu).
SEQ ID NO: 55
is the amino acid sequence of a modified
cupredoxin derived peptide
(Acetylation-Leu Ser Thr Ala Ala Asp Met Gln Gly
Val Val Thr Asp Gly Met Ala Ser Gly Leu Asp Lys
Asp Tyr Leu Lys Pro Asp Asp-amidation).
SEQ ID NO: 56
is the amino acid sequence of a hexapeptide
(Val Ser Pro Pro Ala Arg).
SEQ ID NO: 57
is the amino acid sequence of a hexapeptide
(Tyr Thr Pro Pro Ala Leu).
SEQ ID NO: 58
is the amino acid sequence of a hexapeptide
(Phe Ser Phe Phe Ala Phe).
SEQ ID NO: 59
is the amino acid sequence of a modified
cupredoxin-derived peptide
(Leu Ser Thr Ala Ala Asp Met Gln Gly Val Val Thr
Asp Gly Met Ala Ser Gly Leu Asp Lys Asp Tyr Leu
Thr Pro Gly Cys).
SEQ ID NO: 60
is the amino acid sequence of a modified
cupredoxin-derived peptide
(Leu Ser Thr Ala Ala Asp Cys Gln Gly Val Val Thr
Asp Gly Met Ala Ser Gly Leu Asp Lys Asp Tyr Leu
Lys Pro Asp Asp).
SEQ ID NO: 61
is the amino acid sequence of a modified
cupredoxin-derived peptide
(Leu Ser Thr Ala Ala Cys Met Gln Gly Val Val Thr
Asp Gly Met Ala Ser Gly Leu Asp Lys Asp Tyr Leu
Lys Pro Asp Asp).
SEQ ID NO: 62
is the amino acid sequence of a modified
cupredoxin-derived peptide
(Leu Ser Thr Ala Cys Asp Met Gln Gly Val Val Thr
Asp Gly Met Ala Ser Gly Leu Asp Lys Asp Tyr Leu
Lys Pro Asp Asp).
SEQ ID NO: 63
is the amino acid sequence of a modified
cupredoxin-derived peptide
(Leu Ser Thr Ala Ala Thr Met Gln Cys Val Val Thr
Asp Gly Met Ala Ser Gly Leu Asp Lys Asp Tyr Leu
Lys Pro Asp Asp).
SEQ ID NO: 64
is the amino acid sequence of a modified
cupredoxin-derived peptide
(Leu Ser Thr Ala Ala Thr Met Gln Gly Cys Val Thr
Asp Gly Met Ala Ser Gly Leu Asp Lys Asp Tyr Leu
Lys Pro Asp Asp).
SEQ ID NO: 65
is the amino acid sequence of a modified
cupredoxin-derived peptide
(Leu Ser Thr Ala Ala Asn Thr Gln Gly Cys Val Thr
Asp Gly Met Ala Ser Gly Leu Asp Lys Asp Tyr Leu
Lys Pro Asp Asp).
SEQ ID NO: 66
is the amino acid sequence of a modified
cupredoxin-derived peptide
(Leu Ser Thr Ala Ala Asn Thr Gln Gly Val Cys Thr
Asp Gly Met Ala Ser Gly Leu Asp Lys Asp Tyr Leu
Lys Pro Asp Asp).
SEQ ID NO: 67
is the amino acid sequence of a modified
cupredoxin-derived peptide
(Leu Ser Thr Ala Ala Asp Met Thr Ala Val Cys Thr
Asp Gly Met Ala Ser Gly Leu Asp Lys Asp Tyr Leu
Lys Pro Asp Asp).
SEQ ID NO: 68
is the amino acid sequence of a modified
cupredoxin-derived peptide
(Leu Ser Thr Ala Ala Asp Met Thr Ala Val Val Cys
Asp Gly Met Ala Ser Gly Leu Asp Lys Asp Tyr Leu
Lys Pro Asp Asp).
SEQ ID NO: 69
is the amino acid sequence of a modified
cupredoxin-derived peptide
(Leu Ser Thr Ala Ala Asp Met Gln Thr Val Val Cys
Asp Gly Met Ala Ser Gly Leu Asp Lys Asp Tyr Leu
Lys Pro Asp Asp).
SEQ ID NO: 70
is the amino acid sequence of a modified
cupredoxin-derived peptide
(Leu Ser Thr Ala Ala Asp Met Gln Thr Val Val Thr
Cys Gly Met Ala Ser Gly Leu Asp Lys Asp Tyr Leu
Lys Pro Asp Asp).
SEQ ID NO: 71
is the amino acid sequence of a modified
cupredoxin-derived peptide
(Leu Ser Thr Ala Ala Asp Met Gln Ala Thr Val Thr
Cys Gly Met Ala Ser Gly Leu Asp Lys Asp Tyr Leu
Lys Pro Asp Asp).
SEQ ID NO: 72
is the amino acid sequence of a modified
cupredoxin-derived peptide
(Leu Ser Thr Ala Ala Asp Met Gln Ala Thr Val Thr
Asp Cys Met Ala Ser Gly Leu Asp Lys Asp Tyr Leu
Lys Pro Asp Asp).
SEQ ID NO: 73
is the amino acid sequence of a modified
cupredoxin-derived peptide
(Leu Ser Thr Ala Ala Asp Met Gln Gly Val Thr Ala
Asp Cys Met Ala Ser Gly Leu Asp Lys Asp Tyr Leu
Lys Pro Asp Asp).
SEQ ID NO: 74
is the amino acid sequence of a modified
cupredoxin-derived peptide
(Leu Ser Thr Ala Ala Asp Met Gln Gly Val Thr Ala
Asp Gly Cys Ala Ser Gly Leu Asp Lys Asp Tyr Leu
Lys Pro Asp Asp).
SEQ ID NO: 75
is the amino acid sequence of a modified
cupredoxin-derived peptide
(Leu Ser Thr Ala Ala Asp Met Gln Gly Val Val Thr
Asn Gly Cys Ala Ser Gly Leu Asp Lys Asp Tyr Leu
Lys Pro Asp Asp).
SEQ ID NO: 76
is the amino acid sequence of a modified
cupredoxin-derived peptide
(Leu Ser Thr Ala Ala Asp Met Gln Gly Val Val Thr
Ala Thr Met Gly Ser Gly Leu Cys Lys Asp Tyr Leu
Lys Pro Asp Asp).
SEQ ID NO: 77
is the amino acid sequence of a modified
cupredoxin-derived peptide
(Leu Ser Thr Ala Ala Asp Met Gln Gly Val Val Thr
Asp Leu Thr Ala Ser Gly Leu Cys Lys Asp Tyr Leu
Lys Pro Asp Asp).
SEQ ID NO: 78
is the amino acid sequence of a modified
cupredoxin-derived peptide
(Leu Ser Trp Ala Ala Asp Met Gln Gly Val Val Thr
Asp Gly Met Ala Ser Gly Leu Asp Lys Asp Tyr Leu
Lys Pro Asp Asp).
SEQ ID NO: 79
is the amino acid sequence of a modified
cupredoxin-derived peptide
(Leu Ser Thr Ala Ala Asp Met Trp Gly Val Val Thr
Asp Gly Met Ala Ser Gly Leu Asp Lys Asp Tyr Leu
Lys Pro Asp Asp).
SEQ ID NO: 80
is the amino acid sequence of a modified
cupredoxin-derived peptide
(Leu Ser Thr Ala Ala Asp Met Gln Gly Val Val Trp
Asp Gly Met Ala Ser Gly Leu Asp Lys Asp Tyr Leu
Lys Pro Asp Asp).
SEQ ID NO: 81
is the amino acid sequence of a modified
cupredoxin-derived peptide
(Leu Ser Thr Ala Ala Asp Met Gln Gly Val Val Thr
Asp Trp Met Ala Ser Gly Leu Asp Lys Asp Tyr Leu
Lys Pro Asp Asp).
SEQ ID NO: 82
is the amino acid sequence of a modified
cupredoxin-derived peptide
(Leu Ser Trp Ala Ala Asp Met Trp Gly Val Val Thr
Asp Gly Met Ala Ser Gly Leu Asp Lys Asp Tyr Leu
Lys Pro Asp Asp).
SEQ ID NO: 83
is the amino acid sequence of a modified
cupredoxin-derived peptide
(Leu Ser Trp Ala Ala Asp Met Gln Gly Val Val Trp
Asp Gly Met Ala Ser Gly Leu Asp Lys Asp Tyr Leu
Lys Pro Asp Asp).
SEQ ID NO: 84
is the amino acid sequence of a modified
cupredoxin-derived peptide
(Leu Ser Trp Ala Ala Asp Met Gln Gly Val Val Thr
Asp Trp Met Ala Ser Gly Leu Asp Lys Asp Tyr Leu
Lys Pro Asp Asp).
SEQ ID NO: 85
is the amino acid sequence of a modified
cupredoxin-derived peptide
(Leu Ser Thr Ala Ala Asp Met Trp Gly Val Val Trp
Asp Gly Met Ala Ser Gly Leu Asp Lys Asp Tyr Leu
Lys Pro Asp Asp).
SEQ ID NO: 86
is the amino acid sequence of a modified
cupredoxin-derived peptide
(Leu Ser Thr Ala Ala Asp Met Trp Gly Val Val Thr
Asp Trp Met Ala Ser Gly Leu Asp Lys Asp Tyr Leu
Lys Pro Asp Asp).
SEQ ID NO: 87
is the amino acid sequence of a modified
cupredoxin-derived peptide
(Leu Ser Thr Ala Ala Asp Met Gln Gly Val Val Trp
Asp Trp Met Ala Ser Gly Leu Asp Lys Asp Tyr Leu
Lys Pro Asp Asp).
SEQ ID NO: 88
is the amino acid sequence of a modified
cupredoxin-derived peptide
(Leu Ser Trp Ala Ala Asp Met Trp Gly Val Val Trp
Asp Trp Met Ala Ser Gly Leu Asp Lys Asp Tyr Leu
Lys Pro Asp Asp).
SEQ ID NO: 89
is the amino acid sequence of a modified
cupredoxin-derived peptide
(X₁ Ser X₂ Ala Ala Asp X₃ X₄ X₅ Val Val X₆ Asp X₇
X₈ Ala Ser Gly Leu Asp Lys Asp Tyr Leu Lys Pro Asp
X₉).
SEQ ID NO: 90
is the amino acid sequence of a modified
cupredoxin-derived peptide
(X₁ Asp Pro Lys Leu Tyr Asp Lys Asp Leu Gly Ser
Ala X₂ X₃ Asp X₄ Val Val X₅ X₆ X₇ Asp Ala Ala X₈
Ser X₉).
SEQ ID NO: 91
is the amino acid sequence of p18b, the 60-77
amino acid fragment of wt-azurin from Pseudomonas
aeruginosa
(Val Thr Asp Gly Met Ala Ser Gly Leu Asp Lys Asp
Tyr Leu Lys Pro Asp Asp)
SEQ ID NO: 92
is the amino acid sequence of the 10 C-terminal
amino acids of p28
(Leu Asp Lys Asp Tyr Leu Lys Pro Asp Asp).
SEQ ID NO: 93
is the amino acid sequence of the 12 C-terminal
amino acids of p28
(Ser Gly Leu Asp Lys Asp Tyr Leu Lys Pro Asp Asp).
SEQ ID NO: 94
is the amino acid sequence of Arg₈
(Arg Arg Arg Arg Arg Arg Arg Arg).

### BRIEF DESCRIPTION OF THE FIGURES

FIG. 1. FIG. 1 depicts confocal microscopy images of malignant and normal cells incubated with p28 labeled with Alexafluor® 568 and the cells are then stained with DAPI. The indicated cell lines were incubated in the absence (negative control) or presence (p28) of 20 μM Alexafluor® 568 labeled p28 for 2 h at 37° C. The images are indicative of amount of cellular entry observed. FIG. 1A depicts the Alexafluor® 568 fluorescence and control fluorescence of human melanoma, pancreatic, breast (BCA-1), breast (MCF-7), glioblastoma, astrocytoma, lung and prostrate cancer cells. FIG. 1B depicts the Alexafluor® 568 fluorescence and control fluorescence of human normal fibroblast, pancreas and breast cells. FIG. 1C depicts the Alexafluor® 568 fluorescence and control fluorescence of human umbilical vein endothelial cells (HUVEC).

FIG. 2. FIG. 2 depicts the capillary tube formation by HUVEC cells plated on Matrigel® in the presence or absence of p28. Culture media contained 20 ng/ml VEGF. FIG. 2A shows images of HUVEC cells incubated for 4 h at 37° C. with 0.10 μM, 0.30 μM, 0.92 μM, 2.77 μM, 8.33 μM, 25 μM and 75 μM of p28, and then stained with calcein AM and visualized using fluorescence microscopy. In FIG. 2B, the graph shows the average number of tubes formed in peptide treated and control (untreated) cells.

FIG. 3. FIG. 3 depicts the results of the scratch wound HUVEC migration assay. In FIGS. 3A-C show the fixed cells that were stained for F-actin and nuclei. In FIG. 3A, HUVEC cells at 90% confluence were scratched using a 1 ml plastic pipette tip. In FIG. 3B, the HUVEC cells were scratched and then incubated in the culture media containing 20 ng/ml VEGF for 24 h at 37° C. in the absence of p28. In FIG. 3C, the HUVEC cells were scratched and then incubated for 24 h at 37° C. in the presence of 25 μM p28. The insets of FIGS. 3A-C show the cell density in the area away from the scored area. In FIG. 3D, a bar graph indicates the average # of cells in 20 different fields (20×) of the scratched area in control and p28 treated wells (FIGS. 3B and C). Data represent mean±SEM. * indicates the differences are statistically significant.

FIG. 4. FIG. 4 depicts the images of the localization of cell structural proteins with and without p28 treatment. HUVEC cells were plated on Matrigel®-coated cover slips, incubated in the culture media containing 20 ng/ml VEGF in the presence or absence of p28 peptide (25 μM) for 4 and 24 h, fixed, and processed for staining of CD31/PECAM-1, paxillin, Fak (focal adhesion kinase), vinculin, WASP (Wiskott Aldrich Syndrome protein) and β-catenin. Each figure pertains to the detection of particular structural protein: FIG. 4A is CD31/PECAM-1; FIG. 4B is paxillin; FIG. 4C is Fak; FIG. 4D is WASP; FIG. 4E is vinculin; and FIG. 4F is β-catenin. Each figure is divided into four panes which show the image of the localization of the fluorescent markers used. Each pane is numbered to indicate the fluorescent marker detected: 1=F-actin; 2=DAPI; 3=FITC-Protein of interest; 4=merged image. Arrows indicate the localization of the protein of interest.

FIG. 5. FIG. 5 depicts Mel-2 cells which were treated with increasing concentrations of p28 for 24, 48, and 72 hours. The number of cells in treated and control wells were counted using a Coulter counter. Data represent percentage of cell growth inhibition when compared to control cultures at the time point.

FIG. 6. Depicts the results when Mel-2 cells were injected subcutaneously in the left flank (about 1 million cells/animal). Animals received p28 at the indicated dose at the time of injection. FIG. 6A shows the incidence of tumor occurrence after initiation of treatment with a graph indicating % of tumor free animals at days post treatment with Mel-2 cells. FIG. 6B shows the tumor size after initiation of treatment with a graph indicating the average volume of the tumors (cm³) at days post treatment with Mel-2 cells.

FIG. 7. FIG. 7 depicts surface plasmon resonance binding titrations depicting the interactions of Azurin, H.8-azurin (H.8-Az), Laz, and GST-azurin (GST-Azu) constructs with MSP1-19 and MSP1-42. (A) Binding curves demonstrating the interactions of azurin and its analogues with MSP1-19 immobilized on carboxymethyldextran coated gold sensor chips (MSP1-19-CM5). Concentration dependent binding of the azurin proteins to MSP1-19 was determined via injection of various concentrations (0.05-300 nM) over the sensor surface and the extent of binding was evaluated as a function of the equilibrium resonance response value measured in resonance units (RU). While H.8-Az and Laz bound somewhat more strongly than azurin, no binding was seen with GST or H.8-GST. (B) In vitro binding titrations for immobilized MSP1-42 with azurin and its analogues was followed in a similar manner to that for MSP1-19 as shown in (A). Relative binding affinities were determined via fitting the data to Req=Rmax/(1+(Kd/C)) with the curve fits connecting the data points in the graphs. The MSP1-19 binding Kd values are: 32.2±2.4 nM (azurin), 26.2±2.4 nM (Laz), 11.8±0.3 nM (H.8-Az), and those for MSP1-42 binding are: 54.3±7.6 nM (azurin), 45.6±2.4 nM (Laz) and 14.3±1.7 nM (H.8-Az). (C) Binding titrations for the interactions of GST-Azu fusion proteins over the MSP1-19-CM5 sensors surface demonstrate the recognition of GST-Azu 36-128 and GST-Azu 36-89 with MSP1-19. No binding was seen with GST or GST-Azu 88-113.

FIG. 8. FIG. 8 depicts inhibition of P. falciparum parasitemia (parasite growth within the RBC) by different concentrations, as shown, of Azurin, H.8-azurin (H.8-Az) and Laz. In these experiments, normal red blood cells were infected with schizonts in absence or in presence of the proteins at different concentrations, incubated overnight and the number of intracellular parasites was scored by thin blood smear and Giemsa staining.

FIG. 9. FIG. 9 depicts surface plasmon resonance binding curves for the binding of ICAMs (ICAM-1, ICAM-2, ICAM-3 and NCAM, inset) with immobilized azurin. Due to large nonspecific binding to the bare Au-CM5 chip, CM5 was added as an eluent to the running buffer (1 mg/ml CM5 to HBS-EP buffer). The selective recognition of azurin with ICAM-3, but not with ICAM-1 or ICAM-2, is notable and the binding strength was 19.5±5.4 nM. The Kd for NCAM binding with azurin, as shown in the inset, was 20±5.0 nM.

FIG. 10. FIG. 10 depicts the inhibition of HIV-1 viral growth by azurin, H.8-azurin (H.8-Az) and Laz. These three proteins were incubated at different concentrations with PBMC followed by addition of the three subtypes of HIV-1. After 2 h incubation, the virus was removed but the proteins added back as described in Example 18. Suppression of virus growth was determined by ELISA assays of p24.

FIG. 11. FIG. 11 depicts surface plasmon resonance binding curves depicting the binding patterns of cupredoxins with CD4 and HIV-1 gp120. (A) SPR titration curves showing novel and specific binding of azurin, and GST-Azu 36-128 (shown as an inset) with immobilized CD4 on carboxymethyldextran coated gold sensor chips (CD4-CM5). HIV-1 gp120, HIV-1 gag, and HIV-1 nef served as the positive and negative controls respectively. Relative binding affinities were determined via fitting the data to R_{eq}=Rₘₐₓ/(1+(K_{d}/C)) with the curve fits connecting the data points above. The CD4 binding K_{d} values are: 36.9±2.0 nM (azurin), 0.34±0.04 nM (GST-Azu 36-128), and 48.1±3.1 nM (HIV-1 gp120). (B) The binding titrations when immobilized azurin (Az-CM5) is in contact with HIV proteins. Due to large nonspecific binding to the bare Au-CM5 chip, CM5 was added as an eluent to the running buffer (1 mg/ml CM5 to HBS-EP buffer). Curve fits gave Kd's of 25.1±3.1 nM (CD4), and 8.9±0.8 mM (HIV-1 gp120). (C) SPR curves for the binding of ICAMs (ICAM-1, ICAM-2, ICAM-3 and NCAM, inset) with immobilized azurin were determined under similar conditions as for experiments in part (B). The selective recognition of azurin with ICAM-3, but not with ICAM-1 or ICAM-2, is notable and the binding strength was 19.5±5.4 nM. The Kd for NCAM binding with azurin, as shown in the inset, was 20±5.0 nM. (D) SPR binding competition studies with CD4 immobilized on CM5 sensor chips. Azurin+HIV-1 gp120 solutions were added at different azurin concentrations (0-4500 nM, [HIV-1 gp120] is 242 nM) to the sensor surface and the data were plotted as a ratio of resonances, % total response [R_{eq}(azurin+HIV-1 gp120)/(R_{eq}/(HIV-1 gp120))]. GST-Azu 36-128 was titrated with HIV-1 gp120 to immobilized CD4 and analyzed in a similar manner. Competition data suggests 1:1 stoichiometry of binding between azurin and GST-Azu 36-128 with immobilized CD4.

FIG. 12. FIG. 12 depicts surface plasmon resonance binding titrations depicting the interactions of azurin, and GST-Azurin fusions with DC-SIGN. (A) Concentration dependent binding of azurin, ICAM-3, and GST-Azu 36-89 with DC-SIGN were determined via injection of various concentrations of the proteins (0-100 nM) over a DC-SIGN modified CM5 sensor surface and the extent of binding was evaluated as a function of the equilibrium resonance response value measured in resonance units (RU). (B) The binding titration curve of GST-Azu 88-113 with DC-SIGN using the same sensor chip and protocol as described for azurin in part A. The positive interaction of GST-Azu 88-113 with DC-SIGN suggests its potential role as the recognition sequence for azurin. The binding affinities (Kd) for azurin, ICAM-3 and GST-Azu 88-113 were determined by fitting the data to Req=Rmax/(1+(Kd/C)) and the curve fits connect the data points in these plots. The extrapolated Kd values are 0.83±0.05 nM (azurin), 0.93±0.39 nM (ICAM-3), and 5.98±1.13 nM (GST-Azu 88-113).

FIG. 13. FIG. 13 depicts the effects of cupredoxin peptides on cancer cell viability. In FIG. 13A, effect of azurin (Azu 96-113) and plastocyanin (Plc 70-84) synthetic peptides on cell viability of Astrocytoma CCF-STTG1 and Glioblastoma LN-229 cancer cell lines. In FIG. 13B, effect of different concentrations of plastocyanin (Plc 70-84) synthetic peptide on Melanoma UISO-Mel-2 cell viability. Cancer cells (2×10⁴ cells per well in 96-well plates) were treated with the synthetic peptides at different concentrations for 24 h at 37° C. Data are presented as the percentage of cell viability as compared to that of untreated control (100% viability) In FIG. 13C, cytotoxic activity of Azu 96-113 synthetic peptide towards Glioblastoma LN-229 cells. Cytotoxicity effects were determined by MTT assay. Cancer (2×10⁴ cells per well in 96-well plates) were treated with various concentrations of Azu 96-113 (10, 25, 50, 75, 100 μM) for 24 h at 37° C. Percent cytotoxicity is expressed as percentage of cell death as compared to that of untreated control (0% cytotoxicity).

FIG. 14. Effect of GST-Azu 36-128 and GST-Azu 88-113 on cell viability of MCF-7 cells. GST-Azu peptides were added at increasing concentrations (1.25, 6.25 and 12.5 μM) into 96 well plates containing 8×10³ cancer cells per well, incubated at 37° C. for 48 h and subsequently analyzed using MTT assay. GST and GST-Azu 36-89 at the same concentrations and untreated cells were run in parallel with GST-Azu 36-128 and GST-Azu 88-113 as controls.

FIGS. 15 A-C. Depict photographs showing penetration of azurin derived peptides, p18 and p28, into cancer cell lines of diverse histogenesis and their normal counterparts. (A) Photos showing penetration of Alexafluor 568 labeled p28 or p18 after 2 hrs at 37° C. The cationic Arg₈ (SEQ ID NO: 94) was used as a control. (B) Graphs depicting flow cytometric analysis of the penetration of Alexafluor 568 labeled p28 or p18 into the same cell lines after 2 hrs at 37° C. (C) Graphs depicting fold increase over fluorescence from normal cells. Similar observations of p28 or p18 entry into 4 melanoma cell lines show a several fold increase over fluorescence from normal cells.

FIGS. 16 A and B. Depict photographs showing entry of azu 60-77 (p18b) and azu 66-77 (p12) into cancer and normal cells. Cells were incubated with alexafluor 568 labeled p18b (A) or p12 (B) at 37° C. for 2 hrs and images recorded by confocal microscopy.

FIGS. 17 A and B. Graphs depicting cellular membrane toxicity of azurin and its peptides. (A) LDH leakage assay of UISOMel-2 cells exposure for 10 min to different concentrations of p28, p18 and azurin at 37° C. A standard lysis buffer (cytotox-one reagent) was included as a positive control. Changes in fluorescence following exposure were measured at k,x 560 nm and kem 590 nm. Lysis buffer was defined as 100% LDH release. Data represent % of positive fluorescence of control. Data are shown as mean±SEM. (B) Hemoglobin leakage from human erythrocytes incubated with p28, p18 and azurin. Human erythrocytes were incubated with peptide for 30 min at 37° C. and absorbance at 540 nm determined. Hemoglobin release following 0.1% Triton X-100 was defined as 100% hemoglobin release. Data represent mean±SEM of triplicate determinations.

FIGS. 18 A-D. Depict photographs showing temperature dependent and competitive internalization of p28 and p18 into UISO-Mel-2 cells. Penetration of Alexafluor 568 labeled p28 (A) or p18 (B) at 2011M was evaluated by confocal microscopy at different temperatures. (C) and (D) Confocal analysis of entry of Alexafluor 568 labeled p28 (C) or p18 (D) at 5 μM into UISO-Mel-2 cells after 30 min at 37° C. in the presence/absence of unlabeled peptide (200 fold excess).

FIGS. 19 A-D. (A) Depicts photographs showing confocal analysis of 28, p18 (20 μM) and Arg₈ (SEQ ID NO: 94) (10 μM) entry into UISO-Mel-2 cells after 1 hr at 37° C. in the presence/absence of heparin sulfate (100 μg/ml). (B) Graphs showing flow cytometric analysis of p28 or p18 entry in the presence of inhibitors. Cell fluorescence intensity in the absence of inhibitor (control) was considered as 100%. (C) Graphs depicting FRCS analysis of p28 and p18 entry into fibroblasts in presence of inhibitors. (D) Depicts photographs showing colocalization of p18 and p28 with caveolin I (Panel 1). UISO-Mel-2 cells were incubated with Alexafluor 568 labeled p18 or p28 (20 μM) or media for 2 hrs at 37° C. Cells were fixed and processed for anti-caveolin 1 immunostaining. Confocal analysis of entry of Alexafluor 568 labeled p18 or p28 (20 μM) into UISO-Mel-2 cells after 2 hrs at 37° C. followed by antigolgin 97 antibodies (Panel 2). Colocalization of Alexafluor 568 labeled azurin, p28 and p18 (red) with mitotracker (green) (Panel 3) and Lysotracker (green) (Panel 4) dyes in UISO-Mel-2 cells. Cells were incubated at 37° C. with 20 μM azurin, p28, p18 or media only. After 90 min incubation, mitotracker/lysotracker probes were added and cells incubated for 30 min. Cells were counterstained with DAPI (blue). Colocalization of azurin, p28 or p18 appears as a yellow florescence.

FIGS. 20 A and B. Graphs depicting UISO-Mel-2 cells that were incubated with increasing concentrations of azurin, p28, or p18 at 37° C. for 72 hrs. MTT (A); Direct cell count (B). Cell viability (MTT) or cell number in control wells were considered as 100%. Data represent mean±SEM.

FIG. 21, (A) through (C). Graphs and charts depicting peptide binding and entry into cells. (A) UISO-Mel-2 or fibroblast cells (3×10⁵ cells) were suspended in MEME media without phenol red. Reactions were started by adding Alexafluor 568-conjugated p28 at 10, 50, 100, 150, 250, 300 and 400 μM for 30, 60, 90 and 120 sec on ice. Cells were analyzed by flow cytometry. (B) The Kₘ and Vₘₐₓ were calculated by plotting peptide concentration (μM) vs velocity (MFI/sec). (C) Peptide binding and entry was determined using whole Mel2 cells (50,000 cells/ml), were incubated for 30 min at 37° C. with increasing concentrations (0-175 nM) of radiolabeled azurin in the presence/absence of 1000 fold excess of unlabeled p28, or azurin, and radioactivity remaining in the cell pellet counted using a gamma counter. Radioactivity in cells incubated with ¹²⁵I azurin alone was considered total binding; radioactivity in the presence of unlabeled azurin or p28 was considered nonspecific binding. Specific binding was determined by subtracting nonspecific binding from total binding and Scatchard plots generated.

FIG. 22, (A) through (C). Depict side and back photographs of mice with melanoma MEL-23 tumors taken after injection with p28 dye complex at 60 μmolar concentration in 250 μL scans and after injection with control PBS at (A) 24 hours and (B) 48 hours. (C) depicts side and back photographs of mice with melanoma MEL-23 tumors taken after injection with p28 at 200 μM concentration at 24 and 48 hours.

FIG. 23, (A) through (C). Depict side and back photographs of mice with melanoma MEL-23 tumors taken after injection with p18 at 60 μmolar concentration at (A) 17 hours, (B) 24 hours, and (C) 46 hours. (C) also depicts photographs of mouse organs, including the heart, lung, liver, kidney, spleen, and brain, taken 46 hours after injection of p18.

FIGS. 24, (A) and (B). (A) Depicts side and back photographs of mice with tumors taken 12 hours after injection with p18, p28, and arg-8 (SEQ ID NO: 94) at 60 μmolar concentration. (B) Depicts photographs of mouse organs, including mouse brains, taken 12 hours after injection with p18, p28, and arg-8 (SEQ ID NO: 94).

FIGS. 25, (A) and (B). (A) Depicts side and back photographs of mice with melanoma MEL-6 tumors taken 40 hours after injections of 600 μM concentrations of p18 and arg-8 (SEQ ID NO: 94) into tail veins. Animals treated with p18 received 0.5 million cells, and animals treated with arg-8 (SEQ ID NO: 94) received 1 million cells. (B) Depicts photographs of mouse organs taken 40 hours after injections of 600 μM concentrations of p18 and arg-8 (SEQ ID NO: 94).

FIGS. 26, (A) and (B). (A) Depicts side and back photographs of mice with melanoma MEL-23 tumors taken 16 hours after injections of 60 μM concentrations of p28, p18, and arg-8 (SEQ ID NO: 94). (B) Depicts side and back photographs of mice with melanoma MEL-23 tumors taken 24 hours after injections of 60 μM concentrations of p28, p18, and arg-8 (SEQ ID NO: 94).

FIG. 27. Depicts photographs of mouse organs taken 48 hours after injection of 60 μM concentrations of p28 and p18 dye peptide complex into mice with melanoma MEL-23.

FIG. 28. Depicts photographs of mouse organs taken 24 hours after injection of 60 μM concentrations of p28 into mice with MEL-23 tumors and organs.

FIG. 29. Depicts side and back photographs of mice with melanoma MEL-23 tumors taken 16 hours after injections of 60 μM concentrations of p28 and arg-8 (SEQ ID NO: 94).

FIG. 30. Depicts side and back photographs of mice with melanoma MEL-23 tumors taken 16 hours after injections of 60 μM concentrations of p18.

FIG. 31. Depicts side photographs of mice with tumors taken 10 and 24 hours after high dose treatment with 240 μM concentrations of p18, p28, and arg-8 (SEQ ID NO: 94).

FIG. 32. Depicts side and back photographs of mice with MCF-7 tumors and organs taken 28 hours after high dose treatment with 240 μM concentrations of p18, p28, and arg-8 (SEQ ID NO: 94). Also depicts photographs of mouse organs with MCF-7 taken 28 hours after high dose treatment with 240 μM concentrations of p18, p28, and arg-8 (SEQ ID NO: 94).

FIG. 33. Depicts side and back photographs of mice with tumors taken 50 hours after high dose treatment with 240 μM concentrations of p18, p28, and arg-8 (SEQ ID NO: 94).

FIG. 34. Depicts photographs of mouse organs taken 24 hours after injection of 120 μM concentrations of p18, p28, and arg-8 (SEQ ID NO: 94) into the tail veins of mice with HCT-116 tumors and organs.

FIGS. 35, (A) and (B). (A) Depicts photographs of mouse organs taken 24 hours after injection of 120 μM concentrations of p18, p28, and arg-8 (SEQ ID NO: 94) into the tail veins of mice with HCT-116 tumors and organs. (B) Depicts side photographs of mice with HCT-116 tumors taken 21 hours after injection of 120 μM concentrations of p18, p28, and arg-8 (SEQ ID NO: 94) into their tail veins.

FIGS. 36, (A) and (B). (A) Depicts side and back photographs of mice with HCT-116 24 hours after injection with 120 μM concentrations of p28, 47 days after injection of 1 million cells into tail veins. (B) Depicts photographs of mouse organs taken from mice with HCT-116 4 hours after injection with 120 μM concentrations of p28, 47 days after injection of 1 million cells into tail veins.

FIG. 37. Depicts photographs of organs from MEL-6 mice taken 24 hours after treatment with 120 μM concentrations of p18, p28, and arg-8 (SEQ ID NO: 94).

FIGS. 38, (A) and (B). (A) Depicts side and back photographs of MEL-6 mice taken 22 hours after injection of 120 μM concentrations of p18, p28, and arg-8 (SEQ ID NO: 94), and 60 60 μM concentration of arg-8 (SEQ ID NO: 94). (B) Depicts photographs of MEL-6 mouse organs after treatment with 120 μM concentrations of p18, p28, and arg-8 (SEQ ID NO: 94), and 60 μM concentration of arg-8 (SEQ ID NO: 94).

FIGS. 39, (A) and (B). (A) Depicts photographs of organs from HT-1080 mice taken 22 hours after treatment with 60 and 120 μM concentrations of p18, p28, and arg-8 (SEQ ID NO: 94). (B) Depicts side-by-side photographs of brains from HT-1080 mice taken 22 hours after treatment with 60 and 120 μM concentrations of p18, p28, and arg-8 (SEQ ID NO: 94), demonstrating the differences between uptake of p18 and p28 into the brain.

FIG. 40. Depicts side and back photographs of HT-1080 mice during Doxorubicin vs. p28 study taken 16 hours after treatment with 60 and 120 μM concentrations of p18, p28, and arg-8 (SEQ ID NO: 94).

FIGS. 41, (A) and (B). (A) Depicts photographs of organs from HT-1080 mice taken 22 hours after treatment with 60 and 120 μM concentrations of p28 and arg-8 (SEQ ID NO: 94). (B) Depicts side-by-side photographs of brains from HT-1080 mice taken 22 hours after treatment with 60 and 120 μM concentrations of p28 and arg-8 (SEQ ID NO: 94).

FIGS. 46, (A) and (B). Depicts photographs of (A) organs from mice and (B) back views of mice in Balb-C peptide study taken 12 hours after treatment with 60 and 120 μM concentrations of p18, p28, and arg-8 (SEQ ID NO: 94).

FIGS. 47, (A) and (B). Depicts photographs of (A) organs from mice and (B) side views of mice in Balb-C peptide study taken 24 hours after treatment with 60 and 120 μM concentrations of p18, p28, and arg-8 (SEQ ID NO: 94).

FIG. 48. Depicts side and back photographs of MEL-6 mice (0.5 million cells injected via tail vein) 16 hours after injection into tail veins of 60 μM concentrations of p18 and arg-8 (SEQ ID NO: 94). FIG. 49, (A) through (D). Depicts photographs of mouse organs, and specifically mouse brains, after treatment with p18 and p28.

FIG. 50. Depicts photographs of organs from MEL-6 mice taken 24 hours after treatment with p28, p18, and arg-8 (SEQ ID NO: 94).

FIG. 51, (A) through (C). (A) Depicts side and back photographs of MEL-6 mice 3 hours after injection with 60 μM concentrations of p18, p28, and arg-8 (SEQ ID NO: 94). (B) Depicts side and back photographs of MEL-6 mice, and photographs of organs from MEL-6 mice, taken 22 hours after injection with 60 μM concentrations of p18, p28, and arg-8 (SEQ ID NO: 94). (C) Depicts photographs of organs from MEL-6 mice 24 hours after injection with 60 μM concentrations of p18, p28, and arg-8 (SEQ ID NO: 94). FIGS. 52, (A) and (B). Depict uptake of p18 and p28 into (A) mouse brains and (B) mouse organs).

FIG. 53. Depicts side and back photographs of MEL-6 mice in study whereby 0.5 million cells injected I.V. into tail vein (44 days post), taken 120 hours after injection into tail vein of 24 μM concentrations of p18 and arg-8 (SEQ ID NO: 94).

FIG. 54. Depicts photographs of organs from MEL-6 mice taken 168 hours after treatment with p18.

FIG. 55. Depicts side and back photographs of MEL-6 mice taken after injection of arg-8 (SEQ ID NO: 94) and p18, 72 hrs, day 41 post injection.

FIG. 56. Depicts back photographs of mice taken after injection of arg-8 (SEQ ID NO: 94) and p18.

FIG. 57. Depicts side and front photographs of mice taken 3, 24, and 48 hours after injection of arg-8 (SEQ ID NO: 94) and p18.

### DETAILED DESCRIPTION OF THE INVENTION

### Definitions

As used herein, the term “cell” includes either the singular or the plural of the term, unless specifically described as a “single cell.”

As used herein, the terms “polypeptide,” “peptide,” and “protein” are used interchangeably to refer to a polymer of amino acid residues. The terms apply to amino acid polymers in which one or more amino acid residue is an artificial chemical analogue of a corresponding naturally occurring amino acid. The terms also apply to naturally occurring amino acid polymers. The terms “polypeptide,” “peptide,” and “protein” are also inclusive of modifications including, but not limited to, glycosylation, lipid attachment, sulfation, gamma-carboxylation of glutamic acid residues, hydroxylation and ADP-ribosylation. It will be appreciated that polypeptides are not always entirely linear. For instance, polypeptides may be branched as a result of ubiquitination and they may be circular (with or without branching), generally as a result of post-translation events, including natural processing event and events brought about by human manipulation which do not occur naturally. Circular, branched and branched circular polypeptides may be synthesized by non-translation natural process and by entirely synthetic methods as well.

As used herein, the term “pharmacologic activity” means the effect of a drug or other chemical on a biological system. The effect of chemical may be beneficial (therapeutic) or harmful (toxic). The pure chemicals or mixtures may be of natural origin (plant, animal, or mineral) or may be synthetic compounds.

As used herein, the term “premalignant” means precancerous, or before abnormal cells divide without control.

As used herein, the term “lesion” means an area of abnormal tissue.

As used herein, the term “pathological condition” includes anatomic and physiological deviations from the normal that constitute an impairment of the normal state of the living animal or one of its parts, that interrupts or modifies the performance of the bodily functions, and is a response to various factors (as malnutrition, industrial hazards, or climate), to specific infective agents (as worms, parasitic protozoa, bacteria, or viruses), to inherent defects of the organism (as genetic anomalies), or to combinations of these factors.

As used herein, the term “condition” includes anatomic and physiological deviations from the normal that constitute an impairment of the normal state of the living animal or one of its parts, that interrupts or modifies the performance of the bodily functions. A “condition” may be, but is not limited to an ailment, disease, infection or illness.

As used herein, the term “suffering from” includes presently exhibiting the symptoms of a pathological condition, having a pathological condition even without observable symptoms, in recovery from a pathological condition, or recovered from a pathological condition.

As used herein, the term “chemoprevention” is the use of drugs, vitamins, or other agents to try to reduce the risk of, or delay the development or recurrence of, cancer.

A used herein, the term “treatment” includes preventing, lowering, stopping, or reversing the progression or severity of the condition or symptoms associated with a condition being treated. As such, the term “treatment” includes medical, therapeutic, and/or prophylactic administration, as appropriate. Treatment may also include preventing or lessening the development of a condition, such as cancer.

As used herein, the term “inhibit cell growth” means the slowing or ceasing of cell division and/or cell expansion. This term also includes the inhibition of cell development or increases in cell death.

As used herein, the term “inhibit the growth of HIV infection” means any means by which HIV infection is decreased, or prevented from increasing in the human body. These means can include, but are not limited to, inhibition of replication of the HIV genome, inhibition of synthesis and/or assembly of the HIV coat proteins, and inhibition of HIV entry into uninfected cells. This definition includes any the method of action of any of the currently known HIV therapies.

As used herein, “anti-malarial activity” includes any activity that decreases the infectivity, the reproduction, or inhibits the progress of the lifecycle of a malaria parasite. “Anti-malarial activity” includes inhibition of the growth of malaria infection by all of the means of observed with current anti-malarial drugs.

As used herein, the term “anti-malarial drug” refers to drugs with anti-malarial activity that may be used to decrease the infectivity, the reproduction, or inhibit the progress of the lifecycle of a malaria parasite.

As used herein, the term “anti-HIV drug” refers to drugs with anti-HIV activity HIV by which HIV infection in mammals is decreased, or prevented from increasing in the human body, by any means including, but are not limited to, inhibition of replication of the HIV genome, inhibition of synthesis and/or assembly of the HIV coat proteins, and inhibition of HIV entry into uninfected cells.

As used herein, the term “inhibit angiogenesis” refers to the slowing, ceasing or reverse of the formation of blood vessels in a particular cells, tissues, or location of the body. The inhibition of angiogenesis may be due to direct or indirect effects on endothelial cells. The inhibition may also be at any stage of the angiogenesis process. For example, the inhibition may be due to preventing a tumor from producing Vascular Endothelial Growth Factor (VEGF), direct inhibition of endothelial cell proliferation and/or migration, acting as an antagonist of angiogenesis growth factors, inhibition of endothelial-specific integrin/survival signaling, or chelation of copper. The inhibition of angiogenesis may be by any means by which the formation of blood vessels is slowed, ceased or reversed, including any means currently used by any anti-angiogenesis drug under development or on the market.

As used herein, the term “inappropriate angiogenesis” refers to any occurrence of angiogenesis that is undesirable. Inappropriate angiogenesis may be angiogenesis that is associated with a condition in a mammal. The inappropriate angiogenesis may be either the cause or the symptom of such a condition. Inappropriate angiogenesis in a broader sense may be any angiogenesis that is unwanted, even though it may be within the realm of normal mammalian physiology.

A “therapeutically effective amount” is an amount effective to prevent or slow the development of, or to partially or totally alleviate the existing symptoms in a particular condition for which the subject is being treated. Determination of a therapeutically effective amount is well within the capability of those skilled in the art.

The term “substantially pure,” as used herein, when used to modify a protein or other cellular product of the invention, refers to, for example, a protein isolated from the growth medium or cellular contents, in a form substantially free of, or unadulterated by, other proteins and/or other compounds. The term “substantially pure” refers to a factor in an amount of at least about 75%, by dry weight, of isolated fraction, or at least “75% substantially pure.” More specifically, the term “substantially pure” refers to a compound of at least about 85%, by dry weight, of isolated fraction, or at least “85% substantially pure.” Most specifically, the term “substantially pure” refers to a compound of at least about 95%, by dry weight, of isolated fraction, or at least “95% substantially pure.” The term “substantially pure” may also be used to modify a synthetically-made protein or compound of the invention, where, for example, the synthetic protein is isolated from the reagents and by-products of the synthesis reaction(s).

The term “pharmaceutical grade,” as used herein, when referring to a peptide or compound of the invention, is a peptide or compound that is isolated substantially or essentially from components which normally accompany the material as it is found in its natural state, including synthesis reagents and by-products, and substantially or essentially isolated from components that would impair its use as a pharmaceutical. For example, a “pharmaceutical grade” peptide may be isolated from any carcinogen. In some instances, “pharmaceutical grade” may be modified by the intended method of administration, such as “intravenous pharmaceutical grade,” in order to specify a peptide or compound that is substantially or essentially isolated from any substance that would render the composition unsuitable for intravenous administration to a patient. For example, an “intravenous pharmaceutical grade” peptide may be isolated from detergents, such as SDS, and anti-bacterial agents, such as azide.

The terms “isolated,” “purified” or “biologically pure” refer to material which is substantially or essentially free from components which normally accompany the material as it is found in its native state. Thus, isolated peptides in accordance with the invention preferably do not contain materials normally associated with the peptides in their in situ environment. An “isolated” region of a polypeptide refers to a region that does not include the whole sequence of the polypeptide from which the region was derived. An “isolated” nucleic acid, protein, or respective fragment thereof has been substantially removed from its in vivo environment so that it may be manipulated by the skilled artisan, such as but not limited to, nucleotide sequencing, restriction digestion, site-directed mutagenesis, and subcloning into expression vectors for a nucleic acid fragment as well as obtaining the protein or protein fragment in substantially pure quantities.

The term “substantially pure”, when used to modify the term a polypeptide or other compound, as used herein, refers to a polypeptide or compound, for example, a polypeptide isolated from the growth medium, in a form substantially free of, or unadulterated by, active inhibitory agents. The term “substantially pure” refers to a compound in an amount of at least about 75%, by dry weight, of isolated fraction, or “75% substantially pure.” More specifically, the term “substantially pure” refers to a compound of at least about 85%, by dry weight, active compound, or “85% substantially pure.” Most specifically, the term “substantially pure” refers to a compound of at least about 95%, by dry weight, active compound, or “95% substantially pure.” The substantially pure cupredoxin or cytochrome or a variant or derivative thereof can be used in combination with one or more other substantially pure compounds, or another isolated cupredoxin or cytochrome.

The term “variant” as used herein with respect to a peptide, refers to amino acid sequence variants which may have amino acids replaced, deleted, or inserted as compared to the wild-type polypeptide. Variants may be truncations of the wild-type peptide. A “deletion” is the removal of one or more amino acids from within the polypeptide, while a “truncation” is the removal of one or more amino acids from one or both ends of the polypeptide. Thus, a variant peptide may be made by manipulation of genes encoding the polypeptide. A variant may be made by altering the basic composition or characteristics of the polypeptide, but not at least some of its pharmacologic activities. For example, a “variant” of azurin can be a mutated azurin that retains its ability to inhibit the development of premalignant mammalian cells. In some cases, a variant peptide is synthesized with non-natural amino acids, such as ε-(3,5-dinitrobenzoyl)-Lys residues. Ghadiri & Fernholz, J. Am. Chem. Soc., 112:9633-9635 (1990). In another example, a “variant” of azurin can be a mutated azurin that retains its ability to inhibit the growth of HIV infection in mammalian cells. In another example, a “variant” of azurin can be a mutated azurin that retains its ability to inhibit parasitemia in malaria-infected human red blood cells. In some embodiments, the variant has not more than 20 amino acids replaced, deleted or inserted compared to wild-type peptide or part thereof. In some embodiments, the variant has not more than 15 amino acids replaced, deleted or inserted compared to wild-type peptide or part thereof. In some embodiments, the variant has not more than 10 amino acids replaced, deleted or inserted compared to wild-type peptide or part thereof. In some embodiments, the variant has not more than 6 amino acids replaced, deleted or inserted compared to wild-type peptide or part thereof. In some embodiments, the variant has not more than 5 amino acids replaced, deleted or inserted compared to wild-type peptide or part thereof. In some embodiments, the variant has not more than 3 amino acids replaced, deleted or inserted compared to wild-type peptide or part thereof.

The term “amino acid,” as used herein, means an amino acid moiety that comprises any naturally-occurring or non-naturally occurring or synthetic amino acid residue, i.e., any moiety comprising at least one carboxyl and at least one amino residue directly linked by one, two three or more carbon atoms, typically one (a) carbon atom.

The term “derivative” as used herein with respect to a peptide refers to a peptide that is derived from the subject peptide. A derivation includes chemical modifications of the peptide such that the peptide still retains some of its fundamental activities. For example, a “derivative” of azurin can, for example, be a chemically modified azurin that retains its ability to inhibit angiogenesis in mammalian cells. Chemical modifications of interest include, but are not limited to, amidation, acetylation, sulfation, polyethylene glycol (PEG) modification, phosphorylation or glycosylation of the peptide. In addition, a derivative peptide may be a fusion of a polypeptide or fragment thereof to a chemical compound, such as but not limited to, another peptide, drug molecule or other therapeutic or pharmaceutical agent or a detectable probe.

The term “percent (%) amino acid sequence identity” is defined as the percentage of amino acid residues in a polypeptide that are identical with amino acid residues in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Often publicly available computer software such as BLAST, BLAST2, ALIGN2 or Megalign (DNASTAR) software is used to align peptide sequences. In a specific embodiment, Blastp (available from the National Center for Biotechnology Information, Bethesda Md.) is used using the default parameters of long complexity filter, expect 10, word size 3, existence 11 and extension 1.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:

% amino acid sequence identity=X/Y*100
where
X is the number of amino acid residues scored as identical matches by the sequence alignment program's or algorithm's alignment of A and B and
Y is the total number of amino acid residues in B.

If the length of amino acid sequence A is not equal to the length of amino acid sequence B, the % amino acid sequence identity of A to B will not equal the % amino acid sequence identity of B to A. When comparing longer sequences to shorter sequences, the shorter sequence will be the “B” sequence. For example, when comparing truncated peptides to the corresponding wild-type polypeptide, the truncated peptide will be the “B” sequence.

### General

The present invention provides compositions comprising cupredoxin or cytochrome, and variants, derivatives, truncations, and structural equivalents of cupredoxin or cytochrome, and methods to treat and/or prevent two or more conditions in mammalian cells.

The invention also provides methods to administer to a patient to treat and/or prevent two or more diseases in a patient, comprising administering to the patient with one peptide or at least two peptides that are a cupredoxin, cytochrome and variants, derivatives, truncations, and structural equivalents of cupredoxin or cytochrome.

Specifically, the invention provides compositions comprising Pseudomonas aeruginosa azurin, variants, derivatives, truncations, and structural equivalents of azurin, and their use to concurrently treat and/or prevent two or more conditions in a patient. More specifically, the present invention provides compositions for the concurrent treatment and/or prevention of conditions such as cancer, inappropriate angiogenesis, HIV and malaria, and patients at a higher risk of acquiring these conditions than the general population.

Members of the cupredoxin family, specifically azurin from Pseudomonas aeruginosa, are promising compounds for therapeutic and preventative treatment of numerous diseases or conditions. For example, azurin is known to inhibit angiogenesis in human umbilical vascular endothelium cells (HUVECs). U.S. patent application Ser. No. 11/488,693, filed Jul. 19, 2006, which is hereby incorporated by reference in its entirety herein. Azurin from P. aeruginosa is also known for its ability to inhibit the growth of HIV-1 infection in peripheral blood mononuclear cells and to inhibit parasitemia of malaria-infected mammalian red blood cells. Chaudhari et al., Cell Cycle. 5: 1642-1648 (2006). Azurin from P. aeruginosa is also known to interfere with the ephrin signaling system in various mammalian cells and tissues. U.S. patent application Ser. No. 11/436,592, filed May 19, 2006, which is hereby incorporated by reference in its entirety herein.

Furthermore, two redox proteins elaborated by Pseudomonas aeruginosa, the cupredoxin azurin and cytochrome c₅₅₁, both enter J774 lung cancer cells and show significant cytotoxic activity toward the cancer cells as compared to normal cells. Zaborina et al., Microbiology 146:2521-2530 (2000). Azurin can also selectively enter and kill human melanoma UISO-Mel-2 or human breast cancer MCF-7 cells. Yamada et al., PNAS 99:14098-14103 (2002); Punj et al., Oncogene 23:2367-2378 (2004). Azurin from P. aeruginosa preferentially enters J774 murine reticulum cell sarcoma cells, forms a complex with and stabilizes the tumor suppressor protein p53, enhances the intracellular concentration of p53, and induces apoptosis. Yamada et al., Infection and Immunity 70:7054-7062 (2002). Detailed studies of various domains of the azurin molecule showed that amino acids 50-77 (p28) (SEQ ID NO: 29) represented a protein transduction domain (PTD) critical for internalization and subsequent apoptotic activity. Yamada et al., Cell. Microbial. 7:1418-1431 (2005). Azurin also caused a significant increase of apoptosis in human osteosarcoma cells as compared to non-cancerous cells. Ye et al., Ai Zheng 24:298-304 (2003).

Moreover, other members of the Cupredoxin family are promising compounds for therapeutic and preventative treatment of numerous diseases or conditions. Rusticyanin from Thiobacillus ferrooxidans can also enter macrophages and induce apoptosis. Yamada et al., Cell Cycle 3:1182-1187 (2004); Yamada et al., Cell. Micro. 7:1418-1431 (2005). Plastocyanin from Phormidium laminosum and pseudoazurin form Achromobacter cycloclastes also are cytotoxic towards macrophages. U.S. Pat. Pub. No. 20060040269, published Feb. 23, 2006.

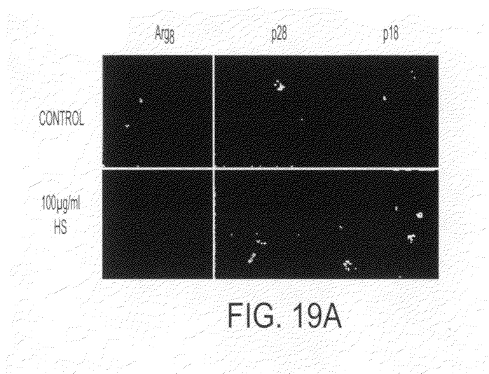

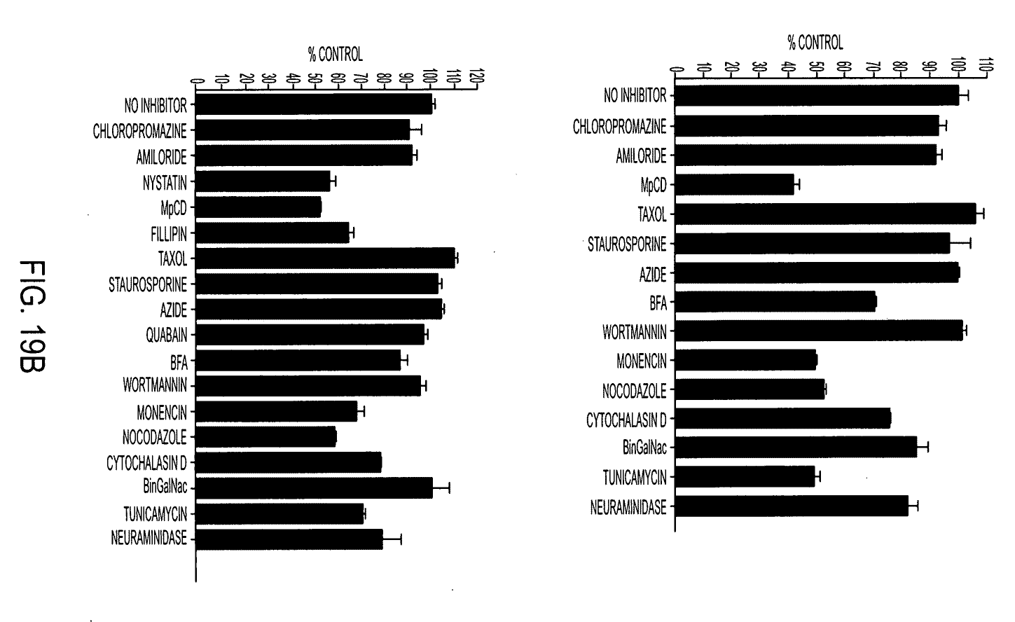

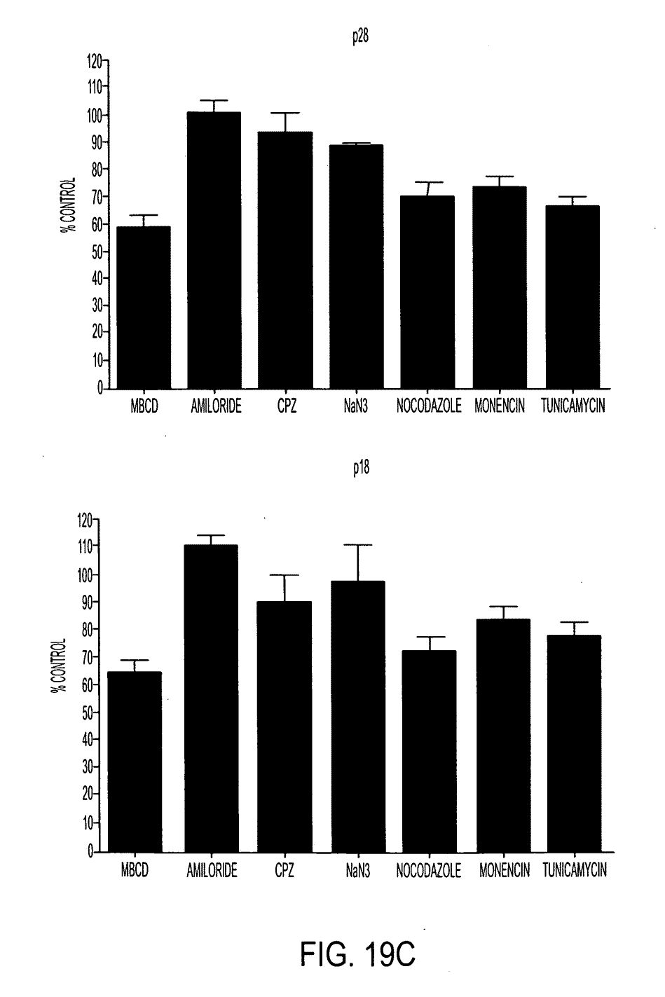

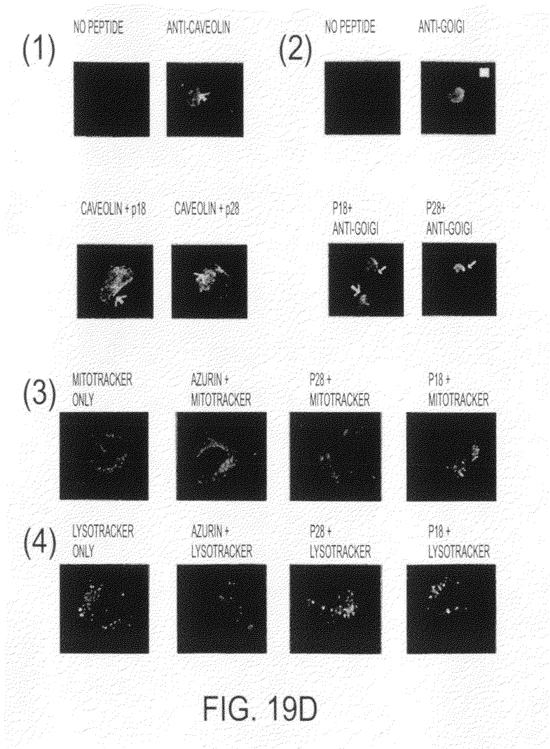

The temperature dependent entry of cationic cell penetrating peptides (“CPPs”), which supports an endocytotic component to cell penetration, is reflected in the entry of azurin and aa fragment 50-77 (p28). Yamada, T., et al., Cell Microbiol 7: 1418-1431 (2005). The entry of 50-67 of azurin (p18) into normal and malignant cells appears accelerated relative to p28. The lower Kₘ and higher Vₘₐₓ of p18 suggest that aa 50-67 define an amphipathic structure when associated with phospholipid membranes that more closely represents the actual PTD of azurin. However, an energy dependent endocytotic or pore related process is not the only entry mechanism available to these peptides. For example, the metabolic and membrane potential inhibitors sodium azide and ouabain (Na⁺ K⁺ ATPase inhibitor), which inhibit the entry of cationic peptides, did not impair the entry of either p18 or p28 into UISO-Mel-2 cells or fibroblasts (FIG. 19 B,C), suggesting that either peptide may penetrate the cell membrane directly.

Depletion of cholesterol from the plasma membrane with β-methylcylodextran, filipin or nystatin to disrupt lipid rafts, plasma membrane domains that provide fluid platforms to segregate membrane components and compartmentalize membranes, significantly inhibited the penetration of p18 (50%) and p28 (˜60%) into UISO-Mel-2 cells and fibroblasts (35% and 42%, respectively) demonstrating that a significant percentage (˜60%) of p18 and p28 penetrates the plasma membrane via caveolae. Caveolae are a 50- to 100-nm omega-shaped subset of lipid raft invaginations of the plasma membrane defined by the presence of caveolin specific proteins (caveolin-1, -2, or -3) that function as regulators of signal transduction.

Brefeldin A disrupts the Golgi apparatus and inhibited p18 accumulation, so it follows that this pathway is also utilized in p18 and p28 entry and intracellular transport. Cell penetration of p18 and p28 via caveolae comports with the evidence that inhibitors of N-glycosylation reduce cell entry by ˜60% in UISO-Mel-2 cells and 25% and 35% respectively in fibroblasts. The percentile differences between p18 and p28 entry relate to the numbers of N-glycosylation membrane structures in cancer vs normal cells and the relative route of entry of p28 and p18 via this mechanism. FIG. 19 B, C.

Azurin, p28, and p18 all bind to cancer cells with high affinity and high capacity relative to many other potential anti-cancer peptides. It is believed that after binding, this protein/receptor complex localizes in caveolae and is internalized, eventually moving (via caveosomes) to the golgi, ER, and nucleus. In addition to caveolar-mediated entry, kinetic analysis also demonstrates that p28 and p18 penetrate the plasma membrane via a non-clathrin caveolae mediated process. A clathrin- and caveolin-independent pathway can exist as a constitutive internalization mechanism, such as for the interleukin 2 receptor and for certain glycosyl-phosphatidylinositol (GPI)-anchored proteins. Lamaze, C., et al., Mol Cell 7: 661-671 (2001); Sabharanjak, S., et al., Dev Cell, 2: 411-423 (2002). An increase in caveolin-1 expression in cancer cells over normal cells is not likely to be the sole basis for the preferential entry of azurin, p28 and p18 into cancer cells. Fibroblasts and a number of other normal cells also have significant numbers of caveolae on their surface.

The findings reflected in Examples 25-31 demonstrate that the cellular penetration of aa 50-67 and 50-77 of azurin is unique relative to all current CPPs in its preference for cancer cells, and show that the C-terminal 10-12 amino acids of p28, aa 50-77 of azurin, contain the domain primarily responsible for cell cycle inhibition and apoptitic activity.

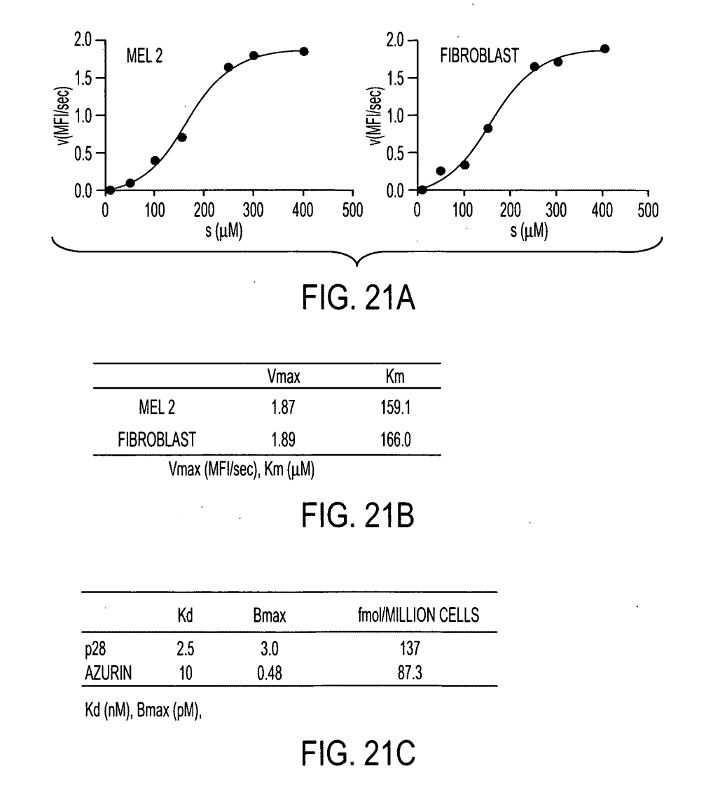

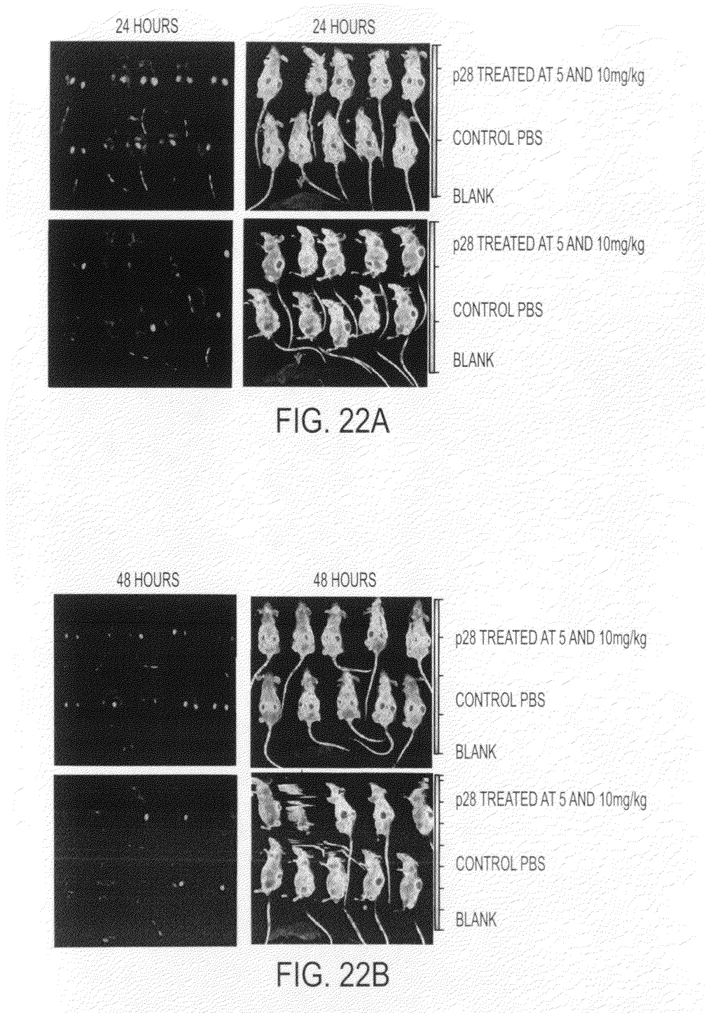

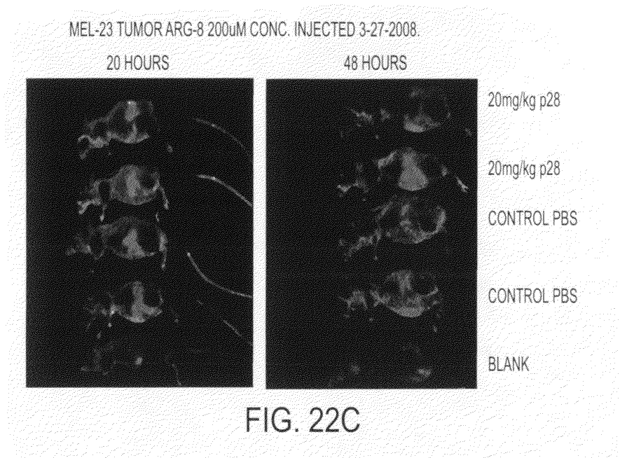

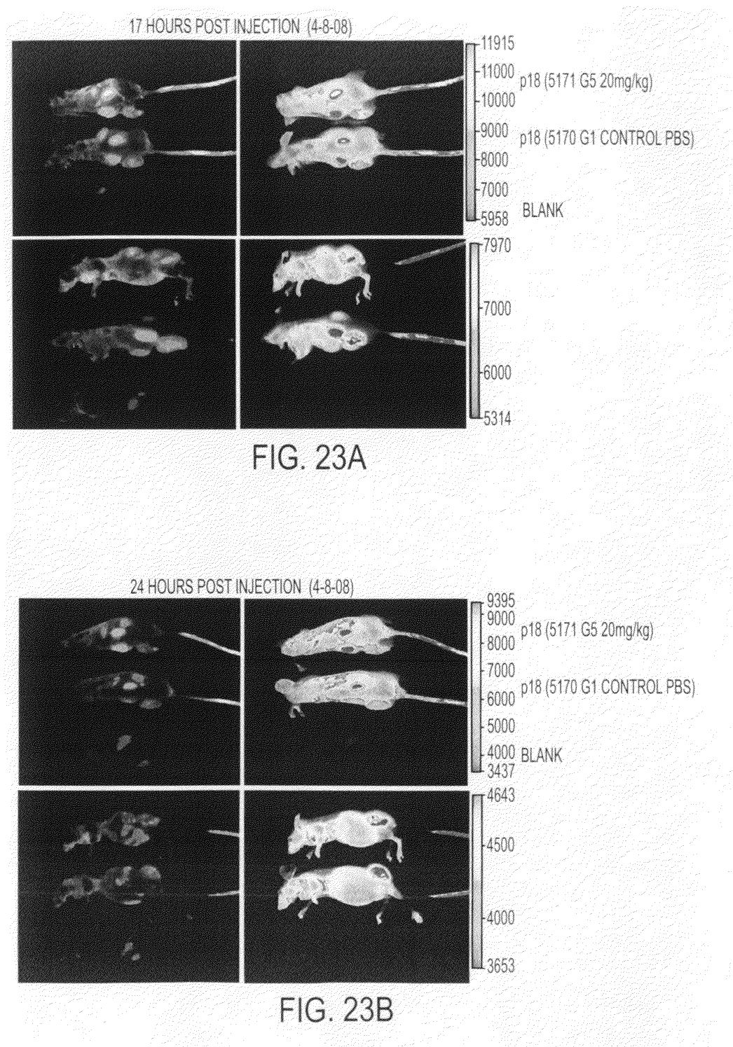

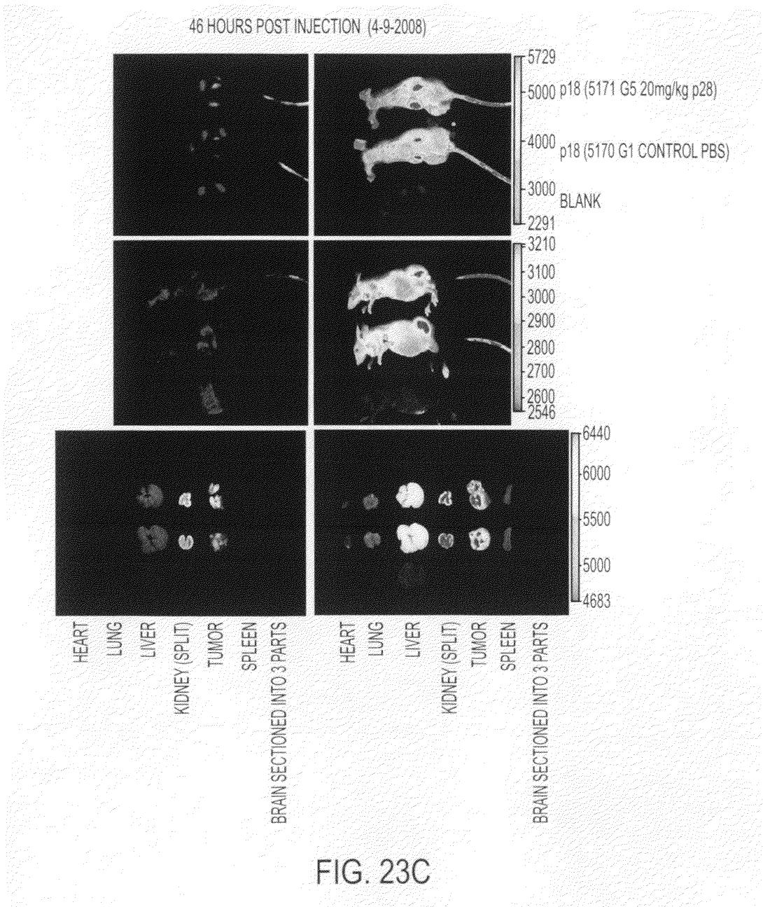

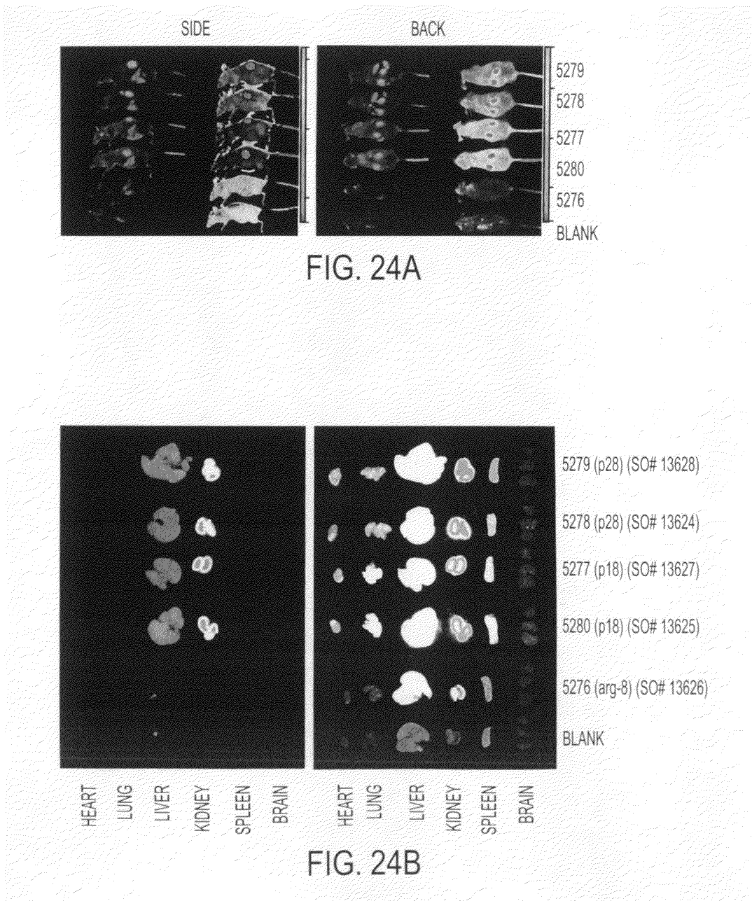

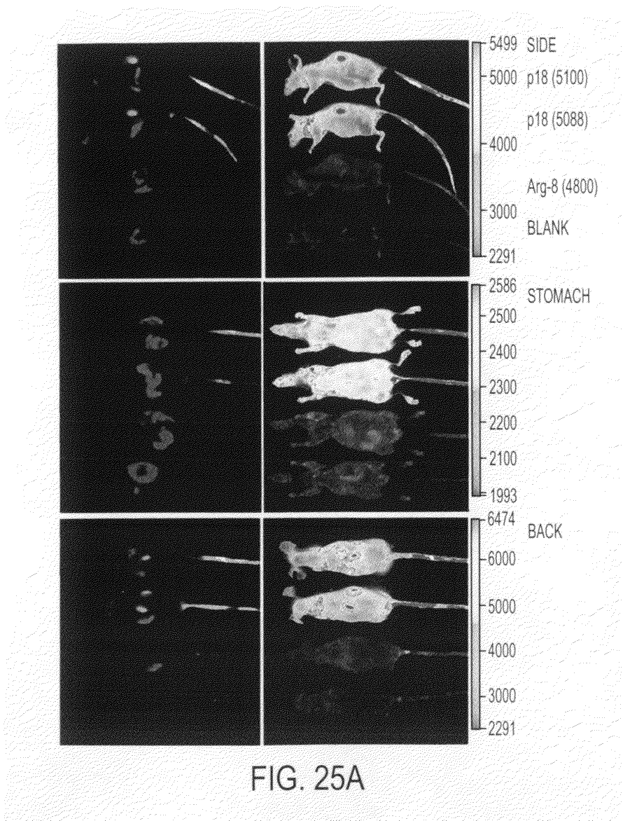

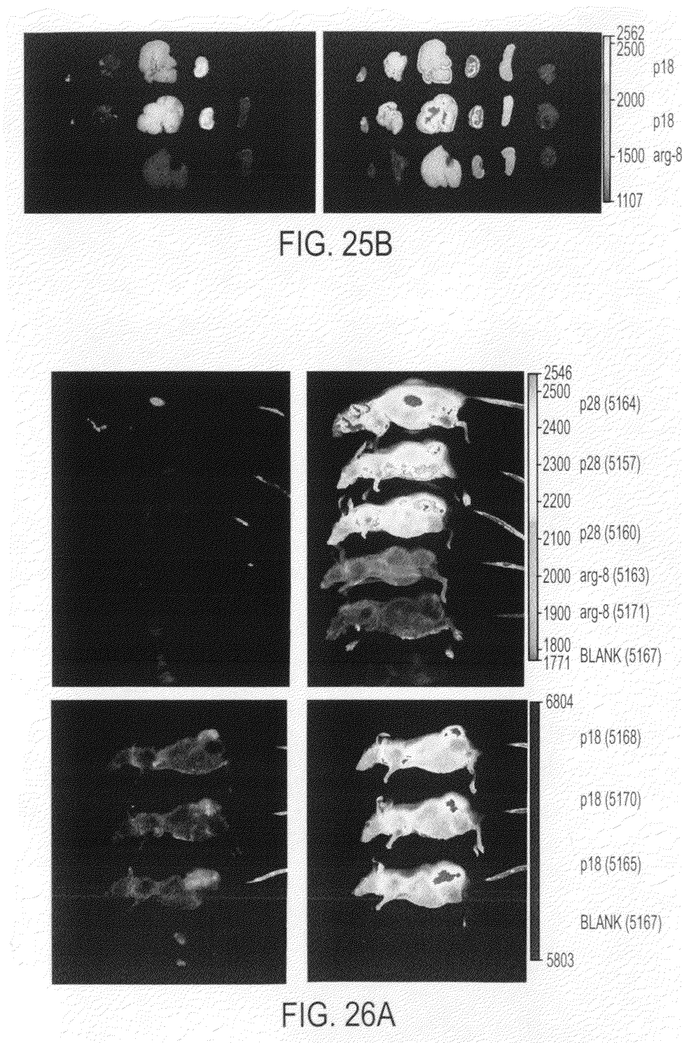

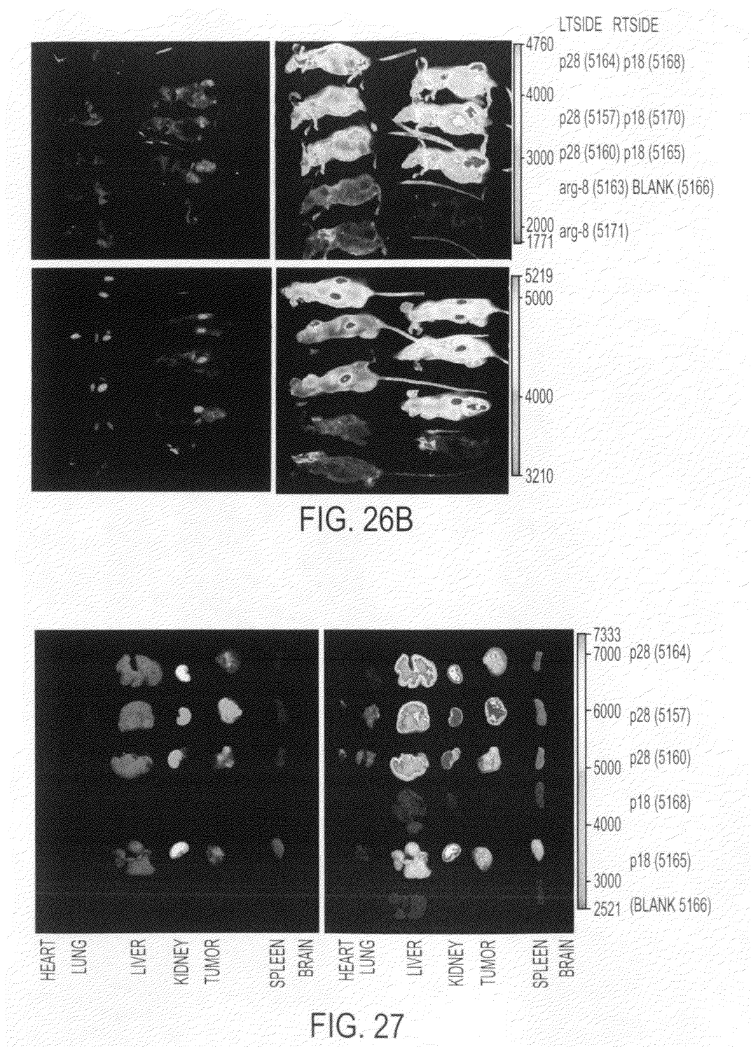

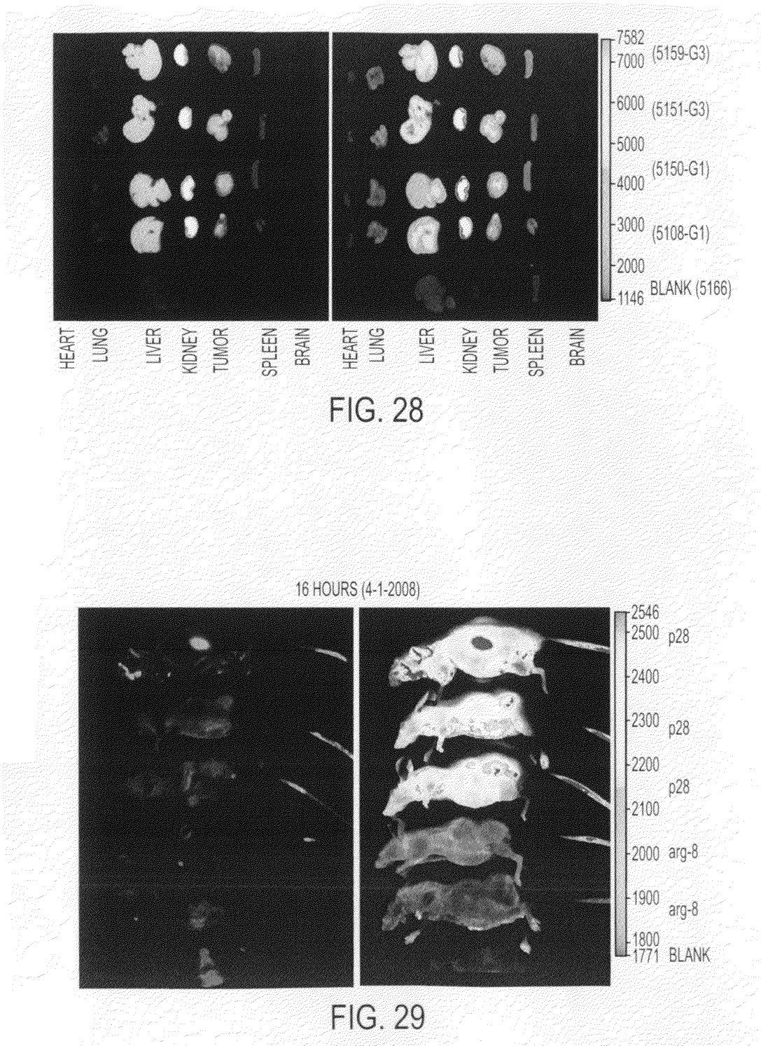

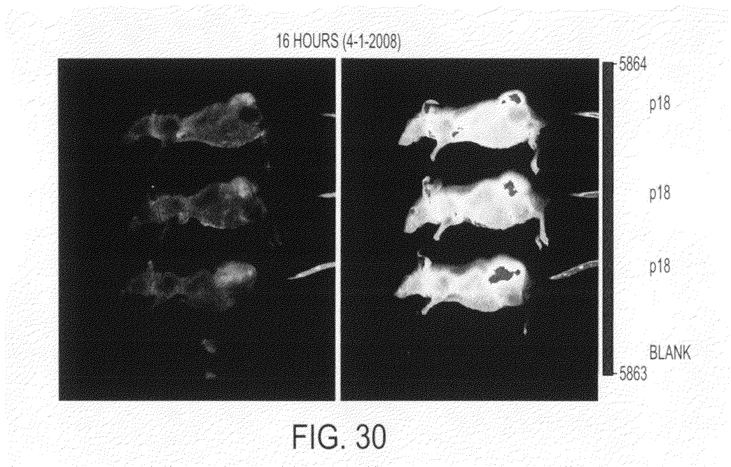

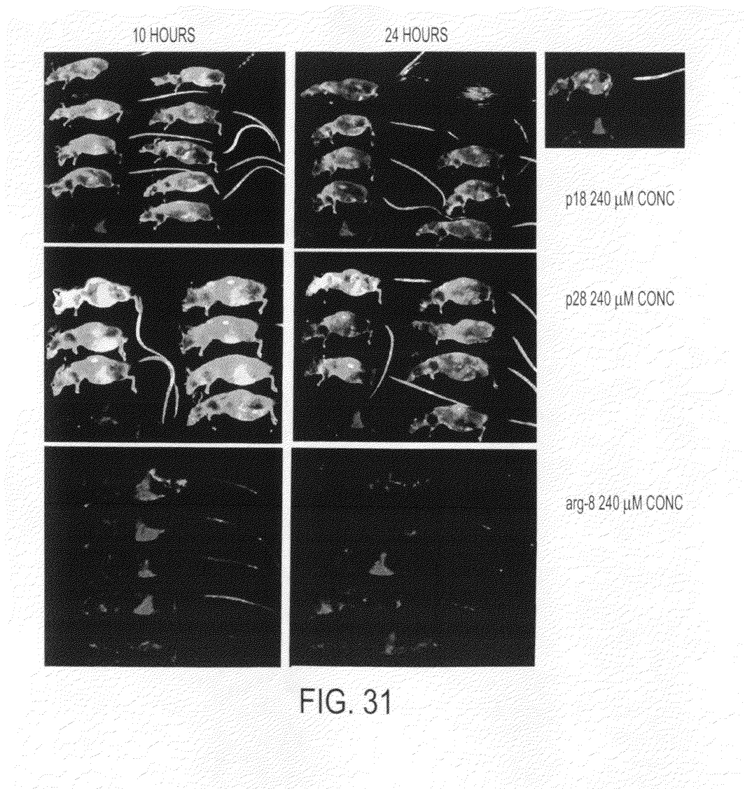

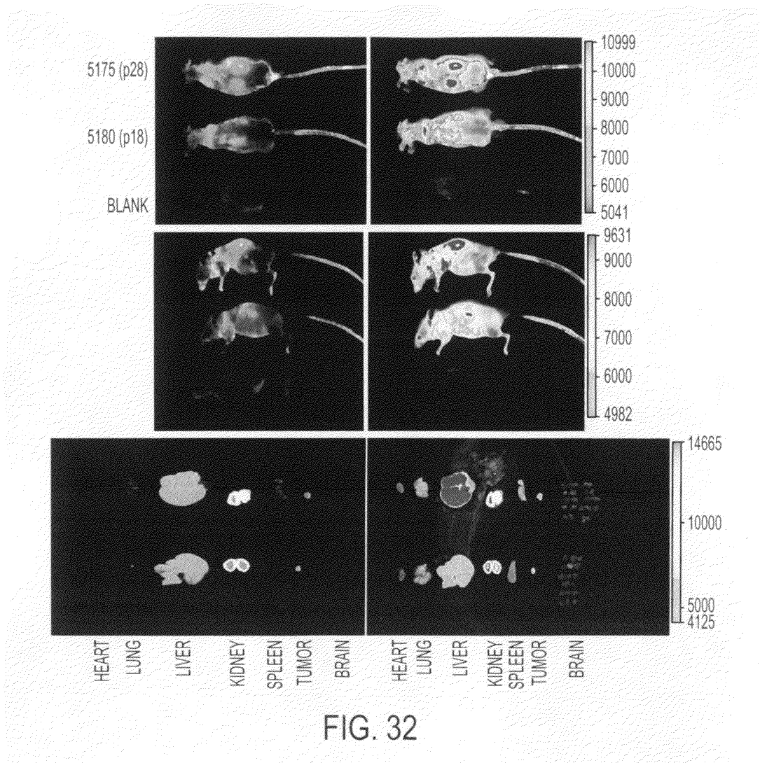

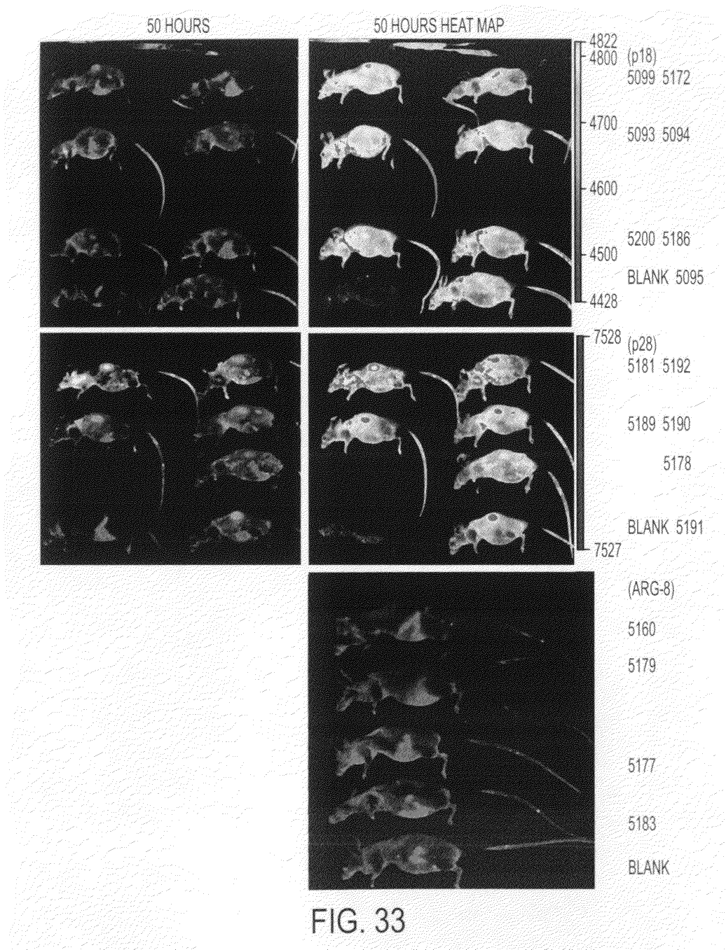

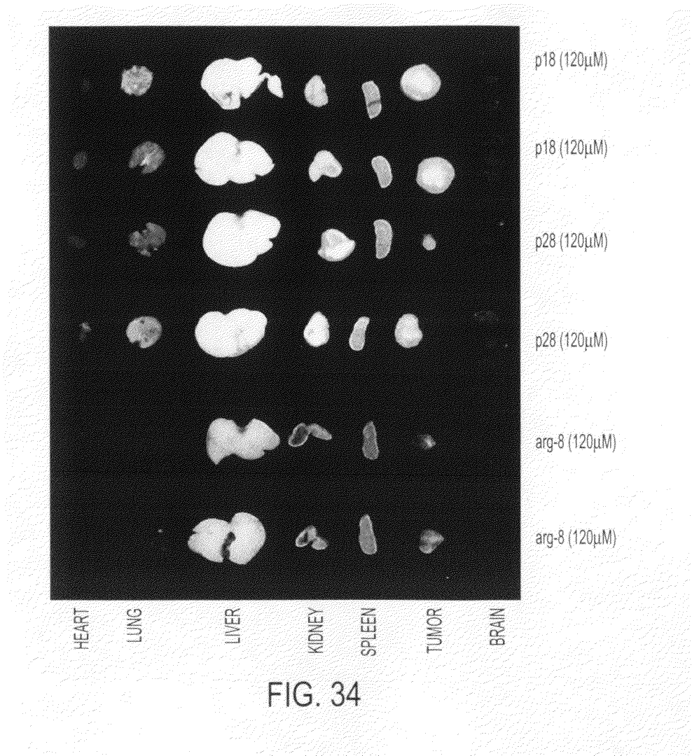

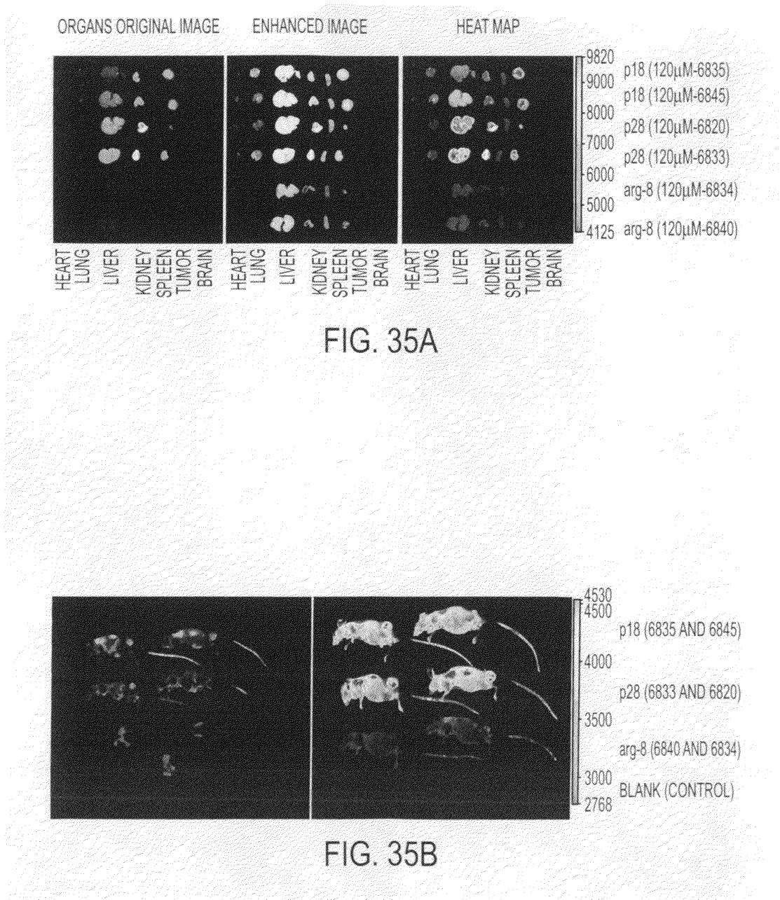

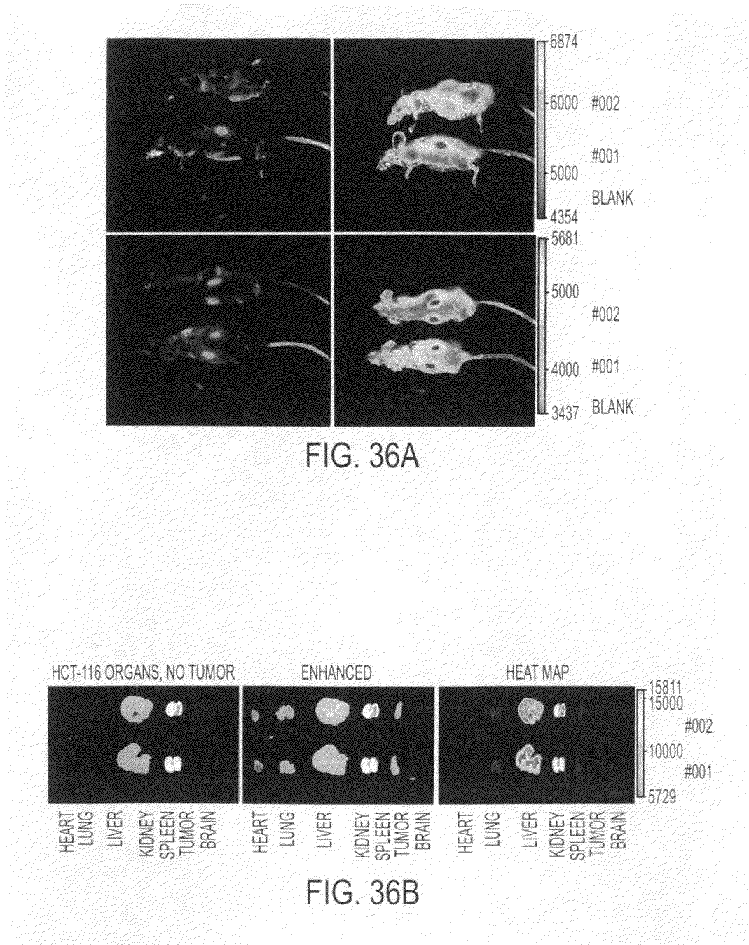

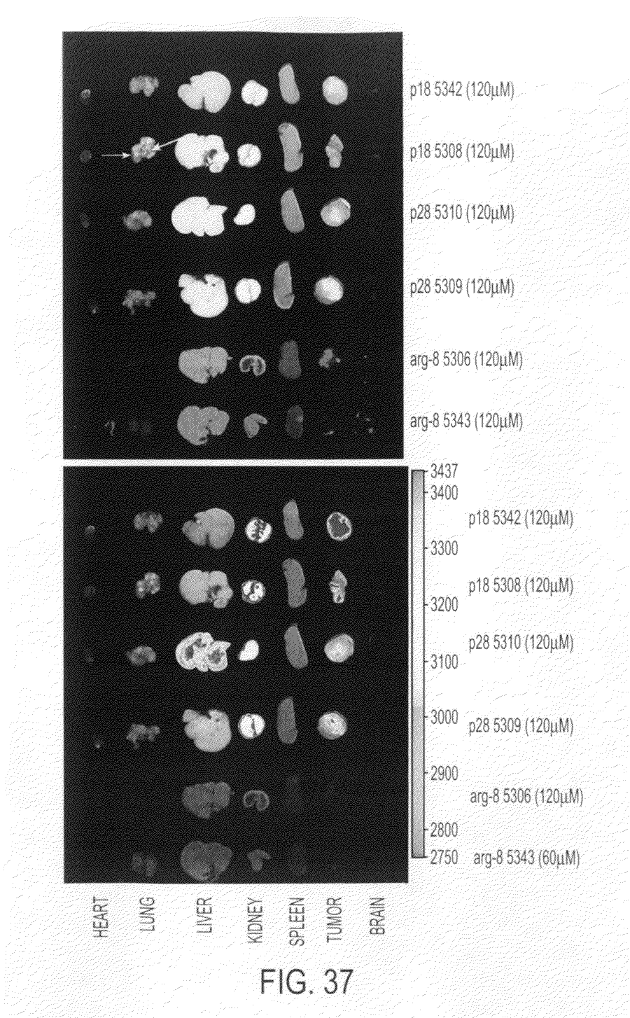

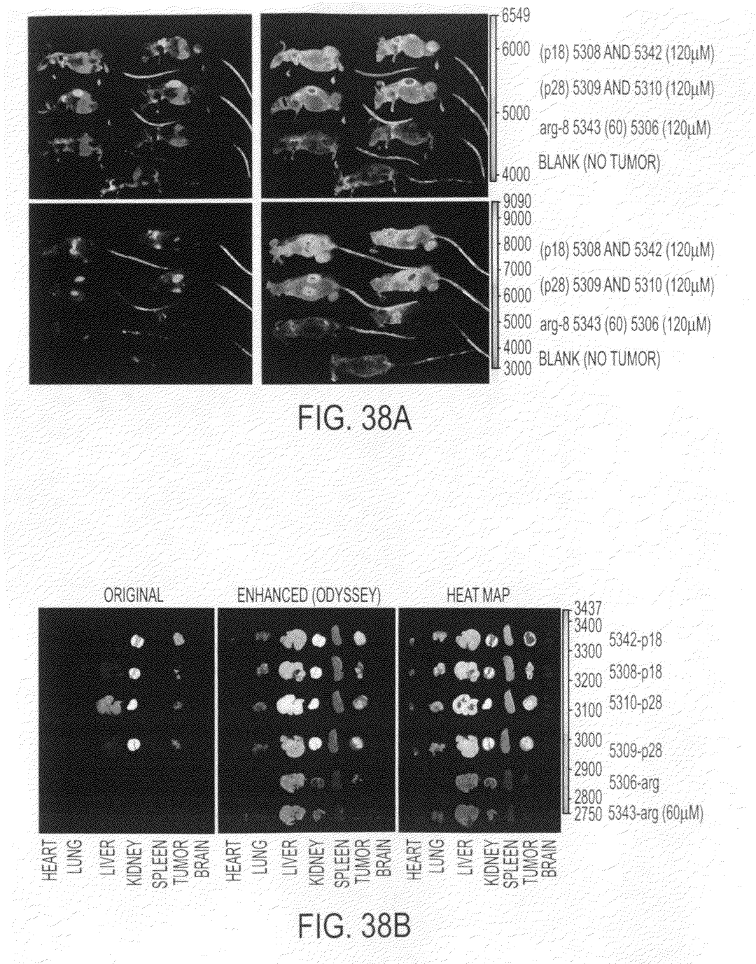

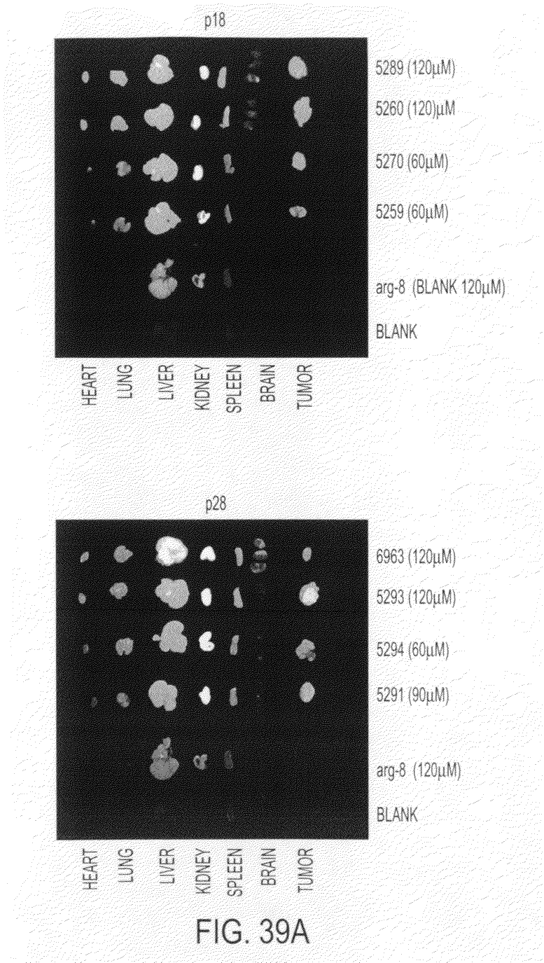

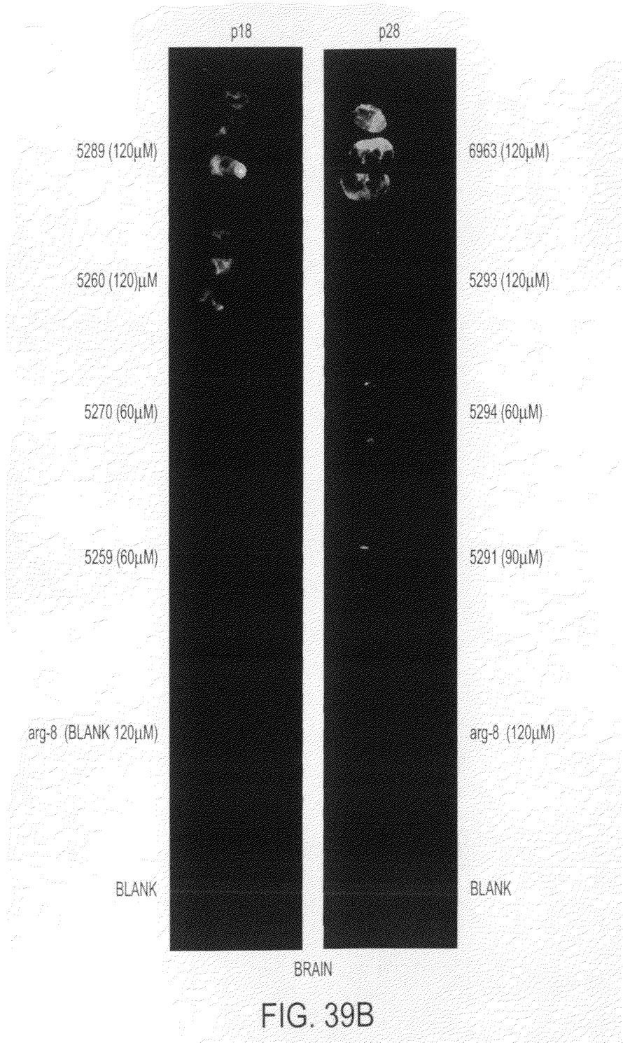

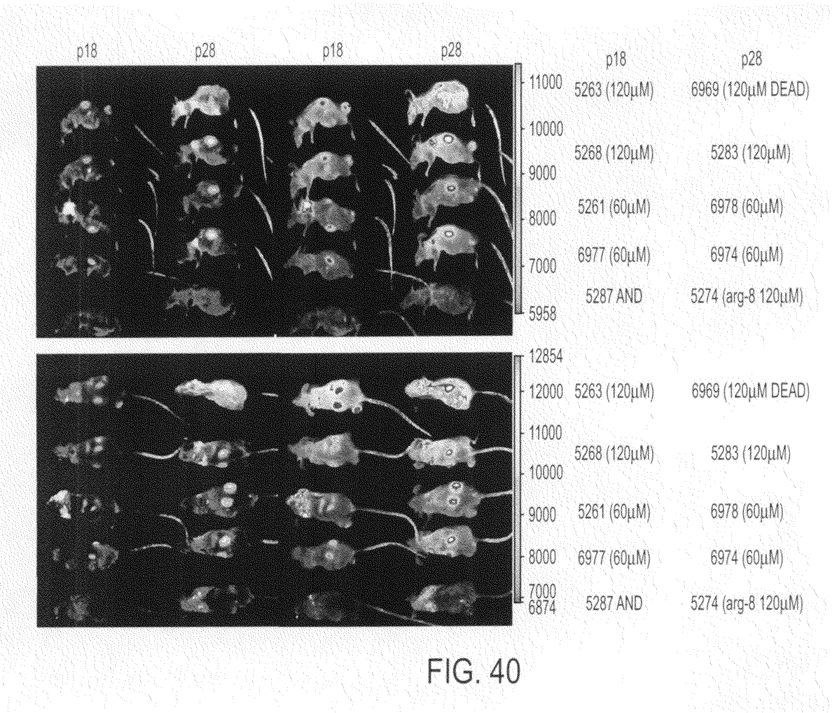

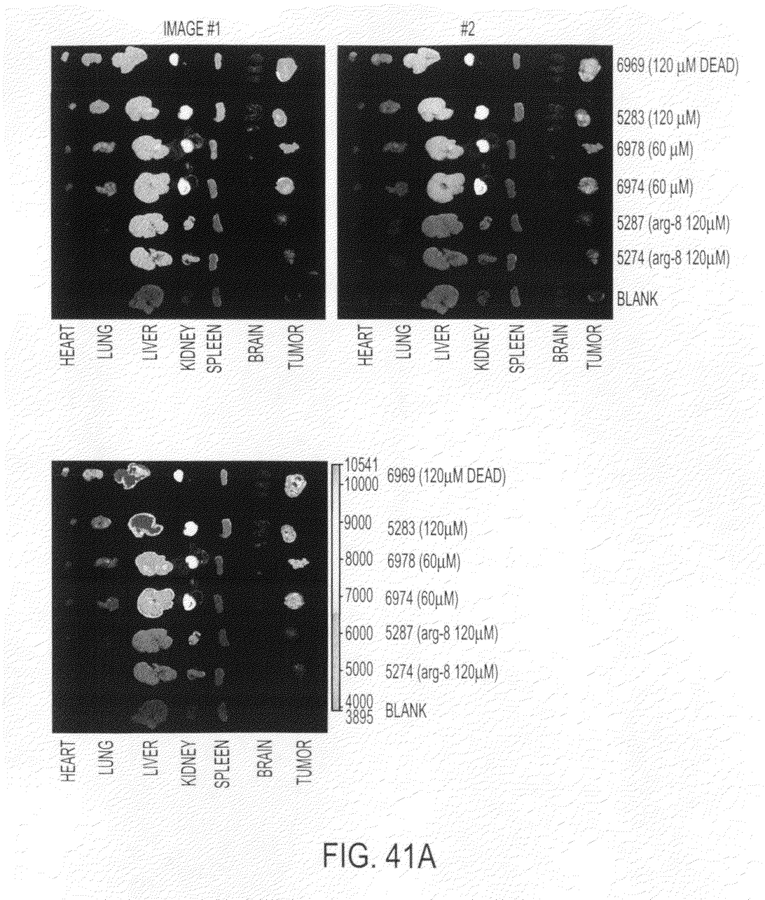

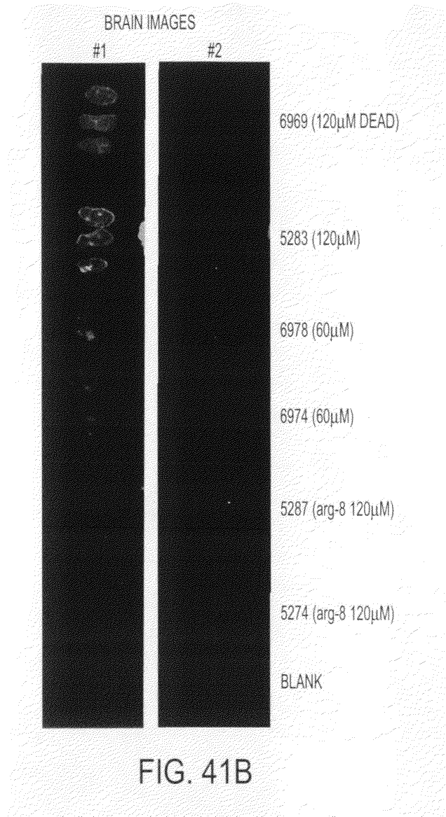

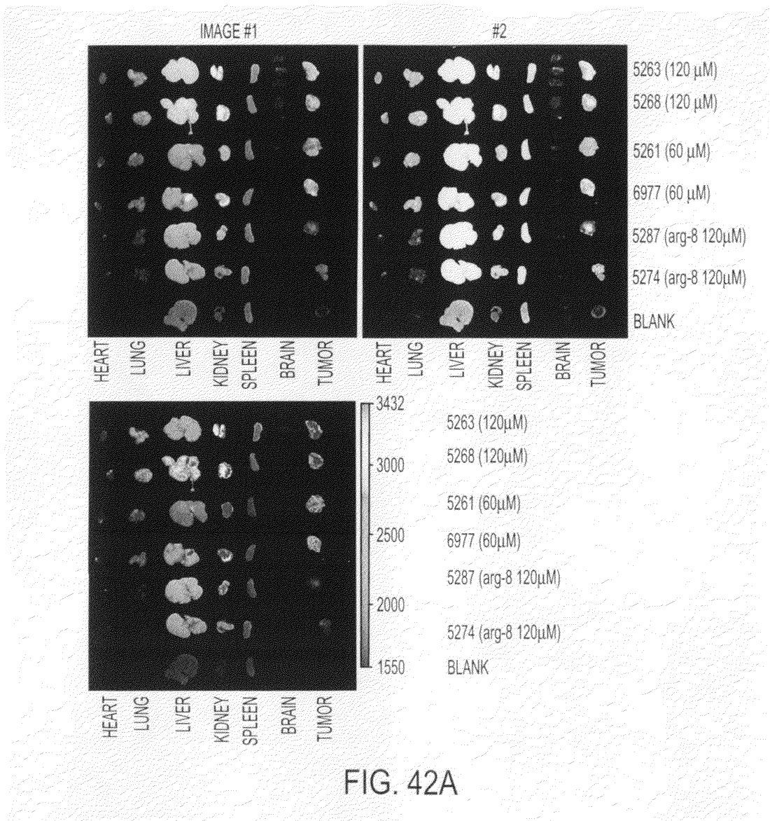

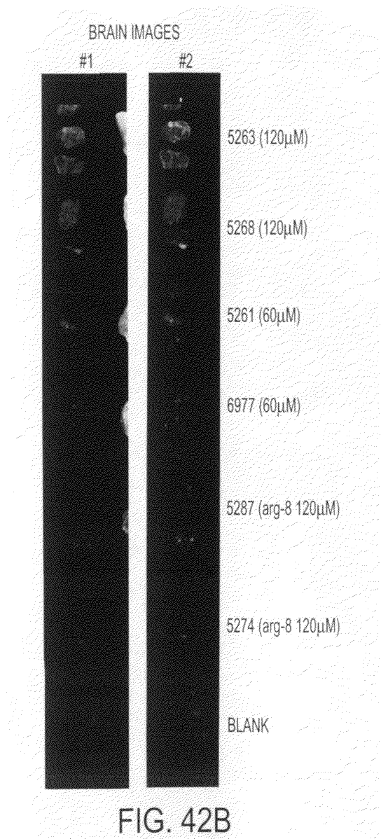

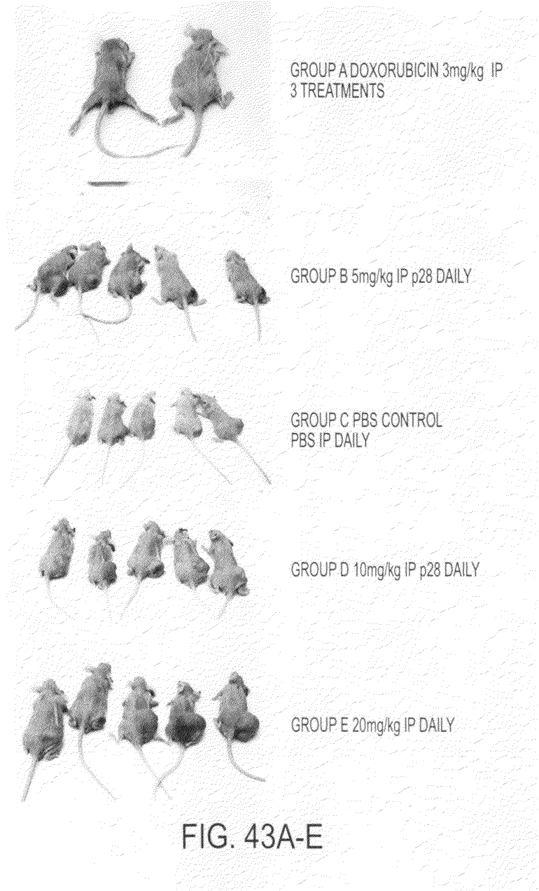

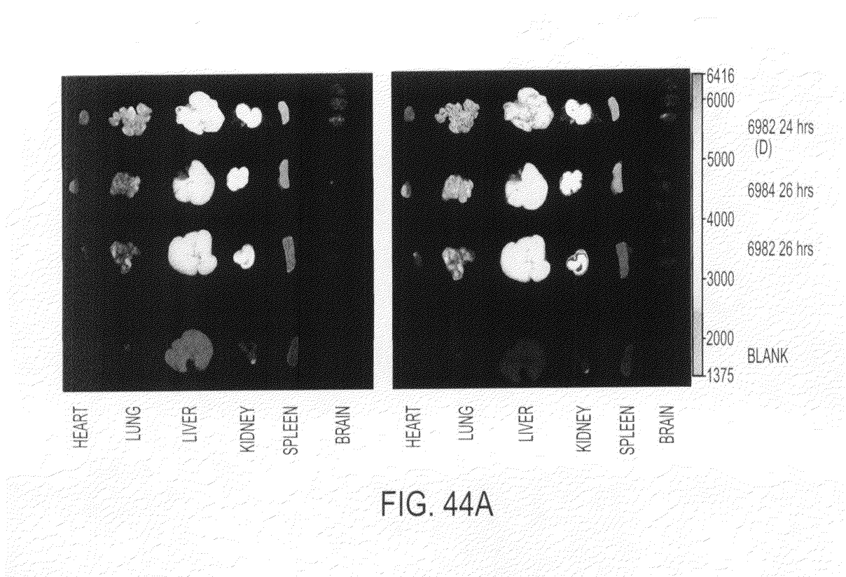

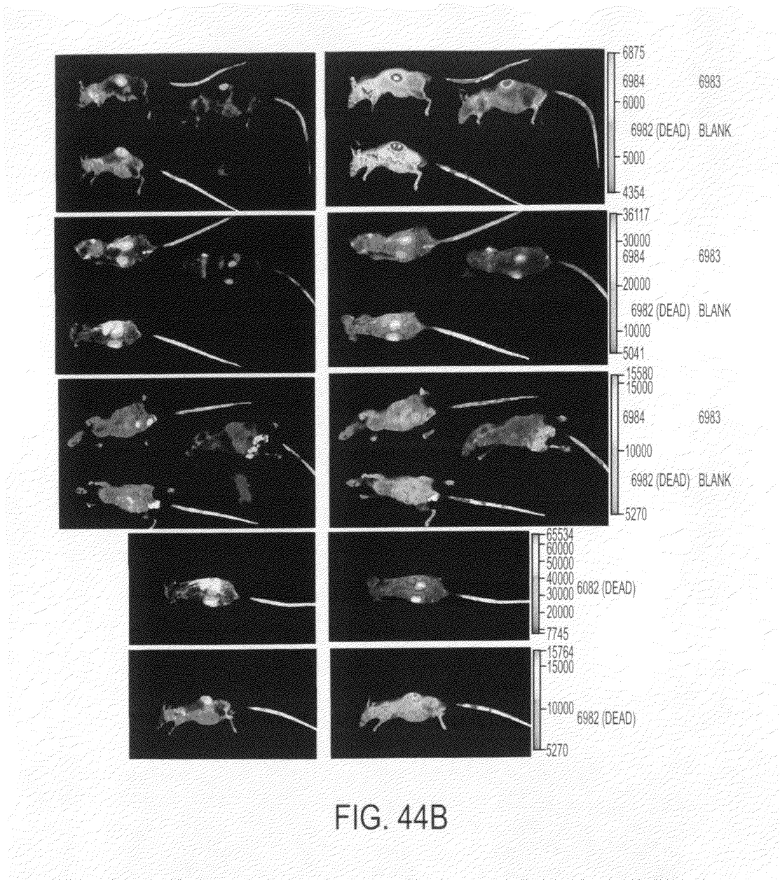

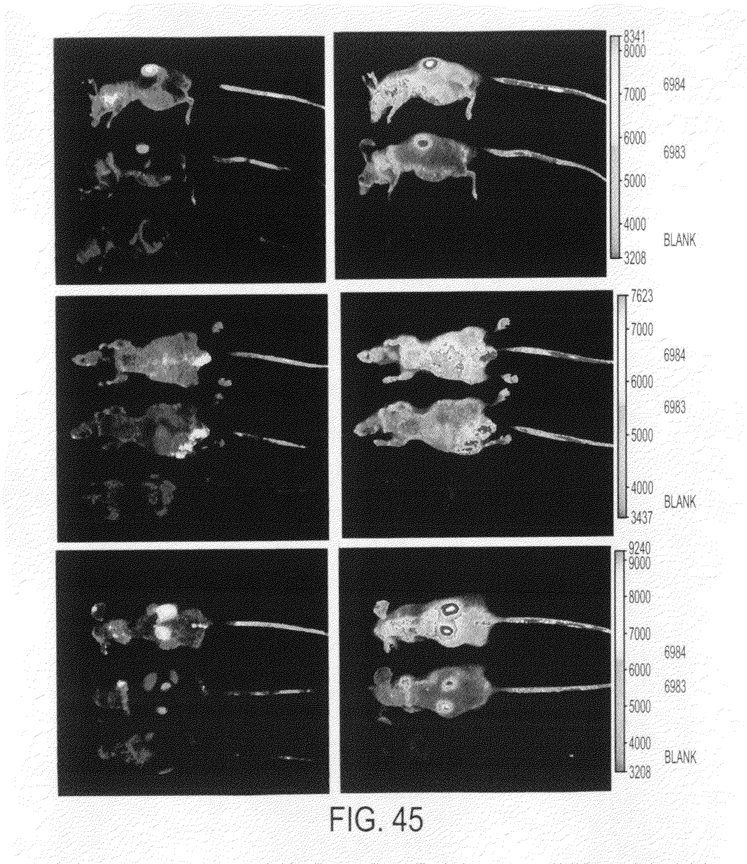

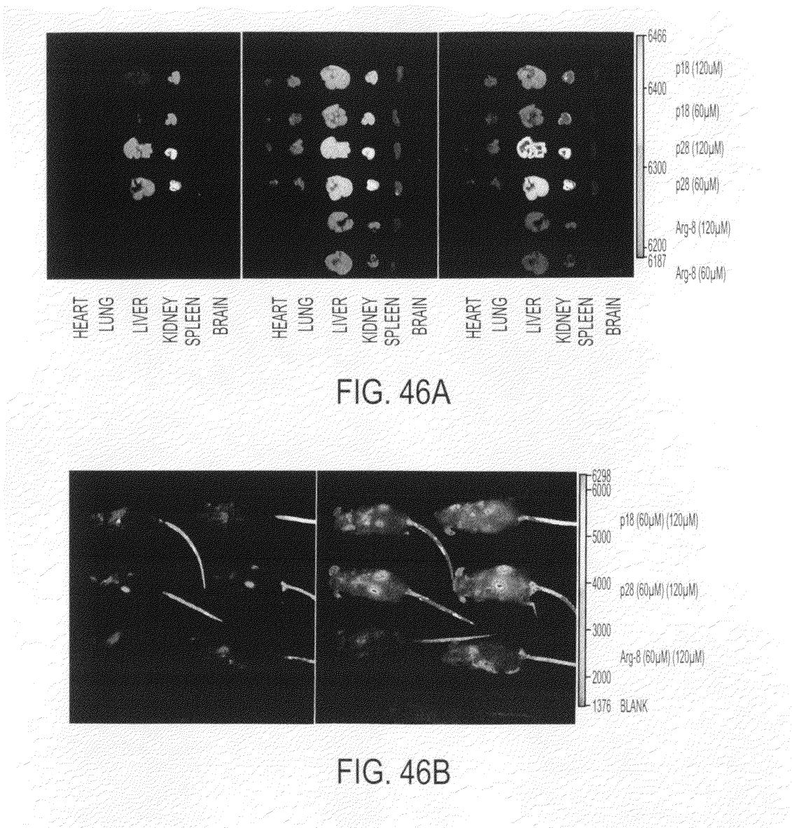

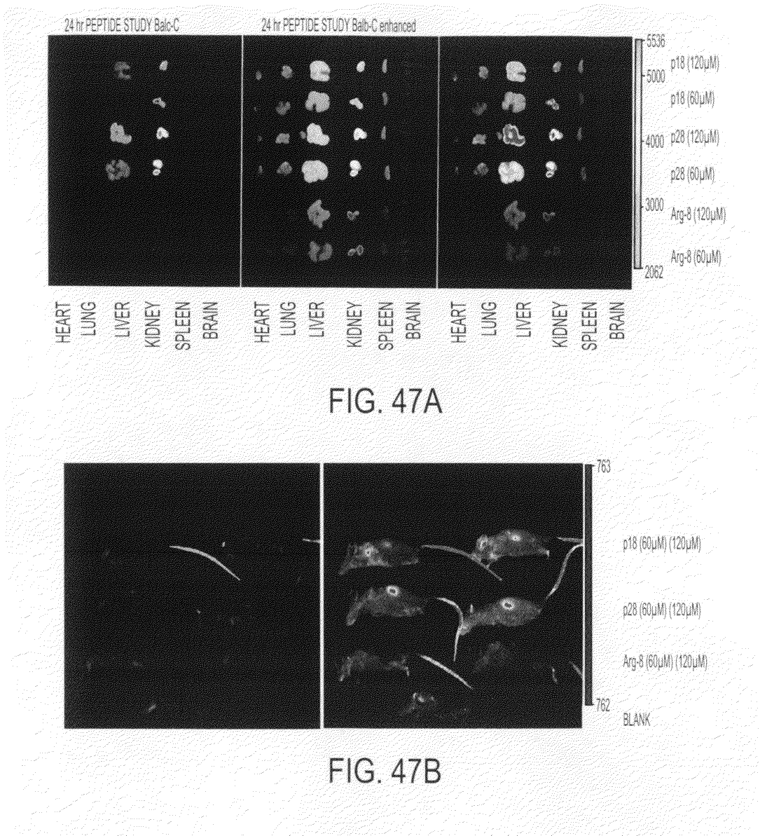

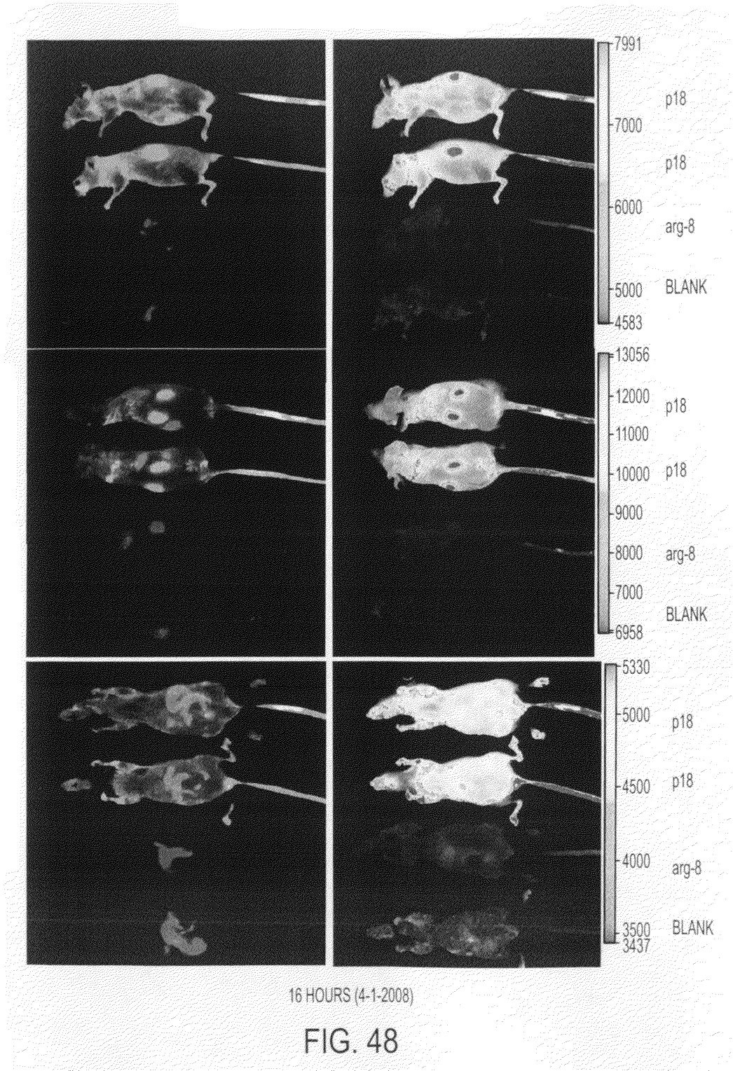

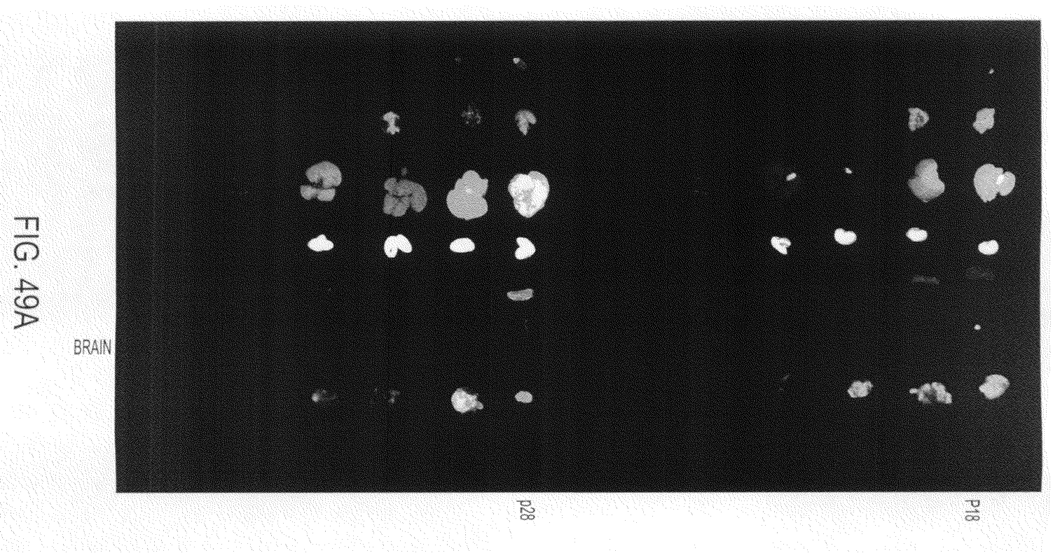

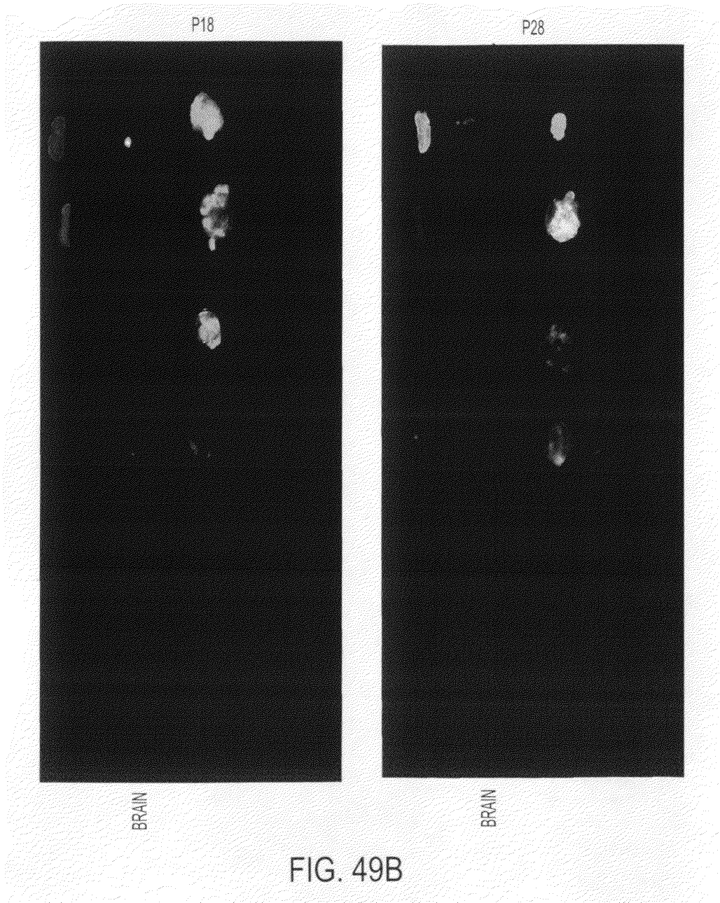

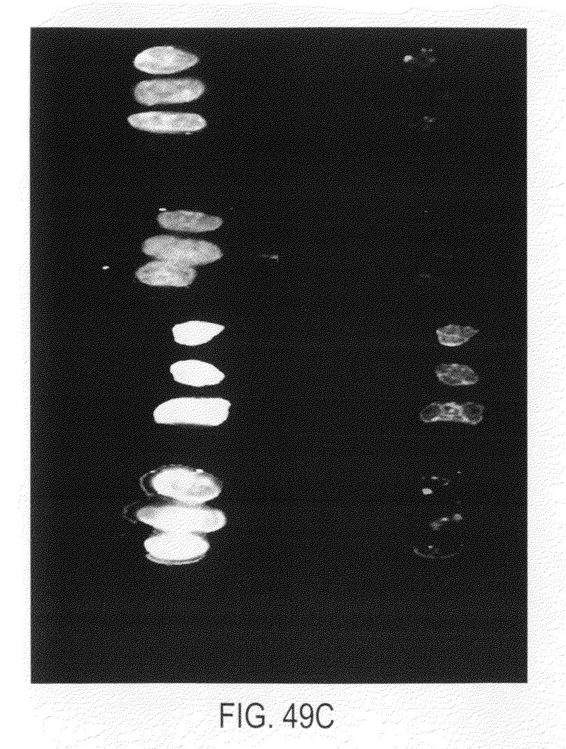

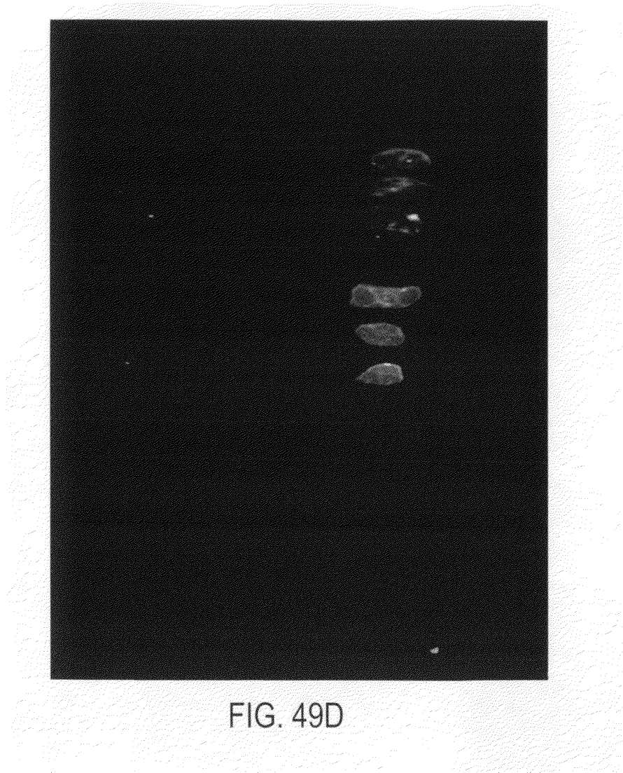

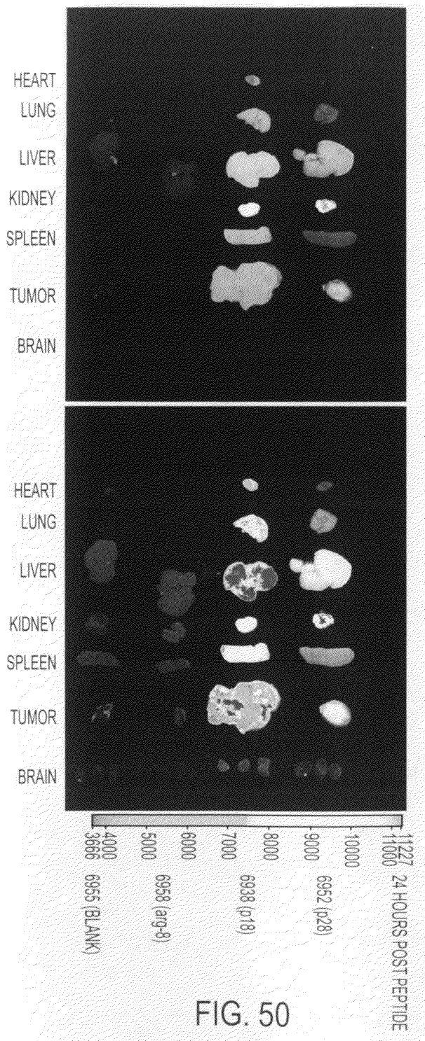

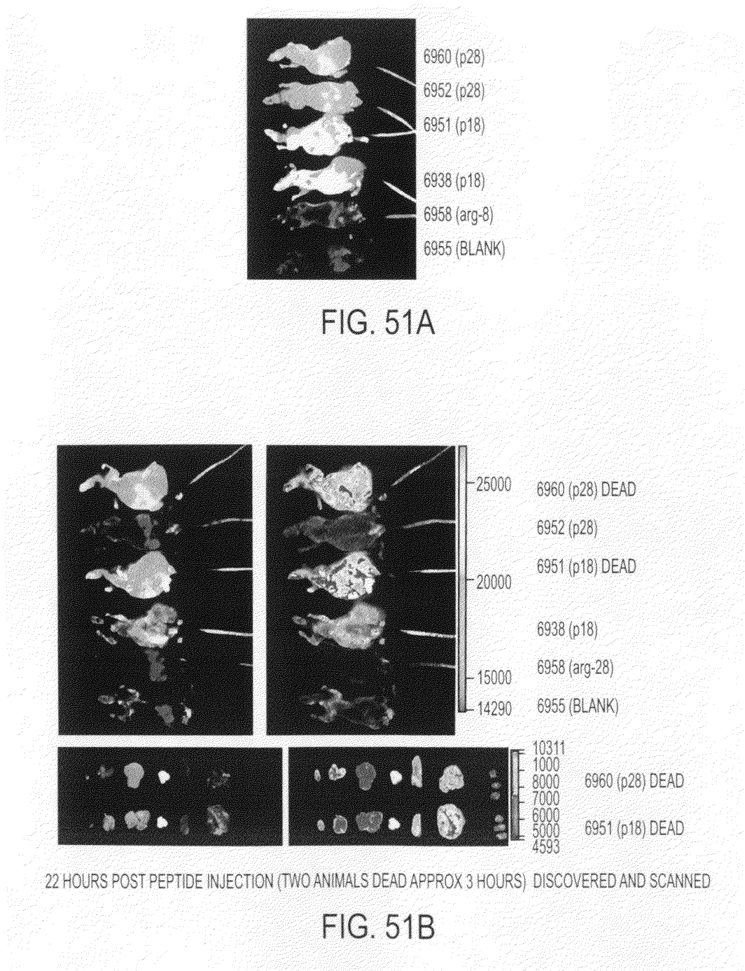

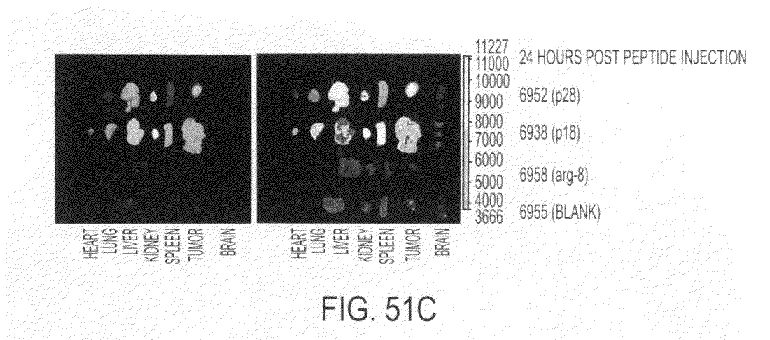

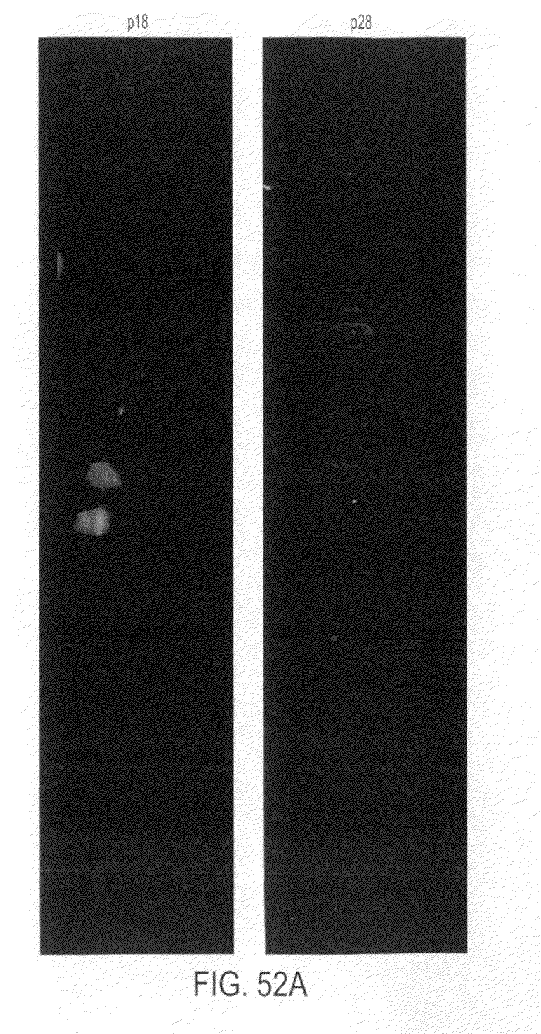

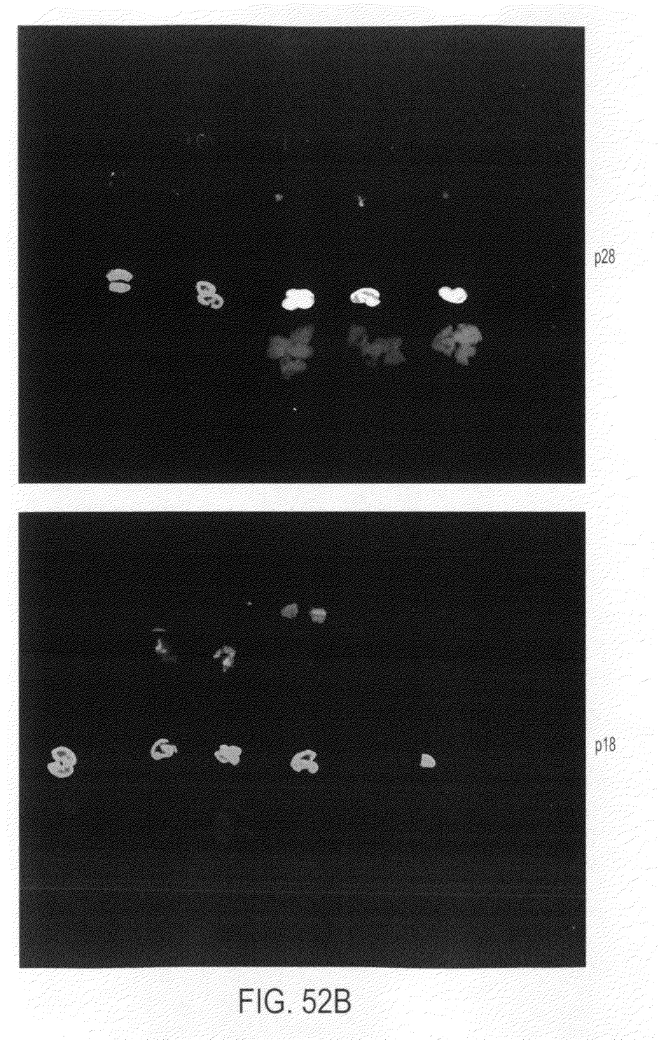

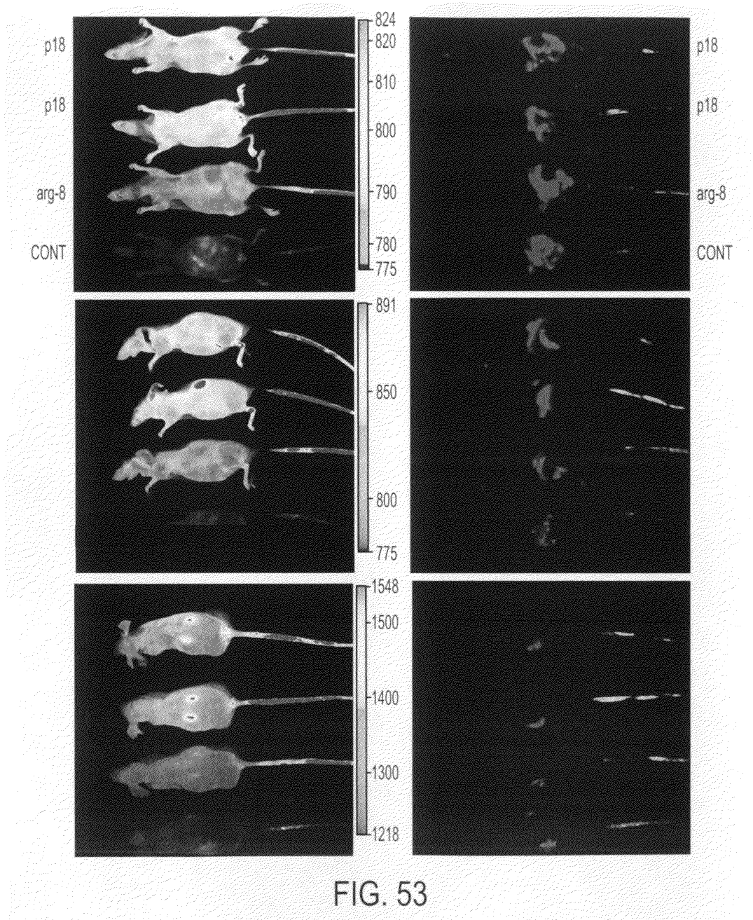

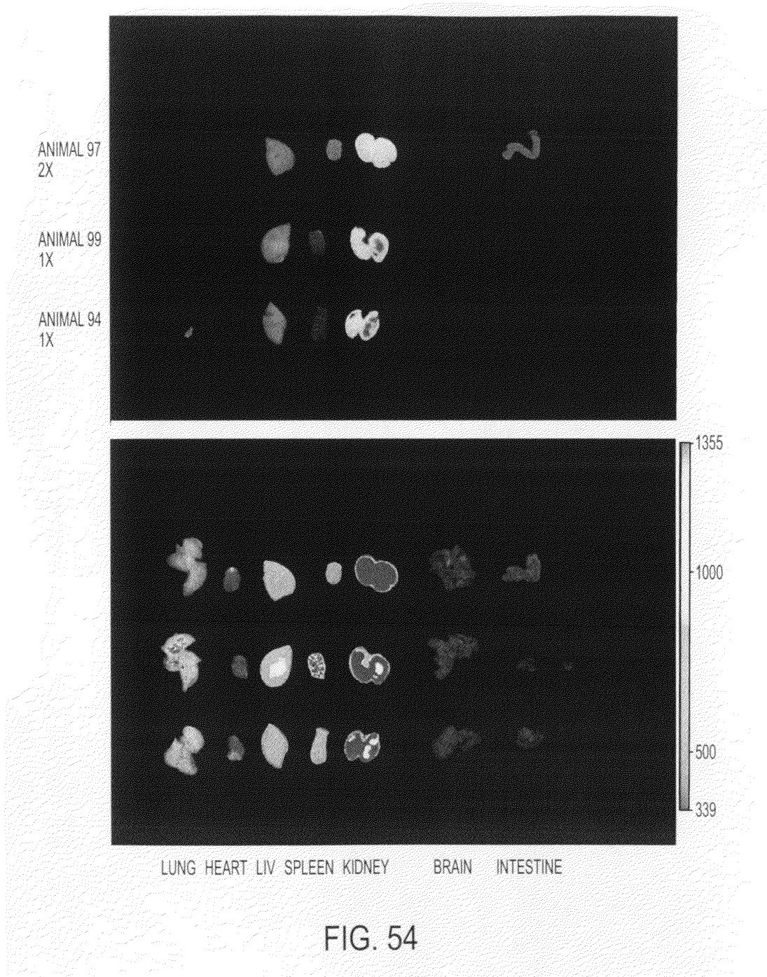

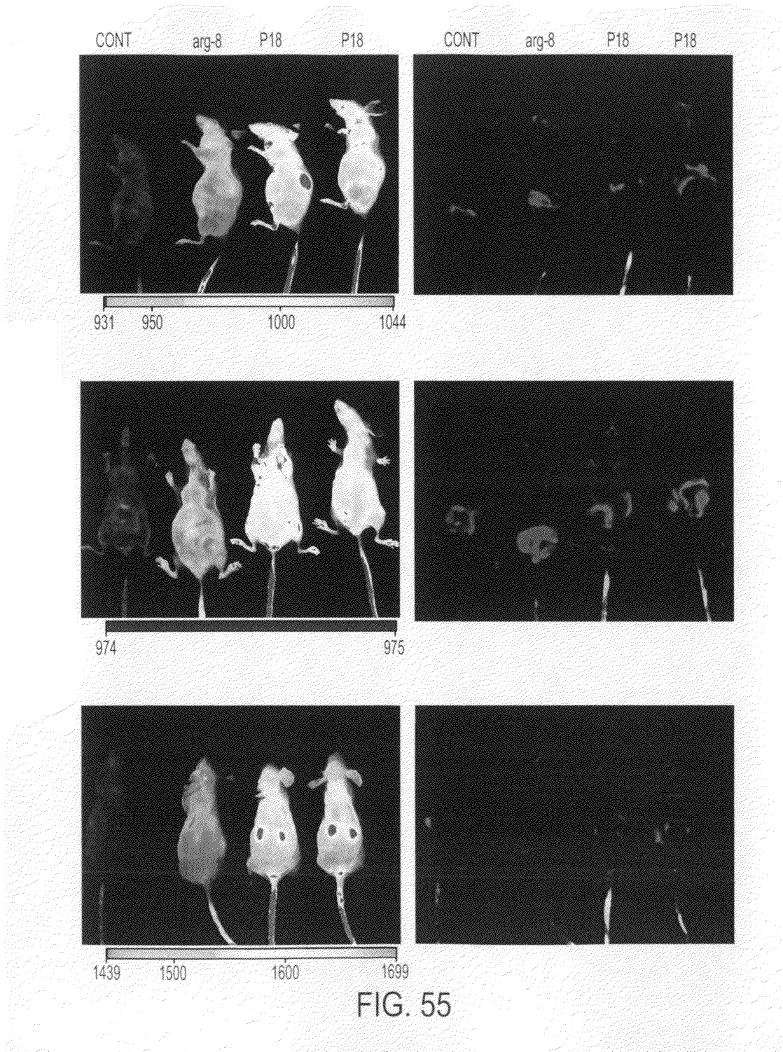

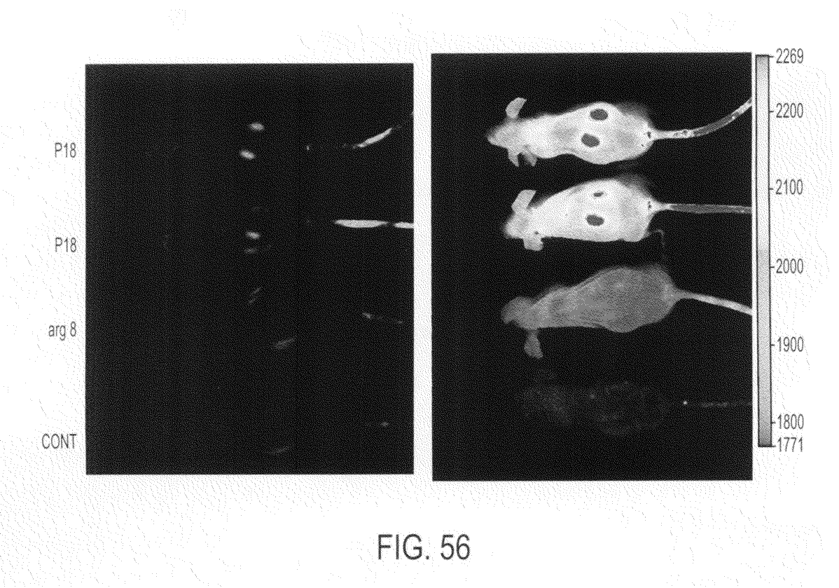

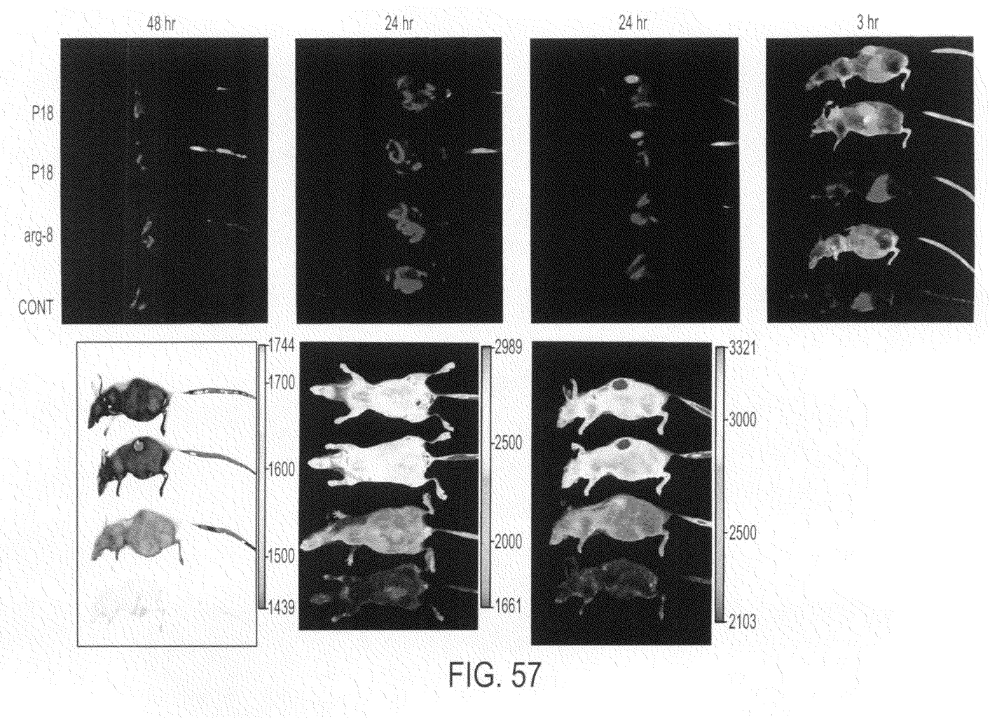

p18 and p28 are able to enter cancer cells, tumors, and mammalian organs, as is shown in FIGS. 21 through 57. Surprisingly, p18 and p28 are also able to penetrate the blood-brain barrier and enter mammalian brains, as demonstrated by, for example, FIGS. 24A, 24B, 25B, 27, 28, 32, 34, 35A, 36B, 37, 38B, 39A-B, 41A-C, 42A-C, 44A, 46A, 47A, 49A-D, 50, 51B, 52A-B, and 54. As such, these peptides may be used to treat conditions in mammalian brains and brain cells.

It is also now known that synthesized p28 not only enters into a variety of malignant cell lines (melanoma (Mel-2), MCF-7, pancreatic, astrocytoma, glioblastoma, among others), but also non-cancerous human umbilical vein endothelial cells (HUVEC). See Example 1. p28 enters into these cells in a temperature dependent manner, but does not enter normal cells (fibroblast, normal mammary epithelium). As HUVEC cells are known to instigate angiogenesis in human embryos, the entry of p28 into HUVEC cells prompted an examination of the effect of p28 on angiogenesis. HUVEC cells (20,000 cells) were plated on Matrigel® coated wells and incubated in media containing 0-75 μM of p28. Cultures were examined under light microscopy at 4 h and 24 h post-treatment. The p28 peptide inhibited capillary tube formation of the HUVEC in a dose dependent manner, suggesting that p28 inhibits the capillary tube formation step of angiogenesis. See Example 2. Further, p28 inhibited the migration of HUVEC cells on Matrigel® in a scratch wound migration assay, indicating that p28 also inhibits the migration step of angiogenesis. See Example 3. Thus, in in vitro studies with an established angiogenesis model system, HUVEC cells on Matrigel®, p28 inhibits two critical steps in angiogenesis, capillary tube formation and cell migration.

It is also now known that azurin, and peptides derived from azurin, such as p28, have chemopreventative properties. It is now known that azurin, and p28, prevent the formation of premalignant preneoplastic lesions in mouse mammary gland organ culture. In a mouse mammary gland organ culture model, azurin at 50 μg/ml was found to inhibit the formation of alveolar lesions by 67%. Likewise, p28 at 25 μg/ml was found to inhibit the formation of alveolar lesions by 67%. Further, azurin at 50 μg/ml was found to inhibit the formation of ductal lesions by 79%, and p28 at 25 μg/ml inhibited the formation of ductal lesions by 71%. Confocal microscopy and FAC showed that azurin and p28 entered normal murine mammary epithelial cells (MM3MG) and mammary cancer cells (4T1). It is therefore now known that azurin and variants of azurin may be used to inhibit the formation of premalignant preneoplastic lesions, and thus the development of cancer, and specifically breast cancer, in mammalian patients.

It is also now known that cupredoxins and cytochromes will inhibit in vitro parasitemia in human red blood cells by the malaria parasite Plasmodium falciparum. In particular, the cupredoxins azurin and Laz inhibit parasitemia in P. falciparum by about 50% and about 75% respectively. See, Example 14. Further, rusticyanin and cytochromes c and f inhibited parasitemia by 20-30%. See, Example 9. Further, it is now known that azurin has a discernable structural homology to the Fab fragment of G17.12 mouse monoclonal antibody when complexed to the PfMSP1-19 fragment of the MSP1 surface protein of P. falciparum. While not limiting the mode of inhibition to any one means, it is thought that azurin may inhibit parasitemia of P. falciparum by interaction with the MSP1 protein on the parasite's surface.

It is also now known that azurin and Laz bind both the PfMSP1-19 and PfMSP1-42 P. falciparum surface proteins in vitro. Further, it is now known that azurin amino acid residues 36-89 are required for binding to PfMSP1-19 and PfMSP1-42. Further, it is now known that the H.8 domain of Laz from N. gonorrhea increases both the binding of a fused azurin to PfMSP1-19 as well as inhibition of parasitemia by P. falciparum. See, Examples 13 and 14.

It has also been learned that P. aeruginosa cytochrome c₅₅₁, human cytochrome c and Phormidium laminosum cytochrome f will inhibit parasitemia in malaria-infected human red blood cells. In a specific embodiment, the cytochrome is cytochrome c₅₅₁ from P. aeruginosa, human cytochrome c or cytochrome f. In other specific embodiments, the cytochrome comprises an amino acid sequence that is SEQ ID NO: 19-21.

It is also now known that azurin can induce about a 90% suppression of growth of HIV-1 in peripheral blood mononuclear cell (PBMC) cultures. See, Example 18. Azurin is now known to inhibit the growth of three strains of HIV-1, Bal (the most predominant clade B circulating in the US and Western Europe), a clade B African isolate RW/92/008/RE1, and a clade C Indian isolate IN/2167 D15. See, Example 18. Additionally, a cupredoxin-like protein from Neisseria, Laz, is now also known to inhibit the growth of these three HIV-1 strains, as well as a fusion of the H.8 region of the Laz protein with P. aeruginosa azurin. See, Example 18. Finally, it is now known that M44KM64E mutant of azurin and cytochrome c551 from P. aeruginosa can inhibit HIV infection in HIV-infected human blood lymphocytes. See, Example 16.

Due to the high degree of structural similarity between cupredoxins, it is likely that other cupredoxins may treat and/or prevent numerous diseases. In some embodiments, the cupredoxin may be, but is not limited to, azurin, pseudoazurin, plastocyanin, auracyanin, Laz, rusticyanin, stellacyanin or cucumber basic protein. In a more specific embodiment, the cupredoxin may be azurin. In a specific embodiment, the cupredoxin or azurin may be derived from Pseudomonas aeruginosa, Alcaligenes faecalis, Achromobacter xylosoxidan, Bordetella bronchiseptica, Methylomonas sp., Neisseria meningitidis, Neisseria gonorrhea, Pseudomonas fluorescens, Pseudomonas chlororaphis, Bordetella pertussis, Pseudomonas syringae, Xylella fastidiosa and Vibrio parahaemolyticus. In a most specific embodiment, the azurin is from P. aeruginosa. In other specific embodiments, the cupredoxin comprises an amino acid sequence that is SEQ ID NOs: 1, 5-12, 18 and 23. Several cupredoxins are known to have pharmacokinetic activities similar to those of azurin from Pseudomonas aeruginosa. For example, rusticyanin from Thiobacillus ferrooxidans can also enter macrophages and induce apoptosis. Yamada et al., Cell Cycle 3:1182-1187 (2004); Yamada et al., Cell. Micro. 7:1418-1431 (2005). Plastocyanin from Phormidium laminosum and pseudoazurin form Achromobacter cycloclastes also are cytotoxic towards macrophages. U.S. Pat. Pub. No. 20060040269, published Feb. 23, 2006. It is therefore contemplated that other cupredoxins may be used in the compositions and methods of the invention. Further, variants, derivatives, and structural equivalents of cupredoxins that retain the ability to inhibit the formation of cancer in mammals may also be used in the compositions and methods of the invention. These variants and derivatives may include, but are not limited to, truncations of a cupredoxin, conservative substitutions of amino acids and proteins modifications such as PEGylation, all-hydrocarbon stabling of α-helices, and other methods and techniques disclosed herein.

Moreover, because of the structural homology between the cytochromes, it is contemplated that other cytochromes will have the same ability to treat and/or prevent more than one condition as P. aeruginosa cytochrome c₅₅₁ and human cytochrome c. In some embodiments, the cytochrome is from a pathogenic bacterium. In another specific embodiment, the cytochrome inhibits parasitism in malaria-infected red blood cells, and more specifically, human red blood cells. In another embodiment, the cytochrome inhibits viral infection such as HIV. In another specific embodiment, the cytochrome inhibits cell cycle progression in a mammalian cancer cell, and more specifically in a J774 cell.

### Compositions of the Invention

The invention provides for peptides that are cupredoxins and/or cytochromes, and/or variants, derivatives or structural equivalents of cupredoxin or cytochrome. In some embodiments, the peptide is isolated. In some embodiments, the peptide is substantially pure or pharmaceutical grade. In other embodiments, the peptide is in a composition that comprises, or consists essentially of, the peptide. In another specific embodiment, the peptide is non-antigenic and does not raise an immune response in a mammal, and more specifically a human. In some embodiments, the peptide is less than a full-length cupredoxin or cytochrome, and retains some of the pharmacologic activities of the cupredoxin or cytochrome. In one specific embodiment, the peptide may retain the ability to concurrently treat and/or prevent two or more conditions in a mammalian cell or a patient.

In some embodiments, the peptide retains the ability to inhibit the growth of viral or bacterial infection. In some embodiments, the peptide retains the ability to inhibit specifically HIV-1 infection in peripheral blood mononuclear cells, or parasitemia in malaria-infected red blood cells, or P. falciparium infection in human red blood cells or inhibit angiogenesis in HUVECs on Matrigel®, or inhibit cancer in malignant cells.

The invention also provides compositions comprising at least one peptide that is a cupredoxin, or variant, derivative, truncation, or structural equivalent of a cupredoxin. The invention also provides compositions comprising at least one peptide that is a cytochrome, or variant, derivative, truncation, or structural equivalent of a cytochrome. In other embodiments, the composition consists essentially of the peptide.

In some embodiments, the cupredoxin is selected from the group consisting of azurin, pseudoazurin, plastocyanin, rusticyanin, Laz, auracyanin, stellacyanin and cucumber basic protein. In some embodiments, the cupredoxin is from an organism selected from the group consisting of Pseudomonas aeruginosa, Alcaligenes faecalis, Achromobacter xylosoxidan, Bordetella bronchiseptica, Methylomonas sp., Neisseria meningitidis, Neisseria gonorrhea, Pseudomonas fluorescens, Pseudomonas chlororaphis, Bordetella pertussis, Pseudomonas syringae, Xylella fastidiosa and Vibrio parahaemolyticus. In a very specific embodiment, the cupredoxin is from Pseudomonas aeruginosa.

In one embodiment, the cupredoxin or cytochrome, or variant, derivative, truncation, or structural equivalent thereof, is fused to a H.8 region of Laz from Neisseria meningitides or Neisseria gonorrhea. One example of such a peptide is the H.8-Paz fusion protein. In a specific embodiment, the H.8 is fused to the C-terminus of the cupredoxin or cytochrome, or variant, derivative, truncation, or structural equivalent thereof. In another specific embodiment, the H.8 region is SEQ ID NO: 22, or a variant, derivative, truncation, or structural equivalent thereof.

In another embodiment, the variant or derivative of cupredoxin has a significant structural homology to the Fab fragment of G17.12 mouse monoclonal antibody. An example of how this structural similarity can be determined can be found in Example 11. Specifically, significant structural homology between a cupredoxin and the Fab fragment of G17.12 mouse monoclonal antibody can be determined by using the VAST algorithm (Gibrat et al., id.; Madej et al., id.). In specific embodiments, the VAST p-value from a structural comparison of a cupredoxin to the Fab fragment of G17.12 mouse monoclonal antibody can be less than about 10⁻⁴, less than about 10⁻⁵, less than about 10⁻⁶, or less than about 10⁻⁷. In other specific embodiments, the VAST score from a structural comparison of a cupredoxin to the Fab fragment of G17.12 mouse monoclonal antibody can be greater than about 9, greater than about 10, greater than about 11 or greater than about 12.

In some embodiments, the variant, derivative, truncation, or structural equivalent thereof has some of the functional characteristics of the P. aeruginosa azurin, P. aeruginosa cytochrome c₅₅₁, human cytochrome c or cyanobacterial cytochrome f. In a specific embodiment, the peptide of the invention inhibits parasitemia by malaria in malaria-infected red blood cells, and more specifically parasitemia by P. falciparum in P. falciparum-infected human red blood cells. The invention also provides for the variants, derivatives and structural equivalents of cupredoxin and cytochrome c₅₅₁ that retain the ability to inhibit parasitemia in malaria-infected red blood cells, and more specifically parasitemia by P. falciparum in P. falciparum-infected human red blood cells. The inhibition of parasitemia by P. falciparum in P. falciparum-infected human red blood cells may be determined by the method described in Example 14.

The invention provides for amino acid sequence variants of a cupredoxin or cytochrome which have amino acids replaced, deleted, or inserted as compared to the wild-type polypeptide. Variants of the invention may be truncations of the wild-type polypeptide. In some embodiments, the composition comprises a peptide that consists of a region of a cupredoxin or cytochrome that is less than the full length wild-type polypeptide. In some embodiments, the composition comprises a peptide that consists of more than about 10 residues, more than about 15 residues or more than about 20 residues of a truncated cupredoxin or cytochrome. In some embodiments, the composition comprises a peptide that consists of not more than about 100 residues, not more than about 50 residues, not more than about 40 residues or not more than about 30 residues of a truncated cupredoxin or cytochrome. In some embodiments, the composition comprises a peptide to which a cupredoxin or cytochrome, and more specifically to SEQ ID NOS.: 1, 5-12, 18 and 23, and has at least about 90% amino acid sequence identity, at least about 95% amino acid sequence identity or at least about 99% amino acid sequence identity or is a mutant of SEQ ID NOS.: 1, 5-12, 18 and 23.

In specific embodiments, the variant of cupredoxin comprises Pseudomonas aeruginosa azurin residues 50-77 (p28, SEQ ID NO: 29), Pseudomonas aeruginosa azurin residues 50-67 (p18, SEQ ID NO: 30), Pseudomonas aeruginosa azurin residues 36-88 (SEQ ID NO: 50), Pseudomonas aeruginosa azurin residues 36-128 (SEQ ID NO: 31), Pseudomonas aeruginosa azurin residues 88-113 (SEQ ID NO: 49), Pseudomonas aeruginosa azurin residues 36-89 (SEQ ID NO: 32), and Pseudomonas aeruginosa azurin residues 96-113 (SEQ ID NO: 48), Vibrio parahaemolyticus azurin residues 52-78 (SEQ ID NO: 27), Pseudomonas syringae azurin residues 51-77 (SEQ ID NO: 25), Bordetella bronchiseptica azurin residues 51-77 (SEQ ID NO: 28), and Pseudomonas aeruginosa azurin residues 36-77 (SEQ ID NO: 33).

In other embodiments, the variant of cupredoxin consists of Pseudomonas aeruginosa azurin residues 50-77 (SEQ ID NO: 29), Pseudomonas aeruginosa azurin residues 50-67 (SEQ ID NO: 30), Pseudomonas aeruginosa azurin residues 36-88 (SEQ ID NO: 50), Pseudomonas aeruginosa azurin residues 36-128 (SEQ ID NO: 31), Pseudomonas aeruginosa azurin residues 88-113 (SEQ ID NO: 49), Pseudomonas aeruginosa azurin residues 36-89 (SEQ ID NO: 32), and Pseudomonas aeruginosa azurin residues 96-113 (SEQ ID NO: 48), Vibrio parahaemolyticus azurin residues 52-78 (SEQ ID NO: 27), Pseudomonas syringae azurin residues 51-77 (SEQ ID NO: 25), Bordetella bronchiseptica azurin residues 51-77 (SEQ ID NO: 28), and Pseudomonas aeruginosa azurin residues 36-77 (SEQ ID NO: 33). In other specific embodiments, the variant consists of the equivalent residues of a cupredoxin.

It is also contemplated that other cupredoxin variants can be designed that have a similar pharmacological activity to Pseudomonas aeruginosa azurin residues 50-77 (SEQ ID NO: 29), Pseudomonas aeruginosa azurin residues 50-67 (SEQ ID NO: 30), Pseudomonas aeruginosa azurin residues 36-88 (SEQ ID NO: 50), Pseudomonas aeruginosa azurin residues 36-128 (SEQ ID NO: 31), Pseudomonas aeruginosa azurin residues 88-113 (SEQ ID NO: 49), Pseudomonas aeruginosa azurin residues 36-89 (SEQ ID NO: 32), and Pseudomonas aeruginosa azurin residues 96-113 (SEQ ID NO: 48), Vibrio parahaemolyticus azurin residues 52-78 (SEQ ID NO: 27), Pseudomonas syringae azurin residues 51-77 (SEQ ID NO: 25), Bordetella bronchiseptica azurin residues 51-77 (SEQ ID NO: 28), and Pseudomonas aeruginosa azurin residues 36-77 (SEQ ID NO: 33). To do this, the subject cupredoxin amino acid sequence will be aligned to the Pseudomonas aeruginosa azurin sequence using BLAST, BLAST2, ALIGN2 or Megalign (DNASTAR), the relevant residues located on the P. aeruginosa azurin amino acid sequence, and the equivalent residues found on the subject cupredoxin sequence, and the equivalent peptide thus designed.

The variants also include peptides made with synthetic amino acids not naturally occurring. For example, non-naturally occurring amino acids may be integrated into the variant peptide to extend or optimize the half-life of the composition in the bloodstream. Such variants include, but are not limited to, D,L-peptides (diastereomer), (Futaki et al., J. Biol. Chem. 276(8):5836-40 (2001); Papo et al., Cancer Res. 64(16):5779-86 (2004); Miller et al, Biochem. Pharmacol. 36(1):169-76, (1987); peptides containing unusual amino acids (Lee et al., J. Pept. Res. 63(2):69-84 (2004)), and incorporation of olefin-containing non-natural amino acid followed by hydrocarbon stapling (Schafmeister et al., J. Am. Chem. Soc. 122:5891-5892 (2000); Walenski et al., Science 305:1466-1470 (2004)), and peptides comprising ε-(3,5-dinitrobenzoyl)-Lys residues.

The invention also provides compositions comprising one peptide or at least two peptides that are a cupredoxin, cytochrome, or variant, derivative, truncation, or structural equivalent of a cupredoxin or cytochrome in a pharmaceutical composition. In some embodiments, the cupredoxin is in a pharmaceutical composition and is from an organism selected from the group consisting of Pseudomonas aeruginosa, Alcaligenes faecalis, Achromobacter xylosoxidans ssp. denitrificans I, Bordetella bronchiseptica, Methylomonas sp., Neisseria meningitides, Neisseria gonorrhea, Pseudomonas fluorescens, Pseudomonas chlororaphis, Bordetella pertussis, Pseudomonas chlororaphis, Xylella fastidiosa, Ulva pertussis or Vibrio parahaemolyticus. In a specific embodiment, the cupredoxin is from Pseudomonas aeruginosa. In another specific embodiment, the cupredoxin or cytochrome is selected from the group consisting of SEQ ID NOS: 1, 5-12, 18, 23, 25, 27-33 and 48-50 in a pharmaceutical composition. In another specific embodiment, the cupredoxin may comprise SEQ ID NO: 30.

In other embodiments, the peptide of the invention is a derivative of a cupredoxin or cytochrome. The derivatives of cupredoxin or cytochrome are chemical modifications of the peptide such that the peptide still retains some of its fundamental activities. For example, a “derivative” of azurin can be a chemically modified azurin that retains its ability to treat and/or prevent more than one condition in a mammalian cell. Chemical modifications of interest include, but are not limited to, amidation, acetylation, sulfation, polyethylene glycol (PEG) modification, phosphorylation, glycosylation of the peptide, and other modifications disclosed herein. In addition, a derivative peptide maybe a fusion of a cupredoxin or cytochrome, or variant, derivative, truncation, or structural equivalent thereof to a chemical compound, such as but not limited to, another peptide, drug molecule or other therapeutic or pharmaceutical agent or a detectable probe. Derivatives of interest include chemical modifications by which the half-life in the bloodstream of the peptides and compositions of the invention can be extended or optimized, such as by several methods well known to those in the art, including but not limited to, circularized peptides (Monk et al., BioDrugs 19(4):261-78, (2005); DeFreest et al., J. Pept. Res. 63(5):409-19 (2004)), N- and C-terminal modifications (Labrie et al., Clin. Invest. Med. 13(5):275-8, (1990)), and incorporation of olefin-containing non-natural amino acid followed by hydrocarbon stapling (Schafmeister et al., J. Am. Chem. Soc. 122:5891-5892 (2000); Walenski et al., Science 305:1466-1470 (2004)).

It is contemplated that the peptide of the composition of invention may be more than one of a variant, derivative and structural equivalent of a cupredoxin or cytochrome. For example, the peptide may be a truncation of azurin that has been PEGylated, thus making it both a variant and a derivative. In one embodiment, the peptides of the invention are synthesized with α,α-disubstituted non-natural amino acids containing olefin-bearing tethers, followed by an all-hydrocarbon “staple” by ruthenium catalyzed olefin metathesis. (Scharmeister et al., J. Am. Chem. Soc. 122:5891-5892 (2000); Walensky et al., Science 305:1466-1470 (2004)). Additionally, peptides that are structural equivalents of azurin may be fused to other peptides, thus making a peptide that is both a structural equivalent and a derivative. These examples are merely to illustrate and not to limit the invention. Variants, derivatives or structural equivalents of cupredoxin or cytochrome may or may not bind copper.

In some embodiments, the cupredoxin may be varied using methods that include, but are not limited to, those which decrease the hydrolysis of the peptide, decrease the deamidation of the peptide, decrease the oxidation, decrease the immunogenicity and/or increase the structural stability of the peptide. It is contemplated that two or more of the modifications described herein may be combined in one modified cupredoxin derived peptide, as well as combinations of one or more modifications described herein with other modification to improve pharmacokinetic properties that are well know to those in the art. Many methods to design such variants and derivatives are well known in the art, and some are discussed below and herein.

### Biotransformation

One approach to improving the pharmacokinetic properties of cupredoxins, cytochromes, and variants, derivatives, truncations, and structural equivalents thereof, particularly cupredoxin-derived peptides such as truncations of azurin, is to create variants and derivatives of the cupredoxin derived peptides that are less susceptible to biotransformation. Biotransformation may decrease the pharmacologic activity of the peptide as well as increase the rate at which it is eliminated from the patient's body. One way of achieving this is to determine the amino acids and/or amino acid sequences that are most likely to be biotransformed and to replace these amino acids with ones that are not susceptible to that particular transformative process.

In some embodiments, the cupredoxin derived peptides may include unnatural amino acids or modified amino acids. In some embodiments, the introduction of certain unnatural amino acids enhances the pharmacokinetic properties of the cupredoxin derived peptide. Such introduction may be site-specific and may be done to avoid certain biochemical modifications in vivo. Exemplary unnatural amino acids include b-amino acids (e.g., b3 and b2), homo-amino acids, cyclic amino acids, aromatic amino acids, Pro and Pyr derivatives, 3-substituted Alanine derivatives, Glycine derivatives, Ring-substituted Phe and Tyr Derivatives, Linear Core Amino Acids and Diamino Acids. Such unnatural amino acids may be incorporated into peptides by site directed modification, ribosomal translation, or by chemical synthesis of the peptide. Each of these methods may be applied in synthesizing cupredoxin derived peptides.

For example, modified cupredoxin derived peptides may be synthesized by the use of wild-type Aminoacyl-tRNA synthetases (AARSs) with unnatural amino acids building for the production of unnatural cupredoxin variants. See Hartman, et al., PLoS One, 2(10): e972 (2007); Miranda, et al., J. Am. Chem. Soc. 129: 13153-13159 (2007). The specificity of the ribosomal translation apparatus limits the diversity of unnatural amino acids that may be incorporated into peptides using ribosomal translation. Over ninety unnatural building blocks that are AARS substates have been uncovered including side chain and backbone analogs. Hartman, et al., PLoS One, 2(10): e972 (2007). Over fifty unnatural amino acids may be incorporated into peptides with high efficiency using an all-enzymatic translation system, with peptides containing up to thirteen different unnatural amino acids. Hartman, et al., PLoS One, 2(10): e972 (2007). In some embodiments, such amino acids may be incorporated in cupredoxin derived peptides.

Other modifications may include the use of optically active α-amino acids. The use of optically active α-amino acids and their derivatives is being expanded for their use in pharmaceuticals, agrochemicals and as chiral ligands. In particular, chiral glycine and alanine equivalents plan an important role. At least one stereoselective strategy for constructing α-amino acids has been proposed, allowing for enantiopure α-amino acids in predetermined stereochemistry. Lu, et al. “Asymmetric Synthesis of α-amino acids: Preparation and alkylation of monocyclic iminolactones derived from α-Methyl trans-cinnamaldehyde” published on the Internet on Sep. 11, 2008 (to be published in J. Org. Chem.), the disclosure of which is incorporated by reference herein. The modified cupredoxin derived peptides may be synthesized using the optically active α-amino acids to produce enantiomerically enriched iterations.

Hydrolysis is generally a problem in peptides containing aspartate. Aspartate is susceptible to dehydration to form a cyclic imide intermediate, causing the aspartate to be converted to the potentially inactive iso-aspartate analog, and ultimately cleaving the peptide chain. For example, in the presence of aspartic acid-proline in the peptide sequence, the acid catalyzed formation of cyclic imide intermediate can result to cleavage of the peptide chain. Similarly, in the presence of aspartic acid-glycine in the peptide sequence, the cyclic intermediate can be hydrolyzed either into the original aspartate form (harmless) or into the iso-aspartate analog. Eventually, all of the aspartate form can be completely converted into the iso-aspartate analog. Similarly sequences with serine can also be dehydrated to form a cyclic imide intermediate that can cleave the peptide chain. Cleavage of the peptide may result in reduced plasma half-life as well as reduced specific pharmacologic activity of the peptide.

It is contemplated that substituting other amino acids for asparagine and/or serine in the sequence of the cupredoxin derived peptide may result in a peptide with improved pharmacokinetic properties such as a longer plasma half-life and increased specific activity of a pharmacologic activity of the peptide. In one contemplated variant, at one or more asparagine residues of the cupredoxin derived peptide may be replaced with another amino acid residue, and specifically a glutamic acid residue. In another contemplated variant, one or more serine residues of the cupredoxin derived peptide may be replaced with another amino acid residue, and specifically a threonine residue. In some variants of cupredoxin derived peptide, one or more asparagine residues and one or more serine residues are substituted. In some embodiments, conservative substitutions are made. In other embodiments, non-conservative substitutions are made.

Deamidation of amino acid residues is a particular problem in biotransformation. This base-catalyzed reaction frequently occurs in sequences containing asparagine-glycine or glutamine-glycine and follows a mechanism analogous to the aspartic acid-glycine sequence above. The de-amidation of the asparagine-glycine sequence forms a cyclic imide intermediate that is subsequently hydrolyzed to form the aspartate or iso-asparate analog of asparagine. In addition, the cyclic imide intermediate can lead to racemization into D-aspartic acid or D-iso-aspartic acid analogs of asparagine, all of which can potentially lead to inactive forms of the peptide.

It is contemplated that deamidation in the cupredoxin peptides may be prevented by replacing a glycine, asparagine and/or glutamine of the asparagine-glycine or glutamine-glycine sequences of the cupredoxin with another amino acid and may result in a peptide with improved pharmacokinetic properties, such as a longer plasma half-life and increased specific activity of a pharmacologic activity of the peptide. In some embodiments, the one or more glycine residues of the cupredoxin derived peptide are replaced by another amino acid residue. In specific embodiments, one or more glycine residues of the cupredoxin derived peptide are replaced with a threonine or an alanine residue. In some embodiments, the one or more asparagine or glutamine residues of the cupredoxin derived peptide are replaced by another amino acid residue. In specific embodiments, one or more asparagine or glutamine residues of the cupredoxin derived peptide are replaced with an alanine residue. In other specific embodiments, the glycine at residues 58 and/or 63 of P. aeruginosa azurin (SEQ ID NO: 1), or equivalent glycines of other cupredoxins, are replaced with an alanine or a threonine. In other specific embodiments, the methionine at residue 59 of P. aeruginosa azurin (SEQ ID NO: 1), or an equivalent methionine residue of another cupredoxin derived peptide, is replaced by an alanine residue. In other specific embodiments, the glycine at residue 63 of P. aeruginosa azurin (SEQ ID NO: 1), or an equivalent glycine residue of another cupredoxin derived peptide, is replaced by a threonine residue. In some embodiments, conservative substitutions are made. In other embodiments, non-conservative substitutions are made. In specific embodiments, the modified cupredoxin derived peptide of the invention comprises the following sequence, wherein the underlined amino acids are substituted into the wildtype Pseudomonas aeruginosa p28 sequence

| | |
|---|---|
| LSTAADMQAVVTDTMASGLDKDYLKPDD. | (SEQ ID NO: 51) |

Reversible and irreversible oxidation of amino acids are other biotransformative processes that may also pose a problem that may reduce the pharmacologic activity, and/or plasma half-life of cupredoxin derived peptides. The cysteine and methionine residues are the predominant residues that undergo reversible oxidation. Oxidation of cysteine is accelerated at higher pH, where the thiol is more easily deprotonated and readily forms intra-chain or inter-chain disulfide bonds. These disulfide bonds can be readily reversed in vitro by treatment with dithiothreitol (DTT) or tris(2-carboxyethylphosphine) hydrochloride (TCEP). Methionine oxidizes by both chemical and photochemical pathways to form methionine sulfoxide and further into methionine sulfone, both of which are almost impossible to reverse.

It is contemplated that oxidation in the cupredoxin derived peptides may be prevented by replacing methionine and/or cysteine residues with other residues. In some embodiments, one or more methionine and/or cysteine residues of the cupredoxin derived peptide are replaced by another amino acid residue. In specific embodiments, the methionine residue is replaced with a leucine or valine residue. In other specific embodiments, one or more of the methionines at residues 56 and 64 of P. aeruginosa azurin (SEQ ID NO: 1), or equivalent methionine residues in other cupredoxin derived peptides, are replaced with leucine or valine. In some embodiments, conservative substitutions are made. In other embodiments, non-conservative substitutions are made. In specific embodiments, the cupredoxin peptides of the invention comprise one of the following sequences, wherein the underlined amino acid is substituted into the wildtype Pseudomonas aeruginosa p28 sequence:

| | |
|---|---|
| LSTAADLQGVVTDGLASGLDKDYLKPDD | (SEQ ID NO: 52) |
| or | |
| LSTAADVQGVVTDGVASGLDKDYLKPDD. | (SEQ ID NO: 53) |

Another biotransformative process that may affect the pharmacologic activity, plasma half-life and/or immunogenicity of the cupredoxin derived peptides is diketopiperazine and pyroglutamic acid formation. Diketopiperazine formation usually occurs when glycine is in the third position from the N-terminus, and more especially if proline or glycine is in position 1 or 2. The reaction involves nucleophilic attack of the N-terminal nitrogen on the amide carbonyl between the second and third amino acid, which leads to the cleavage of the first two amino acids in the form of a diketopiperazine. On the other hand, pyroglutamic acid formation may be almost inevitable if glutamine is in the N-terminus. This is an analogous reaction where the N-terminal nitrogen attacks the side chain carbonyl carbon of glutamine to form a deaminated pyroglutamayl peptide analog. This conversion also occurs in peptide containing asparagine in the N-terminus, but to a much lesser extent.

It is contemplated that diketopiperazine and pyroglutamic acid formation may be decreased in cupredoxin derived peptides by replacing glycine in position 1, 2, or 3 from the N-terminus, proline in position 3 from the N-terminus, or asparagine at the N-terminus of the peptide with another amino acid residue. In some embodiments, a glycine in positions 1, 2, or 3 from the N-terminus of the cupredoxin derived peptide is replaced with another amino acid residue. In specific embodiments, the glycine residue is replaced by a threonine or alanine residue. In another embodiment, a proline at position 3 from the N-terminus of the cupredoxin derived peptide is replaced with another amino acid residue. In specific embodiments, the proline is replaced by an alanine residue. In another embodiment, an asparagine at the N-terminus is replaced with another amino acid residue. In specific embodiments, the asparagine residue is replaced by a glutamine residue. In some embodiments, conservative substitutions are made. In other embodiments, non-conservative substitutions are made.

Another biotransformative process that may affect the pharmacologic activity, plasma half-life and/or immunigenicity of the cupredoxin derived peptide is racemization. This term is loosely used to refer to the overall loss of chiral integrity of the amino acid or peptide. Racemization involves the base-catalyzed conversion of one enantiomer (usually the L-form) of an amino acid into a 1:1 mixture of L- and D-enantiomers. One way to improve stability of the peptide in general is by making a retro-inverso (D-isomer) peptide. The double inversion of peptide structure often leaves the surface topology of the side-chain intact and has been used extensively to stabilize biologically active peptides. Snyder et al., PLoS Biol. 2:0186-0193 (2004). A D-amino acid substituted Tat is internalized into cells as well as the L-amino acid peptide. Futaki et al., J. Biol. Chem. 276:5836-5840 (2001); Huq et al., Biochemistry 38:5172-5177 (1999). In some embodiments, one or more amino acid residues of the cupredoxin derived peptide are replaced by the D-isomer of that amino acid residue. In other embodiments, all of the amino acid residues of the cupredoxin derived peptide are replaced with D-isomers of those residues. In one embodiment, the modified cupredoxin derived peptide is a retro-inverso (D-isomer) version of the cupredoxin derived peptide. In a specific embodiment, the modified cupredoxin derived peptide is

| | |
|---|---|
| DDPKLYDKDLGSAMGDTVVGQMDAATSL. | (SEQ ID NO: 54) |

Other methods to protect a cupredoxin derived peptide from biotransformative degradation are N-acetylation and C-amidation. These derivatives may protect the peptide from degradation and may make the cupredoxin derived peptide more closely mimic the charge state of the alpha amino and carboxyl groups in the native protein. Peptides with the N-acetylation and/or C-amidation can be provided by commercial suppliers. In one embodiment of the invention, the N-terminus of the cupredoxin derived peptide may be acetylated. In another embodiment of the invention, the C-terminus of the cupredoxin derived peptides may be amidated. In one specific embodiment, the modified cupredoxin derived peptide is

(SEQ ID NO: 55)

Acetylation-LSTAADMQGVVTDGMASGLDKDYLKPDD-
amidation.

Cyclization is an additional manner of biotransformation that may be beneficial to therapeutic peptides including the cupredoxins as described herein. Cyclization may stabilize therapeutic peptides, allowing them to be stored longer, be administered at lower doses and be administered less frequently. Cyclization has been shown to protect peptides against peptidase and protease degradation. Cyclization can be done chemically or enzymatically. Enzymatic cyclization is generally less problematic than chemical cyclization, as chemical cyclization can lack in regio- and stereospecificity, can lead to multimerization in lieu of cyclization and can require complicated multistep processes. Indeed, it has been shown that thioether cyclization is more protective and stable than a disulfide bond against proteolytic enzymes.

Enzymatic cyclization has been shown in lantibiotics—(mehtlyl)lanthionine-containing bacterial peptides. E.g., R. Rink, et al., “Lantibiotic Structures as Guidelines for the Design of Peptides That Can Be Modified by Lantibioitic Enzymes” 44 Biochem., 8873-82 (2005); R. Rink, et al., “Production of Dehydroamino Acid-Containing Peptides by Lactococcus lactis” 73:6 Applied and Environmental Microbiology, 1792-96 (2007); R. Rink, et al., “N is C, the Cylcase of the Lantibiotic Nisin, Can Catalyze Cyclization of Designed Nonlantibiotic Peptides” 46 Biochem., 13179-89 (2007) (each of which is hereby incorporated by reference in its entirety). Lantibiotics are produced by and inhibit the growth of gram-positive bacteria. In lantibiotics, dehydroalanine and dehydrobutyrine are created by enzyme mediated dehydration of serine and threonine residues. Cysteines are then enzymatically coupled to the dehydrated serine and threonine residues to form thioether cyclizations. Naturally occurring lantibiotics show such couplings via thioether bonds between residues that are up to 19 residues apart. Thioether ring formation depends upon the leader peptide. The location of the cyclization depends upon the cyclase mediated regio- and stereospecific ring closure and the positions of the dehydratable serine and threonine residues.

The best characterized of the lantibiotics is nisin—a pentacyclic peptide antiobiotic produced by Lactococcus lactis. Nisin is composed of four methyllanthionines, one lanthionine, two dehydroalanines, one dehydrobutyrine, and twenty-six unmodified amino acids. Nisin's five thioether cross-links are formed by the addition of cysteine residues to dehydroalanine and dehydrobutyrine residues that originate from serine and threonine. Nisin contains thioether-containing amino acids that are posttranslationally introduced by a membrane-associated enzyme complex. This enzyme complex includes: transporter NisT, serine and threonine dehydratase NisB, and cyclase NisC. NisB dehydrates serine and threonine residues, converting them into dehydroalanine and dehydrobutyrine, respectively. This is followed by NisC catalyzed enantioselective coupling of cysteines to the formed dehydroresidues. NisT facilitates the export of the modified prenisin. Another enzyme, NisP cleaves the nisin leader peptide from prenisin.

The cyclase N is C has been well characterized. Li et al, “Structure and Mechanism of the Lantibiotic Cylclase Involved in Nisin Biosynthesis” 311 Science, 1464-67 (2006) (hereby incorporated by reference in its entirety).

An analysis of cyclization in lantibiotics has led to the identification of amino acid sequences and characteristics in peptides that favor cyclization. It has been shown that the NisB enzyme dehydrates more often where certain amino acids flank the serine and threonine residues. It has been shown that cyclization occurs more often in lantibiotic propeptides where hydrophobic, nonaromatic residues are in proximity to the serine and threonine residues. The flanking residues of the modified cysteines are typically less hydrophobic than the flanking residues of the modified threonines and serines. Exceptions have been found, including hexapeptides VSPPAR (SEQ ID NO: 56), YTPPAL (SEQ ID NO: 57) and FSFFAF (SEQ ID NO: 58). The hexapeptides suggest that the presence of a proline at position 3 or 4 or having phenylalanine flanking both sides may prohibit dehydration. The rings are typically formed by coupling a dehydrated residue to a C-terminally located cysteine. However, rings may be formed by coupling a dehydrate residue to a N-terminally located cysteine.

It has also been shown that the nisin dehydrating and transport enzymes are not specific to nisin and may, in fact, be used to modify non-nisin peptides (and non-lantibiotic peptides). NisB has been shown to dehydrate serine and threonine residues in peptides such as human peptide hormones when such peptides are N-terminally fused to the lantibiotic leader peptide. On non-lantibiotic peptides, similar ring formation characteristics apply; namely, the extent of dehydration can be controlled by the amino acid context of the flanking region of the dehydratable serine and threonine residues. The presence of hydrophobic flanking residues (e.g., alanine and valine) around the serines and threonines allowed full dehydration and therefore enhanced thioether ring formation. The presence of an N-terminal aspartate and C-terminally flanked arginine prevented dehydration. It also shown that the presence of proline residues and phenylalanine residues is disfavorable for dehydration. Generally, the presence of hydrophilic flanking residues prevented dehydration of the serine and threonine residues. Hydrophobic flanking favors dehydration; hydrophilic flanking disfavors dehydration. Studies have shown that where dehydration does occur, the average hydrophobicity of the flanking residues of serines and threonine is positive—0.40 on the N-terminal side and 0.13 on the C-terminal side. Also, the average hydrophobicity of the residues flanking serines and threonines that are not dehydrated is negative—−0.36 on the N-terminal side and −1.03 on the C-terminal side. Deydration is not restricted by the presence of a series of flanking threonine residues and is not restricted by the distance between the nisin leader peptide and the residue to be dehydrated.

NisC has been shown to catalyze the regiospecific formation of thioether rings in peptides unrelated to naturally occurring lantibiotics. Generally, such peptides must be fused to the nisin leader peptide. In some cases, thioether rings may form spontaneously, for example where a dehydroalanine is spaced by two amino acids from a cysteine. Unlike spontaneous cyclization, N is C catalyzed cyclization is stereospecific for dehydrated pre-nisin. Consequently, the methyllanthionines and lanthionine in nisin are in the DL configuration. It is thought that cyclization in nonlantibiotic peptides will also be stereo specific

These principles can be applied to the compounds described herein, including cupredoxins, cytochromes, and variants derivatives, truncations, and structural equivalents thereof.

### Thioether Bridges

In nature, lantibiotic-enzyme-induced thioether bridges occur with up to 19 amino acids under the bridge. Thioether bridges with 2 to 4 amino acids under the bridge are abundant.

In some embodiments, the cupredoxins and cytochromes and derivatives, variants, truncations, or structural equivalents thereof, such as truncated azurin, may be modified by introducing thioether bridges into the structure. The azurin truncation p28 (SEQ ID NO: 29), for example, may be modified using this method. Extended molecular dynamics simulations (70 ns) using software package GROMACS (www.gromacs.org) suggest that, at 37° C., the region of the p28 alpha helix from position 6 to 16 is unstable, and that the peptide tends to adopt a beta sheet conformation. This, together with the fact that the part of the molecule presumed to be responsible for interaction with p53 remains solvent exposed, suggests that introduction of a thioether bridge in this region of the p28 peptide may not affect its functionality.

[symbol]

Structure 1: Azurin Truncation with Alpha-Helical Structure

[symbol]

### Structure 2: Result of 70 ns Simulation.

The amino acid sequence of p28 is SEQ ID NO: 29 (LSTAADMQGVVIDGMASGLDKDYLKPDD). The amino acid sequence known as p18 is SEQ ID NO: 30 (LSTAADMQGVVTDGMASG). Thioether bridges can be formed between Ser/Thr on the N-side to Cys on the C-side. The serine/threonine is dehydrated and subsequently coupled to the cysteine. Threonines are preferred since they are more easily dehydrated than serines. Generally, hydrophobic flanking residues (at least one) to the threonine are preferred since they enhance the extent of dehydration. Negatively charged amino acids, glutamate and aspartate, that are flanking residues have a strong negative effect on dehydration. Generally, hydrophilic flanking residues, especially glycin, do not favor dehydration. Preceding the Cys there is a slight preference for charged hydrophilic residues, especially glutamate/aspartate. Depending on the size of the thioether ring, the bulkiness of the amino acids that participate in the ring matters.

In one embodiment, the truncated azurin sequence is LSTAADMQGVVTDGMASGLDKDYLTPGC (SEQ ID NO: 59). A thioether bridge is formed between positions 25 and 28 of p28, and will be fully protected against carboxyetidases. Positions 2, 3 and 25 will be dehydrated, but neither the import sequence, nor the sequence thought to be relevant for interaction with p53, is altered by thioether ring introduction. As such, peptide activity should not be altered. The threonine is between two hydrophobic amino acids and hence is expected to be fully dehydrated by dehydratase, NisB, according to specific guidelines. See Rink et al., Biochemistry 2005. The same guidelines also predict cyclization involving positions 25 and 28 by cyclase NisC, especially because of the aspartate located before the cysteine.

In another embodiment, the truncated azurin sequence is LSTAADCQGVVTDGMASGLDKDYLKPDD (SEQ ID NO: 60) and the thioether bridge is formed between positions 3 and 7. The ring between position 3 and 7 mimics ring A of nisin and makes use of the existing threonine at position 2. The aspartate at position 6 will favor cyclization.

In another embodiment, the truncated azurin sequence is LSTAACMQGVVTDGMASGLDKDYLKPDD (SEQ ID NO: 61), and the threonine in position 2 is utilized to form a thioether bridge.

In another embodiment, two or more of the thioether rings in the truncated azurins described in the paragraphs above are combined into one peptide.

In another embodiment, many truncated azurin sequences can be created and screened for threonine rings by analyzing the peptides with a ring of one lanthionine and two to three additional amino acids under the sulfur bridge. This might involve one or combinations of the sequences below:

| | |
|---|---|
| LSTACDMQGVVTDGMASGLDKDYLKPDD | (SEQ ID NO: 62) |
| LSTAATMQCVVTDGMASGLDKDYLKPDD | (SEQ ID NO: 63) |
| LSTAATMQGCVTDGMASGLDKDYLKPDD | (SEQ ID NO: 64) |
| LSTAANTQGCVTDGMASGLDKDYLKPDD | (SEQ ID NO: 65) |
| LSTAANTQGVCTDGMASGLDKDYLKPDD | (SEQ ID NO: 66) |
| LSTAADMTAVCTDGMASGLDKDYLKPDD | (SEQ ID NO: 67) |
| LSTAADMTAVVCDGMASGLDKDYLKPDD | (SEQ ID NO: 68) |
| LSTAADMQTVVCDGMASGLDKDYLKPDD | (SEQ ID NO: 69) |
| LSTAADMQTVVTCGMASGLDKDYLKPDD | (SEQ ID NO: 70) |
| LSTAADMQATVTCGMASGLDKDYLKPDD | (SEQ ID NO: 71) |
| LSTAADMQATVTDCMASGLDKDYLKPDD | (SEQ ID NO: 72) |
| LSTAADMQGVTADCMASGLDKDYLKPDD | (SEQ ID NO: 73) |
| LSTAADMQGVTADGCASGLDKDYLKPDD | (SEQ ID NO: 74) |
| LSTAADMQGVVTNGCASGLDKDYLKPDD | (SEQ ID NO: 75) |

A practical approach would be to genetically make a large number of such sequences and select a group for purification on the basis of extent of modification and level of production.

In another embodiment, a thioether bridge is formed between a threonine at position 12 in p28 (SEQ ID NO: 29) and the c-terminus of the peptide. The distance between the Cα of position 13 and the aspartate at position 28 might be 17.52 angstroms, larger than 1.5 nanometers, implying significant alteration of the structure of the peptide.

[symbol]

### Structure 3: Measurement of Thioether Bridge Positions Based on Distances Between Cα Atoms in a Simulated Structure.

In another embodiment, the peptide sequence is LSTAADMQGVVTATMGSGLCKDYLKPDD (SEQ ID NO: 76), with a thioether bridge from position 14 to position 2 at a distance of 4.38 angstroms. The mutation of aspartate at position 13 to alanine favors dehydration of threonine at position 14. Mutation of alanine at position 16 to glycine completely prevents dehydration of serine at position 17 and enhances cyclization.

In another embodiment, the peptide sequence is LSTAADMQGVVTDLTASGLCKDYLKPDD (SEQ ID NO: 77), with the thioether bridge from position 15 to position 20 at a distance of 5.83 angstroms. In this situation, mutation of glycine at position 14 to leucine favors dehydration of threonine at position 15.

### Tertiary Structure Stabilization

The stability of the tertiary structure of the cupredoxin, cytochrome, or variant, derivative, truncation, or structural equivalent thereof will affect most aspects of the pharmacokinetics, including the pharmacologic activity, plasma half-life, and/or immunogenicity among others. See Kanovsky et al., Cancer Chemother. Pharmacol. 52:202-208 (2003); Kanovsky et al., PNAS 23:12438-12443 (2001). Peptide helices often fall apart into random coils, becoming more susceptible to protease attack and may not penetrate cell membrane well. Schafmeister et al., J. Am. Chem. Soc. 122:5891-5892 (2000). Therefore, one way to stabilize the overall structure of a peptide such as a cupredoxin is to stabilize the α-helix structure of the peptide. The intra-molecular hydrogen bonding associated with helix formation reduces the exposure of the polar amide backbone, thereby reducing the barrier to membrane penetration in a transport peptide, and thus increasing related pharmacologic activities and increasing the resistance of the peptide to protease cleavage. Id. Pseudomonas aeruginosa azurin (SEQ ID NO: 1) has α-helices at residues 53-56, 58-64 and 68-70.

One method to stabilize an α-helix is to replace in the α-helix helix breaking amino acid residues such as glycine, proline, serine and aspartic acid, or helix neutral amino acid residues such as alanine, threonine, valine, glutamine, asparagine, cysteine, histidine, lysine or arginine, with helix forming residues, such as leucine, isoleucine, phenylalanine, glutamic acid, tyrosine, tryptophan and methionine or helix favoring amino acid residue substitutions, for example α-amino-isobutyric acid (Aib). See Miranda et al., J. Med. Chem., 51, 2758-2765 (2008), the disclosure of which is incorporated by reference herein. It is contemplated that the α-helix of cupredoxin derived peptides may be stabilized by replacing one or more glycine, proline, serine and/or aspartic acid residues with other amino acids. In specific embodiments, the glycine, proline, serine, aspartic acid, alanine, threonine, valine, glutamine, asparagine, cysteine, histidine, lysine and/or arginine residues are replaced by leucine, isoleucine, phenylalanine, glutamic acid, tyrosine, tryptophan, Aib and/or methionine residues. See Lee et al., Cancer Cell Intl. 11:21 (2005). In other specific embodiments, one or more serine or glutamine residues in the α-helices of a cupredoxin derived peptide may be substituted. In still more specific embodiments, the serine and/or glutamine residues in residues 53-56, 58-64 and 68-70 of P. aeruginosa azurin (SEQ ID NO: 1), or equivalent residues of other cupredoxin derived peptides, may be replaced. In another specific embodiment, the glutamine residue at amino acid residue 57 of P. aeruginosa azurin (SEQ ID NO: 1), or an equivalent residue of another cupredoxin derived peptide, may be replaced, more specifically replaced with tryptophan. In another specific embodiment, the threonine residue at amino acid residue 52 of P. aeruginosa azurin (SEQ ID NO: 1), or an equivalent residue of another cupredoxin derived peptide, may be replaced, more specifically replaced with tryptophan. In another specific embodiment, the threonine residue at amino acid residue 61 of P. aeruginosa azurin (SEQ ID NO: 1), or an equivalent residue of another cupredoxin derived peptide, may be replaced, more specifically replaced with tryptophan. In another specific embodiment, the glycine residue at amino acid residue 63 of P. aeruginosa azurin (SEQ ID NO: 1), or an equivalent residue of another cupredoxin derived peptide, may be replaced, more specifically replaced with tryptophan. In another specific embodiment, one or more threonine, glutamine or glycine residues at amino acid residues 52, 57, 61 or 63 of P. aeruginosa azurin (SEQ ID NO: 1), or an equivalent residue of another cupredoxin derived peptide, may be replaced, more specifically replaced with tryptophan. In specific embodiments, the cupredoxin peptide comprises one of the following sequences wherein the underlined amino acid is substituted into the wildtype Pseudomonas aeruginosa p28 sequence:

| | |
|---|---|
| LSWAADMQGVVTDGMASGLDKDYLKPDD; | (SEQ ID NO: 78) |
| LSTAADMWGVVTDGMASGLDKDYLKPDD; | (SEQ ID NO: 79) |
| LSTAADMQGVVWDGMASGLDKDYLKPDD; | (SEQ ID NO: 80) |
| LSTAADMQGVVTDWMASGLDKDYLKPDD; | (SEQ ID NO: 81) |
| LSWAADMWGVVTDGMASGLDKDYLKPDD; | (SEQ ID NO: 82) |
| LSWAADMQGVVWDGMASGLDKDYLKPDD; | (SEQ ID NO: 83) |
| LSWAADMQGVVTDWMASGLDKDYLKPDD; | (SEQ ID NO: 84) |
| LSTAADMWGVVWDGMASGLDKDYLRPDD; | (SEQ ID NO: 85) |
| LSTAADMWGVVTDWMASGLDKDYLKPDD; | (SEQ ID NO: 86) |
| LSTAADMQGVVWDWMASGLDKDYLKPDD; | (SEQ ID NO: 87) |
| or | |
| LSWAADMWGVVWDWMASGLDKDYLKPDD. | (SEQ ID NO: 88) |

In other embodiments, equivalent amino acids in other cupredoxin derived peptides are substituted with tryptophan.

Another method to stabilize an α-helix tertiary structure involves using unnatural amino acid residues capable of π-stacking. For example, in Andrews and Tabor (Tetrahedron 55:11711-11743 (1999)), pairs of ε-(3,5-dinitrobenzoyl)-Lys residues were substituted into the α-helix region of a peptide at different spacings. The overall results showed that the i,(i+4) spacing was the most effective stabilizing arrangement. Increasing the percentage of water, up to 90%, increased the helical content of the peptide. Pairs of ε-acyl-Lys residues in the same i,(i+4) spacing had no stabilizing effect, indicating that the majority of the stabilization arises from π-π interactions. In one embodiment, the cupredoxin derived peptide may be modified so that the lysine residues are substituted by ε-(3,5-dinitrobenzoyl)-Lys residues. In a specific embodiment, the lysine residues may be substituted by ε-(3,5-dinitrobenzoyl)-Lys in a i,(i+4) spacing.

Another method to stabilize an α-helix tertiary structure uses the electrostatic interactions between side-chains in the α-helix. When His-Cys or His-His residue pairs were substituted in into peptides in an i,(i+4) arrangement, the peptides changed from about 50% helical to about 90% helical on the addition of Cu, Zn or Cd ions. When ruthenium (Ru) salts were added to the His-His peptides, an exchange-inert complex was formed, a macrocyclic cis-[Ru—(NH₃)₄L₂]³⁺ complex where L₂ are the side chains of two histidines, which improved the helix stability. Ghadiri and Femholz, J. Am. Chem. Soc. 112, 9633-9635 (1990). In some embodiments, the cupredoxin derived peptides may comprise macrocyclic cis-[Ru—(NH₃)₄L₂]³⁺ complexes where L₂ is the side chains of two histidines. In some embodiments, one or more histidine-cysteine or histidine-histidine residue pairs may be substituted an i,(i+4) arrangement into the α-helices of the cupredoxin derived peptide. In other embodiments, one or more histidine-cysteine or histidine-histidine residue pairs may be substituted an i,(i+4) arrangement in residues 53-56, 58-64 and 68-70 of P. aeruginosa azurin (SEQ ID NO: 1), or equivalent residues of other cupredoxin derived peptides. In some embodiments, the cupredoxin derived peptide may further comprise Cu, Zn, Cd and/or Ru ions.

Another method to stabilize an α-helix tertiary structure involves disulfide bond formation between side-chains of the α-helix. It is also possible to stabilize helical structures by means of formal covalent bonds between residues separated in the peptide sequence. The commonly employed natural method is to use disulfide bonds. Pierret et al., Intl. J. Pept. Prot. Res., 46:471-479 (1995). In some embodiments, one or more cysteine residue pairs are substituted into the α-helices of the cupredoxin derived peptide. In other embodiments, one or more cysteine residue pairs are substituted at residues 53-56, 58-64 and 68-70 of P. aeruginosa azurin (SEQ ID NO: 1), or equivalent residues of other cupredoxin derived peptides.

Another method to stabilize an α-helical tertiary structure involves the use of side chain lactam bridges. A lactam is a cyclic amide which can form from the cyclisation of amino acids. Side chain to side chain bridges have been successfully used as constraints in a variety of peptides and peptide analogues, such as amphipathic or model α-helical peptides, oxytocin antagonists, melanoptropin analogues, glucagon, and SDF-1 peptide analogues. For example, the Glucagon-like Peptide-1 (GLP-1) gradually assumes a helical conformation under certain helix-favoring conditions and can be stabilized using lactam bridging. Miranda et al., J. Med. Chem., 51, 2758-2765 (2008). These lactam bridges may be varied in size, effecting stability and binding affinity. Id. Such modifications improved the stability of the compounds in plasma. Id. Depending on the space between the cyclization sites and choice of residues; lactam bridges can be used to induce and stabilize turn or helical conformations. In some embodiments, one or more cupredoxin or variant analogues are prepared with lactam bridging between nearby amino acids (such as i to i+4 glutamic acid-lysine constraints). In some embodiments, the cupredoxin derived peptide may comprise such modifications to enhance α-helix content.

Another method to stabilize an α-helix tertiary structure is the all-carbon cross-link method. The all-hydrocarbon cross-link method is proven to increase the stabilization of helical structure, protease resistant and cell-permeability. Walensky et al., Science, 305, 1466-1470 (2004). α,α-disubstituted non-natural amino acids containing olefin-bearing tethers are incorporated into peptides. Ruthenium catalyzed olefin metathesis generates an all-hydrocarbon “staple” to cross-link the helix. Schafmeister et al., J. Am. Chem. Soc., 122, 5891-5892 (2000); Walensky et al., id. Non-natural amino acids containing olefin-bearing tethers may be synthesized according to methodology provided in Schafmeister et al. (id.) and Williams and Im (J. Am. Chem. Soc., 113:9276-9286 (1991)). In some embodiments, the cupredoxin derived peptides are stabilized by all-hydrocarbon staples. In specific embodiments, one or more pairs of α,α-disubstituted non-natural amino acids containing olefin-bearing tethers corresponding to the native amino acids are substituted into the α-helices of the cupredoxin derived peptide. In other embodiments, one or more pairs of α,α-disubstituted non-natural amino acids containing olefin-bearing tethers corresponded to the native amino acids are substituted into residues 53-56, 58-64 and 68-70 of P. aeruginosa azurin (SEQ ID NO: 1), or equivalent residues of other cupredoxin derived peptides.

In some embodiments, the modified cupredoxin derived peptide may comprise X₁SX₂AADX₃X₄X₅VVX₆DX₇X₈ASGLDKDYLKPDX₉ (SEQ ID NO: 89), where X₁ is L or acetylated-L, X₂ is T or W, X₃ is M, L or V, X₄ is Q or W, X₅ is G or A, X₆ is T or W, X₇ is G, T or W, X₈ is M, L or V, and X₉ is D or amidated-D. In other embodiments, the modified cupredoxin derived peptide may consist of X₁SX₂AADX₃X₄X₅VVX₆DX₇X₈ASGLDKDYLKPDX₉ (SEQ ID NO: 89), where X₁ is L or acetylated-L, X₂ is T or W, X₃ is M, L or V, X₄ is Q or W, X₅ is G or A, X₆ is T or W, X₇ is G, T or W, X₈ is M, L or V, and X₉ is D or amidated-D. In other embodiments, the modified cupredoxin derived peptide may comprise X₁DPKLYDKDLGSAX₂X₃DX₄VVX₅X₆X₇DAAX₈SX₉ (SEQ ID NO: 90), where X₁ is D or acetylated-D, X₂ is M, L or V, X₃ is G, T or W, X₄ is T or W, X₅ is G or A, X₆ is Q or W, X₇ is M, L or V, X₈ is T or W, and X₉ is L or amidated-L. In other embodiments, the modified cupredoxin derived peptide may consist of X₁DPKLYDKDLGSAX₂X₃DX₄VVX₅X₆X₇DAAX₈SX₉ (SEQ ID NO: 90), where X₁ is D or acetylated-D, X₂ is M, L or V, X₃ is G, T or W, X₄ is T or W, X₅ is G or A, X₆ is Q or W, X₇ is M, L or V, X₈ is T or W, and X₉ is L or amidated-L. Specific peptides of interest are listed in Table 3.

PEGylation

Covalent attachment of PEG to drugs of therapeutic and diagnostic importance has extended the plasma half-life of the drug in vivo, and/or reduced their immunogenicity and antigenicity. Harris and Chess, Nature Reviews Drug Discovery 2:214-221 (2003). For example, PEG attachment has improved the pharmacokinetic properties of many therapeutic proteins, including interleukins (Kaufman et al., J. Biol. Chem. 263:15064 (1988); Tsutsumi et al., J. Controlled Release 33:447 (1995)), interferons (Kita et al., Drug Des. Delivery 6:157 (1990)), catalase (Abuchowski et al., J. Biol. Chem. 252:3582 (1977)), superoxide dismutase (Beauchamp et al., Anal. Biochem. 131:25 (1983)), and adenosine deaminase (Chen et al., Biochem. Biophys. Acta 660:293 (1981)), among others. The FDA has approved PEG for use as a vehicle or base in foods, cosmetics and pharmaceuticals, including injectable, topical, rectal and nasal formulations. PEG shows little toxicity, and is eliminated from the body intact by either the kidneys (for PEGs<30 kDa) or in the feces (for PEGs>20 kDa). PEG is highly soluble in water.

PEGylation of cupredoxins, cytochromes, and/or variants, derivatives, truncations, and structural equivalents thereof, particularly cupredoxin-derived peptides such as truncations of azurin, may be used to increase the lifetime of the peptide in the bloodstream of the patient by reducing renal ultrafiltration, and thus reduce elimination of the drug from the body. Charge masking may affect renal permeation. Charge masking may be a consequence of the paramchemical modification of protein ionizable functional group, namely amines or carboxyls. In particular, the most common procedures for producing protein-PEG derivatives involves the conversion of protein amino groups into amides with the consequent loss of positive charges, and this can alter protein ultrafiltration. Since anionic macromolecules have been found to be cleared by renal ultrafiltration more slowly than neutral or positive ones, it could be expected that PEG conjugation to amino groups prolongs the permanence of the PEGylated peptide in the bloodstream.

Molecular size and globular ultrafiltration may also affect renal ultrafiltration of therapeutic peptides. The molecular weight cut off for kidney elimination of native globular proteins is considered to be about 70 kDa, which is close to the molecular weight of serum albumin. Thus, proteins with molecular weight exceeding 70 kDa are mainly eliminated from the body by pathways other than renal ultrafiltration, such as liver uptake, proteolytic digestion and clearance by the immune system. Therefore, increasing the size of a therapeutic peptide by PEGylation may decrease renal ultrafiltration of that peptide form the bloodstream of the patient.

Additionally, PEGylation of a peptide may decrease the immunogenicity of that peptide, as well as protect the peptide from proteolytic enzymes, phagocytic cells, and other factors that require direct contact with the therapeutic peptide. The umbrella-like structure of branched PEG in particular has been found to give better protection than linear PEG towards approaching proteolytic enzymes, antibodies, phagocytic cells, etc. Caliceti and Veronese, Adv. Drug. Deliv. Rev. 55:1261-12778 (2003).

In some embodiments, the cupredoxin derived peptides are modified to have one or more PEG molecules covalently bonded to a cysteine molecule. The covalent bonding does not necessarily need to be a covalent bond directly from the PEG molecule to the cupredoxin derived peptide, but may be covalently bonded to one or more linker molecules which in turn are covalently bonded to each other and/or the cupredoxin derived peptide. In some embodiments, the cupredoxin derived peptide have site-specific PEGylation. In specific embodiments, the PEG molecule(s) may be covalently bonded to the cysteine residues 3, 26 and/or 112 of P. aeruginosa azurin (SEQ ID NO: 1). In other embodiments, one or more cysteine residues may be substituted into the cupredoxin derived peptide and is PEGylated. In some embodiments, the method to PEGylate the cupredoxin derived peptide may be NHS, reductive animation, malimid or epoxid, among others. In other embodiments, the cupredoxin derived peptides may be PEGylated on one or more lysine, cysteine, histidine, arginine, aspartic acid, glutamic acid, serine, threonine, or tyrosine, or the N-terminal amino group or the C-terminal carboxylic acid. In more specific embodiments, the cupredoxin derived peptides may be PEGylated on one or more lysines or N-terminal amino groups. In other embodiments, one or more lysine, cysteine, histidine, arginine, aspartic acid, glutamic acid, serine, threonine, or tyrosine residue are substituted into the cupredoxin derived peptides and are PEGylated. In other embodiments, the cupredoxin derived peptides may be PEGylated on one or more amino groups. In other embodiments, the cupredoxin derived peptides may be PEGylated in a random, non-site specific manner. In some embodiments, the cupredoxin derived peptides may have an average molecular weight of PEG-based polymers of about 200 daltons to about 100,000 daltons, about 2,000 daltons to about 20,000 daltons, or about 2,000 daltons to about 5,000 daltons. In other embodiments, the cupredoxin derived peptides may be comprised of one or more PEG molecules that is branched, specifically a branched PEG molecule that is about 50 kDa. In other embodiments, the cupredoxin derived peptides may comprise one or more linear PEG molecules, specifically a linear PEG molecule that is about 5 kDa.

In another embodiment, the chemopreventive agent is a peptide that is a cupredoxin, or variant, truncation, structural equivalent, or derivative thereof that is a conjugate of Pep42, a cyclic 13-mer oligopeptide that specifically binds to glucose-regulated protein 78 (GRP78) and is internalized into cancer cells. The cupredoxin or variant, structural equivalent, or derivative of cupredoxin may be conjugated with Pep42 pursuant to the synthesis methods disclosed in Yoneda et al., “A cell-penetrating peptidic GRP78 ligand for tumor cell-specific prodrug therapy,” Bioorganic & Medicinal Chemistry Letters 18: 1632-1636 (2008), the disclosure of which is incorporated in its entirety herein.

In another embodiment, the peptide is a structural equivalent of a cupredoxin or cytochrome. Examples of studies that determine significant structural homology between cupredoxins and cytochromes and other proteins include Toth et al. (Developmental Cell 1:82-92 (2001)). Specifically, significant structural homology between a cupredoxin or cytochrome and its structural equivalents are determined by using the VAST algorithm (Gibrat et al., Curr Opin Struct Biol 6:377-385 (1996); Madej et al., Proteins 23:356-3690 (1995)). In specific embodiments, the VAST p value from a structural comparison of a cupredoxin or cytochrome to the structural equivalent is less than about 10⁻³, less than about 10⁻⁵, or less than about 10⁻⁷. In other embodiments, significant structural homology between a cupredoxin or cytochrome and its structural equivalents are determined by using the DALI algorithm (Holm & Sander, J. Mol. Biol. 233:123-138 (1993)). In specific embodiments, the DALI Z score for a pairwise structural comparison is at least about 3.5, at least about 7.0, or at least about 10.0.

In some embodiments, the cupredoxin, or variant, derivative, truncation, or structural equivalent thereof has some of the pharmacologic activities of the P. aeruginosa azurin, and p28. In a specific embodiment, the cupredoxins and variants, derivatives and structural equivalents of cupredoxins that may inhibit prevent the development of premalignant lesions in mammalian cells, tissues or animals, and specifically but not limited to, mammary gland cells. The invention also provides for the cupredoxins and variants, derivatives and structural equivalents of cupredoxins that may have the ability to inhibit the development of mammalian premalignant lesions, and specifically but not limited to, melanoma, breast, pancreas, glioblastoma, astrocytoma, lung, colorectal, neck and head, bladder, prostate, skin and cervical cancer cells. Inhibition of the development of cancer cells is any decrease, or lessening of the rate of increase, of the development of premalignant lesions that is statistically significant as compared to control treatments.

In some embodiments, the cupredoxin or cytochrome, or variant, derivative, truncation, or structural equivalent thereof has some of the functional characteristics of the P. aeruginosa azurin or cytochrome. In a specific embodiment, the cupredoxin or cytochrome inhibits the growth of viral or bacterial infection, and specifically HIV infection in mammalian cells, more specifically in peripheral blood mononuclear cells infected with HIV. The invention also provides for the variants, derivatives and structural equivalents of cupredoxin and cytochrome c₅₅₁ that retain the ability to inhibit the growth of viral or bacterial infection, and specifically HIV infection in mammalian cells. The growth of HIV-1 infection in the cells may be determined by measuring the change in the production of HIV-1 p24 antigen in the cell culture supernatant by a commercial p24 enzyme immunoassay (PerkinElmer Life Sciences, Inc., Wellesley, Mass.). Inhibition of a growth of infection is any decrease or lessening of the rate of increase of that infection that is statistically signification as compared to control treatments.

In some specific embodiments, the peptide of the invention may also induce apoptosis in a mammalian cancer cell, more specifically a J774 cell. The ability of a cupredoxin or other polypeptide to induce apoptosis may be observed by mitosensor ApoAlert confocal microscopy using a MITOSENSOR™ APOLER™ Mitochondrial Membrane Sensor kit (Clontech Laboratories, Inc., Palo Alto, Calif., U.S.A.), by measuring caspase-8, caspase-9 and caspase-3 activity using the method described in Zou et al. (J. Biol. Chem. 274: 11549-11556 (1999)), and by detecting apoptosis-induced nuclear DNA fragmentation using, for example, the APOLERT™ DNA fragmentation kit (Clontech Laboratories, Inc., Palo Alto, Calif., U.S.A.).

In another specific embodiment, the peptide of the invention may also induce cellular growth arrest in a mammalian cancer cell, more specifically a J774 cell. Cellular growth arrest can be determined by measuring the extent of inhibition of cell cycle progression, such as by the method found in Yamada et al. (PNAS 101:4770-4775 (2004)). In another specific embodiment, the cupredoxin or cytochrome c₅₅₁, or variant, derivative, truncation, or structural equivalent thereof inhibits cell cycle progression in a mammalian cancer cell, more specifically a J774 cell.

In some specific embodiments, the cupredoxin, cytochrome or variant, derivative, truncation, or structural thereof, is administered to a patient for the concurrent treatment and/or prevention of two or more conditions such as interstitial cystitis (IC), lesions associated with inflammatory bowel disease (IBD), HIV infection, AIDS, central nervous system disorders, peripheral vascular diseases, viral diseases, degeneration of the central nervous system (Christopher Reeve's disease), Alzheimer's disease, malaria, inappropriate angiogenesis, cardiovascular disease, hypertension, Cytomegalovirus infection, human papilloma virus infection; Muscular Dystrophy, encephalopathy, dementia, Parkinson's disease, neuropathy, macular degeneration, diabetic retinopathy, rheumatoid arthritis, psoriasis, herpes simplex virus (HSV), Ebola virus, cytomeglovirus (CMV), Para influenza viruses types A, B and C, hepatitis virus A, B, C, and G, the delta hepatitis virus (HDV), mumps virus, measles virus, respiratory syncytial virus, bunyvirus, arena virus, Dhori virus, poliovirus, rubella virus, dengue virus; SIV, Mycobacterium tuberculosis and cancer. More specifically, the cancer may be melanoma, leukemia, breast cancer, ovarian cancer, lung cancer, mesenchymal cancer, colon cancer, aerodigestive tract cancer, cervical cancer, brain tumors or prostate cancer.

In a specific embodiment, the cupredoxin, cytochrome or variant, derivative, truncation, or structural thereof, is administered to a patient for the concurrent treatment and/or prevention of two or more conditions selected from the group consisting of cancer, HIV, malaria and inappropriate angiogenesis.

In another specific embodiment, the cupredoxin, cytochrome or variant, derivative, truncation, or structural equivalent thereof, may be in a composition as a therapeutic agent for the treatment of malaria, wherein the patient is additionally suffering from HIV, cancer or inappropriate angiogenesis or has a higher risk than the general population of acquiring a condition such as HIV, cancer or inappropriate angiogenesis.

In another specific embodiment, the cupredoxin, cytochrome or variant, derivative, truncation, or structural equivalent thereof, may be in a composition as a therapeutic agent for the treatment of HIV, wherein the patient is additionally suffering from malaria, cancer or inappropriate angiogenesis or has a higher risk than the general population of acquiring a condition such as malaria, cancer or inappropriate angiogenesis.

In another specific embodiment, the cupredoxin, cytochrome or variant, derivative, truncation, or structural equivalent thereof, may be in a composition as a therapeutic agent for the treatment of cancer, wherein the patient is additionally suffering from HIV, malaria or inappropriate angiogenesis or has a higher risk than the general population of acquiring a condition such as HIV, malaria or inappropriate angiogenesis.

In another specific embodiment, the cupredoxin, cytochrome or variant, derivative, truncation, or structural equivalent thereof, may be in a composition as a therapeutic agent for the treatment of inappropriate angiogenesis, wherein the patient is additionally suffering from HIV, cancer or malaria or has a higher risk than the general population of acquiring a condition such as HIV, cancer or malaria.

In another specific embodiment, the cupredoxin, cytochrome or variant, derivative, truncation, or structural equivalent thereof, may be in a composition with, may be co-administered, or may be administered at about the same time as another drug. Such drugs may include, but are not limited to an anti-malarial drug, an anti-HIV drug, an anti-cancer drug, or an anti-angiogenesis drug.

In another specific embodiment, the cupredoxin, cytochrome or variant, derivative, truncation, or structural equivalent thereof, may be in a composition that is administered by a mode of intravenous injection, intramuscular injection, subcutaneous injection, inhalation, topical administration, transdermal patch, suppository, vitreous injection and oral.

### Cupredoxins

These small blue copper proteins (cupredoxins) are electron transfer proteins (10-20 kDa) that participate in bacterial electron transfer chains or are of unknown function. The copper ion is solely bound by the protein matrix. A special distorted trigonal planar arrangement to two histidine and one cystine ligands around the copper gives rise to very peculiar electronic properties of the metal site and an intense blue color. A number of cupredoxins have been crystallographically characterized at medium to high resolution.

The cupredoxins in general have a low sequence homology but high structural homology. (Gough & Clothia, Structure 12:917-925 (2004); De Rienzo et al., Protein Science 9:1439-1454 (2000)). For example, the amino acid sequence of azurin is 31% identical to that of auracyanin B, 16.3% to that of rusticyanin, 20.3% to that of plastocyanin, and 17.3% to that of pseudoazurin. See Table 1. However, the structural similarity of these proteins is more pronounced. The VAST p value for the comparison of the structure of azurin to auracyanin B is 10^{-7.4}, azurin to rusticyanin is 10⁻⁵, azurin to plastocyanin is 10^{-5.6}, and azurin to psuedoazurin is 10^{-4.1}.

All of the cupredoxins possess an eight-stranded Greek key beta-barrel or beta-sandwich fold and have a highly conserved site architecture. (De Rienzo et al., Protein Science 9:1439-1454 (2000)). A prominent hydrophobic patch, due to the presence of many long chain aliphatic residues such as methionines and leucines, is present around the copper site in azurins, amicyanins, cyanobacterial plastocyanins, cucumber basic protein and to a lesser extent, pseudoazurin and eukaryotic plastocyanins. Id. Hydrophobic patches are also found to a lesser extent in stellacyanin and rusticyanin copper sites, but have different features. Id.

TABLE 1
Sequence and structure alignment of azurin (1JZG) from
P. aeruginosa to other proteins using VAST algorithm.

| | Alignment | % aa | P- | | | |
|---|---|---|---|---|---|---|
| PDB | length¹ | identity | value² | Score³ | ⁽ⁱ⁾ RMSD⁴ | (ii) Description |
| 1AOZ A2 | 82 | 18.3 | 10e−7 | 12.2 | 1.9 | Ascorbate oxidase |
| 1QHQ_A | 113 | 31 | 10e−7.4 | 12.1 | 1) 1.9 | 2) AuracyaninB |
| 1V54 B 1 | 79 | 20.3 | 10e−6.0 | 11.2 | 2.1 | Cytocrome c oxidase |
| 1GY2 A | 92 | 16.3 | 10e−5.0 | 11.1 | 3) 1.8 | 4) Rusticyanin |
| 3MSP A | 74 | 8.1 | 10e−6.7 | 10.9 | 2.5 | Motile Major Sperm |
| | | | | | | Protein⁵ |
| 1IUZ | 74 | 20.3 | 10e−5.6 | 10.3 | 5) 2.3 | 6) Plastocyanin |
| 1KGY E | 90 | 5.6 | 10e−4.6 | 10.1 | 7) 3.4 | 8) Ephrinb2 |
| 1PMY | 75 | 17.3 | 10e−4.1 | 9.8 | 9) 2.3 | 10) Pseudoazurin |

¹Aligned Length: The number of equivalent pairs of C-alpha atoms superimposed between the two structures, i.e. how many residues have been used to calculate the 3D superposition.
²P-VAL: The VAST p value is a measure of the significance of the comparison, expressed as a probability. For example, if the p value is 0.001, then the odds are 1000 to 1 against seeing a match of this quality by pure chance. The p value from VAST is adjusted for the effects of multiple comparisons using the assumption that there are 500 independent and unrelated types of domains in the MMDB database. The p value shown thus corresponds to the p value for the pairwise comparison of each domain pair, divided by 500.
³Score: The VAST structure-similarity score. This number is related to the number of secondary structure elements superimposed and the quality of that superposition. Higher VAST scores correlate with higher similarity.
⁴RMSD: The root mean square superposition residual in Angstroms. This number is calculated after optimal superposition of two structures, as the square root of the mean square distances between equivalent C-alpha atoms. Note that the RMSD value scales with the extent of the structural alignments and that this size must be taken into consideration when using RMSD as a descriptor of overall structural similarity.
⁵C. elegans major sperm protein proved to be an ephrin antagonist in oocyte maturation (Kuwabara, 2003 “The multifaceted C. elegans major sperm protein: an ephrin signaling antagonist in oocyte maturation” Genes and Development, 17: 155-161.

Azurin

The azurins are copper containing proteins of 128 amino acid residues which belong to the family of cupredoxins involved in electron transfer in plants and certain bacteria. The azurins include those from P. aeruginosa (PA) (SEQ ID NO: 1), A. xylosoxidans, and A. denitrificans (SEQ ID NO: 6). (Murphy et al., J. Mol. Biol. 315:859-871 (2002)) The amino acid sequence identity between the azurins varies between 60-90%, these proteins showed a strong structural homology. All azurins have a characteristic β-sandwich with Greek key motif and the single copper atom is always placed at the same region of the protein. In addition, azurins possess an essentially neutral hydrophobic patch surrounding the copper site. Id.

Plastocyanins

The plastocyanins are soluble proteins of cyanobacteria, algae and plants that contain one molecule of copper per molecule and are blue in their oxidized form. They occur in the chloroplast, where they function as electron carriers. Since the determination of the structure of poplar plastocyanin in 1978, the structure of algal (Scenedesmus, Enteromorpha, Chlamydomonas) and plant (French bean) plastocyanins has been determined either by crystallographic or NMR methods, and the poplar structure has been refined to 1.33 Å resolution. SEQ ID NO: 2 shows the amino acid sequence of plastocyanin from Phormidium laminosum, a thermophilic cyanobacterium.

Despite the sequence divergence among plastocyanins of algae and vascular plants (e.g., 62% sequence identity between the Chlamydomonas and poplar proteins), the three-dimensional structures are conserved (e.g., 0.76 Å rms deviation in the C alpha positions between the Chlamydomonas and Poplar proteins). Structural features include a distorted tetrahedral copper binding site at one end of an eight-stranded antiparallel beta-barrel, a pronounced negative patch, and a flat hydrophobic surface. The copper site is optimized for its electron transfer function, and the negative and hydrophobic patches are proposed to be involved in recognition of physiological reaction partners. Chemical modification, cross-linking, and site-directed mutagenesis experiments have confirmed the importance of the negative and hydrophobic patches in binding interactions with cytochrome f, and validated the model of two functionally significant electron transfer paths involving plastocyanin. One putative electron transfer path is relatively short (approximately 4 Å) and involves the solvent-exposed copper ligand His-87 in the hydrophobic patch, while the other is more lengthy (approximately 12-15 Å) and involves the nearly conserved residue Tyr-83 in the negative patch, Redinbo et al., J. Bioenerg. Biomembr. 26:49-66 (1994).

Rusticyanins

Rusticyanins are blue-copper containing single-chain polypeptides obtained from a Thiobacillus (now called Acidithiobacillus). The X-ray crystal structure of the oxidized form of the extremely stable and highly oxidizing cupredoxin rusticyanin from Thiobacillus ferrooxidans (SEQ ID NO: 3) has been determined by multiwavelength anomalous diffraction and refined to 1.9 Å resolution. The rusticyanins are composed of a core beta-sandwich fold composed of a six- and a seven-stranded b-sheet. Like other cupredoxins, the copper ion is coordinated by a cluster of four conserved residues (His 85, Cys138, His143, Met148) arranged in a distorted tetrahedron. Walter, R. L. et al., J. Mol. Biol., vol. 263, pp-730-51 (1996).

Pseudoazurins

The pseudoazurins are a family of blue-copper containing single-chain polypeptide. The amino acid sequence of pseudoazurin obtained from Achromobacter cycloclastes is shown in SEQ ID NO: 4. The X-ray structure analysis of pseudoazurin shows that it has a similar structure to the azurins although there is low sequence homology between these proteins. Two main differences exist between the overall structure of the pseudoazurins and azurins. There is a carboxy terminus extension in the pseudoazurins, relative to the azurins, consisting of two alpha-helices. In the mid-peptide region azurins contain an extended loop, shortened in the pseudoazurins, which forms a flap containing a short α-helix. The only major differences at the copper atom site are the conformation of the MET side-chain and the Met-S copper bond length, which is significantly shorter in pseudoazurin than in azurin.

Phytocyanins

The proteins identifiable as phytocyanins include, but are not limited to, cucumber basic protein, stellacyanin, mavicyanin, umecyanin, a cucumber peeling cupredoxin, a putative blue copper protein in pea pods, and a blue copper protein from Arabidopsis thaliana. In all except cucumber basic protein and the pea-pod protein, the axial methionine ligand normally found at blue copper sites is replaced by glutamine.

Auracyanin

Three small blue copper proteins designated auracyanin A, auracyanin B-1, and auracyanin B-2 have been isolated from the thermophilic green gliding photosynthetic bacterium Chloroflexus aurantiacus. The two B forms are glycoproteins and have almost identical properties to each other, but are distinct from the A form. The sodium dodecyl sulfate-polyacrylamide gel electrophoresis demonstrates apparent monomer molecular masses as 14 (A), 18 (B-2), and 22 (B-1) kDa.

The amino acid sequence of auracyanin A has been determined and showed auracyanin A to be a polypeptide of 139 residues. (Van Dreissche et al., Protein Science 8:947-957 (1999).) His58, Cys123, His128, and Met132 are spaced in a way to be expected if they are the evolutionary conserved metal ligands as in the known small copper proteins plastocyanin and azurin. Secondary structure prediction also indicates that auracyanin has a general beta-barrel structure similar to that of azurin from Pseudomonas aeruginosa and plastocyanin from poplar leaves. However, auracyanin appears to have sequence characteristics of both small copper protein sequence classes. The overall similarity with a consensus sequence of azurin is roughly the same as that with a consensus sequence of plastocyanin, namely 30.5%. The N-terminal sequence region 1-18 of auracyanin is remarkably rich in glycine and hydroxy amino acids. Id. See exemplary amino acid sequence SEQ ID NO: 14 for chain A of auracyanin from Chloroflexus aurantiacus (NCBI Protein Data Bank Accession No. AAM12874).

The auracyanin B molecule has a standard cupredoxin fold. The crystal structure of auracyanin B from Chloroflexus aurantiacus has been studied. (Bond et al., J. Mol. Biol. 306:47-67 (2001).) With the exception of an additional N-terminal strand, the molecule is very similar to that of the bacterial cupredoxin, azurin. As in other cupredoxins, one of the Cu ligands lies on strand 4 of the polypeptide, and the other three lie along a large loop between strands 7 and 8. The Cu site geometry is discussed with reference to the amino acid spacing between the latter three ligands. The crystallographically characterized Cu-binding domain of auracyanin B is probably tethered to the periplasmic side of the cytoplasmic membrane by an N-terminal tail that exhibits significant sequence identity with known tethers in several other membrane-associated electron-transfer proteins. The amino acid sequences of the B forms are presented in McManus et al. (J. Biol Chem. 267:6531-6540 (1992).). See exemplary amino acid sequence SEQ ID NO: 15 for chain B of auracyanin from Chloroflexus aurantiacus (NCBI Protein Data Bank Accession No. 1QHQA).

Stellacyanin

Stellacyanins are a subclass of phytocyanins, a ubiquitous family of plant cupredoxins. An exemplary sequence of a stellacyanin is included herein as SEQ ID NO: 13. The crystal structure of umecyanin, a stellacyanin from horseradish root (Koch et al., J. Am. Chem. Soc. 127:158-166 (2005)) and cucumber stellacyanin (Hart et al., Protein Science 5:2175-2183 (1996).). The protein has an overall fold similar to the other phytocyanins. The ephrin B2 protein ectodomain tertiary structure bears a significant similarity to stellacyanin. (Toth et al., Developmental Cell 1:83-92 (2001).) An exemplary amino acid sequence of a stellacyanin is found in the National Center for Biotechnology Information Protein Data Bank as Accession No. 1JER, SEQ ID NO: 13.

Cucumber Basic Protein

An exemplary amino acid sequence from a cucumber basic protein is included herein as SEQ ID NO: 16. The crystal structure of the cucumber basic protein (CBP), a type 1 blue copper protein, has been refined at 1.8 Å resolution. The molecule resembles other blue copper proteins in having a Greek key beta-barrel structure, except that the barrel is open on one side and is better described as a “beta-sandwich” or “beta-taco”. (Guss et al., J. Mol. Biol. 262:686-705 (1996).) The ephrinB2 protein ectodomain tertiary structure bears a high similarity (rms deviation 1.5 Å for the 50 α carbons) to the cucumber basic protein. (Toth et al., Developmental Cell 1:83-92 (2001).)

The Cu atom has the normal blue copper NNSS' co-ordination with bond lengths Cu—N(His39)=1.93 A, Cu—S(Cys79)=2.16 A, Cu—N(His84)=1.95 A, Cu—S(Met89)=2.61 A. A disulphide link, (Cys52)-S—S-(Cys85), appears to play an important role in stabilizing the molecular structure. The polypeptide fold is typical of a sub-family of blue copper proteins (phytocyanins) as well as a non-metalloprotein, ragweed allergen Ra3, with which CBP has a high degree of sequence identity. The proteins currently identifiable as phytocyanins are CBP, stellacyanin, mavicyanin, umecyanin, a cucumber peeling cupredoxin, a putative blue copper protein in pea pods, and a blue copper protein from Arabidopsis thaliana. In all except CBP and the pea-pod protein, the axial methionine ligand normally found at blue copper sites is replaced by glutamine. An exemplary sequence for cucumber basic protein is found in NCBI Protein Data Bank Accession No. 2CBP, SEQ ID NO: 16.

### Cytochromes

Cytochrome C₅₅₁

Cytochrome C₅₅₁ from P. aeruginosa (Pa-C551) is a monomeric redox protein of 82 amino-acid residues (SEQ ID NO: 21), involved in dissimilative denitrification as the physiological electron donor of nitrite reductase. The functional properties of Pa-C551 have been extensively investigated. The reactions with non-physiological small inorganic redox reactants and with other macromolecules, like blue copper proteins, eukaryotic cytochrome c and the physiological partner nitrite reductase have provided a test for protein-protein electron transfer.

The three-dimensional structure of Pa-C551, which is a member of bacterial class I cytochromes, shows a single low-spin heme with His-Met ligation and the typical polypeptide fold which however leaves the edges of pyrrole rings II and III of the heme exposed (Cutruzzola et al., J. Inorgan. Chem. 88:353-61 (2002)). The lack of a 20-residue omega loop, present in the mammalian class I cytochromes, causes further exposure of the heme edge at the level of propionate 13. The distribution of charged residues on the surface of Pa-C551 is very anisotropic: one side is richer in acidic residues whereas the other displays a ring of positive side chains, mainly lysines, located at the border of a hydrophobic patch which surrounds the heme crevice. This patch comprises residues Gly11, Val13, Ala14, Met22, Val23, Pro58, Ile59, Pro60, Pro62, Pro63 and Ala65. The anisotropic charge distribution leads to a large dipolar moment which is important for electron transfer complex formation.

The charge distribution described above for Pa-C551 has been reported for other electron transfer proteins and their electron acceptors. Moreover, modification by site-directed mutagenesis of residues within the hydrophobic or charged patch has shown for different proteins the importance of surface complementarity for binding and electron transfer. As an example, evidence for the relevance of the hydrophobic patch for the electron transfer properties of azurin from P. aeruginosa came from the studies carried out on mutants of residues Met44 and Met64 changed to positively and negatively charged amino acids. Id.

The cytochrome c-type domain has a fold consisting of a series of alpha helices and reverse turns that serve to envelop the covalently bound haem within a hydrophobic pocket. This domain can be found in monodomain cytochrome c proteins, such as cytochrome c6, cytochrome c₅₅₂, cytochrome c₄₅₉ and mitochondrial cytochrome c. The cytochrome c-type domain occurs in a number of other proteins, such as in cytochrome cd1-nitrite reductase as the N-terminal haem c domain, in quinoprotein alcohol dehydrogenase as the C-terminal domain, in Quinohemoprotein amine dehydrogenase A chain as domains 1 and 2, and in the cytochrome bc₁ complex as the cytochrome bc₁ domain. Structural analysis with VAST algorithm (cytochrome c₅₅₁ from Pseudomonas aeruginosa as a query) showed significant structural neighbors (P values between 10^{-10.3} to 10^{-4.5}) only for cytochromes.

### Methods of Use

The invention provides methods to administer to a patient the compositions comprising cupredoxin or cytochrome, and variants, derivatives and structural equivalents of cupredoxin or cytochrome. Specifically, the invention provides methods to administer to a patient a composition comprising at least one peptide, or at least two peptides that are a cupredoxin, cytochrome and variants, derivatives and structural equivalents of cupredoxin or cytochrome. More specifically, the invention provides methods to administer to a human a composition comprising at least one peptide that is a cupredoxin, cytochrome and variants, derivatives and structural equivalents of cupredoxin or cytochrome.

The invention provides methods to administer to a patient compositions comprising cupredoxin or cytochrome and variants, derivatives and structural equivalents of cupredoxin or cytochrome, and their use to concurrently treat and/or prevent two or more conditions in a patient. In a specific embodiment, the methods may utilize pharmaceutical compositions for the administration to a patient. In another specific embodiment, the invention provides methods for the concurrent prevention and/or treatment of two or more conditions such as interstitial cystitis (IC), lesions associated with inflammatory bowel disease (IBD), HIV infection, AIDS, central nervous system disorders, peripheral vascular diseases, viral diseases, degeneration of the central nervous system (Christopher Reeve's disease), Alzheimer's disease, malaria, inappropriate angiogenesis, cardiovascular disease, hypertension, Cytomegalovirus infection, human papillomavirus infection; Muscular Dystrophy, encephalopathy, dementia, Parkinson's disease, neuropathy, macular degeneration, diabetic retinopathy, rheumatoid arthritis, psoriasis, herpes simplex virus (HSV), Ebola virus, cytomeglovirus (CMV), Para influenza viruses types A, B and C, hepatitis virus A, B, C, and G, the delta hepatitis virus (HDV), mumps virus, measles virus, respiratory syncytial virus, bunyvirus, arena virus, Dhori virus, poliovirus, rubella virus, dengue virus; SIV, Mycobacterium tuberculosis, melanoma, leukemia, breast cancer, ovarian cancer, lung cancer, mesenchymal cancer, colon cancer, aerodigestive tract cancer, cervical cancer, brain tumors and prostate cancer. In another specific embodiment, the methods may utilize compositions administered to a patient for the concurrent prevention and/or treatment of two or more conditions selected from one or more of the group consisting of HIV, malaria, cancer and inappropriate angiogenesis.

Members of the Cupredoxin family, specifically azurin from Pseudomonas aeruginosa, are promising compounds for therapeutic and preventative treatment of numerous conditions. Such conditions may include, but are not limited to HIV, malaria, cancer and inappropriate angiogenesis. For example, two redox proteins elaborated by P. aeruginosa, the cupredoxin azurin and cytochrome c₅₅₁ (Cyt c₅₅₁), both enter J774 cells and show significant cytotoxic activity towards the human cancer cells as compared to normal cells. Zaborina et al., Microbiology 146: 2521-2530 (2000). Azurin can also enter human melanoma UISO-Mel-2 or human breast cancer MCF-7 cells. Yamada et al., PNAS 99:14098-14103 (2002); Punj et al., Oncogene 23:2367-2378 (2004); Yamada et al., Cell. Biol. 7:14181431 (2005). In addition, azurin from P. aeruginosa preferentially enters J774 murine reticulum cell sarcoma cells, forms a complex with and stabilizes the tumor suppressor protein p53, enhances the intracellular concentration of p53, and induces apoptosis. Yamada et al., Infection and Immunity, 70:7054-7062 (2002). Azurin also caused a significant increase of apoptosis in human osteosarcoma cells as compared to non-cancerous cells. Ye et al., Ai Zheng 24:298-304 (2003). Rusticyanin from Thiobacillus ferrooxidans can also enter macrophages and induce apoptosis. Yamada et al., Cell Cycle 3:1182-1187 (2004); Yamada et al., Cell. Micro. 7:1418-1431 (2005). Plastocyanin from Phormidium laminosum and pseudoazurin form Achromobacter cycloclastes also are cytotoxic towards macrophages. U.S. Pat. Pub. No. 20060040269, published Feb. 23, 2006.

Azurin is also known to have other pharmacologic activities of therapeutic importance. It is known to inhibit angiogenesis in human umbilical vascular endothelium cells (HUVECs). U.S. patent application Ser. No. 11/488,693, filed Jul. 19, 2006. Azurin from P. aeruginosa is also known for its ability to inhibit the growth of HIV-1 infection in peripheral blood mononuclear cells and to inhibit parasitemia of malaria-infected mammalian red blood cells. Chaudhari et al., Cell Cycle. 5: 1642-1648 (2006). Azurin from P. aeruginosa is also known to interfere with the ephrin signaling system in various mammalian cells and tissues. U.S. patent application Ser. No. 11/436,592, filed May 19, 2006.

In another specific embodiment, the methods may utilize a composition comprising a cupredoxin, cytochrome or variant, derivative, truncation, or structural equivalent thereof, wherein the patient has at least one “high risk feature.” “High risk features” may be factors of the patient that increase the risk of a patient developing one or more conditions or where the patient has a higher risk than the general population.

The increased risk may be due to numerous variables or factors such as, but not limited to, environmental and behavioral factors, increased risk caused from other conditions, and genetic predisposition.

For example, an HIV infected patient is associated with an increased risk of acquiring large cell lymphoma or Kaposi's sarcoma. The Merck Manual of Diagnosis and Therapy (Beers et al., 18^{th} edition, Merck Research Laboratories, 2006). For another example, a female patient that acquires human papillomavirus has an increased risk of acquiring cervical carcinoma. Id.

Environmental factors may include, but are not limited to, a patient's lifestyle, eating habits and/or geographic location. For example, co-infections with HIV and malaria are very common in many areas of the world, and in particular sub-Saharan Africa.

Behavioral factors may include actions by the patient that predispose a patient to many conditions. For example, the risk of acquiring cancer and heart disease may be increased due to factors such as, but not limited to, smoking, diet, alcohol consumption, hormone replacement therapy and higher body mass index.

Genetic predisposition may play a factor in a patient acquiring numerous conditions. For example, it is known that when a person carries a particular cystic fibrosis transmembrane regulator (CFTR) mutation, that person has a higher risk for cystic fibrosis and pancreatic cancer. Weiss et al., Gut; 54: 1456-1460 (2005). For another example, genetic factors that predispose a patient to various forms of cancer include, but are not limited to, a family history of cancer, gene carrier status of BRCA1 and BRCA2, prior history of breast neoplasia, familial adenomatous polyposis (FAP), hereditary nonpolyposis colorectal cancer (HNPCC), red or blond hair and fair-skinned phenotype, xeroderma pigmentosum, and ethnicity.

Patients with high risk features, such as higher risk to develop cancer than the general population may be patients with premalignant lesions, and patients that have been cured of their initial cancer or definitively treated for their premalignant lesions. See generally Tsao et al., CA Cancer J Clin 54:150-180 (2004). Additionally, patients at a higher risk of developing cancer may be determined by the use of various risk models that have been developed for certain kinds of cancer. For example, patients predisposed to breast cancer may be determined using the Gail risk model, or the Claus model, among others. See Gail et al., J Natl Cancer Inst 81:1879-1886 (1989); Cuzick, Breast 12:405-411 (2003); Huang et al., Am J Epidemiol 151:703-714 (2000).

In a specific embodiment, the methods may utilize compositions to be administered to a patient for the concurrent treatment and/or prevention of two or more conditions where the patient has a higher risk than the general population of acquiring a condition. Such conditions may include, but are not limited to, cancer, HIV, malaria or inappropriate angiogenesis.

In a specific embodiment, the methods may comprise a composition including a cupredoxin, cytochrome or variant, derivative, truncation, or structural equivalent thereof, as a therapeutic agent for the treatment of malaria, wherein the patient is additionally suffering from HIV, cancer or inappropriate angiogenesis or has a higher risk than the general population of acquiring a condition such as HIV, cancer or inappropriate angiogenesis.

In another specific embodiment, the methods may utilize a composition comprising a cupredoxin, cytochrome or variant, derivative, truncation, or structural equivalent thereof, as a therapeutic agent for the treatment of HIV, wherein the patient is additionally suffering from malaria, cancer or inappropriate angiogenesis or has a higher risk than the general population of acquiring a condition such as malaria, cancer or inappropriate angiogenesis.

In another specific embodiment, the methods may utilize a composition comprising a cupredoxin, cytochrome or variant, derivative, truncation, or structural equivalent thereof, as a therapeutic agent for the treatment of cancer, wherein the patient is additionally suffering from HIV, malaria or inappropriate angiogenesis or has a higher risk than the general population of acquiring a condition such as HIV, malaria or inappropriate angiogenesis.

In another specific embodiment, the methods may utilize a composition comprising a cupredoxin, cytochrome or variant, derivative, truncation, or structural equivalent thereof, as a therapeutic agent for the treatment of inappropriate angiogenesis, wherein the patient is additionally suffering from HIV, cancer or malaria or has a higher risk than the general population of acquiring a condition such as HIV, cancer or malaria.

The compositions comprising a cupredoxin, cytochrome or variant, derivative, truncation, or structural equivalent thereof can be administered to the patient by many routes and in many regimens that will be well known to those in the art. In specific embodiments, the cupredoxin, cytochrome or variant, derivative, truncation, or structural equivalent thereof is administered intravenously, intramuscularly, subcutaneously, topically, orally, or by inhalation.

In another specific embodiment, the methods may utilize compositions that additionally comprise another drug. In a specific embodiment, the additional drug may be an anti-malarial drug, an anti-HIV drug, an anti-cancer drug and an anti-angiogenesis drug.

In one specific embodiment, the methods may comprise co-administering to a patient one unit dose of a composition comprising a cupredoxin, cytochrome or a variant, derivative, truncation, or structural equivalent of cupredoxin or cytochrome and one unit dose of a composition comprising another drug, in either order, administered at about the same time, or within about a given time following the administration of the other, for example, about one minute to about 60 minutes following the administration of the other drug, or about 1 hour to about 12 hours following the administration of the other drug. In another embodiment, the other drug may be, but is not limited to an anti-malarial drug, an anti-HIV drug, an anti-cancer drug, and an anti-angiogenesis drug.

Anti-malarial drugs of interest include, but are not limited to, proguanil, chlorproguanil, trimethoprim, chloroquine, mefloquine, lumefantrine, atovaquone, pyrimethamine-sulfadoxine, pyrimethamine-dapsone, halofantrine, quinine, quinidine, amodiaquine, amopyroquine, sulphonamides, artemisinin, arteflene, artemether, artesunate, primaquine, pyronaridine, proguanil, chloroquine, mefloquine, pyrimethamine-sulfadoxine, pyrimethamine-dapsone, halofantrine, quinine, proguanil, chloroquine, mefloquine, 1,16-hexadecamethylenebis(N-methylpyrrolidinium)dibromide, and combinations thereof.

Anti-HIV drugs include, but are not limited to, reverse transcriptase inhibitors: AZT (zidovudine [Retrovir]), ddC (zalcitabine [Hivid], dideoxyinosine), d4T (stavudine [Zerit]), and 3TC (lamivudine [Epivir]), nonnucleoside reverse transcriptase inhibitors (NNRTIS): delavirdine (Rescriptor) and nevirapine (Viramune), protease inhibitors: ritonavir (Norvir), a lopinavir and ritonavir combination (Kaletra), saquinavir (Invirase), indinavir sulphate (Crixivan), amprenavir (Agenerase), and nelfinavir (Viracept). In some embodiments, a combination of several drugs called highly active antiretroviral therapy (HAART) is used to treat people with HIV.

Anti-cancer and/or anti-angiogenesis drugs of interest include, but are not limited to, tamoxifen, aromatase inhibitors such as letrozole and anastrozole (Arimidex®), retinoids such as N-[4-hydroxyphenyl] retinamide (4-HPR, fenretinide), nonsteriodal anti-inflammatory agents (NSAIDs) such as aspirin and sulindac, celecoxib (COX-2 inhibitor), defluoromethylornithing (DFMO), ursodeoxycholic acid, 3-hydroxy-3-methylglutaryl coenzyme A reductase inhibitors, EKI-785 (EGFR inhibitor), bevacizumab (antibody to VEGF-receptor), cetuximab (antibody to EGFR), retinol such as vitamin A, beta-carotene, 13-cis retinoic acid, isotretinoin and retinyl palmitate, α-tocopherol, interferon, oncolytic adenovirus dl520 (ONYX-015), gefitinib, etretinate, finasteride, indole-3-carbinol, resveratrol, chlorogenic acid, raloxifene, and oltipraz.

### Pharmaceutical Compositions Comprising Cupredoxin, or Variant, Derivative, Truncation, or Structural Equivalent Thereof

Pharmaceutical compositions comprising cupredoxin or cytochrome, or variant, derivative, truncation, or structural equivalents thereof, can be manufactured in any conventional manner, e.g., by conventional mixing, dissolving, granulating, dragee-making, emulsifying, encapsulating, entrapping, or lyophilizing processes. The substantially pure or pharmaceutical grade cupredoxin, cytochrome or variants, derivatives and structural equivalents thereof can be readily combined with a pharmaceutically acceptable carrier well-known in the art. Such carriers enable the preparation to be formulated as a tablet, pill, dragee, capsule, liquid, gel, syrup, slurry, suspension, and the like. Suitable carriers or excipients can also include, for example, fillers and cellulose preparations. Other excipients can include, for example, flavoring agents, coloring agents, detackifiers, thickeners, and other acceptable additives, adjuvants, or binders. In some embodiments, the pharmaceutical preparation is substantially free of preservatives. In other embodiments, the pharmaceutical preparation may contain at least one preservative. General methodology on pharmaceutical dosage forms is found in Ansel et al., Pharmaceutical Dosage Forms and Drug Delivery Systems (Lippencott Williams & Wilkins, Baltimore Md. (1999)).

The composition comprising a cupredoxin, cytochrome or variant, derivative, truncation, or structural equivalent thereof used in the invention may be administered in a variety of ways, including by injection (e.g., intradermal, subcutaneous, intramuscular, intraperitoneal and the like), by inhalation, by topical administration, by suppository, by using a transdermal patch or by mouth. General information on drug delivery systems can be found in Ansel et al., id. In some embodiments, the composition comprising a cupredoxin, cytochrome or variant, derivative, truncation, or structural equivalent thereof can be formulated and used directly as injectables, for subcutaneous and intravenous injection, among others. The injectable formulation, in particular, can advantageously be used to prevent and/or treat patients with more than one condition. The composition comprising a cupredoxin, cytochrome or variant, derivative, truncation, or structural equivalent thereof can also be taken orally after mixing with protective agents such as polypropylene glycols or similar coating agents.

When administration is by injection, the cupredoxin, cytochrome or variant, derivative, truncation, or structural equivalent thereof may be formulated in aqueous solutions, specifically in physiologically compatible buffers such as Hanks solution, Ringer's solution, or physiological saline buffer. The solution may contain formulatory agents such as suspending, stabilizing and/or dispersing agents. Alternatively, the cupredoxin or variant, derivative, truncation, or structural equivalent thereof may be in powder form for constitution with a suitable vehicle, e.g., sterile pyrogen-free water, before use. In some embodiments, the pharmaceutical composition does not comprise an adjuvant or any other substance added to enhance the immune response stimulated by the peptide. In some embodiments, the pharmaceutical composition comprises a substance that inhibits an immune response to the peptide.

When administration is by intravenous fluids, the intravenous fluids for use administering the cupredoxin, cytochrome or variant, derivative, truncation, or structural equivalent thereof may be composed of crystalloids or colloids. Crystalloids as used herein are aqueous solutions of mineral salts or other water-soluble molecules. Colloids as used herein contain larger insoluble molecules, such as gelatin. Intravenous fluids may be sterile.

Crystalloid fluids that may be used for intravenous administration include but are not limited to, normal saline (a solution of sodium chloride at 0.9% concentration), Ringer's lactate or Ringer's solution, and a solution of 5% dextrose in water sometimes called D5W, as described in Table 2.

TABLE 2
Composition of Common Crystalloid Solutions

| Solution | Other Name | [Na ⁺] | [Cl⁻] | [Glucose] |
|---|---|---|---|---|
| D5W | 5% Dextrose | 0 | 0 | 252 |
| ⅔ & ⅓ | 3.3% Dextrose/ | 51 | 51 | 168 |
| | 0.3% saline | | | |
| Half-normal | 0.45% NaCl | 77 | 77 | 0 |
| saline | | | | |
| Normal saline | 0.9% NaCl | 154 | 154 | 0 |
| Ringer's | Ringer's | 130 | 109 | 0 |
| lactate* | solution | | | |

*Ringer's lactate also has 28 mmol/L lactate, 4 mmol/L K⁺ and 3 mmol/L Ca²⁺.

When administration is by inhalation, the cupredoxin, cytochrome or variant, derivative, truncation, or structural equivalent thereof may be delivered in the form of an aerosol spray from pressurized packs or a nebulizer with the use of a suitable propellant, e.g., dichlorodifluoromethane, trichlorofluoromethane, carbon dioxide or other suitable gas. In the case of a pressurized aerosol, the dosage unit may be determined by providing a valve to deliver a metered amount. Capsules and cartridges of, e.g., gelatin, for use in an inhaler or insufflator may be formulated containing a powder mix of the proteins and a suitable powder base such as lactose or starch.

When administration is by topical administration, the cupredoxin, cytochrome or variant, derivative, truncation, or structural equivalent thereof may be formulated as solutions, gels, ointments, creams, jellies, suspensions, and the like, as are well known in the art. In some embodiments, administration is by means of a transdermal patch. When administration is by suppository (e.g., rectal or vaginal), cupredoxin, cytochrome or variants and derivatives thereof compositions may also be formulated in compositions containing conventional suppository bases.

When administration is oral, a cupredoxin, cytochrome or variant, derivative, truncation, or structural equivalent thereof can be readily formulated by combining the cupredoxin, cytochrome or variant, derivative, truncation, or structural equivalent thereof with pharmaceutically acceptable carriers well known in the art. A solid carrier, such as mannitol, lactose, magnesium stearate, and the like may be employed; such carriers enable the cupredoxin and variants, derivatives or structural equivalent thereof to be formulated as tablets, pills, dragees, capsules, liquids, gels, syrups, slurries, suspensions and the like, for oral ingestion by a subject to be treated. For oral solid formulations such as, for example, powders, capsules and tablets, suitable excipients include fillers such as sugars, cellulose preparation, granulating agents, and binding agents.

Other convenient carriers, as well-known in the art, also include multivalent carriers, such as bacterial capsular polysaccharide, a dextran or a genetically engineered vector. In addition, sustained-release formulations that include a cupredoxin, cytochrome or variant, derivative, truncation, or structural equivalent thereof allow for the release of cupredoxin, cytochrome or variant, derivative, truncation, or structural equivalent thereof over extended periods of time, such that without the sustained release formulation, the cupredoxin, cytochrome or variant, derivative, truncation, or structural equivalent thereof would be cleared from a subject's system, and/or degraded by, for example, proteases and simple hydrolysis before eliciting or enhancing a therapeutic effect.

The half-life in the bloodstream of the compositions of the invention can be extended or optimized by several methods well known to those in the art, including but not limited to, circularized peptides (Monk et al., BioDrugs 19(4):261-78, (2005); DeFreest et al., J. Pept. Res. 63(5):409-19 (2004)), D,L-peptides (diastereomer), (Futaki et al., J. Biol. Chem. February 23; 276(8):5836-40 (2001); Papo et al., Cancer Res. 64(16):5779-86 (2004); Miller et al., Biochem. Pharmacol. 36(1):169-76, (1987)); peptides containing unusual amino acids (Lee et al., J. Pept. Res. 63(2):69-84 (2004)), N- and C-terminal modifications (Labrie et al., Clin. Invest. Med. 13(5):275-8, (1990)), and hydrocarbon stapling (Schafmeister et al., J. Am. Chem. Soc. 122:5891-5892 (2000); Walenski et al., Science 305:1466-1470 (2004)). Of particular interest are d-isomerization (substitution) and modification of peptide stability via D-substitution or L-amino acid substitution and hydrocarbon stapling

In various embodiments, the pharmaceutical composition includes carriers and excipients (including but not limited to buffers, carbohydrates, mannitol, proteins, polypeptides or amino acids such as glycine, antioxidants, bacteriostats, chelating agents, suspending agents, thickening agents and/or preservatives), water, oils, saline solutions, aqueous dextrose and glycerol solutions, other pharmaceutically acceptable auxiliary substances as required to approximate physiological conditions, such as buffering agents, tonicity adjusting agents, wetting agents and the like. It will be recognized that, while any suitable carrier known to those of ordinary skill in the art may be employed to administer the compositions of this invention, the type of carrier will vary depending on the mode of administration. Compounds may also be encapsulated within liposomes using well-known technology. Biodegradable microspheres may also be employed as carriers for the pharmaceutical compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252.

The pharmaceutical compositions may be sterilized by conventional, well-known sterilization techniques, or may be sterile filtered. The resulting aqueous solutions may be packaged for use as is, or lyophilized, the lyophilized preparation being combined with a sterile solution prior to administration.

Administration of Cupredoxin and/or Cytochrome and Variants and Derivatives and Structural Equivalents Thereof

The cupredoxin, cytochrome or variant, derivative, truncation, or structural equivalent thereof can be administered formulated as pharmaceutical compositions and administered by any suitable route, for example, by oral, buccal, inhalation, sublingual, rectal, vaginal, transurethral, nasal, topical, percutaneous, i.e., transdermal or parenteral (including intravenous, intramuscular, subcutaneous and intracoronary) or vitreous administration. The pharmaceutical formulations thereof can be administered in any amount effective to achieve its intended purpose. More specifically, the composition is administered in a therapeutically effective amount. In specific embodiments, the therapeutically effective amount is generally from about 0.01-20 mg/day/kg of body weight.

The compounds comprising cupredoxin or variant, derivative, truncation, or structural equivalent thereof are useful for the prevention and/or treatment of more than one condition, alone or in combination with other active agents. The appropriate dosage will, of course, vary depending upon, for example, the compound of cupredoxin or variant, derivative, truncation, or structural equivalent thereof employed, the host, the mode of administration and the nature and severity of the potential cancer. However, in general, satisfactory results in humans are indicated to be obtained at daily dosages from about 0.01-20 mg/kg of body weight. An indicated daily dosage in humans is in the range from about 0.7 mg to about 1400 mg of a compound of cupredoxin or variant, derivative, truncation, or structural equivalent thereof conveniently administered, for example, in daily doses, weekly doses, monthly doses, and/or continuous dosing. Daily doses can be in discrete dosages from 1 to 12 times per day. Alternatively, doses can be administered every other day, every third day, every fourth day, every fifth day, every sixth day, every week, and similarly in day increments up to 31 days or over. Alternatively, dosing can be continuous using patches, i.v. administration and the like.

The exact formulation, route of administration, and dosage is determined by the attending physician in view of the patient's condition. Dosage amount and interval can be adjusted individually to provide plasma levels of the active cupredoxin or variant, derivative, truncation, or structural equivalent thereof which are sufficient to maintain therapeutic effect. Generally, the desired cupredoxin or variant, derivative, truncation, or structural equivalent thereof is administered in an admixture with a pharmaceutical carrier selected with regard to the intended route of administration and standard pharmaceutical practice.

In one aspect, the cupredoxin, cytochrome or variant, derivative, truncation, or structural equivalent thereof is delivered as DNA such that the polypeptide is generated in situ. In one embodiment, the DNA is “naked,” as described, for example, in Ulmer et al., (Science 259:1745-1749 (1993)) and reviewed by Cohen (Science 259:1691-1692 (1993)). The uptake of naked DNA may be increased by coating the DNA onto a carrier, e.g., biodegradable beads, which are then efficiently transported into the cells. In such methods, the DNA may be present within any of a variety of delivery systems known to those of ordinary skill in the art, including nucleic acid expression systems, bacterial and viral expression systems. Techniques for incorporating DNA into such expression systems are well known to those of ordinary skill in the art. See, e.g., WO90/11092, WO93/24640, WO 93/17706, and U.S. Pat. No. 5,736,524.

Vectors, used to shuttle genetic material from organism to organism, can be divided into two general classes: Cloning vectors are replicating plasmid or phage with regions that are essential for propagation in an appropriate host cell and into which foreign DNA can be inserted; the foreign DNA is replicated and propagated as if it were a component of the vector. An expression vector (such as a plasmid, yeast, or animal virus genome) is used to introduce foreign genetic material into a host cell or tissue in order to transcribe and translate the foreign DNA, such as the DNA of a cupredoxin. In expression vectors, the introduced DNA is operably-linked to elements such as promoters that signal to the host cell to highly transcribe the inserted DNA. Some promoters are exceptionally useful, such as inducible promoters that control gene transcription in response to specific factors. Operably-linking a cupredoxin and variants and derivatives thereof polynucleotide to an inducible promoter can control the expression of the cupredoxin and variants and derivatives thereof in response to specific factors. Examples of classic inducible promoters include those that are responsive to α-interferon, heat shock, heavy metal ions, and steroids such as glucocorticoids (Kaufman, Methods Enzymol. 185:487-511 (1990)) and tetracycline. Other desirable inducible promoters include those that are not endogenous to the cells in which the construct is being introduced, but, are responsive in those cells when the induction agent is exogenously supplied. In general, useful expression vectors are often plasmids. However, other forms of expression vectors, such as viral vectors (e.g., replication defective retroviruses, adenoviruses and adeno-associated viruses) are contemplated.

Vector choice is dictated by the organism or cells being used and the desired fate of the vector. In general, vectors comprise signal sequences, origins of replication, marker genes, polylinker sites, enhancer elements, promoters, and transcription termination sequences.

The exact formulation, route of administration, and dosage is determined by the attending physician in view of the patient's condition. Dosage amount and interval can be adjusted individually to provide plasma levels of the active cupredoxin and/or cytochrome and variants and derivatives thereof which are sufficient to treat the patient and/or maintain therapeutic effect. Generally, the desired cupredoxin and/or cytochrome and variants and derivatives thereof can be administered in an admixture with a pharmaceutical carrier selected with regard to the intended route of administration and standard pharmaceutical practice. Pharmaceutical compositions used in accordance with the present invention can be formulated in a conventional manner using one or more physiologically acceptable carriers comprising excipients and auxiliaries that facilitate processing of the cupredoxin and/or cytochrome and variants and derivatives thereof, active agents, for inhibiting or stimulating the secretion of cupredoxin and/or cytochrome and variants and derivatives thereof, or a mixture thereof into preparations which can be used therapeutically.

Kits Comprising Cupredoxin, and/or Cytochrome, or Variant, Derivative, Truncation, or Structural Equivalent Thereof

In one aspect, the invention provides regimens or kits comprising one or more of the following in a package or container: (1) a pharmacologically active composition comprising at least one cupredoxin, cytochrome or variant, derivative, truncation, or structural equivalent thereof; (2) an additional chemopreventive drug, (3) apparatus to administer the biologically active composition to the patient, such as a syringe, nebulizer etc.

When a kit is supplied, the different components of the composition may be packaged in separate containers, if appropriate, and admixed immediately before use. Such packaging of the components separately may permit long-term storage without losing the active components' functions.

The reagents included in the kits can be supplied in containers of any sort such that the life of the different components are preserved and are not adsorbed or altered by the materials of the container. For example, sealed glass ampoules may contain lyophilized cupredoxin and variants, derivatives and structural equivalents thereof, or buffers that have been packaged under a neutral, non-reacting gas, such as nitrogen. Ampoules may consist of any suitable material, such as glass, organic polymers, such as polycarbonate, polystyrene, etc., ceramic, metal or any other material typically employed to hold similar reagents. Other examples of suitable containers include simple bottles that may be fabricated from similar substances as ampoules, and envelopes, that may comprise foil-lined interiors, such as aluminum or an alloy. Other containers include test tubes, vials, flasks, bottles, syringes, or the like. Containers may have a sterile access port, such as a bottle having a stopper that can be pierced by a hypodermic injection needle. Other containers may have two compartments that are separated by a readily removable membrane that upon removal permits the components to be mixed Removable membranes may be glass, plastic, rubber, etc.

Kits may also be supplied with instructional materials. Instructions may be printed on paper or other substrate, and/or may be supplied as an electronic-readable medium, such as a floppy disc, CD-ROM, DVD-ROM, Zip disc, videotape, audiotape, flash memory device etc. Detailed instructions may not be physically associated with the kit; instead, a user may be directed to an internet web site specified by the manufacturer or distributor of the kit, or supplied as electronic mail.

### Modification of Cupredoxin and Variants, Derivatives and Structural Equivalents Thereof

Cupredoxin, cytochrome or variant, derivative, truncation, or structural equivalents thereof may be chemically modified or genetically altered to produce variants and derivatives as explained above. Such variants and derivatives may be synthesized by standard techniques.

In addition to naturally-occurring allelic variants of cupredoxin, changes can be introduced by mutation into cupredoxin coding sequence that incur alterations in the amino acid sequences of the encoded cupredoxin that do not significantly alter the ability of cupredoxin to inhibit the development of premalignant lesions. A “non-essential” amino acid residue is a residue that can be altered from the wild-type sequences of the cupredoxin without altering pharmacologic activity, whereas an “essential” amino acid residue is required for such pharmacologic activity. For example, amino acid residues that are conserved among the cupredoxins are predicted to be particularly non-anenable to alteration, and thus “essential.”

Amino acids for which conservative substitutions that do not change the pharmacologic activity of the polypeptide can be made are well known in the art. Useful conservative substitutions are shown in Table 3, “Preferred substitutions.” Conservative substitutions whereby an amino acid of one class is replaced with another amino acid of the same type fall within the scope of the invention so long as the substitution does not materially alter the pharmacologic activity of the compound.

TABLE 3
Preferred substitutions

| Original | | Preferred |
|---|---|---|
| residue | Exemplary substitutions | substitutions |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, | Leu |
| | Norleucine | |
| Leu (L) | Norleucine, Ile, Val, Met, Ala, | Ile |
| | Phe | |
| Lys (K) | Arg, Gln, Asn | Arg |
| Met (M) | Leu, Phe, Ile | Leu |
| Phe (F) | Leu, Val, Ile, Ala, Tyr | Leu |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, | Leu |
| | Norleucine | |

Non-conservative substitutions that affect (1) the structure of the polypeptide backbone, such as a β-sheet or α-helical conformation, (2) the charge, (3) hydrophobicity, or (4) the bulk of the side chain of the target site can modify the pharmacologic activity. Residues are divided into groups based on common side-chain properties as denoted in Table 4. Non-conservative substitutions entail exchanging a member of one of these classes for another class. Substitutions may be introduced into conservative substitution sites or more specifically into non-conserved sites.

TABLE 4
Amino acid classes

| Class | Amino acids |
|---|---|
| hydrophobic | Norleucine, Met, Ala, Val, Leu, Ile |
| neutral hydrophilic | Cys, Ser, Thr |
| acidic | Asp, Glu |
| basic | Asn, Gln, His, Lys, Arg |
| disrupt chain | Gly, Pro |
| conformation | |
| aromatic | Trp, Tyr, Phe |

The variant polypeptides can be made using methods known in the art such as oligonucleotide-mediated (site-directed) mutagenesis, alanine scanning, and PCR mutagenesis. Site-directed mutagenesis (Carter, Biochem J. 237:1-7 (1986); Zoller and Smith, Methods Enzymol. 154:329-350 (1987)), cassette mutagenesis, restriction selection mutagenesis (Wells et al., Gene 34:315-323 (1985)) or other known techniques can be performed on the cloned DNA to produce the cupredoxin variant DNA.

Known mutations of cupredoxins and cytochrome can also be used to create variant cupredoxin and cytochrome to be used in the methods of the invention. For example, the C112D and M44KM64E mutants of azurin are known to have cytotoxic and growth arresting activity that is different from the native azurin, and such altered activity can be useful in the treatment and/or prevention methods of the present invention.

A more complete understanding of the present invention can be obtained by reference to the following specific Examples. The Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitations. Modifications and variations of the invention as hereinbefore set forth can be made without departing from the spirit and scope thereof.

### EXAMPLES

### Example 1

### Entry of p28 into Human Umbilical Vein Endothelial Cells

p28 was labeled with 20 μM Alexafluor® 568 (Molecular Probes, Eugene, Oreg.). Indicated cell lines were cultured on cell culture coated cover slips overnight at 37° C. Pre-warmed media containing labeled peptide was added at indicated concentrations. After incubation with the labeled peptide, the cover slips were washed 3× with PBS and fixed in formalin for 5 minutes. Cover slips were then mounted in media containing 1.5 μg ml⁻¹ DAPI for nuclear staining (VECTASHIELD®, Vector Laboratories, Burlingame, Calif.). Analysis was performed with a confocal microscope (Model LC510, Carl Zeiss, Thornwood, N.Y.).

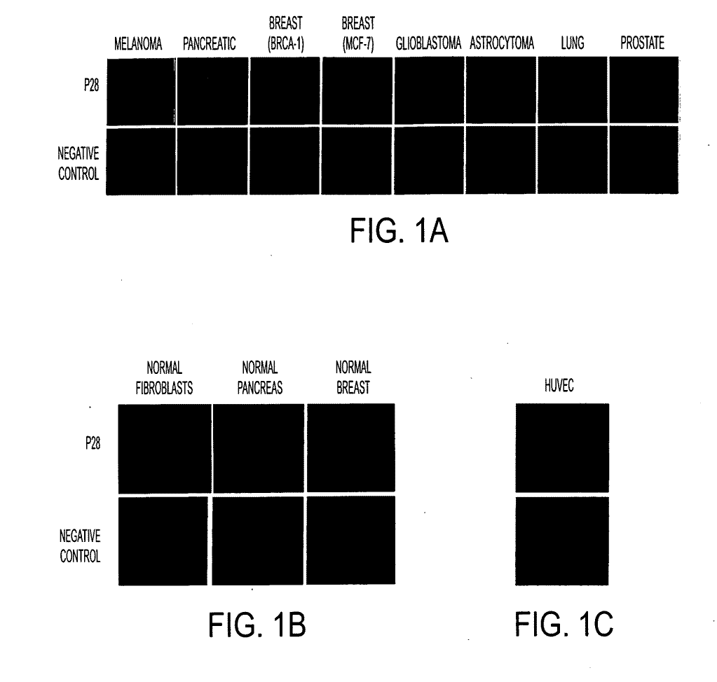

p28 effectively entered malignant cell lines originating from melanoma, breast, pancreas, glioblastoma, astrocytoma, and lung (FIG. 1A). p28 was also efficiently entered HUVEC cells (FIG. 1C). No significant entry was observed in other “normal” cell lines originating from skin fibroblasts, breast and pancreas FIG. 1B). Therefore, in addition to specifically entering mammalian cancer cells, p28 also specifically enters HUVEC cells.

This experiment shows that the P. aeruginosa azurin 50-77 peptide has activity that inhibits capillary tube formation in endothelial cells, one step in angiogenesis. The P. aeruginosa azurin 50-77 peptide can therefore be used to control angiogenesis and hence be utilized as a cancer treatment, and treatment of other conditions related to inappropriate angiogenesis.

### Example 2

### Effects of p28 on HUVEC Capillary Tube Formation on Matrigel®

Matrigel® Matrix (Becton Dickinson Biosciences, San Jose Calif.) is a solubulized basement membrane preparation extracted from EHS mouse sarcoma, a tumor rich in ECM proteins. Its major component is laminin, followed by collagen IV, heparan sulfate proteoglycans, and entactin 1. At room temperature, Matrigel® Matrix polymerizes to produce biologically active matrix material resembling the mammalian cellular basement membrane. Cells behave as they do in vivo when they are cultured on Matrigel® Matrix. It provides a physiologically relevant environment for studies of cell morphology, biochemical function, migration or invasion, and gene expression. Matrigel® Matrix serves as a substrate for in vitro endothelial cell invasion and tube formation assays.

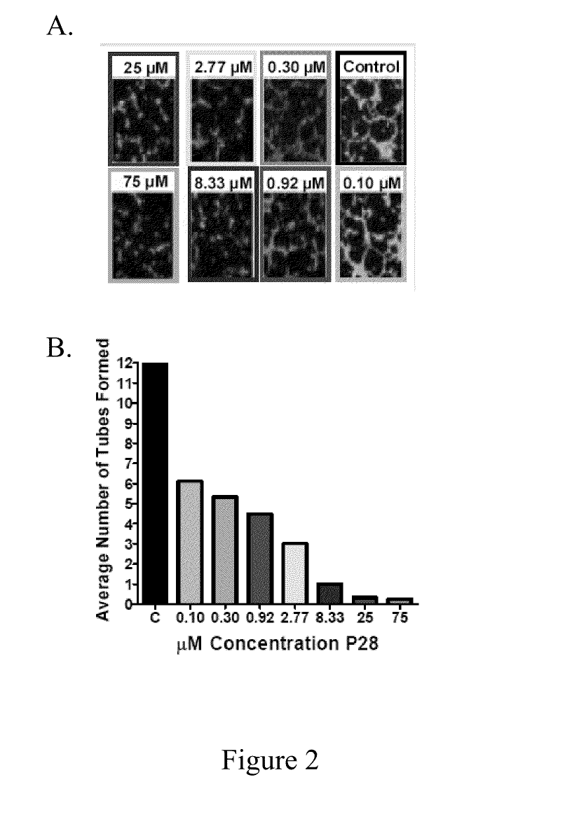

The effects of p28 on the capillary tube formation of HUVEC cells were investigated using Matrigel®. HUVEC cells were plated (15,000 cells/well) on Matrigel® coated 8 well chamber slides with 20 ng/ml VEGF and in the presence or absence of peptide. p28 concentrations of 0 μM (control), 0.10 μM, 0.30 μM, 0.92 μM, 2.77 μM, 8.33 μM, 25 μM and 75 μM were used. Cells were stained 4 h and 24 h post-treatment with calcein AM, and capillary tube formation was examined using a fluorescence microscope (FIG. 2A). The results show that as little as 0.10 μM prevented capillary tube formation by HUVEC cells by about 50% (FIG. 2A). p28 therefore inhibits tube formation of HUVEC cells, and will therefore also inhibit the capillary tube formation related to angiogenesis.

### Example 3

### Effects of p28 on HUVEC Motility

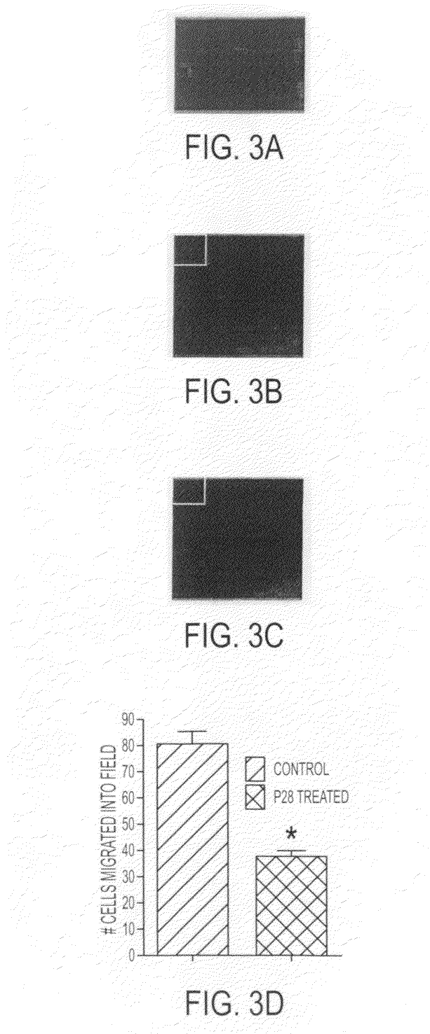

The effects of p28 on HUVEC motility was investigated with the scratch wound migration assay. HUVEC cells were plated in 60 mm tissue culture dishes and allowed to reach 90% confluence. After removing the media, cell layers were wounded using a 1 ml sterile plastic pipette tip. Plates were rinsed with culture media. Media with 20 ng/ml VEGF alone or media with 20 ng/ml VEGF and containing p28 peptide was then added to the plates. One dish was scratched as above and fixed immediately in order to mark exact wound area. FIG. 3A. After 24 h, cultures were fixed and stained for F-actin and nuclei using Phalloidin and Hoechst stain. Scratched areas were examined using a florescence microscope and photographed. The number of cells that migrated into the scratched area was counted in the control (FIG. 3B) and peptide treated dishes (FIG. 3C).

The number of HUVECs that migrated into the scratch wound in the cells treated with p28 was about half that of those that migrated into the scratch wound in the control. Figure D. Therefore, the presence of p28 inhibited the motility of HUVECs undergoing angiogenesis

### Example 4

### Effects of p28 on HUVEC Structural Proteins

The effects of p28 on HUVEC structural proteins was studied to gain a better understanding of the way p28 affects these cells. HUVEC cells plated on Matrigel® coated cover slips were incubated with 20 ng/ml VEGF in the presence or absence of 25 μM p28 peptide for 4 h or 24 h. After incubation, cells were rinsed in PBS, fixed in buffered formalin and permeablized in 0.2% triton in PBS. Cells were incubated with indicated antibodies for 90 min, and if necessary incubated with a specific secondary antibody, and then mounted in DAPI containing mounting media. Analysis was performed with a confocal microscope (model LC510, Carl Zeiss). Proteins examined are as follows: CD-31 (protein present at intercellular junctions that is necessary for cell to cell attachment), Fak (focal adhesion kinase), Paxillin, Vinculin (critical adhesion assembly proteins), WASP (Wiskott Aldrich Syndrome protein, required for nucleation and elongation of F-actin fibers), β-catenin (required for cell survival, regulation of cell surface proteins).

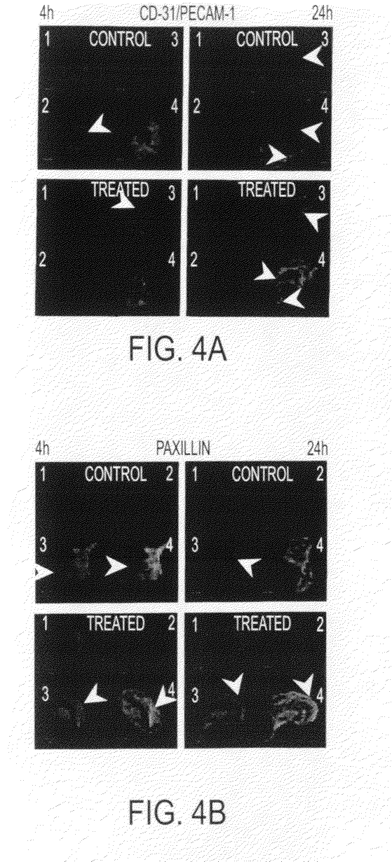

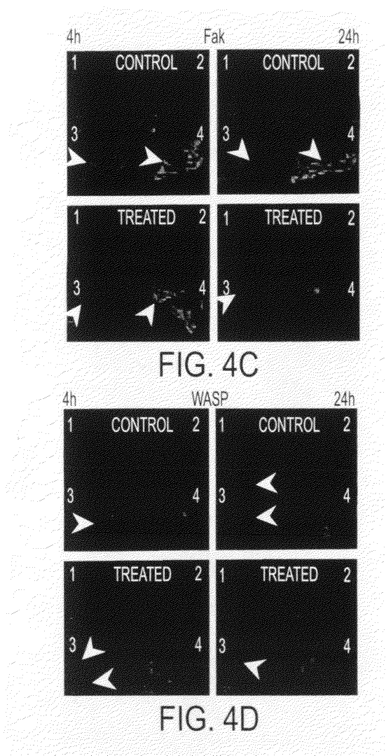

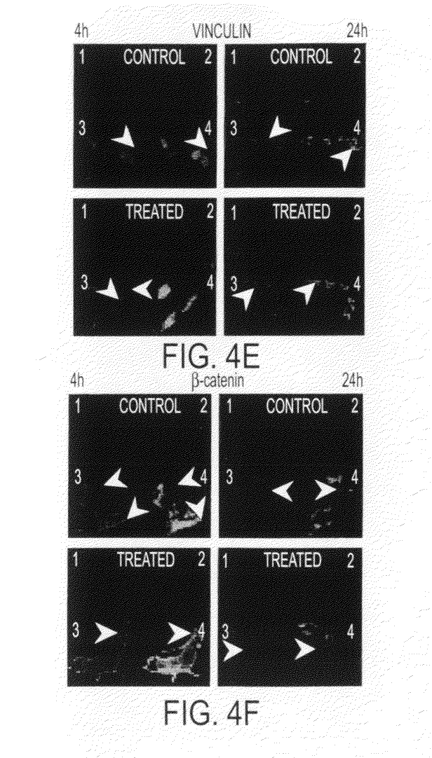

In the CD31/PECAM1 detected cells, pronounced CD31/PECAM localization was found at cell/cell junctions in p28 treated cells as compared to control (FIG. 4A). In the paxillin detected cell, the paxillin was mainly localized on cell surface of the control cells, however it was more often found on F-actin fibers in the p28 treated cells (FIG. 4B). In the Fak detected cells, Fak was mainly on localized cell surface of the control cells, while it was more often found on F-actin fibers of the p28 treated cells (FIG. 4C). In the WASP detected cells, at 4 h WASP localization was mostly nuclear in control cells, while WASP was localized on the nucleus and at the cell surface in p28 treated cells (FIG. 4 D). At 24 h, WASP was mostly localized at the cell surface in control cells, while it was mostly localized in the nucleus in p28 treated cells (FIG. 4D). In the vinculin detected cells, vinculin was localized mainly on the cell surface in control cells, while vinculin was more often localized on F-actin fibers in p28 treated cells (FIG. 4E). In β-catenin detected cells, at 4 h, β-catenin localization was mostly cytoplasmic with some on the cell surface in the control cells, while β-catenin was mostly localized on the cell membrane with some in the perinuclear space in the p28 treated cells. At 24 h, β-catenin localization was mostly on the cell membrane and in the nucleus in the control cells, while β-catenin was localized on the cell membrane and perinuclear area in p28 treated cells. Therefore, the presence of p28 prevented the structural changes normally found in HUVECs undergoing angiogenesis.

### Example 5

### In Vitro Growth Inhibition of Human Melanoma Cells by p28

The ability of p28 to inhibit the growth of human melanoma Mel-2 cells in vitro was determined. Mel-2 cells were plated in 24 well culture plates at 10,000-12,000 cells/well and allowed to attach to the plate overnight. Cells were then incubated at 37° C. in media alone (MEM-E with 10% FBS) or media containing p28 peptide. p28 was added at 5 μM, 50 μM, and 100 μM. The number of cells in each well was counted at 0 h, 24 h, 48 h and 72 h. The number of cells in each well was counted using a Coulter counter at the indicated time.

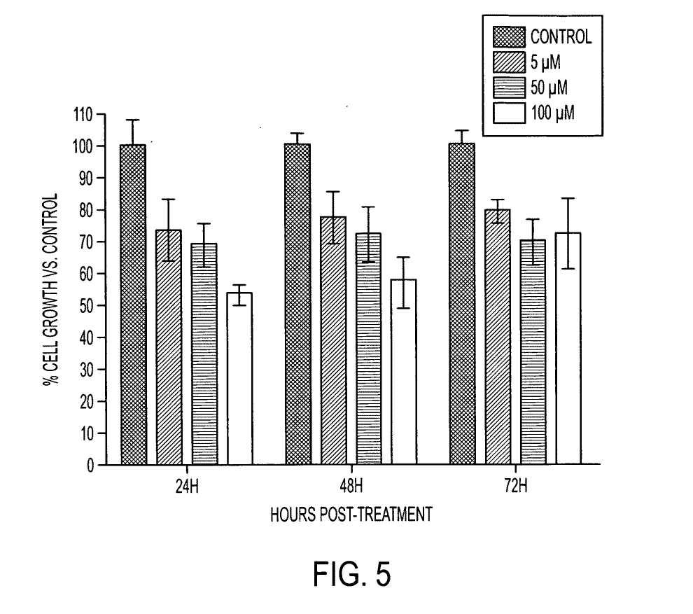

The results show that p28 inhibits growth of Mel-2 cells in a concentration dependent manner. p28 inhibited the Mel-2 cell growth by about 50% at 100 μM and 24 h (FIG. 5). These results indicate that p28 inhibits the growth of cancer cells, specifically human melanoma-2 cells.

### Example 6

### In Vivo Anti-Tumor Activity of p28 Peptides

One million Mel-2 cells were injected subcutaneously into the dorsal flank of 3-4 week old athymic mice (n=13 per group). Animals received daily i.p. injections of PBS only, 8 mg, or 16 mg per kg body weight (b.w.) of p28 peptide in PBS. Animals were examined daily for the development of palpable tumors. Once the tumor developed, tumor size was measured using a caliper and tumor volume was determined.

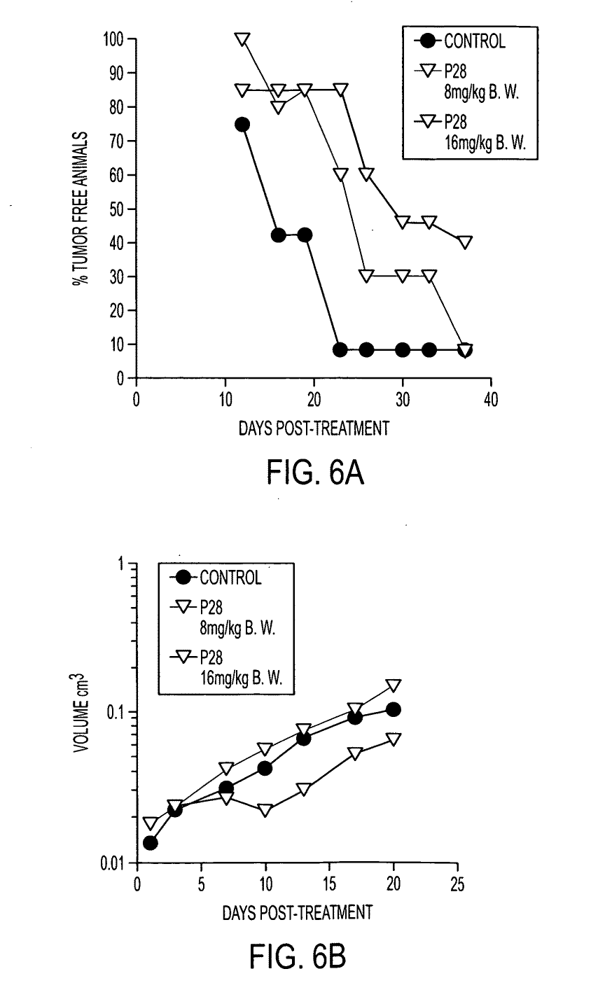

p28 inhibited the tumor incidence and growth in the mice. With the treatment of 16 mg/kg b.w., about 50% of the animal were tumor-free 40 days after the mel-2 cells were injected, while only about 95% of the control animals had tumors 22 days after the mel-2 cells were injected (FIG. 6A). p28 also inhibited the growth of the tumors by about 30% at 20 days post treatment with 16 mg/kg b.w. p28 (FIG. 6B). These results indicate that p28 can prevent the slow and prevent the develop of tumors, as well as slow the growth of existing tumors in vivo, and thus would make an effective therapeutic for cancer prevention and treatment in humans.

### Example 7

### Efficacy of the Synthetic Peptides Derived from Azurin and Plastocyanin

The efficacy of the synthetic peptides derived from azurin and plastocyanin have been analyzed. An 18-mer azurin peptide with the following sequence has been synthesized by standard techniques:

| | |
|---|---|
| TFDVSKLKEGEQYMFFCT | (SEQ ID NO: 48) |

MCF-7 breast cancer cells were incubated in 16-well plates with 5 and 50 ug/ml of the 18-mer azurin peptide for 0, 24, 48 and 72 hours, after which the number of MCF-7 cells were counted in a coulter counter. The peptide was seen at 50 ug/ml to inhibit MCF-7 cell growth by 50% in 48 to 72 hours, as compared to cells without the synthetic peptide treatment. The extent of cell growth inhibition was about 25% at 5 ug/ml of the 18-mer synthetic peptide as compared to untreated control. This experiment shows that the synthetic peptide d does in fact inhibit the cancer cell progression promoted by the B-2 ephrin.

### Example 8

### In Vitro Measurement of Effect of Cupredoxins on the Growth of Mel-2 and MCF-7 Cells

The growth of cells treated with cupredoxins was measured using a 16-well plate. Mel-2 or MCF-7 cells (5×10⁵ cells per well) were allowed to adhere to multiwell (16-well, in this instance) plates for 24 hours. After adherence, the growth medium was siphoned off. PBS (phosphate-buffered saline) or various cupredoxins/cytochromes at concentrations of 0.1 to 10 μM in PBS were then added to the wells containing fresh growth media and the growth of the cancer cells was followed for 24, 48 and 72 hours. After the incubation period, trypan blue was added to the culture and the number of dead floating cells was counted. Both live and dead floating cells were counted to determine the IC50 at various cupredoxin doses. The IC50 is the concentration of protein that inhibits the cell culture growth by 50%. At 500,000 cells per well at 24 hours of growth, enough cells were present for reproducible counts. In the cupredoxin-minus control cell cultures, as the cells grew, they had less space to adhere to the bottom of the well, began to die and became floating cells. In the cupredoxin-treated cell cultures, both the Mel-2 and MCF-7 cell line growth was inhibited leading to very few floating cells.

### Example 9

### In Vitro Inhibition of P. falciparum Parasitemia by Cupredoxin and Cytochrome

The cupredoxins bacterial wt azurin, M44KM64E azurin, rusticyanin and cyanobacterial plastocyanin, as well as the cytochromes Pseudomonas aeruginosa cytochrome c₅₅₁, human cytochrome c and Phormidium laminosum cytochrome f were tested in a normal red blood cell (RBC) assay at 200 μg/ml concentrations at 30 hours post inoculation. In these experiments, the normal RBCs were washed twice in serum free media and resuspended to 10% hematocrit in complete RPMI. 200 μl of 10% Hct RBCs were added to each of 24 wells (final 2% Hct at 1 ml) in addition to 30 μl complete RPMI containing recombinant cupredoxin or cytochrome proteins at 666 μM for a final concentration of 200 μM. Schizont-stage parasites were prepared by centrifuging a late-stage culture through a Percoll cushion at 3200 rpm for 10 minutes. For infection, 4×10⁶ parasites/well in 500 μl volume were added at t=0 hr. The plate was incubated for 30 hours and scored by thin blood smear and Giemsa stain at that time.

The control showed 9.5% parasitemia (standard error 1.3%), wt azurin 6.9% (s.e. 1.4%), M44KM64E azurin 9.1% (s.e. 1.0%), rusticyanin 7.2% (s.e. 0.7%), cytochrome c₅₅₁ 7.5% (s.e. 1.5%), human cytochrome c 8.4% (s.e. 0.4%), plastocyanin 8.1% (s.c. 1.3%) and cytochrome f 6.6% (s.e. 1.0%), suggesting that cupredoxins such as wt azurin and rusticyanin and cytochromes such as cytochrome f or cytochrome c₅₅₁ demonstrated 20 to 30% inhibition of parasitemia.

When the cupredoxins were tested for their effects at various stages of the parasite life cycle (0-24 hours, ring formation; 24-36 hours, trophozoite; 36-48 hours, schizont), the control showed 0.1% average ring formation and 9.4% trophozoite formation while wt azurin showed no ring formation but 6.9% trophozoite formation; cytochrome f showed 0.2% ring formation but had significantly low (6.3%) trophozoite formation. Remarkably, rusticyanin exhibited very high (2.0%) ring formation and significantly reduced (5.2%) trophozoite formation. The others had no significant effect. The parasites in rusticyanin-treated samples looked sick and dying as compared to the rest of the samples, showing a significant inhibitory and toxic effect of rusticyanin on parasite development.

### Example 10

### Inhibition In Vitro of P. falciparum Intracellular Replication by Rusticyanin

To determine if the bacterial redox proteins can inhibit intracellular replication of the malarial parasites, red blood cells were loaded to an intracellular recombinant protein concentration of 200 μg/ml using a hypotonic ghost preparation. Cells where then washed, resuspended and infected with schizont-stage parasites (P. falciparum) as described in Example 9. The red blood cell ghosts were incubated for 19 hours and 40 hours and giemsa smears were made.

Compared to the infections of normal red blood cells in Example 9, only rusticyanin decreased total parasitemia in loaded cell ghost cultures. At 19 hours, there was no significant difference in invasion and ring formation, with empty ghosts at 5.0±0.4% and rusticyanin-loaded ghosts at 4.5±1.0%. However, at 40 hours, rusticyanin-loaded ghosts had a lower level of infection. No major effects were seen at 19 hour with any of the bacterial proteins. However, at 40 hours, control untreated ghosts showed 4.6±0.3% parasitemia while rusticyanin-treated ghosts had 2.7±0.8% parasitemia, an almost 50% reduction. See, Table 5. Wt azurin, M44KM64E mutant azurin, plastocyanin, cytochrome c₅₅₁, human cytochrome c and cynobacterial cytochrome f proteins showed parasitemia varying from 4.2 to 5.4%.

TABLE 5
Cupredoxin and cytochrome inhibition of P. falciparum
infection of red blood cell ghosts.

| | Mean | |
|---|---|---|
| | Parasitemia | |
| Treatment | at 40 hr | Std. Error |
| Empty | 4.6% | 0.3% |
| Wild Type Azurin | 5.4% | 1.0% |
| M44KM64E Azurin | 4.7% | 0.5% |
| Rusticyanin | 2.7% | 0.8% |
| Cytochrome c₅₅₁ | 4.2% | 0.4% |
| Human Cytochrome c | 4.6% | 0.8% |
| Plastocyanin | 4.3% | 0.3% |
| Cytochrome f | 4.5% | 0.9% |

### Example 11

### Structural Homology Between Azurin and Fab Fragment of G17.12 Monoclonal Antibody Complexed with Pf MSP1-19

Previous studies have shown that cupredoxins show structural similarity to the variable domains of the immunoglobulin superfamily members. (Gough & Chothia, Structure 12:917-925 (2004); Stevens et al., J. Mol. Recognit. 18:150-157 (2005)) The DALI algorithm (Holm & Park, Bioinformatics 16:566-567 (2000)) was used to search the 3D databases for structural homologs of azurin (1JZG) from P. aeruginosa. Azurin exhibits structural similarity to the Fab fragment of G17.12 monoclonal antibody in complexation with Pf MSP1-19 fragment of the MSP1 merozoite surface protein of P. falciparum. (Pizarro et al., J. Mol. Biol. 328:1091-1103 (2003).) (Table 6) Azurin also demonstrates structural similarity to CD4 (Table 5), the primary host cell surface receptor for HIV-1. (Maddon et al., Cell 47:333-348 (1986)). Azurin also exhibits a structural similarity to ICAM-1 (Table 6), which is involved in cerebral malaria and implicated as a receptor on the endothelial cells in the microvasculture of the brain and other tissues for sequestering P. falciparum-infected erythrocytes. (Smith et al., Proc. Natl. Acad. Sci. USA 97:1766-1771 (2000); Franke-Fayard et al., Proc. Natl. Acad. Sci. USA 102:11468-11473 (2005)). ICAM-1 is also found in HIV-1 particles during their passage through the host cells and is known to enhance HIV-1 infectivity by enhancing cytosolic delivery of the viral materials. (Fortin et al., J. Virol. 71:3588-3596 (1997); Tardif & Tremblay, J. Virol. 77:12299-12309 (2003)) ICAM-1 is known also to be subverted as receptors for major groups of rhinoviruses and coxsackie viruses. (Bella & Rossmann, J. Struct. Biol. 128:69-74 (1999))

This example shows that cupredoxins including azurin demonstrate structural similarities in having two anti-parallel β sheets packed face to face and linked by a disulfide bridge to the variable domains of the immunoglobulin superfamily members as well as extracellular domains of the intercellular adhesion molecules (ICAM) and their ligands.

TABLE 6
Structural similarity of P. aeruginosa azurin with various
pathogenesis-related proteins

| | | | Azurin (1jzg) |
|---|---|---|---|
| PDB | Annotation | Reference | DALI z score⁽¹⁾ |
| 1VCA | Human Vascular Cell Adhesion | 17 | 3.5 |
| B1 | Molecule-1, VCAM-1 | | |
| 1ZXQ1 | The Crystal Structure of ICAM-2 | 19 | 3.3 |
| 1IAM1 | Structure of The Two Amino- | 20 | 3.0 |
| | Terminal Domains of, ICAM-1 | | |
| 1OB1 | Crystal Structure of a Fab complex | 21 | 2.9 |
| A1 | with Plasmodium falciparum | | |
| | MSP1-19 | | |
| 1T0P B | The complex Structure of | 22 | 2.5 |
| | Binding Domains of ICAM-3 | | |
| | and Alphabeta2 | | |
| 1 CDH | CD4 (D1D2 Fragment) | 18 | 3.4 |
| | Type 1 Crystal Form | | |
| 2NCM | Neural Cell Adhesion Molecule, | 23 | 2.4 |
| | NCAM | | |

⁽¹⁾Structural alignment to azurin were made using DALI (16). Structure pairs with DALI z scores <2 are considered dissimilar.

### Example 12

### Cloning and Expression of the Laz and H.8-Azurin Fusion Genes

The m/z gene from Neisseria gonorrhoeae was cloned based on its known sequence (SEQ ID NO: 22). The P. aeruginosa azurin gene (SEQ ID NO: 1), termed paz, and the sequence of the H.8 epitope of m/z from N. gonorrhoeae (SEQ ID NO: 23), were used to clone in frame the H.8 epitope gene in the 5′-end of paz to produce H.8-paz or in the 3′-end of paz to generate paz-H.8.

TABLE 7
Cancer cells, bacterial strains and genetic constructs

| Cells/strains/ | | |
|---|---|---|
| plasmids | Relevant characteristics* | Reference |
| P. aeruginosa | Prototroph, FP-(sex factor minus) | Holloway, et al., |
| PAO1 | | Microbiol. Rev. 43: 73-102 |
| | | (1979) |
| E. coli JM109 | Cloning and azurin expression strain | Yanisch-Perron, et al., |
| | | Gene 33: 103-119 (1985) |
| E. coli BL21 | GST expression strain | Novagen |
| (DE3) | | |
| N. gonorrhoeae | Prototroph used for DNA isolation | American Type Culture |
| | | Collection |
| F62 | | |
| pUC18 | General cloning vector, Ap^{r} | Yanisch-Perron, et al., id. |
| pUC19 | General cloning vector, Ap^{r} | Yanisch-Perron, et al., id. |
| pUC18-laz | A 1 kb PCR fragment from genomic | Herein |
| | DNA of N. gonorrhoeae F62 cloned | |
| | into pUC18 | |
| pUC19-paz | A 0.55 kb PCR fragment from P. aeruginosa | Yamada, et al., Proc. Natl. |
| | PAO1 cloned into HindIII | Acad. Sci. USA 99: 14098-14103 |
| | and PstI digested pUC19, Ap^{r} | (2002); Yamada, et |
| | | al., Proc. Natl. Acad. Sci. |
| | | USA 101: 4770-4775 |
| | | (2004) |
| pUC18-H.8- | Fusion plasmid encoding H.8 from | Herein |
| paz | N. gonorrhoeae and azurin from | |
| | P. aeruginosa PAO1, Ap^{r} | |
| pGEX-5X-3 | GST gene fusion vectors, Ap^{r} | Amersham |
| pET29a | E. coli expression vector, Km^{r} | Novagen |
| pET29a-gst | pET29a derivative containing the gst | Herein |
| | gene, Km^{r} | |
| pGEX-5X-3- | pGEX-5X-3 derivative containing H.8- | Herein |
| H.8 | encoding region, Ap^{r} | |
| pET29a-gst- | pET29a derivative containing gst-H.8 | Herein |
| H.8 | gene, Km^{r} | |

*Ap, ampicillin; Km, kanamycin; GST, Glutathione S-transferase.

Cloning and Expression of the paz and m/z Genes. The cloning and hyperexpression of the azurin gene has been described. (Yamada, et al., Proc. Natl. Acad. Sci. USA 99:14098-14103 (2002); Punj, et al., Oncogene 23:2367-2378 (2004)) The Laz-encoding gene (m/z) of Neisseria gonorrhoeae was amplified by PCR with genomic DNA of N. gonorrhoeae strain F62 as template DNA. The forward and reverse primers used were

(SEQ ID NO: 34)

5′-CCGGAATTCCGGCAGGGATGTTGTAAATATCCG-3′
and

(SEQ ID NO: 35)

5′-GGGGTACCGCCGTGGCAGGCATACAGCATTTCAATCGG-3′

where the additionally introduced restriction sites of EcoRI and KpnI sites are underlined respectively. The amplified DNA fragment of 1.0 kb, digested with EcoRI and KpnI, was inserted into the corresponding sites of pUC18 vector (Yanisch-Perron, et al., Gene 33:103-119 (1985)) so that the m/z gene was placed downstream of the lac promoter to yield an expression plasmid pUC18-laz (Table 7).

The plasmids expressing fusion H.8 of N. gonorrhoeae Laz and azurin of P. aeruginosa (Paz) were constructed by PCR with pUC19-paz and pUC18-m/z as templates. For H.8-Paz fusion, a 3.1 kb fragment was amplified with pUC18-m/z as a template and primers, 5′-(phosphorylated)GGCAGCAGGGGCTTCGGCAGCATCTGC-3′ (SEQ ID NO: 36) and 5′-CTGCAGGTCGACTCTAGAGGATCCCG-3′ (SEQ ID NO: 37) where a SalI site is underlined. A PCR amplified a 0.4 kb fragment was obtained from pUC19-paz as a template and primers, 5′-(phosphorylated)GCCGAGTGCTCGGTGGACATCCAGG-3′ (SEQ ID NO: 38) and 5′-TACTCGAGTCACTTCAGGGTCAGGGTG-3′ (SEQ ID NO: 39) where a XhoI site is underlined. A SalI digested PCR fragment from pUC18-m/z and XhoI digested PCR fragment from pUC19-paz were cloned to yield an expression plasmid pUC18-H.8-paz (Table 7).

E. coli JM109 was used as a host strain for expression of azurin and its derivative genes. Recombinant E. coli strains were cultivated in 2×YT medium containing 100 μg/ml ampicillin, 0.1 mM IPTG and 0.5 mM CuSO₄ for 16 h at 37° C. to produce the azurin proteins.

When E. coli strains harboring these plasmids were grown in presence of IPTG, cells lysed and the proteins purified as described for azurin (Yamada, et al., Proc. Natl. Acad. Sci. USA 99:14098-14103 (2002); Punj, et al., Oncogene 23:2367-2378 (2004); Yamada, et al., Cell. Microbiol. 7:1418-1431 (2005)), the various azurin derivatives migrated on SDS-PAGE as single components, although the H.8 containing proteins (about 17 kDa) showed anomalous migrations, as noted before (Cannon, Clin. Microbiol. Rev. 2:S1-S4 (1989); Fisette, et al., J. Biol. Chem. 278:46252-46260 (2003)).

Plasmid Construction for Fusion GST Proteins. Plasmids expressing fusion glutathione S-transferase (GST)-truncated wt-azurin (azu) derivatives were constructed by a polymerase chain reaction using proofreading DNA polymerase. For pGST-azu 36-128, an amplified PCR fragment was introduced into the BamH1 and EcoR1 sites of the commercial GST expression vector pGEX-5× (Amersham Biosciences, Piscataway, N.J.). The fragment was amplified with pUC 19-azu as a template and primers, 5′-CGGGATCC CCG GCA ACC TGC CGA AGA ACG TCA TGG GC-3′ (SEQ ID NO: 40) and 5′-CGGAATTC GCA TCA CTT CAG GGT CAG GG-3′ (SEQ ID NO: 41), where the additionally introduced BamHI and EcoRI sites are underlined respectively. Carboxyl-terminus truncation of azu gene was cumulatively performed by introducing a stop codon using QuickChange site-direct mutagenesis kit (Stratagene, La Jolla, Calif.).

For pGST-azu 36-89, a stop codon were introduced into Gly90. The plasmid carrying pGST-azu 36-128 was used as template DNA. Three sets of oligonucleotides for site-direct mutagenesis are shown as follows. For pGST-azu 36-89: 5′-CCA AGC TGA TCG GCT CGT GAG AGA AGG ACT CGG TGA CC-3′ (SEQ ID NO: 42), and 5′-GGT CAC CGA GTC CTT CTC TCA CGA GCC GAT CAG CTT GG-3 (SEQ ID NO: 43).

For pGST-azu 88-113, carboxyl terminus truncation of azu gene was cumulatively performed by introducing stop codon using QuickChange site directed mutagenesis kit (Stratagene, La Jolla, Calif.). For pGST-azu 88-113, a stop codon was introduced into Phe114. The plasmid carrying pGST-azu 88-128 was used as the template. For pGST-azu 88-128 an amplified PCR fragment was introduced into the BamH1 and EcoR1 sites of the commercial GST expression vector pGEX-5× (Amersham Biosciences). The fragment was amplified with pUC19-azu as the template and primers, 5′-CGGGGATCC CCG GCT CGG GCG AGA AGG AC-3′ (SEQ ID NO: 44) and 5′-CGGGAATTC TCC ACT TCA GGG TCA GGG TG-3′ (SEQ ID NO: 45) where the additionally introduced BamH1 and EcoR1 sites are underlined respectively.

One set of oligonucleotides for site directed mutagenesis are shown as follows for the preparation of pGST-azu 88-113: 5′-GTT CTT CTG CAC CTA GCC GGG CCA CTC CG-3′ (SEQ ID NO: 46) and 5′-CGG AGT GGC CCG GCT AGG TGC AGA AGA AC-3′ (SEQ ID NO: 47). pGST-azu 88-113 was used to transform E. coli XL-1 Blue strains. Plasmid extraction was performed using a commercial kit (Qiagen, Venlo, The Netherlands) and PCR sequencing were performed to assess plasmid insertion and transfection.

E. coli BL21 (DE3) was used as a host strain for expression of the gst and its fusions derivatives. E. coli strain XL 1-Blue transformed with pGST-azu plasmids was grown in LB media with ampicillin for three hours at 37° C. upon which IPTG induction (0.4 mM) was performed an subsequent incubation for 2-4 h at 37° C. to maximize the expression levels. Cells were isolated by centrifugation, resuspended in 25 mL of 1×PBS buffer. Subsequent cell lysis involved two sequential treatments of the cell suspension via sonication (20 min on ice) and heat-cold shock in acetone-dry ice bath (using the appropriate protease inhibitors). Supernatants of the cell lysis mixture were isolated and passed through a freshly packed and PBS equilibrated 1 mL glutathione-sepharose 4B (Amersham Biosciences) column. After column washing and subsequent elution of GST-azu product using 10 mM glutathione in 20 mM Tris-HCl pH 8. GST-Azu 88-113 purity was tested via electrophoresis using a 10% SDS-PAGE Tris-Gly gel stained with Coomassie Brilliant Blue R reagent. Protein concentration was determined using the Bradford Method.

### Example 13

### Azurin Binds to the C-Terminal Fragments MSP1-19 and MSP1-42 of the P. falciparum Merozoite Surface Protein MSP1

Given the structural similarity (Table 6) between azurin and the fab fragment of the monoclonal antibody G17.12 in complexation with Pf MSP1-19 (Pizarro et al., id), the ability of azurin to form a complex with Pf MSP1-42 or Pf MSP1-19 was determined. Two derivatives of azurin, Laz, an azurin-like protein from gonnococci and meningococci such as Neisseria meningitides with an additional 39 amino acid epitope called an H.8 epitope (Gotschlich & Seiff, FEMS Microbiol. Lett. 43:253-255 (1987); Kawula et al., Mol. Microbiol. 1: 179-185 (1987)) and H.8-azurin, where the H.8 epitope of Laz has been fused in the N-terminal part of P. aeruginosa azurin in frame (as described in Example 12) were tested.

In vitro protein-protein interactions were evaluated using a Biacore X spectrometer from Biacore AB International. All experiments were conducted at 25° C. in HBS-EP running buffer (0.01 M HEPES, pH 7.4, 0.15 M NaCl, 3 mM EDTA, 0.005% v/v Surfactant P20) using Au-CM5 sensor chips (Biacore). Protein immobilizations on CM5 chips were conducted according to the amine coupling procedure. Proteins were immobilized after NHS/EDC preactivation of the CM5 surface: 50 μl injections of azurin (510 μM). Subsequent treatment of CM5 surface with ethanolamine (1M, pH 8.8) removed uncrosslinked proteins. Binding studies were performed by injecting protein eluents (50 μl) over the protein-CM5 surface at flow rates of 30 μl/min with a 120 sec time delay at the end of the injections. Protein eluents included GST-azurin fusion proteins (GST, GST-Azu 36-128, GST-Azu 36-89, and GST-Azu 88-113, as described in Example 12). Sensor chip surfaces were regenerated between protein injections using 100 mM NaOH (10 μl injection pulse). All binding studies were run in parallel against a negative flow channel with bare Au-CM5 sensor surface to correct for nonspecific binding to the chips. To generate binding constant data, titration experiments were designed via injection of increasing concentrations of protein eluents (0.05-2000 nM). The SPR data were fit to a Langmuir (1:1) equilibrium binding model [Req=Rmax/(1+Kd/C] as specified in the Biacore software from which binding constants (Kd) were extrapolated.

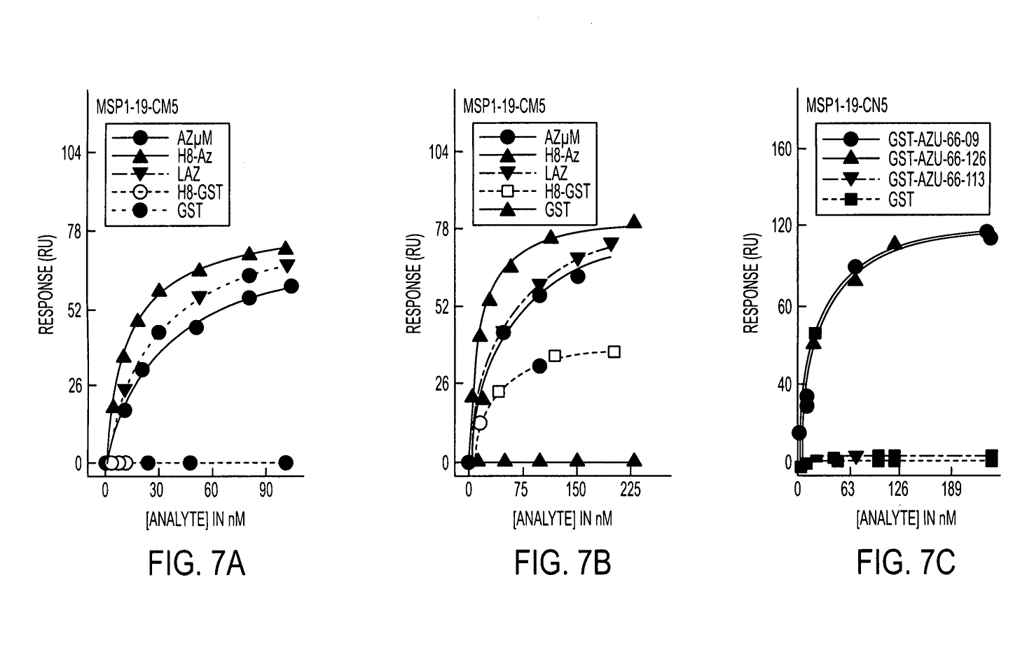

Specific interactions of the Pf MSP1-19 and Pf MSP1-42 proteins with azurin, H.8-azurin and Laz were determined by surface plasmon resonance (SPR) analysis and the data are presented in FIG. 7. SPR sensorgrams for binding of immobilized PfMSP1-19 and Pf MSP1-42 with azurin and its derivatives indicated selective recognition among these proteins. While nanomolar concentrations of azurin allowed significant binding with the immobilized MSP1-19 (FIG. 7A) or MSP1-42 (FIG. 7B), both H.8-azurin and Laz demonstrated a higher affinity of binding with the merozoite surface protein MSP1 cleavage products, with characteristic Kd values of 32.2 nM between azurin and MSP1-19 and 54.3 nM between azurin and MSP1-42. The Kd values between H.8-azurin and MSP1-19 and MSP1-42 were 11.8 nM and 14.3 nM while such values between Laz and MSP1-19 and MSP1-42 ranged from 26.2 nM and 45.6 nM respectively.

To examine if the H.8 epitope might facilitate binding of the H.8-azurin or Laz to the PFMSP1-19 or PFMSP1-42 moieties, the ability of glutathione S-transferase (GST) and a fusion derivative H.8-GST where the H.8 epitope was fused in the N-terminal of GST (see Example 12), to bind MSP1-19 or MSP1-42 was tested. Neither the GST nor the H.8-GST bound PfMSP1-19 (FIG. 7A) or MSP1-42 (FIG. 7B), although H.8-GST showed a weak binding with MSP1-42.

Glutathione S transferase (GST) and some of the fusion proteins where parts of azurin were fused to GST (Yamada et al., Cell. Microbiol. 7:1418-1431 (2005), and Example 4) were tested for their ability to bind to MSP1-19. GST alone, or GST-Azu 88-113, where the azurin amino acid sequence 88 to 113 out of 128 amino acids of azurin was fused to GST in frame, did not show any binding (FIG. 7C) while GST-Azu 36-89 with amino acid sequence 36 to 89 and GST-Azu 36-128 with amino acid sequence 36 to 128 showed significant binding with MSP1-19 with Kd values of 20.9 nM and 24.5 nM respectively.

### Example 14

### Inhibition of Plasmodium falciparum Parasitemia by Azurin, H.8-Azurin and Laz

The extent of parasitemia was determined using schizont stage parasites and normal red blood cells (RBC). Normal red blood cells (RBCs) were washed twice in serum-free medium and resuspended to 10% hematocrit in complete RPMI. 200 μl of 10% hematocrit RBCs were added to each of 24 wells in addition to 300 μl complete RPMI without or with azurin, H.8-azurin or Laz at various concentrations. Schizont stage P. falciparum parasites were prepared by centrifuging a late-stage culture through a Percoll cushion at 3200 rpm for 10 min. For infection, 4×10⁶ parasites per well in 500 μl volume were added at time zero. The plate was incubated overnight (about 16 h) and then scored by thin blood smear and Giemsa stain at that time.

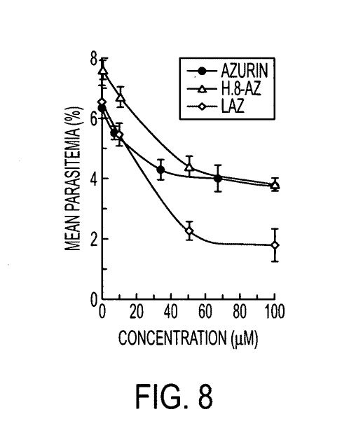

Azurin, H.8-azurin or Laz all demonstrated significant inhibition of parasitemia in a dose-dependent manner (FIG. 8), although at relatively high concentrations (about 50 μM). Such high concentrations presumably reflect the multiple ways the malarial parasites invade the erythrocytes (Cowman et al., FEBS Lett. 476:84-88 (2000); Baum et al., J. Biol. Chem. 281:5197-5208 (2006)) and a high concentration of azurin or Laz is necessary to interfere in the entry process. As indicated by their enhanced binding affinities to MSP1-19, both H.8-azurin and Neisserial Laz protein showed a higher level of inhibition of P. falciparum parasitemia as compared to azurin (FIG. 8).

When azurin was labeled with the red fluorescent dye Alexafluor 568 and used during the invasion assay, very little red fluorescence was detectable inside the RBC, suggesting that azurin seems not to enter the RBC as part of bound MSP1-19, or more likely, that the RBCs that showed the presence of the schizonts were the ones where azurin failed to bind with the MSP1-19. These data fully agree with our previous observation (Yamada et al., Cell. Microbiol. 7:1418-1431 (2005)) that azurin does not enter normal cells such as macrophages, mast cells, etc, and the effect of azurin, H.8-azurin or Laz is at the entry level rather than the intracellular replication of the parasite. Taken together, the data in FIG. 8 demonstrate the potential antimalarial action of azurin, H.8-azurin and Laz through interference in the invasion of the RBC by the parasites.

### Example 15

### Azurin Binds ICAMs

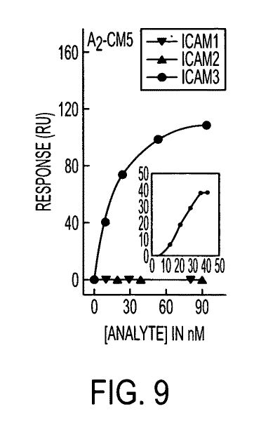

An interesting structural similarity between azurin and ICAMs (Table 6) that are known to be involved as receptors for P. falciparum-infected erythrocytes (Wassmer et al., PloS Med. 2:885-890 (2005); Dormeyer et al., Antimicrob. Agents Chemotherap. 50:724-730 (2006)) prompted test analysis of protein-protein interactions as measured by SPR between azurin and ICAMs such as ICAM-1, ICAM-2, ICAM-3 and NCAM. With immobilized azurin on the CM5 chip, ICAM-3 (FIG. 9, Kd=19.5+5.4 nM) and NCAM (FIG. 9, inset), but interestingly not ICAM-1 and ICAM-2, showed strong binding. While not limiting the manner in which the invention operates, part of effect of azurin on inhibition of P. falciparum parasitemia might also be mediated through its interaction with ICAM-3 or NCAM.

### Example 16

### In Vivo Inhibition of HIV Infection of Lymphocytes by Azurin Mutant and Cytochrome C₅₅₁

The M44KM64E mutant of azurin was mixed with cytochrome c₅₅₁ on a 1:1 basis (1 μM azurin:1 μM cytochrome c₅₅₁). HIV-infected human blood lymphocytes were incubated with the mixed azurin/cytochrome c₅₅₁ proteins at concentrations of 0, 500 to 1000 μg/ml protein for 7 days. The HIV p24 levels were then measured in the infected lymphocytes. p24 levels are known to be colinear with HIV virus levels in infected blood. Measuring the change in p24 concentrations in blood will indicate the change of HIV virus titer in the blood. Controls with non-infected human blood lymphocytes were also run in a parallel manner. After the 7 day incubation, the HIV p24 levels in the infected lymphocytes were reduced by 25% to 90% as compared to the control infected lymphocytes with 0 μg/ml azurin and cytochrome c₅₅₁. In the non-infected control cells, after 7 days of incubation with the protein mixture, neither cell death nor cytotoxicity was found.

### Example 17

### Effect of Azurin, H.8-Azurin and Laz on HIV-1 Entry and Viral Growth

The effect of various concentrations of azurin, H.8-azurin and Laz on the growth of three subtypes of HIV-1 in peripheral blood mononuclear cells (PBMCs), Bal, RW/92/008/RE1 clade A and IN/2157 D15 clade C.

### Example 18

### Effect of Azurin, H.8-Azurin and Laz on HIV-1 Entry and Viral Growth

The effect of various concentrations of azurin, H.8-azurin and Laz on the growth of three subtypes of HIV-1 in peripheral blood mononuclear cells (PBMCs), Bal, RW/92/008/RE1 clade A and IN/2157 D15 clade C. Plasmid construction and expression of Azurin, H.8-Azurin and Laz were performed as in Example 12.

HIV-1 Suppression Assay. Azurin, H.8-azurin and Laz were filter sterilized through a 0.45 μM filter. Peripheral blood mononuclear cells (PBMC) were treated with polybrene (5 μg/ml) for 1 h and seeded at 250,000 cells/well in a microtiter plate. The plate was spun at 800 rpm for 5 min to collect the cells. The supernatant was taken off and media with protein (at concentrations of 0.3, 0.6, 1.2, 6.0 and 30 μM) was added (100 μl). The cells were then incubated for 1 h. AZT (25 μM) was used as a control. The proteins were left on cells and 100 μl of virus (Bal, 2167, or RW/92/008/RE1) was added and incubated for 2 h. The plate was spun again at 800 rpm for 5 minutes and protein and virus was removed. Protein and media were added back for a total volume of 100 μl and incubated for 5 days. At the end of the 5 day period, the culture supernatant was tested for HIV/p24 by ELISA.

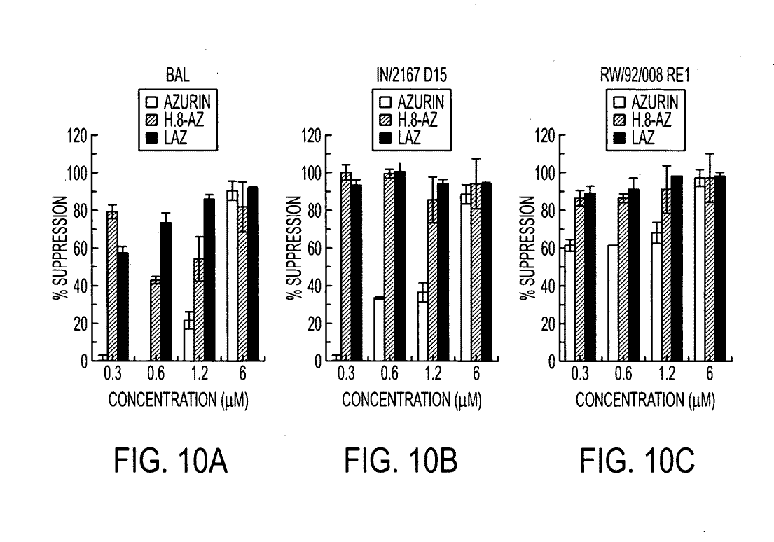

The results in FIG. 10 show that azurin at a concentration of 6.0 μM shows about 90% suppression of the growth of HIV-1 Bal, the most predominant clade B circulating in the US and Western Europe, a clade B African isolate RW/92/008/RE1 and a clade C Indian isolate IN/2167 D15. However, H.8-azurin (azurin with the H.8 epitope in the N-terminal) had high inhibitory activity against all the three subtypes at concentrations as low as 0.3 μM, particularly for the African and the Indian subtypes (FIG. 10).

The Neisserial protein Laz, which also harbors the H.8 epitope in the N-terminal part of the Neisserial azurin homolog (Gotschlich & Seiff FEMS Microbiol. Lett. 43:253-255 (1987); Kawula et al., Mol. Microbiol. 1:179-185 (1987)), had similar inhibitory activity for the three subtypes, particularly for the African and the Indian subtypes (FIG. 10), demonstrating a role of the H.8 epitope in promoting enhanced anti-HIV-1 activity by azurin. No effect on host cell (PBMC) death by MTT assay (Yamada et al., Proc. Natl. Acad. Sci. USA 99:14098-14103 (2002); Punj et al., Oncogene 23:2367-2378 (2004)) was discernible for all concentrations of these three proteins, suggesting that inhibition of HIV-1 growth was not due to death of the host cells.

### Example 19

### Azurin Binding with gp120 and CD4 as Studied by Surface Plasmon Resonance

Surface Plasmon Resonance experiments were conducted to determine the extent of azurin binding not only to CD4 but also to HIV-1 surface proteins such as gp120 or gp41 known to be involved in HIV-1 entry and other proteins such as Nef or Gag that are involved in intracellular virus multiplication.

Surface Plasmon Resonance (SPR) Studies. In vitro protein-protein interactions were evaluated using a Biacore X spectrometer from Biacore AB International (Uppsala, Sweden). All experiments were conducted at 25° C. in HBS-EP running buffer (0.01 M HEPES, pH 7.4, 0.15 M NaCl, 3 mM EDTA, 0.005% v/v Surfactant P20)) using Au-CM5 sensor chips purchased from Biacore. Protein stock solutions were prepared in PBS after desalting on G-75 column and lyophilizaiton in order to preconcentrate and exchange the buffer.

Protein immobilizations on CM5 chips were conducted according to the amine coupling procedure. Due to differences in protein crosslinking efficiencies, proteins were immobilized under various conditions after NHS/EDC preactivation of the CM5 surface: 50 μl injections of azurin (510 μM), or 35 μl injections of CD4 (25 μM, 2×), or HIV-1 gp120 (10 μM). Subsequent treatment of CM5 surface with ethanolamine (1M, pH 8.8) removed uncrosslinked proteins prior to binding studies. Binding studies were performed by injecting protein eluents (50 μl) over the protein-CM5 surface at flow rates of 30 μl/min with a 120 sec time delay at the end of the injections. Protein eluents included CD4 (Protein Sciences Corp., Meriden, Conn.), HIV-1 gp120 (Immunodiagnostics Inc., Woburn, Mass.), HIV-1 gp41 (Bioclone Inc., San Diego, Calif.), HIV-1 gag and HIV-1-nef (Chemicon International, Temecula, Calif.) and GST-azurin fusion proteins (GST, GST-Azu 36-128, GST-Azu 36-89, and GST-Azu 88-113, expressed in inventor's laboratory). Sensor chip surfaces were regenerated between protein injections using 100 mM NaOH (10 μl injection pulse). All binding studies were run against a negative flow channel containing bare Au-CM5 to correct for nonspecific binding effects. For the binding experiments wherein CD4 and HIV-1 gp120 served as the eluents (not immobilized), 1 mg/mL of carboxymethyldextran (CarboMer Inc., San Diego Calif.) was added to the running buffer in order to reduce nonspecific protein binding to the bare Au-CM5 flow channel surface.

To generate binding constant data, titration experiments were designed via injection of increasing concentrations of protein eluents (0.05-2000 nM) and the data collected. The SPR data could be fit to a Langmuir equilibrium binding model [Req=Rmax/(1+Kd/C] form which binding constants (Kd) were determined. Similar to the binding constant studies described above, competition studies with CD4-CM5 were performed using similar protocols but with injections of HIV-1 gp120+the competitor proteins (azurin, GST-Azu 36-128 and GST-Azu 88-113).

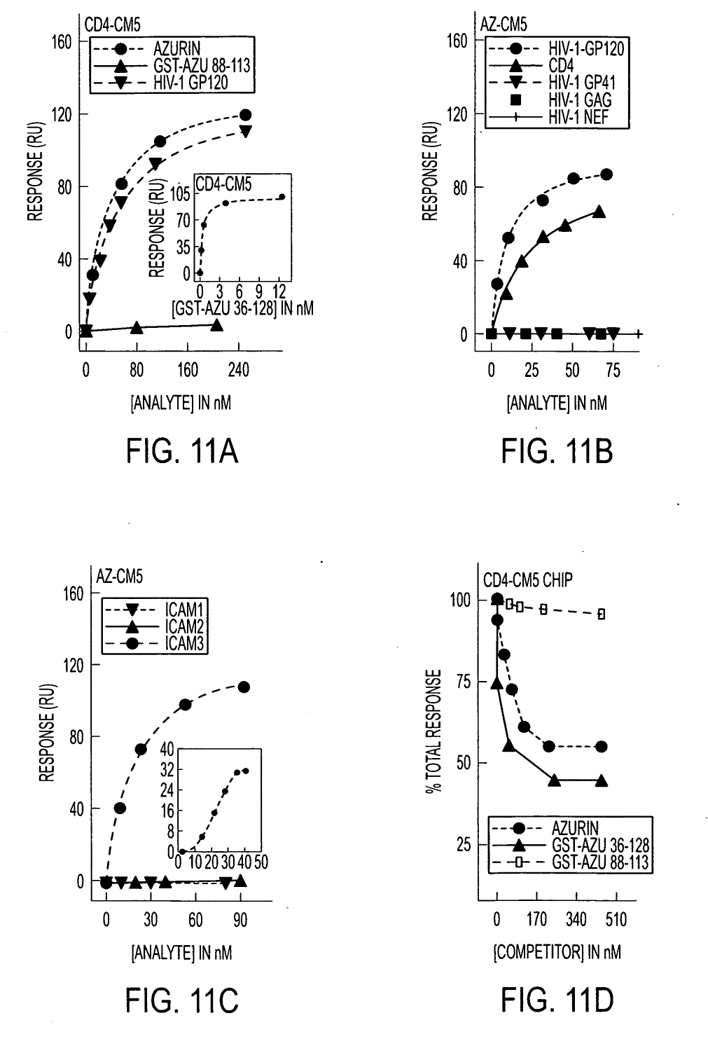

With CD4 immobilized in the sensor chip, both azurin and gp120 showed significant binding to CD4 (FIG. 11A). Azurin demonstrated a higher affinity for binding CD4 (Kd=36.9 nM) than the HIV-1 ligand gp120 (Kd=48.1 nM). While a GST-azurin fusion such as GST-Azu 88-113 showed no binding (FIG. 11A), another GST-azurin fusion protein, GST-Azu 36-128 showed even stronger binding than azurin itself with a Kd value of 0.34 nM, suggesting that parts of azurin might retain a stronger binding affinity than the full length protein. When azurin was immobilized on the sensor chip, gp120 showed somewhat stronger binding to azurin than CD4 (FIG. 11B), clearly demonstrating that azurin binds both to gp120 and CD4 with a high affinity. Interestingly, gp41, also involved in HIV-1 entry into the host cell, did not show any binding to azurin (FIG. 11B). Similar lack of binding was demonstrated for Gag and Nef.

### Example 20

### Azurin Binding with ICAMs and CD5 as Studied by Surface Plasmon Resonance

There is a structural similarity between azurin and ICAMs (Table 6) that are known to be involved as receptors HIV-1 infections. (Liao et al., AIDS Res. Hum. Retroviruses 16:355-366 (2000); Hioe et al., J. Virol. 75:1077-1082 (2001)) ICAM-3 has been implicated in stimulating HIV-1 transcription and viral production, thereby contributing additionally to intracellular viral growth. (Barat et al., J. Virol. 78, 6692-6697 (2004))

Protein-protein interactions as measured by SPR between azurin and ICAMs such as ICAM-1, ICAM-2, ICAM-3 and NCAM were therefore studied. With immobilized azurin on the CM5 chip, ICAM-3 (FIG. 2C, K_{d}=19.5±5.4 nM) and NCAM (FIG. 11C, inset), but not ICAM-1 and ICAM-2, showed strong binding. While not limiting the operation of the invention to any one mechanism, part of azurin suppression of HIV-1 growth might also be mediated through its interaction with ICAM-3 or NCAM.

### Example 21

### Azurin Competition with gp120 for CD4 as Studied by Surface Plasmon Resonance

Due to the higher affinity of binding of azurin to CD4, as compared to gp120 (FIG. 11A), a competition experiment was performed to see if azurin can interfere in gp120 binding with its cognate receptor CD4. As the concentration of the competitor protein (azurin, GST-Azu 36-128 or GST-Azu 88-113) was increased in presence of a fixed concentration of gp120 adsorbed to the immobilized CD4 chip, both azurin and GST-Azu 36-128 demonstrated significant decrease in the total protein binding of gp120 from the CD4-CM5 chip (FIG. 11D). Such apparent displacement of gp120 from the chip was not observed in case of GST-Azu 88-113 (FIG. 11D). GST-Azu 88-113 is known not to bind CD4 (FIG. 11A). While not limiting the operation of the invention to any one mechanism, this indicates that azurin or GST-Azu 36-128 fusion protein may successfully inhibit the complex formation between gp120 and CD4.

### Example 22

### Azurin and ICAM-3 Binding with DC-SIGN as Studied by Surface Plasmon Resonance

The strong binding of azurin with gp120, CD4 and ICAM-3 (FIG. 11) mimics the binding of another very important HIV-1 binding protein present on the surface of dendritic cells (DC) known as DC-SIGN (DC-specific intercellular adhesion molecule 3-grabbing nonintegrin) and a related protein called DC-SIGN/R. DC-SIGN is expressed abundantly on DC while DC-SIGN/R is expressed primarily on sinusoidal and endothelial cells. DC-SIGN plays a major role in HIV-1 immunopathogenesis by allowing DC, which are professional antigen presenting cells, to capture and present pathogens including HIV-1 to resting T cells through their interactions with ICAM-3 on the T cell surface. (Geijtenbeek et al., Cell 100, 575-585 (2000); Soilleux, Clin. Sci. 104, 437-446 (2003); Geijtenbeek et al., Placenta 22, S19-S23 (2001)). DC-SIGN has also been shown to bind avidly to HIV-1 envelope protein gp120, thereby capturing HIV-1 and transporting it to CD4⁺ T cells, where HIV-1 can replicate freely. (Snyder et al., J. Virol. 79:4589-4598 (2005))

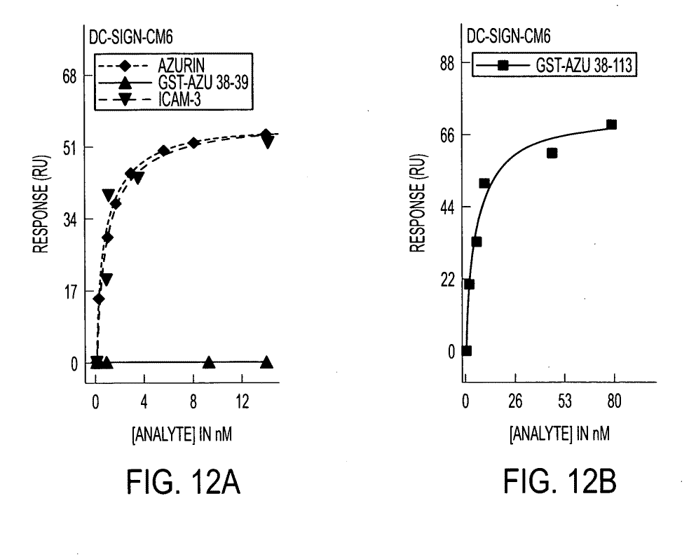

In SPR experiments with immobilized DC-SIGN on the sensor chip, both azurin (K_{d}=0.83±0.05 nM) and ICAM-3 (K_{d}=0.93±0.39 nM) bound strongly to DC-SIGN (FIG. 12A). While the GST-fusion derivative GST-Azu 36-89 showed very little binding (FIG. 12A), another GST-fusion derivative GST-Azu 88-113 exhibited relatively strong binding (K_{d}=5.98±1.13 nM), demonstrating the involvement of the C-terminal part of azurin in DC-SIGN binding (FIG. 12B). GST-Azu 88-113, however, does not bind with CD4 (FIG. 11A), suggesting that different parts of azurin have different binding specificities.

While not limiting the operation of the invention to any one mechanism, such binding with DC-SIGN demonstrates azurin's potential ability to interfere in the binding of HIV-1 with DCs. Thus DC-SIGN, a critical molecule on DC surface responsible for transmitting HIV-1 from the mucosal cells to the lymphoid T cells, may well find a strong competitor in azurin or Laz that can also avidly bind gp120, CD4 and ICAM-3.

### Example 23

### Azurin/Laz Acts in the Entry Stage of HIV-1 Infection

To determine if azurin acts at the entry or post entry step of HIV-1 infection, the effect of Laz on the Indian isolate IN/2167 of HIV-1 was investigated. In one experiment, activated PBMC (25,000 cells/well) were incubated with 6.0 μM Laz and HIV-1 for 2 h. The mixture was centrifuged to remove Laz and HIV-1, fresh medium without Laz was added back, and the culture was grown for 5 days. HIV-1 growth was monitored by measurement of p24 in the culture supernatant. Under this condition, Laz (6.0 μM) suppressed the HIV-1 growth by 43%. With higher concentration of Laz (30 μM), the extent of suppression was 76%. In a parallel experiment, when the Laz (6 or 30 μM) was added to the PBMC after the HIV-1 infection and its removal, very little suppression of viral growth was observed. As a positive control, when Laz (6.0 μM) was present both during infection and after removal of the virus with fresh medium during 5 days of the culture, the extent of inhibition was about 93%. While not limiting the operation of the invention to any one mechanism, such data clearly indicate that azurin or Laz exerts its effect primarily at the entry stage of infection.

### Example 24

### Treatment of More than One Disease Such as Patients Infected with Malaria and HIV

Twenty four patients, aged 22-50, who exhibit a history of preexisting antibodies to blood-stage P. falciparum parasites (as determined by immunofluorescent assay) and infected with AIDS presents with low to non-detectable HIV viral loads (RNA PCR) in the plasma as measured by PCR techniques, and increased CD4+ counts. Next, CD4+RO+ cells are enriched by magnetic separation and FACS sorting, and assayed to determine infectivity with respect to naive and uninfected cell co-culture experiments. This analysis of CD4+RO+ memory cells shows the presence of infective HIV.

The patients are injected with a pharmaceutical preparation of purified P. aeruginosa azurin. Two such patients serve as treated controls.

The sterile pharmaceutical preparation is in the form of 0.5 ml single-dose ampoules of sterile P. aeruginosa azurin in a pharmaceutical preparation designed for intravenous administration, as will be well known to those in the art. The pharmaceutical preparation is stored at 4° C. and protected from light before administration. In one clinical trial, P. aeruginosa azurin is prepared at five different concentrations: 10 μg, 30 μg, 100 μg, 300 μg and 800 μg azurin/cytochrome c₅₅₁ (1:1 on molecule basis) per 0.5 ml dose. The pharmaceutical preparation is given intravenously to twenty two patients for each 10 doses. Patients receive primary treatment at day 0 and subsequent doses identical doses for a period of 3 months until CD4+ cells, including memory cells, are at low levels Volunteers are observed for immediate toxic effects for twenty minutes after injection. Two patients receive placebo injections. During administration of azurin and for a period of approximately 1-2 months thereafter, or until CD4+ cells recover, the patients are maintained with antibiotics and antifungal therapy. Stem cell or precursor cell replacement is provided through a bone marrow transplant and cytokine therapy, both of which are performed according to conventional techniques. Twenty-four and forty-eight hours later, they are examined for evidence of fever, local tenderness, erythema, warmth, induration and lymphadenopathy, and are asked about complaints of headache, fever, chills, malaise, local pain, nausea and joint pain. Before each dose, blood and urine samples are taken for full laboratory examination. Complete blood count and serum chemistry profiles are rechecked two days after each dose. The presence of the malaria parasite are determined by light microscopic examination (ME) of the stained blood smears, or the ICT Malaria P.f./P.v. test kits (Binax, Inc., Portland, Me.). The patients are also followed at frequent intervals and monitored for CD34 cell level, reestablishment of CD4+ cells and quantitation of CD4+RO+ cells. Additionally, the patients' plasma is assayed for viral load by cell co-culture experiments. On reducing virus load in active and memory CD4+ T cells to low or non-detectable concentrations, the patients are weaned from azurin. After 3 months, the patients are weaned from antibiotic and antifungal therapy. Following this, the patients are followed at 6 month intervals and assayed for viral content. The results demonstrate the effectiveness of azurin therapy for patients with HIV infection. The results demonstrate the effectiveness of the therapy.

### Example 25

### Entry of p18 and p28 into Human Cell Lines

Cell Culture and Cell Lines: Human cancer and non-cancer (immortalized and non-immortalized) cell lines were obtained from ATCC [lung cancer (A549 and NCI-H23 adenocarcinoma), normal lung (CCD-13Lu), prostate cancers (DU145 and LN-CAP), normal prostate (CRL11611), breast cancer (MCF-7), normal breast (MCF-10A), colon cancer (HCT116), normal colon (CCD33Co), fibrosarcoma (HT1080), and ovarian cancer (SK-OV3 adenocarcinoma)]. Normal fibroblasts isolated from skin were established. Normal ovarian cells (HOSE6-3) were donated by Dr. S. W. Tsao (University of Hong Kong). Melanoma lines (UISO-Mel-2, 23, 29) were established and characterized. All cells except UISO-Mel-2 were cultured in MEM-E (Invitrogen, Carlsbad, Calif.) supplemented with 10% heat-inactivated fetal bovine serum (Atlanta Biological Inc., Lawrenceville, Ga.), 100 units/ml penicillin and 100 g/ml streptomycin at 37 C in 5% CO2 or air.

Proliferation assays/Cell growth: Melanoma cells were seeded (four replicates) in flat bottom 24 well plates (Becton Dickinson, Franklin Lakes, N.J.) at a density of 12×103 cells/well. After 24 hrs media was changed and fresh p18, p28, azurin or a similar volume of media without peptide (eight replicates) added daily for 72 hr. Cells were then counted in a Beckman Coulter (Z1 coulter particle counter). Values represent the mean±SD of 4 replicates.

MITT Assay: Melanoma cells were seeded at a density of 2000 cells/well in flat-bottomed 96 well plates (Becton Dickinson, Franklin Lakes, N.J.) and allowed to attach for 24 hrs. Freshly prepared peptide (10 μl) or culture medium was then added to each well. After 24 hrs, medium was changed and p18, p28 or azurin added daily. After 72 hr incubation, 10 μl of MTT reagent (Trevigen, Gaithersburg, Md.) was added to each well, the samples incubated for 3 hr, RT/sig 100 μl of detergent added to each well, and the samples incubated for an additional 3 hr at 37° C. Absorbance was measured with a SpectraMax 340 plate reader (Molecular Devices Corporation, Sunnyvale, Calif.) and percent change in the absorbance at 570 nm in treated cells relative to untreated controls determined. Values represent the mean±SD. Significance between control and treated groups was determined by Student's t-test.

Peptide synthesis: All azurin derived peptides including p18, Leu⁵⁰-Gly⁶⁷ LSTAADMQGVVTDGMASG (SEQ ID NO. 30), p28 Leu⁵⁰-Asp⁷⁷ LSTAADMQGVVTDGMASGLDKDYLKPDD (SEQ ID NO. 29), p18b Val⁶⁰-Asp⁷⁷ VTDGMASGLDKDYLKPDD (SEQ ID NO. 91), MAP, Mastoparan-7, and poly arginine (Arg₈ (SEQ ID NO: 94)) were synthesized by C S Bio, Inc. (Melo Park, Calif.). Peptides were received as lyophilized powder aliquoted and stored at −20° C. in air-tight desiccators. All peptides were subsequently analyzed by mass spectrometry and reverse phase HPLC as >95% purity and mass balance.

Predictive modeling for azurin peptides: GENETYX software (ver. 6.1) was used to generate Robson structure models for azurin derived peptides. Gamier, J., Osguthorpe, D. J., and Robson, B., J Mol Biol, 120: 97-120 (1978). The MAPAS Software was used to predict a given protein structure for strong membrane contacts and define regions of the protein surface that most likely form such contacts. Sharikov, Y. et al, Nat Methods, 5: 119 (2008). If a protein, i.e., azurin, has a membranephilic residue score (MRS)>3, membranephilic area score (MAS)>60%, and coefficient of membranephilic asymmetry (Kₘₚₕₐ)>2.5, there is a high probability that the protein has a true membrane-contacting region.

Peptide/Protein labeling: Peptides were dissolved in 1 ml PBS mixed with Alexafluor 568 dye (Molecular Probes, Eugene, Oreg.) at a 1:2 protein:dye ratio, 100 μl sodium bicarbonate added, and the mixture incubated overnight at 4° C. with continuous stirring. Labeled peptide was separated from free dye by dialyzing against cold-PBS using Slide-A-Lyzerg Dialysis Cassettes 1000 MWCO for p12 and 2000 MWCO for others (Pierce Biotechnology, Rockford, Ill.).

Cell penetrationfconfocal analysis: Cells were seeded on glass coverslips and allowed to attach overnight at 37° C. under 5% CO₂. Cells were rinsed with fresh media and incubated at 37° C. for 2 hrs in pre-warmed media containing Alexafluor 568 labeled azurin peptides (20 μM) or Arg8 (SEQ ID NO: 94) (5 μM), or media alone. Following incubation, coverslips were rinsed 3× with PBS, cells fixed in 2.5% formalin for 5 min, and washed 2× in PBS, once in d.i. H₂O, and coverslips mounted in media containing 1.5 μg/ml DAPI for nuclear counter staining (VECTASHIELD® Vector Laboratories, Burlingame Calif.). Cellular uptake and distribution were photographed under an inverted confocal laser scanning microscope (‘Model LC510, Carl Zeiss Inc., Gottingen, Germany).

Peptide co-localization with lysosomes or mitochondria was determined by incubating cells growing on a glass coverslip for 2 hrs at 37° with Alexafluor 568 labeled azurin or peptides. Mitrotracker (MitroTracker® Green FM Invitrogen Corporation, Carlsbad, Calif.) or lysotracker (LysoTracker® Green DND-26 Invitrogen Corporation, Carlsbad, Calif.) was added (final concentration 1 μM) for the last 30 mins of incubation. Cells were rinsed 3× with PBS, fixed in 2.5% formalin for 5 mins, washed 2× with PBS and incubated in 0.1% Triton-X100 in PBS for 15 min. Cells were then incubated with 1 μg/ml rabbit anti-human golgin 97 or anti-human caveolin I (Abcam, Cambridge, Mass.) in PBS with 1% BSA. After 1 hr incubation at 4° C., coverslips were washed once with PBS, incubated 10 min in PBS containing Alexafluor 468 conjugated goat anti-rabbit antibody, washed 2× in PBS and once in d.i.H20. Coverslips were then mounted in media containing 1.5 μg/mlDAPI for nuclear counter staining. Colocalization (yellow) of Alexafluor 568 (red) and Alexafluor 468 (green) was analyzed and photographed.

UISO-Mel-2 cells on coverslips were preincubated in MEM-E containing 100 μg/ml heparin sulfate (Sigma-Aldrich, St. Louis, Mo.) for 30 min and p18, p28 or Arg₈ (SEQ ID NO: 94) added to bring the final concentration to 20 μM. After 1 hr, coverslips were washed, fixed, and analyzed as described above.

Cell penetration by FFACS: Cells (1.0×10⁶/500 μl PBS) were incubated for 2 hrs at 37° C. with Alexafluor 568 labeled p18 or p28 (20 μM), Arg₈ (SEQ ID NO: 94) (5 μM), or media alone, washed 3× in PBS, fixed in 2.5% formalin for 5 min, washed twice in PBS, resuspended in 200 μl PBS, and passed through a screen to obtain a single cell suspension. Samples were analyzed with a MoFlo Cell Sorter (Dako, Glostrup, Denmark) λₑₓ 568 nm and λₑₘ 603 nm and the fold increase of the mean fluorescence intensity over background levels calculated. Results represent mean fluorescence of three separate experiments.

Entry inhibitors: UISO-Mel-2 cells (3×10⁵ per 300 μl), maintained in phenol red-, serum-free MEM-E at 37° C., were pretreated with inhibitors, including: Chloropromazine (inhibitor of clathrin-mediated endocytosis, 10 μg/ml, 60 min); Amiloride (macropinocytosis inhibitor, 50 μM, 30 min); Nystatin (50 μg/ml, 30 min); Methyl-β-cyclodextrin (MβCD, 5 mM, 60 min); Filipin (inhibitor of caveolae-mediated endocytosis, 3 μg/ml, 60 min); Taxol (microtubule stabilizer, 20 μM, 30 min); Staurosporine (cell cycle inhibitor, 250 nM, 10 min); Sodium azide (metabolic inhibitor, 1 mM, 60 min); Oauabain (ATPase-dependent Na⁺/K⁺ pump inhibitor, 50 mM, 60 min); Brefeldin A (BFA; Golgi apparatus disrupter, 100 μM, 60 min); Wortmannin (early endosome inhibitor, 100 nM, 30 min); Monensin (inhibits at late endosome/lysosome, 10 μM, 60 min); Nocodazole (inhibits caveosome formation, 10 μM, 60 min); Cytochalasin D (actin filament and microtubule disruptor, 5 μM, 30 min); Benzyl 2-acetamido-2-deoxy-α-D-galactopyranoside (BnGalNac; O-linked glycosylation inhibitor, 3 mM, 48 hrs); Tunicamycin (N-linked glycosylation inhibitor, 20 μg/ml, 48 hrs); and Neuraminidase (cleave sialic acid residues from proteins, IU/ml, 30 min). Final concentrations were derived from the dose response curves of individual inhibitors. Alexafluor 568 labeled p18 or p28 (20 μM) were then added, incubated for 1 hr, and the cells washed, fixed and prepared for flow cytometric analysis as described above.

Cell Membrane Toxicity Assays/LDH Leakage Assay: An LDH leakage assay was performed according to the manufacturer's instructions (CytoTox-One, Promega, Wis.) with 100 μl of UISO-Mel-2 cells (5×10³). Cells without peptides/proteins were used as a negative control. Experiments were carried out in triplicate (data represent mean±SEM).

Hemolysis assay: Human whole blood samples (2-3 ml) were centrifuged for 10 min at 1000×g, and the pellets washed once with PBS and once with HKR buffer pH7.4 (18). Cell pellets were then resuspended in HKR buffer to 4% erythrocytes, 50 μl transferred to a 1.5 ml tube with 950 μl of peptides, azurin (5, 50 and 100 μM) or 0.1% Triton X-100 in HRK buffer to completely disrupt the RBC membrane. MAP and Mastoparan7 (Bachem California, Inc., Torrance, Calif.) were used as positive controls. After 30 min incubation at 37° C. with rotation, tubes were centrifuged for 2 min at 1000×g, 300 μl of supernatants transferred to a 96-well plate and absorbance recorded at 540 nm.

Kinetics of Entry: UISO-Mel-2 cells (5×10⁵ cells) in 1.5 ml tubes were suspended in MEME media without phenol red. Reactions were started by adding either Alexa fluor 568-conjugated p18 at 0, 10, 20, 50, 100, 150 and 200 μM for 5, 10, 15 and 20 sec., or Alexafluor 568-conjugated p28 at 1, 10, 25, 50, 100, 150 and 200 μM for 30, 60, 90 and 120 sec on ice. After incubation, 1 ml of cold-PBS was added to the 250 μl reaction in mixture. Cells were centrifuged twice at 600×g for 2 min at 4° C. At least 10,000 fixed cells were analyzed by flow cytometry in each reaction and their background and relative fluorescence calculated.

I¹²⁵ Labeling of Azurin and Competition Assays: Peptide binding and entry was determined using a whole cell assay with UISO-Mel-2 cells in HEPES solution (50,000 cells/ml), were incubated for 30 min at 37° C. with increasing concentrations (0-175 nM) of radiolabeled a zurin in the presence/absence of 1000 fold excess of unlabeled p18, p28, or azurin, then washed 3 times with ice cold PBS, and radioactively remaining in the cell pellet counted using a gamma counter. Radioactivity in cells incubated with I¹²⁵ azurin alone was considered total binding; radioactivity in the presence of unlabeled azurin, p18, or p28 was considered nonspecific binding. Specific binding was determined by subtracting nonspecific binding from total binding and Scatchard plots generated.

### Example 26

### Domain of p28 Responsible for Preferential Entry into Cancer Cells

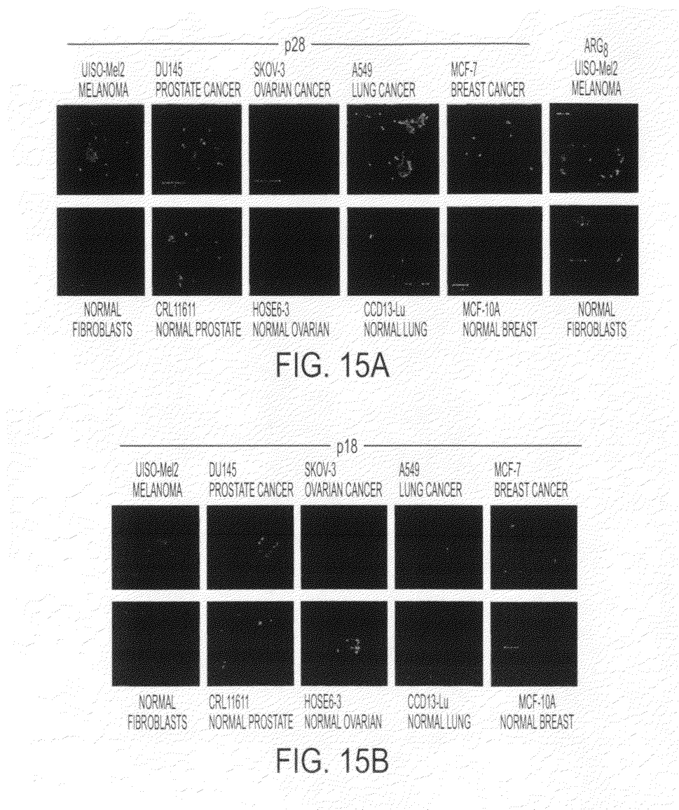

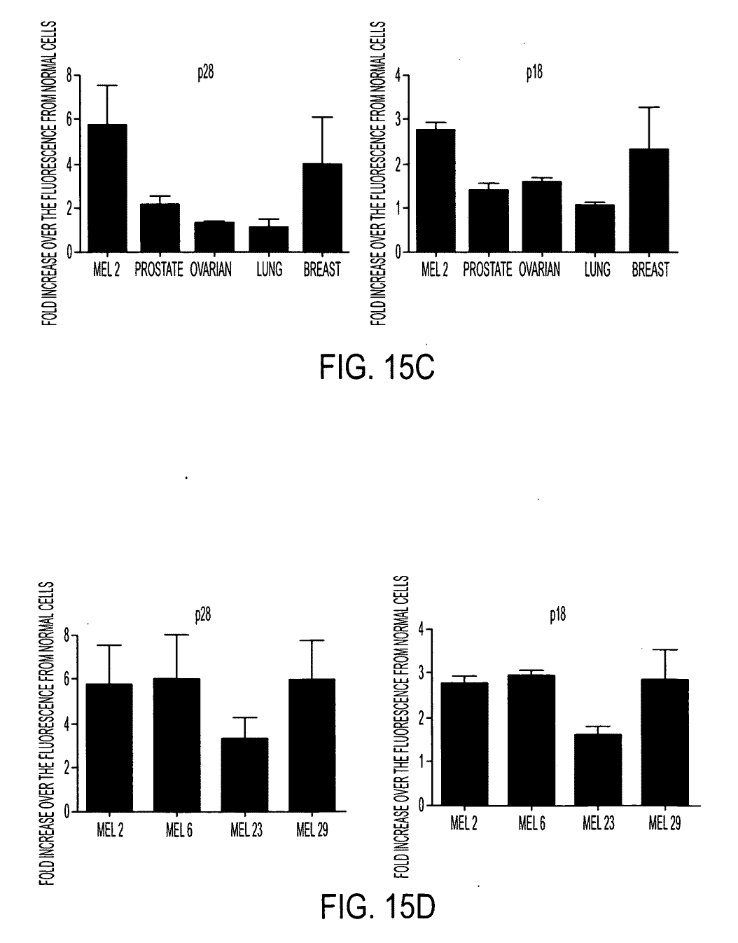

Initial data from peptide-GST constructs defined aa 50-77 of azurin as a putative PTD for cell penetration, which fits well with structural evidence for an α-helical region encompassing residues 54-67 of azurin stabilizing the azurin molecule. Confocal analyses initially suggested that p28 and p18 of p28/azurin (FIG. 15 A) penetrated human melanoma, prostate, lung, breast and ovarian cancer cells with relatively similar efficiency, but did not penetrate histologically matched normal cell lines to the same degree (FIG. 15 A). A singular exception was CCD13-Lu, a cell line derived from lung fibroblasts. The cationic Arg₈ (SEQ ID NO: 94) was rapidly and efficiently taken up into fibroblasts (FIG. 15 A) and all other normal cell lines tested (data not shown).

These observations were essentially confirmed by a more sensitive FACs analyses (FIG. 15 B) where p28 fluorescence was about 0.5-6 and p18 about 0.5-3 fold higher than the corresponding normal cell line, with the exception of lung cancer. A similar pattern in intracellular fluorescence intensity was observed within a histopathologic subtype, melanoma, where the relative intensity of p18 was about 50% of that observed with p28 (FIG. 15 C). Fluorescence intensity over background was also consistently lower in normal and cancer cell pairs exposed to p18 than p28 (data not shown), again suggesting less p18 entered individual cells. In all cases, the degree of entry of p18 and p28 into either cancer or normal cells was significantly less than that observed with Arg₈ (SEQ ID NO: 94), where no preference for entry was observed (FIG. 15 A). The predicted Robson structure (data not shown) of p18 suggests that the C-terminal amino acids form a partial β-sheet. This and the shorter length of p18, which lacks the hydrophilic C-terminal 10 amino acids (aa 68-77, SEQ ID NO: 92) of p28, suggests that p18, as a putative PTD for azurin, may have a more rapid entry into cancer and normal cells via a non-endocytotic over an endocytotic or membrane receptor mediated process. MAPAS data (MRS 3.74, MAS 87.1, Kₘₚₕₐ 2.37) predict that aa's 69, 70, 75, 76, 85 of azurin provide the best opportunity for membrane contact, suggesting the C-terminal region of p28, not present on p18 (aa 50-67) is most likely to contact specific residues on the cell membrane, irrespective of a cell's status.

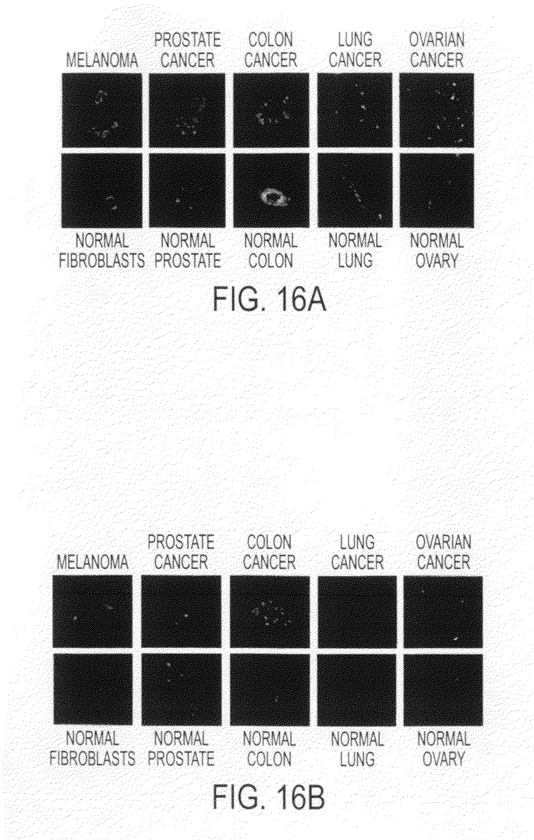

The preferential penetration of p18 and p28 was confirmed by exposing the same cell lines to azurin 60-77 (p18b), or aa 66-77 (SEQ ID NO: 93), the C-terminal 12 aa of p28 (FIG. 16 A, B). Here, the preferential penetration observed with p18 and p28 was completely abolished. p18b (theoretical pI 4.13) has a short α-helix and partial β-sheet, and is extremely hydrophilic which together may negate preferential entry. p12 (theoretical pI 4.33) lacks a secondary α-helical structure, but is also hydrophilic suggesting overall hydrophilicity may be a major contributor to the decrease in selectivity of cell penetration.

### Example 27

### Cell Penetration is not a Result of Membrane Disruption

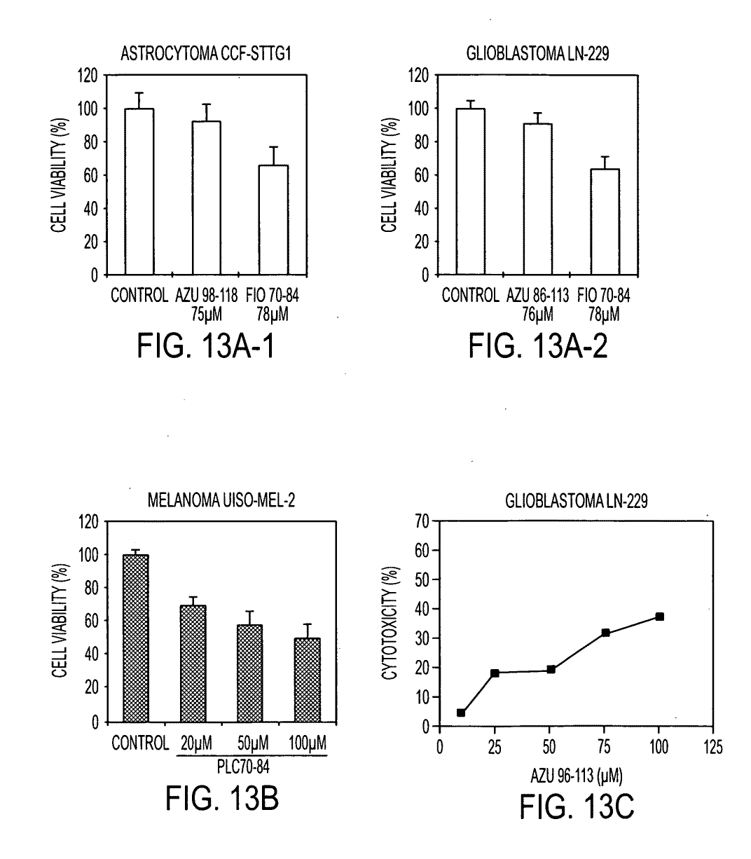

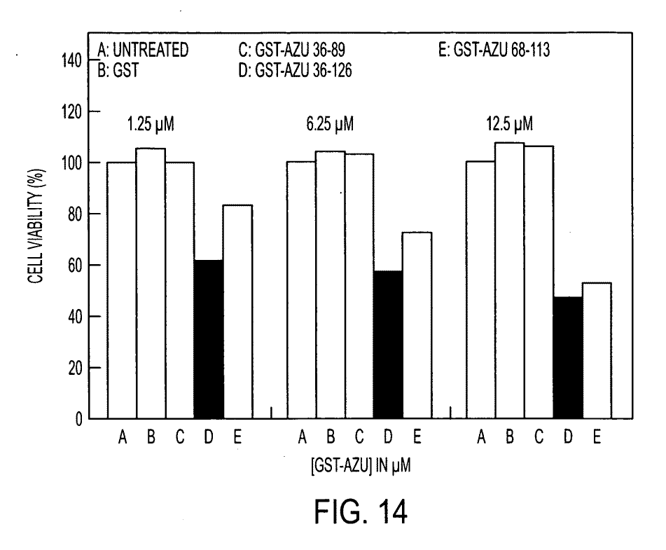

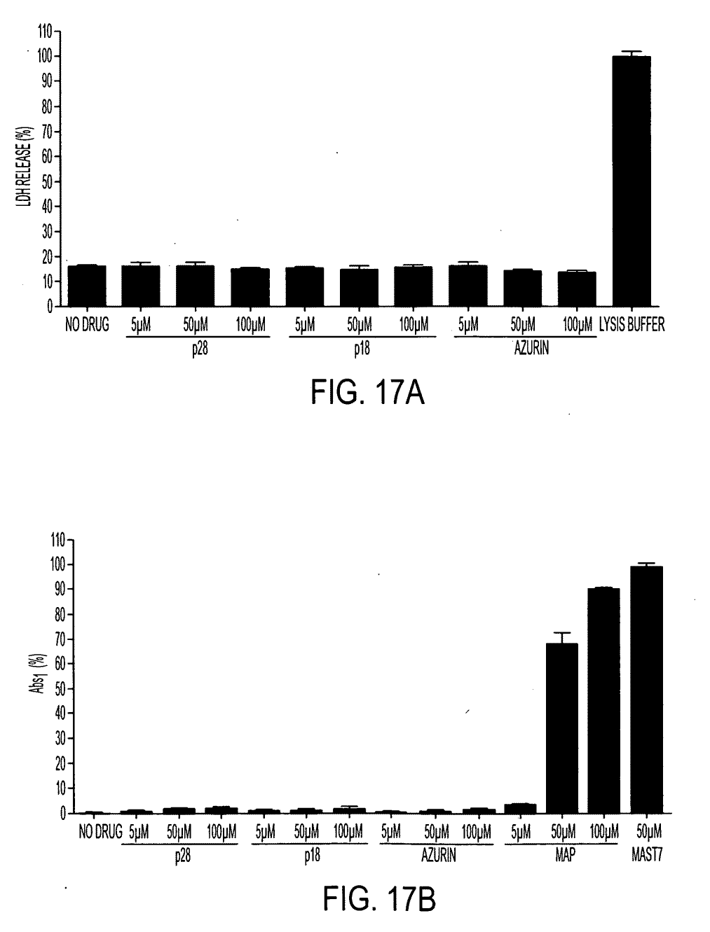

Cell penetration by azurin, p28, and p18 does not result from membrane disruption. An LDH leakage assay using UISO-Mel-2 cells in the presence of 5-100 μM p28, p18 or azurin (FIG. 17 A) suggested that neither peptide nor azurin entered cells by altering plasma membrane integrity. The lack of membrane disruption was confirmed by determining the hemolytic activity of azurin, p28, and p18 on human erythrocytes against the receptor mimetic MAP and mast cell degranulating peptide mastoparan 7, which translocates cell membranes as an amphipathic alpha-helix, and activates heterotrimeric G proteins. Mastoparan 7 caused complete cell lysis at 25 μM, while azurin, p28, and p18 had no hemolytic effect when compared to control (no peptide) (FIG. 13 B).

### Example 28

### p18/p28 Penetration is Energy Dependent and Saturable

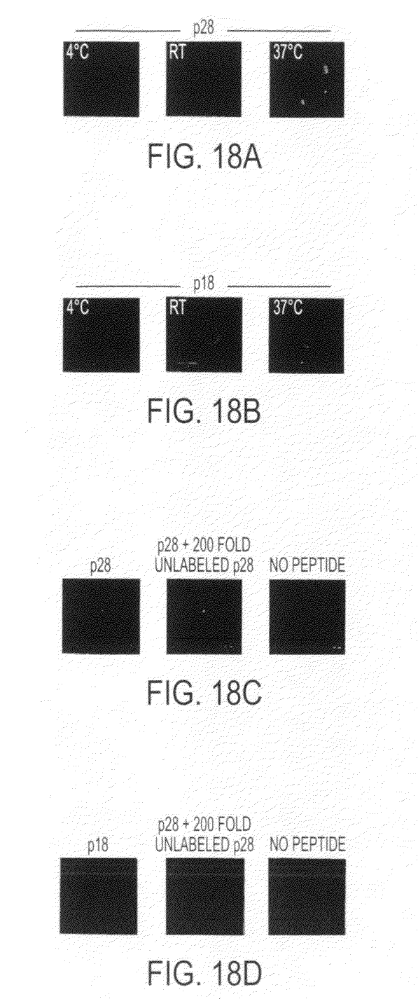

The penetration of p28 (FIG. 18 A) and p18 (FIG. 18 B) into UISO-Mel-2 cells is temperature dependent. Cell penetration and intracellular transport occurs relatively slowly over 3 hr at 4° C., while entry and intracellular transport through various compartments is rapid at 22 and 37° C. as p18 and p28 were present in the nucleus of UISO-Mel-2 cells within 2 hrs post exposure. The penetration of 5 μM p28 (FIG. 18 C) or p18 (FIG. 18 D) into UISO-Mel-2 cells after 30 min in the presence of a 200 fold excess of unlabeled peptide was severely curtailed, suggesting that entry was a saturable process and specific receptors or cell surface proteins or specific residues were, at least in part, responsible for initial entry.

### Example 29

### Kinetics of p28 and p18

The kinetics of p28 and p18 entry into UISO-Mel-2 cells relative to human fibroblasts was calculated after incubation, when cells were fixed and mean fluorescence intensity (MFI) determined. The Km and Vmax of each peptide were calculated by plotting peptide concentration (μM) vs velocity (MFI/sec) or by Scatchard analysis. Although the penetration of azurin fragments 50-67 (p18: Vmax 2.46, Km 101.6) and 50-77 (p28: Vmax 1.87, Km 159.1) into cancer and normal cells (Vmax 2.88, Km 102.1 and Vmax 1.89, Km 166.0, respectively) differs significantly from each other, with p18 entering −42% faster, the rate of the entry of each peptide into normal and cancer cells is virtually identical. The increase in amount of fluorescence following exposure of cancer cells to p28 relative to p18 is likely due to the increase in the amount of p28 entering malignant cells. ¹²⁵I azurin and p18 bound to UISO-Mel-2 cells with a similar affinity. In contrast, significantly more p28 (K_{d} 2.5 μcm, Bmax 3.0 pm) bound to UISO-Mel-2 cells with a higher affinity when exposed for a longer period of time (20 min vs 2 min) at a higher temperature (37° C. vs 4° C.) than either p18 (K_{d} 18 min, Bmax 0.51 pm) or azurin (K_{d} 10 nm and 0.48 pm). These results suggest that azurin, p28, and p18 all bind with relatively high affinity and capacity to a site on the cancer and normal cell surface prior to entry, but may enter via more than one mechanism.

### Example 30

### p18/p28 Penetration Involves Caveolae and the Golgi Complex

Peptides called cell-penetrating peptides (CPPs) or cell-delivery vectors (CDVs), such as penetratin, transportan, Tat (amino acids 47-57 or 48-60), and the model amphipathic peptide MAP, are short, amphipathic and cationic peptides and peptide derivatives, usually containing multiple lysine and arginine residues. Fischer, P. M., Med Res Rev, 27: 755-795 (2007). They form a class of small molecules receiving significant attention as potential transport agents or delivery vehicles for a variety of cargoes, including cytotoxic drugs, anti-sense oligo-nucleotides, proteins, and peptides, in gene therapy, and as decoy peptides. Hallbrink, M. et al. Biochim. Biophys. Acta 1515: 101-109 (2001); Lindgren, M., et al. Trends Pharmacol. Sci. 21: 99-103 (2000); Gusarova, et al, J Clin Invest, 117: 99-111 (2007); Melnick, A., Biochem Soc Trans, 35: 802-806 (2007); Astriab-Fisher et al., Pharm Res, 19: 744-754 (2002); El-Andaloussi et al., J Gene Med, 8: 1262-1273 (2006); Cashman et al., Mol Ther, 6: 813-823 (2002).

As a class, cationic CPPs such as pTat and Arg₈ (SEQ ID NO: 94) enter cells by initially binding to anionic, sulfated proteoglycans prior to endocytosis. Incubation of p28 and p18 and Arg₈ (SEQ ID NO: 94) with UISO-Mel-2 cells under serum free conditions in the presence/absence of 100 μg/ml heparin sulfite (HS) significantly reduced the amount of intracellular Arg₈ (SEQ ID NO: 94), but did not alter the entry of either p28 or p18 (FIG. 19 A). The penetration of p18 and p28 into UISO-Mel-2 cells in the presence or absence of a specific inhibitor of O-linked glycosylation, BnGalNac, and neruaminidase, which cleaves sialic acid residues, was further characterized (FIG. 19 B), and no inhibition of penetration was observed. However, tunicamycin, an inhibitor of N-linked glycosylation, significantly reduced the penetration of p18 and p28 across the cell membrane.

The entry of p18 and p28 into UISO-Mel-2 cells was also analyzed using inhibitors of energy dependent transport mechanisms, i.e., ATP. Sodium azide (FIG. 19 B) and ouabain (Na⁺ K⁺ ATPase pump) did not significantly inhibit the penetration of either peptide suggesting non endocytosic pathways might also be involved in the penetration of these peptides. Chlorpromazine (CPZ), a specific inhibitor of clathrin mediated endocytosis, also had no effect on penetration, nor did the macropinocytosis inhibitor amiloride. (FIG. 15 B). Stabilization of microtubules with taxol had no effect on penetration, but disruption of actin filaments and macropinocytosis with Cytochalasin D produced a small (−20%), reproducible inhibition of the penetration of p18 and p28. The lack of effect of amiloride suggests that the inhibitory activity of Cytochalasin D is probably through its effect on actin filaments.

Inhibition of the cell cycle with staurosporine did not block penetration, suggesting that penetration was not cell cycle specific. The lack of effect of staurosporine on p18 and p28 penetration of the cancer cell plasma membrane also suggests that a Src kinase/tyrosine kinase dependent pathway was not involved in penetration, was dynamin independent, and hence independent of caveolae budding. Neither p18 nor p28 co-localized with flotillin-1 (data not shown) a protein that resides within the plasma membrane and in a specific population of endocytic intermediates, again arguing against a role for flotillin and dynamin in internalization. In contrast, nocodazole, which disrupts caveolae transport and inhibitors of cholesterol mobilization and hence, caveolae-mediated endocytosis, inhibited penetration 50-65%.

The intracellular disposition of p18 and p28 was then analyzed using wortmannin, an inhibitor of early endosome formation, monensin, which inhibits late endosome/lysosome, and brefeldin A (BFA), a disruptor of the Golgi apparatus. Wortmannin did not block the intracellular accumulation of either p18 or p28 suggesting that, unlike cholera toxin, a caveolae to early endosome pathway is not involved in the intracellular trafficking of p18 and p28. The lack of early endosome involvement in the intracellular trafficking of p18 and p28 also suggests that clathrin mediated endocytosis is not involved in internalization of these peptides.

However, monensin (FIG. 19 B) and BFA reduced the intracellular accumulation of both peptides with a greater inhibitory effect on p28 (˜30%) than p18 (˜10%) (FIG. 19 B). The penetration of p28 and p18 into fibroblasts was also inhibited by MβCD, nocodazole, monensin and tunicamycin, but not by amiloride, sodium azide, and CPZ (FIG. 19 C). This suggests that at least one mechanism of entry into cancer and normal cells may be similar, but additional preferential accumulation into cancer cells may be a function of the number of common membrane receptors or structures, ie., caveolae (FIG. 19 D, panels 1, 2). Alexafluor 568 labeled p18 and p28 co-localized with caveolin-1 and golgin 97 antibodies (FIG. 19 D panels 1,2). This confirms that these organelles are involved in the intracellular trafficking ofpl 8 and p28. Interestingly, azurin, but neither p18 nor p28 colocalized with mitochondrial specific fluorescence (FIG. 19 D panel 3). In contrast, p28 and azurin, but not p18, co-localized with lysosomes (FIG. 19 D panel 4).

### Example 31

### Functional Analysis of p28 and p18

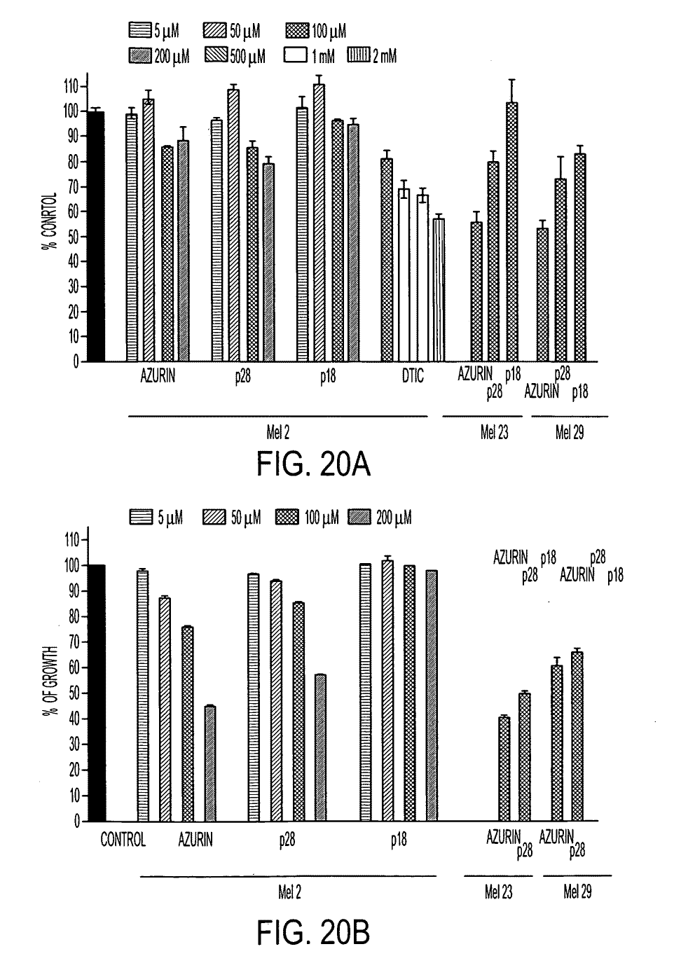

Azurin inhibits the growth of several human cancer cell lines in vitro and in vivo. FIGS. 20 A and B illustrate the effect of p18 and p28 relative to azurin and dacarbazine (DTIC) on UISO-Mel-2 cells as determined by MTT and cell count. After 72 hrs exposure, azurin decreased (p<0.05) cell survival at 100 and 200 μM −15% (FIG. 20 A). p28 had inhibited cell survival 14 and 22% (p<0.05) at 100 and 200 μM, respectively. In contrast, p18 had no effect, while dacarbazine (DTIC) produced a significant dose-related decrease on UISO-Mel-2 survival. Azurin and p28 (200 μM) also significantly decreased the survival of UISO-Mel-23 and 29 cells. p18 had no effect on UISO-Mel-2 cell proliferation.

The apparent increase (˜30-35%; UISO-Mel-2) in p28 and azurin inhibition of melanoma cell proliferation, as measured by direct cell counting, suggests that the inhibitory effect may reside primarily at the level of cell cycle with apoptosis subsequent to any delay. Although p18 penetrated cancer cells preferentially, unlike p28, it had virtually no inhibitory activity on cell proliferation. This result indicates that the cytostatic and cytotoxic activity of p28 likely lies in the C-terminal 10-12 aa of the sequence.

What is claimed is:

1. An isolated peptide that is a cupredoxin or cytochrome or a variant, derivative or truncation thereof and that may treat and/or prevent two or more conditions in mammalian cells.

2. The isolated peptide of claim 1, wherein said cupredoxin is azurin.

3. The isolated peptide of claim 1, wherein said cupredoxin is from an organism selected from the group consisting of Pseudomonas aeruginosa, Alcaligenes faecalis, Achromobacter xylosoxidan, Bordetella bronchiseptica, Methylomonas sp., Neisseria meningitidis, Neisseria gonorrhea, Pseudomonas fluorescens, Pseudomonas chlororaphis, Bordetella pertussis, Pseudomonas syringae, Xylella fastidiosa and Vibrio parahaemolyticus.

4. The isolated peptide of claim 1, wherein the peptide is selected from the group consisting of SEQ ID NOS: 1, 5-12, 18 and 23.

5. The isolated peptide of claim 1, to which a sequence selected from the group consisting of SEQ ID NOS: 1, 5-12, 18 and 23 is a mutant or has at least 90% amino acid sequence identity.

6. The isolated peptide of claim 1, wherein the peptide is a truncation of a peptide selected from the group consisting of SEQ ID NOS: 1, 5-12, 18 and 23.

7. The isolated peptide of claim 6, wherein the peptide comprises the sequence and/or the equivalent residues of a cupredoxin as a region selected from the group consisting of SEQ ID NOS: 25, 27-33, and 48-50.

8. A composition, comprising one or more cupredoxins, cytochromes or peptides of claim 1 in a pharmaceutical composition.

9. The composition of claim 8, wherein the cupredoxin is selected from one or more of the group consisting of SEQ ID NOS: 1, 5-12, 18, 23, 25, 27-33 and 48-50.

10. The composition of claim 8, wherein the cupredoxin comprises SEQ ID NO: 30.

11. The composition of claim 8, wherein the composition is administered to a patient for the concurrent prevention and/or treatment of two or more conditions selected from the group consisting of interstitial cystitis (IC), lesions associated with inflammatory bowel disease (IBD), HIV infection, AIDS, central nervous system disorders, peripheral vascular diseases, viral diseases, degeneration of the central nervous system (Christopher Reeve's disease), Alzheimer's disease, malaria, inappropriate angiogenesis, cardiovascular disease, hypertension, bacterial infection, Cytomegalovirus infection, human papillomavirus infection; Muscular Dystrophy, encephalopathy, dementia, Parkinson's disease, neuropathy, macular degeneration, diabetic retinopathy, rheumatoid arthritis, psoriasis, herpes simplex virus (HSV), Ebola virus, cytomeglovirus (CMV), parainfluenza viruses types A, B and C, hepatitis virus A, B, C, and G, the delta hepatitis virus (HDV), mumps virus, measles virus, respiratory syncytial virus, bunyvirus, arena virus, Dhori virus, poliovirus, rubella virus, dengue virus; SIV, Mycobacterium tuberculosis and cancer.

12. The composition of claim 8, wherein the composition is administered to a patient for the concurrent prevention and/or treatment of two or more conditions selected from the group consisting of HIV, malaria, cancer and inappropriate angiogenesis.

13. The composition of claim 12, wherein the patient has a higher risk than the general population of acquiring a condition selected from one or more of the group consisting of HIV, malaria, cancer and inappropriate angiogenesis.

14. The composition of claim 8, which additionally comprises another drug selected from the group consisting of an anti-malarial drug, an anti-HIV drug, an anti-cancer drug and an anti-angiogenesis drug.

15. The composition of claim 8, wherein the pharmaceutical composition is co-administered with at least one other drug.

16. The composition of claim 15, wherein the other drug is selected from the group consisting of an anti-malarial drug, an anti-HIV drug, an anti-cancer drug and an anti-angiogenesis drug.

17. A method to administer to a patient the pharmaceutical composition of claim 8.

18. The method of claim 17, wherein the patient is human.

19. The method of claim 17, wherein the composition is administered to a patient for the concurrent prevention and/or treatment of two or more conditions selected from the group consisting of interstitial cystitis (IC), lesions associated with inflammatory bowel disease (IBD), HIV infection, AIDS, central nervous system disorders, peripheral vascular diseases, viral diseases, degeneration of the central nervous system (Christopher Reeve's disease), Alzheimer's disease, malaria, inappropriate angiogenesis, cardiovascular disease, hypertension, Cytomegalovirus infection, human papillomavirus infection; Muscular Dystrophy, encephalopathy, dementia, Parkinson's disease, neuropathy, macular degeneration, diabetic retinopathy, rheumatoid arthritis, psoriasis, herpes simplex virus (HSV), Ebola virus, cytomeglovirus (CMV), parainfluenza viruses types A, B and C, hepatitis virus A, B, C, and G, the delta hepatitis virus (HDV), mumps virus, measles virus, respiratory syncytial virus, bunyvirus, arena virus, Dhori virus, poliovirus, rubella virus, dengue virus; SIV, Mycobacterium tuberculosis and cancer.

20. The method of claim 17, wherein said composition is administered to a patient for the concurrent prevention and/or treatment of two or more conditions selected from the group consisting of HIV, malaria, cancer and inappropriate angiogenesis.

21. The method of claim 20, wherein said patient has a higher risk than the general population of acquiring a condition selected from one or more of the group consisting of HIV, malaria, cancer and inappropriate angiogenesis.

22. The method of claim 17, wherein said composition additionally comprises another drug selected from the group consisting of an anti-malarial drug, an anti-HIV drug, an anti-cancer drug and an anti-angiogenesis drug.

23. The method of claim 17, wherein said pharmaceutical composition is co-administered with at least one other drug.

24. The method of claim 23, wherein said other drug is selected from the group consisting of an anti-malarial drug, an anti-HIV drug, an anti-cancer drug and an anti-angiogenesis drug.

25. A kit comprising the composition of claim 8.

26. The isolated peptide of claim 1, wherein the cupredoxin is selected from the group consisting of azurin, pseudoazurin, plastocyanin, rusticyanin, Laz, auracyanin, stellacyanin and cucumber basic protein.

27. The isolated peptide of claim 1, which can inhibit parasitemia by malaria in P. falciparum-infected human red blood cells.

28. The isolated peptide of claim 1, which is fused to a H.8 region of Laz.

29. The isolated peptide of claim 1, which is a structural equivalent of monoclonal antibody G17.12.

30. The isolated peptide of claim 1, wherein the cytochrome is selected from one or more of the group consisting of cytochrome c, cytochrome f and cytochrome c₅₅₁.

31. The isolated peptide of claim 30, wherein the cytochrome c is from an organism selected from the group consisting of human and Pseudomonas aeruginosa.

32. The isolated peptide of claim 30, wherein the cytochrome f is from a cyanobacteria.

33. The isolated peptide of claim 1, which is a truncation of cupredoxin or cytochrome.

34. The isolated peptide of claim 33, wherein the peptide is more than about 10 residues and not more than about 100 residues.

35. The isolated peptide of claim 6, wherein the peptide consists of a sequence selected from the group consisting of SEQ ID NOS: 25, 27-33, and 48-50.

36. The composition of claim 8, wherein the cupredoxin is from an organism selected from the group consisting of Pseudomonas aeruginosa, Alcaligenes faecalis, Achromobacter xylosoxidan, Bordetella bronchiseptica, Methylomonas sp., Neisseria meningitidis, Neisseria gonorrhea, Pseudomonas fluorescens, Pseudomonas chlororaphis, Bordetella pertussis, Pseudomonas syringae, Xylella fastidiosa and Vibrio parahaemolyticus.

37. The composition of claim 36, wherein the cupredoxin is from Pseudomonas aeruginosa.

38. The composition of claim 8, wherein the pharmaceutical composition is administered by a mode selected from the group consisting of intravenous injection, intramuscular injection, subcutaneous injection, inhalation, topical administration, transdermal patch, suppository, vitreous injection and oral.

39. The composition of claim 8, wherein the pharmaceutical composition is administered at about the same time as another drug.

40. The composition of claim 39, wherein the other drug is selected from the group consisting of an anti-malarial drug, an anti-HIV drug, an anti-cancer drug and an anti-angiogenesis drug

41. The composition of claim 11, wherein the cancer is selected from the group consisting of melanoma, leukemia, breast cancer, ovarian cancer, lung cancer, mesenchymal cancer, colon cancer, aerodigestive tract cancer, cervical cancer, brain tumors and prostate cancer.

42. The method of claim 17, wherein the pharmaceutical composition is administered by a mode selected from the group consisting of intravenous injection, intramuscular injection, subcutaneous injection, inhalation, topical administration, transdermal patch, suppository, vitreous injection and oral.

43. The method of claim 17, wherein the pharmaceutical composition is administered at about the same time as another drug.

44. The method of claim 43, wherein the other drug is selected from the group consisting of an anti-malarial drug, an anti-HIV drug, an anti-cancer drug and an anti-angiogenesis drug.

45. The method of claim 19, wherein the cancer is selected from the group consisting of melanoma, leukemia, breast cancer, ovarian cancer, lung cancer, mesenchymal cancer, colon cancer, aerodigestive tract cancer, cervical cancer, brain tumors and prostate cancer.
